(19) United States
(12) Patent Application Publication
(10) Pub. No.: US 2025/0318483 A1
(43) Pub. Date: Oct. 16, 2025

(54) METHODS AND COMPOSITIONS FOR MODIFYING SEED COMPOSITION

(71) Applicant: ZEAKAL, INC., San Diego, CA (US)

(72) Inventors: AMY CURRAN, Kula, HI (US); GREGORY BRYAN, Ashurst (NZ); NICHOLAS JOHN ROBERTS, Feilding (NZ); HAN CHEN, San Diego, CA (US)

(21) Appl. No.: 18/862,834

(22) PCT Filed: May 4, 2023
(86) PCT No.: PCT/IB2023/054626

§ 371 Date: Nov. 4, 2024

(30) Foreign Application Priority Data
May 6, 2022 (AU) 2022901207

(51) Int. Cl. A01H 1/00; A01H 1/04; A23J 1/14; A23K 10/30; A23K 20/147; C11B 1/10

(52) U.S. Cl. CPC A01H 1/104; A01H 1/04; A23J 1/14; A23K 10/30; A23K 20/147; C11B 1/10

(57) ABSTRACT

The invention provides a method for increasing the production of oil in the seed of a plant relative to that in a control plant, without significantly decreasing the production of protein in the seed, by ectopically expressing an oil-synthesising enzyme and an oil-encapsulating protein in the plant, wherein expression is not seed-preferred or seed-specific expression. The invention also provides plants and seeds produced or selected by the methods, and methods for processing the seeds into oil and a protein-enriched co-product.

### CROSS REFERENCE TO RELATED APPLICATIONS

The contents of Australian provisional patent application number 2022901207, filed 6 May 2022, is incorporated herein by reference in its entirety.

### TECHNICAL FIELD

The invention relates to compositions and methods for the manipulation of seed composition.

### BACKGROUND

Oilseed crops are the major source of plant oils in the world and the demand for vegetable oils is becoming increasingly important, as it becomes the main input for food, animal feed and increasingly, energy and materials. While traditional breeding has made significant gains in elevating seed oil content in certain crops, innovation is beginning to hit a plateau, and additional increases in oil are speculated to come at the expense of other important features such as seed protein content (Mahmoud et al., 2006). Genetic modification, including transgenic and gene editing approaches, have been used to introduce new genetic diversity but face similar challenges where higher seed oil levels are at the expense of protein content, or other important agronomic features.

A good example of these trade-offs in an effort to improve oil content can be seen in soybeans, one of the world's largest oilseed crops. As both an oil and protein crop, researchers have long known that total oil content of soybean seeds is negatively correlated with protein content (Hymowitz et al., 1972). The widely accepted inverse relationship between total oil and protein content in soybean is that a 1% reduction in total oil leads to a 2% increase in total protein. The average protein content of soybean meal in major soy producing countries such as the United States has declined from 49% to historical lows of 45% over the past decade.

At the plant level, soybean seed composition is the result of complex genotype and environment interactions. During seed filling (reproductive stages R5-R7), central carbon and nitrogen sources (i.e., sucrose and amino acids) from maternal tissue are distributed to lipids, carbohydrates, and proteins through glycolysis, tricarboxylic acid cycle, and amino acid metabolic pathways. As the seed transitions to maturation phase (reproductive stages R7 to R8), some accumulated proteins are degraded by proteolysis, while a portion of the lipids are degraded via beta-oxidation and potentially used for raffinose family oligosaccharide (RFO) biosynthesis through gluconeogenesis (Kambhampati et al., 2020).

As such, the challenge facing oilseed crops has been to improve seed oil content while maintaining other important nutritional and economic features such as seed protein levels. New methods and approaches that can increase seed oil composition while balancing the plants ability to maintain its protein levels are therefore required.

It is an object of the invention to provide methods for production and/or selection of plants that overcome one or more of the limitations of the prior art and/or at least provide the public with a useful choice.

### SUMMARY OF THE INVENTION

Increasing plant oil content in either the seed or vegetative organs through genetic modification has focused on overexpression or knockdowns of key transcription factors or genes involved in the fatty acid synthesis and degradation pathways within the seed or vegetative organ (within whichever is the target for oil accumulation).

This has often included strategies to “push” more carbon into the fatty acid biosynthesis pathway by upregulating transcription factors and enzymes involved in the first steps of the fatty acid biosynthetic pathway; and by “pulling” more carbon through the fatty acid biosynthesis pathway by upregulating enzymes involved in triacyl glyceride biosynthesis or protecting the oil from degradation either by over expressing oil encapsulation proteins or down regulating lipid degrading proteins (for review see Xu et al. 2018). Promoters regulating the expression of these constructs have predominantly resided in two broad categories, seed specific expression for oil production in seeds and constitutive or green tissue promoters for oil production in vegetative organs or green tissues.

The present applicants have now surprisingly demonstrated increased oil accumulation in the seed without penalising protein accumulation in the seed, by expressing an oil synthesising enzyme and an oil encapsulating protein in a plant, without targeting seed-preferred or seed-specific expression of these proteins.

The temporal and spatial expression profile used to produce these surprising results, differs from that typical of seed-preferred and seed-specific genes and promoters, as discussed further herein.

The applicants have also shown that the composition of the oil accumulating in the seeds of the plants of the invention, is characteristic of activity of the oil synthesising enzyme, suggesting that the oil synthesising enzyme may be primarily responsible for the phenotype demonstrated.

These results are surprising for the following reasons:
Oil in the mature seed increased significantly, although expression was not targeted to the seed (seed-preferred),
The promoters used by the applicants have very different temporal and spatial expression profiles relative to the seed-preferred genes and promoters that would generally be used, when targeting manipulation of oil in the seed.
Seed oil accumulation was increased significantly while protein content was at least maintained, which contrasts with the widely accepted inverse relationship between total oil and protein content.

### Method

In the first aspect the invention provides a method for increasing the production of oil in the seeds of a plant, relative to that in a control plant, without reducing the protein content of the seeds of the plant, wherein the method comprises the step of ectopically expressing an oil-synthesising enzyme in the plant, wherein expression of the oil-synthesising enzyme is not seed-preferred expression.

In one embodiment the method also includes the step of ectopically expressing an oil-encapsulating protein in the plant. Preferably expression of the oil-encapsulating protein is not seed-preferred expression.

In a further aspect the invention provides a method for producing seed with increased oil content relative to that in seed of a control plant, without reduced protein content, the method comprising the step of ectopically expressing an oil-synthesising enzyme in the plant, wherein expression of the oil-synthesising enzyme is not seed-preferred expression.

In one embodiment the method also includes the step of ectopically expressing an oil-encapsulating protein in the plant. Preferably expression of the oil-encapsulating protein is not seed-preferred expression.

### Increased Seed Oil Production Content

In one embodiment production or oil content of oil in the seeds of the plant is increased by at least 1%, preferably at least 2%, more preferably at least 3%, more preferably at least 4%, more preferably at least 5%, more preferably at least 6%, more preferably at least 7%, more preferably at least 8%, more preferably at least 9%, more preferably at least 10%, more preferably at least 11%, more preferably at least 12%, more preferably at least 13%, more preferably at least 14%, more preferably at least 15%, more preferably at least 16%, more preferably at least 17%, more preferably at least 18%, more preferably at least 19%, more preferably at least 20%, more preferably at least 21%, more preferably at least 22%, more preferably at least 23%, more preferably at least 24%, more preferably at least 25%, more preferably at least 26%, more preferably at least 27%, more preferably at least 28%, more preferably at least 29%, more preferably at least 30%, more preferably at least 31%, more preferably at least 32%, more preferably at least 33%, more preferably at least 34%, more preferably at least 35%, more preferably at least 36%, more preferably at least 37%, more preferably at least 38%, more preferably at least 39%, more preferably at least 40%, relative to that in a control plant.

In one embodiment there is a significant increase in the oil content of the seeds of the plant, relative to that in the control plant.

In one embodiment the increase in the oil content of the seeds is assessed using near infra red spectroscopy, NIR, (Zhu, Z, Chen, S, Wu X, Xing C, 2018, Food Sci Nutr. 6(4): 1109-1118. Determination of soybean routine quality parameters using near—infrared spectroscopy.) or by gas chromatography (GC) of fatty acid methyl esters (FAMES) (Shantha N C and Napolitano G E, 1992, Gas chromatography of fatty acids, Journal of Chromatography A. 624, 1-2:37-51). Analysis of total fatty acids (crude) AOAC Official Method 996.06 and OACS Official Method Ca 5b-71. Fatty Acid Profile, AOAC Official Methods 996-06 [Analysis of methyl esters by Capillary GLC], AOAC Official Methods Ce 2-66 [Preparation of Methyl Esters of Fatty Acids], AOAC Official Methods 965.49 [Preparation of Methyl Esters of Fatty Acids], AOAC Official Methods 969.33 [Oils and fat, Boron Trifluoride method]. Folch Extraction for Total Lipids from Animal Tissues (Folch et al. 1957, J. Biol. Chem 226:497). It would also be understood by those skilled in the art that a determination of oil content of the seeds could be made by quantifying the oil following industrial processing, including (but not limited to): solvent extraction, crushing, and critical point extraction.

In one embodiment the significance of the increase is at the less than 20% probability level, preferably at the less than 15% probability level, more preferably at the less than 10% probability level, more preferably at the less than 5% probability level, more preferably at the less than 1% probability level.

In one embodiment the significance of the increase in the oil content of the seeds is assessed using ANOVA (SAS Institute, 2016). Preferably the means are separated using Fisher's Protected LSD at P=0.1.

In a further embodiment the significance of the increase in the oil content of the seeds is assessed using Student's T-test (Microsoft Excel V2108). Preferably the means separated by Fishers Least Significant Difference Test at P=0.05.

### Oil

In one embodiment the oil is triacylglycerol (TAG).

### Altered Oil Composition

In one embodiment the fatty acid profile of the seed of the plant is altered relative to that in the control plant.

In one embodiment there is an increase in C18:0 fatty acid.

In a further embodiment there is an increase in C18:1 fatty acid.

In a further embodiment there is an increase in both C18:0 and C18:1 fatty acids.

In one embodiment there is a decrease in C18:2 fatty acid.

In a further embodiment there is a decrease in C18:3 fatty acid.

In a further embodiment there is a decrease in both C18:2 and C18:3 fatty acid.

In a further embodiment there is an increase in both C18:0 and C18:1, and a decrease in both C18:2 and C18:3 fatty acids.

Those skilled in the art will know that fatty acid profile with an increase in the proportions of C18:0 and C18:1 fatty acids and decrease in the proportions of C18:2 and C18:3 fatty acid is characteristic of the activity of an oil synthsising enzyme, such as DGAT1.

In one embodiment the altered fatty acid profile of the seed is a consequence of the increase in oil as described herein.

### No Reduction in Seed Protein Content

In one embodiment the there is no significant reduction in the protein content of the seeds of the plant, relative to that in a control plant.

In one embodiment the protein content in the seeds is assessed using NIR (Zhu Z, Chen S, Wu X, Xing C, Yuan J, 2018, Determination of soybean routine quality parameters using near-infrared spectroscopy. Food Sci. Nutr. 6:1109-1118) or Kjeldahl or Dumas methods (Jung S, Rickert D A, Deak N A, Aldin E D, Recknor J, Johnson L A, Murphy P A, 2003, Comparison of kjeldahl and dumas methods for determining protein contents of soybean products. Journal of the American Oil Chemists Society, 80, 1169), total nitrogen or crude protein (CP) Combustion Analysis (LECO) AOAC Official Method 990.03, 2006, and Kjeldahl, AOAC Official Method 984. 13 (A-D), 2006.

In one embodiment any reduction in the protein content in the seeds is at the more than 10% probability level, preferably the more than 20% probability level, more preferably the more than 30% probability level.

In one embodiment the significance of any reduction in the protein content of the seeds is assessed using ANOVA (SAS Institute, 2016). Preferably the means are separated using Fisher's Protected LSD at P=0.1.

In a further embodiment the significance of any reduction in the protein content of the seeds is assessed using Student's T-test (Microsoft Excel V2108). Preferably the means separated by Fishers Least Significant Difference Test at P=0.05.

### Increase in Seed Protein Content

In a further embodiment there is an increase in the protein content in the seeds relative to that in the control plant.

In one embodiment production of protein in the seeds of a plant is increased by at least 0.10%, preferably at least 0.2%, more preferably at least 0.3%, more preferably at least 0.4%, more preferably at least 0.5%, more preferably at least 0.6%, more preferably at least 0.7%, more preferably at least 0.8%, more preferably at least 0.9%, more preferably at least 1%, more preferably at least 1.1%, more preferably at least 1.2%, more preferably at least 1.3%, more preferably at least 1.4%, more preferably at least 1.5%, more preferably at least 1.6%, more preferably at least 1.7%, more preferably at least 1.8%, more preferably at least 1.9%, more preferably at least 2%, more preferably at least 2.2%, preferably at least 2.4%, preferably at least 2.6%, preferably at least 2.8%, more preferably at least 3%, more preferably at least 3.5%, more preferably at least 4%, more preferably at least 5%, more preferably at least 6%, more preferably at least 7%, more preferably at least 8%, more preferably at least 9%, more preferably at least 10%, more preferably at least 11%, more preferably at least 12%, more preferably at least 13%, more preferably at least 14%, more preferably at least 15%, more preferably at least 16%, more preferably at least 17%, more preferably at least 18%, more preferably at least 19%, more preferably at least 20%, relative to that in a control plant.

In one embodiment there is a significant increase in the protein content of the seeds of the plant, relative to that in a control plant.

In one embodiment the increase in the protein content of the seeds is assessed using NIR (Zhu Z, Chen S, Wu X, Xing C, Yuan J, 2018, Determination of soybean routine quality parameters using near-infrared spectroscopy. Food Sci. Nutr. 6:1109-1118) or Kjeldahl or Dumas methods (Jung S, Rickert D A, Deak N A, Aldin E D, Recknor J, Johnson L A, Murphy P A, 2003, Comparison of kjeldahl and dumas methods for determining protein contents of soybean products. Journal of the American Oil Chemists Society, 80, 1169), total nitrogen or crude protein (CP) Combustion Analysis (LECO) AOAC Official Method 990.03, 2006, and Kjeldahl, AOAC Official Method 984. 13 (A-D), 2006.

In one embodiment the significance of the increase is at the less than 20% probability level, preferably at the less than 15% probability level, more preferably at the less than 10% probability level, more preferably at the less than 5% probability level, more preferably at the less than 1% probability level.

In one embodiment the significance of the increase in the protein content of the seeds is assessed using ANOVA (SAS Institute, 2016). Preferably the means are separated using Fisher's Protected LSD at P=0.1.

In a further embodiment the significance of the increase in the protein content of the seeds is assessed using Student's T-test (Microsoft Excel V2108). Preferably the means separated by Fishers Least Significant Difference Test at P=0.05.

### Oil Synthesising Enzyme

Numerous oil synthesising enzymes are known to those skilled in the art, and may be conveniently selected for use in the invention.

In one embodiment the oil synthesising enzyme is a “triacylglycerol synthesising enzyme” or “TAG synthesising enzyme”.

In a preferred embodiment the TAG synthesising enzyme is acyl CoA: diacylglycerol acyltransferase1 (DGAT1).

### Constitutive Expression

In one embodiment expression of the oil synthesising enzyme is constitutive expression.

In one embodiment expression of the oil-encapsulating protein is constitutive expression.

In one embodiment expression of both of the oil synthesising enzyme and the oil-encapsulating protein is constitutive expression.

### Green-Tissue Preferred Expression

In one embodiment expression of the oil synthesising enzyme is green-tissue preferred expression.

In one embodiment expression of the oil-encapsulating protein is green-tissue preferred expression.

In one embodiment expression of both of the oil synthesising enzyme and the oil-encapsulating protein is green-tissue preferred expression.

### Light-Induced Expression

In one embodiment expression of the oil synthesising enzyme is light-induced expression.

In one embodiment expression of the oil-encapsulating protein is light-induced expression.

In one embodiment expression of both of the oil synthesising enzyme and the oil-encapsulating protein is light-induced expression.

Method with Polynucleotides, Constructs and Transformation

In one embodiment expression of the oil synthesising enzyme is from a polynucleotide encoding the oil synthesising enzyme.

In one embodiment the polynucleotide is heterologous with respect to the plant.

In a further embodiment polynucleotide is part of a construct comprising a promoter operably linked to the polynucleotide.

In one embodiment the promoter is heterologous with respect to the polynucleotide.

In a further embodiment the plant is transformed with the polynucleotide or construct.

In a further embodiment the method includes the step of transforming the plant with the polynucleotide or construct.

In one embodiment expression of the oil-encapsulating protein is from a polynucleotide encoding the oil-encapsulating protein.

In one embodiment the polynucleotide is heterologous with respect to the plant.

In one embodiment polynucleotide is part of a construct comprising a promoter operably linked to the polynucleotide.

In one embodiment the promoter is heterologous with respect to the polynucleotide.

In a further embodiment the plant is transformed with the polynucleotide or construct.

In a further embodiment the method includes the step of transforming the plant with the polynucleotide or construct.

Those skilled in the art will understand that polynucleotides and constructs for expressing polypeptides/proteins in cells, plants and other organisms can include various other modifications including restriction sites, recombination/excision sites, codon optimisation, tags to facilitate protein purification, etc. Those skilled in the art will understand how to utilise such modifications, some of which may influence transgene expression, stability and translation. However, an art skilled worker would also understand that these modifications are not essential, and do not limit the scope of the invention.

### Method Including the Step of Measuring the Production or Accumulation of Oil in the Seed

In a further embodiment the method includes the step of measuring the production or accumulation of oil in the seed of the plant.

### Method Including the Step of Measuring the Composition of Oil Produced or Accumulated in the Seed

In a further embodiment the method includes the step of measuring the composition of oil produced or accumulated in the seed of the plant.

### Method Including the Step of Selecting the Plant Based on Measuring the Production or Accumulation of Oil in the Seed

In a further embodiment the method includes the step of selecting the plant based on measuring the composition of oil produced or accumulated in the seed.

### Method Including the Step of Selecting the Plant Based on Measuring the Composition of Oil Produced or Accumulated in the Seed

In a further embodiment the method includes the step of selecting the plant based on measuring the composition of oil produced or accumulated in the seed of the plant.

### Promoters

Those skilled in the art will appreciate that a promoter is used to control expression of an operably linked polynucleotide/s, such as those referred to above.

In various embodiments of the invention, the promoter operably linked to the polynucleotide encoding the oil synthesising enzyme may be the same as, or different from, the promoter operably linked to the polynucleotide encoding the oil-encapsulating protein.

### Non-Seed Preferred Promoters

In one embodiment the promoter operably linked to the polynucleotide, or in the construct, to drive expression of either, or both, of the oil synthesising enzyme and the oil-encapsulating protein is not a seed preferred promoter.

In a further embodiment the promoter operably linked to the polynucleotide, or in the construct, to drive expression of either, or both, of the oil synthesising enzyme and the oil-encapsulating protein is a non-seed preferred promoter.

The term seed-preferred promoter also encompassed seed-specific promoters.

In one embodiment the seed preferred promoter is a seed-specific promoter.

### Constitutive Promoters

In one embodiment the promoter operably linked to the polynucleotide, or in the construct, to drive expression of either, or both, of the oil synthesising enzyme and the oil-encapsulating protein is a constitutive promoter.

Numerous constitutive promoters are known to those skilled in the art.

In one embodiment the constitutive promoter is a cauliflower mosaic virus (CaMV) promoter

In one embodiment the CaMV promoter is a CaMV 35S promoter.

In a further embodiment the constitutive promoter is a ubiquitin promoter.

In a further embodiment the constitutive promoter is an actin promoter.

### Green Tissue Preferred Promoters

In one embodiment the promoter operably linked to the polynucleotide, or in the construct, to drive expression of either, or both, of the oil synthesising enzyme and the oil-encapsulating protein is a green tissue preferred promoter.

Numerous green tissue preferred promoters are known to those skilled in the art.

### Cab Promoters

In one embodiment the green tissue preferred promoter is a chlorophyll a/b (Cab) binding protein promoter, also known as a cab promoter.

### RbcS Promoters

In a further embodiment the green tissue preferred promoter is a promoter from a small subunit of ribulose-bisphosphate carboxylase (Rubisco) promoter, also known as an rbcS promoter.

### GSE Promoters

In a further embodiment the green tissue-preferred promoter is a promoter from a green special express (GSE) gene, also known as a GSE promoter (Xue M et al., 2018, Int. J. Mol. Sci. 2018).

### C4 PEPC Promoters

In a further embodiment the green tissue-preferred promoter is a promoter from a phosphoenol pyruvate carboxylase (C4 PEPC) gene, also known as an C4 PEPC promoter.

### C4 PPDK Promoters

In a further embodiment the green tissue-preferred promoter is a promoter from a pyruvate phosphate dikinase (C4 PPDK) gene, also known as an C4 PPDK promoter.

The term green tissue-preferred also encompassed green tissue-specific promoters.

In one embodiment the green tissue-preferred promoter is a green tissue-specific promoter.

### Light-Induced Promoters

In one embodiment the promoter operably linked to the polynucleotide, or in the construct, to drive expression of either, or both, of the oil synthesising enzyme and the oil-encapsulating protein is a light-induced promoter.

Light-induced promoters are known to those skilled in the art and include but are not limited to the green-tissue-preferred promoters described above.

### Plants, Plant Parts and Seeds

In a further aspect the invention provides a plant produced by a method of the invention.

In a further aspect the invention provides a plant with increased oil content in its seeds, relative to that in a control plant, without reduced protein content of the seeds, wherein the plant ectopically expresses an oil-synthesising enzyme, and wherein expression of the oil-synthesising enzyme is not seed-preferred expression.

In one embodiment the increased seed oil content is a result of the ectopic expression of the oil-synthesising enzyme.

In a further embodiment the plant also ectopically expresses an oil-encapsulating protein. Preferably expression of the oil-encapsulating protein is not seed-preferred.

In one embodiment the increased seed oil content is a result of the ectopic expression of the oil-synthesising enzyme and the ectopic expression of the oil-encapsulating protein.

### Plants and Seeds Produced by Crossing

In a further aspect the invention provides a method for producing a plant with increased production or content of oil in its seed relative to that in a control plant, without significantly decreased production or content of protein in its seed relative to that in the control plant, the method comprising crossing a plant of any preceding claim with another plant.

In a further aspect the invention provides a method for producing a seed with increased oil production or oil content relative to that in a control plant, without significantly decreased oil production or oil content relative to that in the control plant, the method comprising:
a) crossing a plant of any preceding claim with another plant.
b) harvesting the seed produced.

### Increased Seed Oil Content

In one embodiment the oil content in the seeds of a plant is increased by at least 1%, preferably at least 2%, more preferably at least 3%, more preferably at least 4%, more preferably at least 5%, more preferably at least 6%, more preferably at least 7%, more preferably at least 8%, more preferably at least 9%, more preferably at least 10%, more preferably at least 11%, more preferably at least 12%, more preferably at least 13%, more preferably at least 14%, more preferably at least 15%, more preferably at least 16%, more preferably at least 17%, more preferably at least 18%, more preferably at least 19%, more preferably at least 20%, more preferably at least 21%, more preferably at least 22%, more preferably at least 23%, more preferably at least 24%, more preferably at least 25%, more preferably at least 26%, more preferably at least 27%, more preferably at least 28%, more preferably at least 29%, more preferably at least 30%, more preferably at least 31%, more preferably at least 32%, more preferably at least 33%, more preferably at least 34%, more preferably at least 35%, more preferably at least 36%, more preferably at least 37%, more preferably at least 38%, more preferably at least 39%, more preferably at least 40%, relative to that in a control plant.

In one embodiment there is a significant increase in the oil content of the seeds of the plant, relative to that in a control plant.

In one embodiment the increase in the oil content of the seeds is assessed using near infra red spectroscopy, NIR, (Zhu, Z, Chen, S, Wu X, Xing C, 2018, Food Sci Nutr. 6(4): 1109-1118. Determination of soybean routine quality parameters using near—infrared spectroscopy.) or by gas chromatography (GC) of fatty acid methyl esters (FAMES) (Shantha N C and Napolitano G E, 1992, Gas chromatography of fatty acids, Journal of Chromatography A. 624, 1-2:37-51). Analysis of total fatty acids (crude) AOAC Official Method 996.06 and OACS Official Method Ca 5b-71. Fatty Acid Profile, AOAC Official Methods 996-06 [Analysis of methyl esters by Capillary GLC], AOAC Official Methods Ce 2-66 [Preparation of Methyl Esters of Fatty Acids], AOAC Official Methods 965.49 [Preparation of Methyl Esters of Fatty Acids], AOAC Official Methods 969.33 [Oils and fat, Boron Trifluoride method]. Folch Extraction for Total Lipids from Animal Tissues (Folch et al., 1957, J. Biol. Chem 226:497). It would also be understood by those skilled in the art that a determination of oil content of the seeds could be made by quantifying the oil following industrial processing, including (but not limited to): solvent extraction, crushing, and critical point extraction.

In one embodiment the significance of the increase is at the less than 20% probability level, preferably at the less than 15% probability level, more preferably at the less than 10% probability level, more preferably at the less than 5% probability level, more preferably at the less than 1% probability level.

In one embodiment the significance of the increase in the oil content of the seeds is assessed using ANOVA (SAS Institute, 2016). Preferably the means are separated using Fisher's Protected LSD at P=0.1.

In a further embodiment the significance of the increase in the oil content of the seeds is assessed using Student's T-test (Microsoft Excel V2108). Preferably the means separated by Fishers Least Significant Difference Test at P=0.05.

### Oil

In one embodiment the oil is triacylglycerol (TAG).

### Altered Oil Composition

In one embodiment the fatty acid profile of the seed of the plant changes relative to that in the control plant.

In one embodiment there is an increase in C18:0 fatty acid.

In a further embodiment there is an increase in C18:1 fatty acid.

In a further embodiment there is an increase in both C18:0 and C18:1 fatty acids.

In one embodiment there is a decrease in C18:2 fatty acid.

In a further embodiment there is a decrease in C18:3 fatty acid.

In a further embodiment there is a decrease in both C18:2 and C18:3 fatty acid.

In a further embodiment these is an increase in both C18:0 and C18:1, and a decrease in both C18:2 and C18:3 fatty acids.

Those skilled in the art will know that fatty acid profile with an increase in the proportions of C18:0 and C18:1 fatty acids and decrease in the proportions of C18:2 and C18:3 fatty acid is characteristic of the activity of an oil synthsising enzyme, such as DGAT1.

### No Reduction in Seed Protein Content

In one embodiment there is no significant reduction in the protein content of the seeds of the plant, relative to that in a control plant.

In one embodiment the significance of any reduction in the protein content in the seeds is assessed using NIR (Zhu Z, Chen S, Wu X, Xing C, Yuan J, 2018, Determination of soybean routine quality parameters using near-infrared spectroscopy. Food Sci. Nutr. 6:1109-1118) or Kjeldahl or Dumas methods (Jung S, Rickert D A, Deak N A, Aldin E D, Recknor J, Johnson L A, Murphy P A, 2003, Comparison of kjeldahl and dumas methods for determining protein contents of soybean products. Journal of the American Oil Chemists Society, 80, 1169), total nitrogen or crude protein (CP) Combustion Analysis (LECO) AOAC Official Method 990.03, 2006, and Kjeldahl, AOAC Official Method 984. 13 (A-D), 2006.

In one embodiment any reduction in the protein content in the seeds is at the more than 10% probability level, preferably the more than 20% probability level, more preferably the more than 30% probability level.

### Increase in Seed Protein Content

In a further embodiment there is an increase in the protein content in the seeds relative to that in the control plant.

In one embodiment production of protein in the seeds of a plant is increased by at least 0.10%, preferably at least 0.2%, more preferably at least 0.3%, more preferably at least 0.4%, more preferably at least 0.5%, more preferably at least 0.6%, more preferably at least 0.7%, more preferably at least 0.8%, more preferably at least 0.9%, more preferably at least 1%, more preferably at least 1.1%, more preferably at least 1.2%, more preferably at least 1.3%, more preferably at least 1.4%, more preferably at least 1.5%, more preferably at least 1.6%, more preferably at least 1.7%, more preferably at least 1.8%, more preferably at least 1.9%, more preferably at least 2%, more preferably at least 2.2%, preferably at least 2.4%, preferably at least 2.6%, preferably at least 2.8%, more preferably at least 3%, more preferably at least 3.5%, more preferably at least 4%, more preferably at least 5%, more preferably at least 6%, more preferably at least 7%, more preferably at least 8%, more preferably at least 9%, more preferably at least 10%, more preferably at least 11%, more preferably at least 12%, more preferably at least 13%, more preferably at least 14%, more preferably at least 15%, more preferably at least 16%, more preferably at least 17%, more preferably at least 18%, more preferably at least 19%, more preferably at least 20%, relative to that in a control plant.

In one embodiment the there is a significant increase in the protein content of the seeds of the plant, relative to that in a control plant.

In one embodiment the significance of increase in the protein content of the seeds is assessed using NIR (Zhu Z, Chen S, Wu X, Xing C, Yuan J, 2018, Determination of soybean routine quality parameters using near-infrared spectroscopy. Food Sci. Nutr. 6:1109-1118) or Kjeldahl or Dumas methods (Jung S, Rickert D A, Deak N A, Aldin E D, Recknor J, Johnson L A, Murphy P A, 2003, Comparison of kjeldahl and dumas methods for determining protein contents of soybean products. Journal of the American Oil Chemists Society, 80, 1169), total nitrogen or crude protein (CP) Combustion Analysis (LECO) AOAC Official Method 990.03, 2006, and Kjeldahl, AOAC Official Method 984. 13 (A-D), 2006.

In one embodiment the significance of the increase is at the less than 20% probability level, preferably at the less than 15% probability level, more preferably at the less than 10% probability level, more preferably at the less than 5% probability level, more preferably at the less than 1% probability level.

### Oil Synthesising Enzyme

In one embodiment the oil synthesising enzyme is a “triacylglycerol synthesising enzyme” or “TAG synthesising enzyme”.

In a preferred embodiment the TAG synthesising enzyme is acyl CoA: diacylglycerol acyltransferase1 (DGAT1).

### Constitutive Expression

In one embodiment expression of the oil synthesising enzyme is constitutive expression.

In one embodiment expression of the oil-encapsulating protein is constitutive expression.

In one embodiment expression of both of the oil synthesising enzyme and the oil-encapsulating protein is constitutive expression.

### Green-Tissue Preferred Expression

In one embodiment expression of the oil synthesising enzyme is green-tissue preferred expression.

In one embodiment expression of the oil-encapsulating protein is green-tissue preferred expression.

In one embodiment expression of both of the oil synthesising enzyme and the oil-encapsulating protein is green-tissue preferred expression.

### Light-Induced Expression

In one embodiment expression of the oil synthesising enzyme is light-induced expression.

In one embodiment expression of the oil-encapsulating protein is light-induced expression.

In one embodiment expression of both of the oil synthesising enzyme and the oil-encapsulating protein is light-induced expression.

Plant with Polynucleotide, Constructs and Transformation

In one embodiment expression of the oil synthesising enzyme is from a polynucleotide encoding the oil synthesising enzyme.

In one embodiment the polynucleotide is heterologous with respect to the plant.

In a further embodiment polynucleotide is part of a construct comprising a promoter operably linked to the polynucleotide.

In one embodiment the promoter is heterologous with respect to the polynucleotide.

In a further embodiment the plant is transformed with the polynucleotide or construct.

In one embodiment expression of the oil-encapsulating protein is from a polynuclotide encoding the oil-encapsulating protein.

In one embodiment the polynucleotide is heterologous with respect to the plant.

In one embodiment polynucleotide is part of a construct comprising a promoter operably linked to the polynucleotide.

In one embodiment the promoter is heterologous with respect to the polynucleotide.

In a further embodiment the plant is transformed with the polynucleotide or construct.

### Plant Selected Based on Measuring the Production or Accumulation of Oil in the Seed

In a further embodiment the plant has been selected based on measuring the production or accumulation of oil in the seed of the plant.

Plant Selected Based on Measuring the Composition of Oil Produced or Accumulated in the Seed

In a further embodiment the plant has been selected based on measuring the composition of oil produced or accumulated in the seed of the plant.

### Promoters

Those skilled in the art will appreciate that a promoter is used to control expression of an operably linked polynucleotide/s, such as those referred to above.

In various embodiments of the invention, the promoter operably linked to the polynucleotide encoding the oil synthesising enzyme may be the same as, or different from, the promoter operably linked to the polynucleotide encoding the oil-encapsulating protein.

### Non-Seed Preferred-Promoters

In one embodiment the promoter operably linked to the polynucleotide, or in the construct, to drive expression of either, or both, of the oil synthesising enzyme and the oil-encapsulating protein is not a seed-preferred promoter.

In a further embodiment the promoter operably linked to the polynucleotide, or in the construct, to drive expression of either, or both, of the oil synthesising enzyme and the oil-encapsulating protein is a non-seed-preferred promoter.

### Constitutive Promoters

In one embodiment the promoter operably linked to the polynucleotide, or in the construct, to drive expression of either, or both, of the oil synthesising enzyme and the oil-encapsulating protein is a constitutive promoter.

Numerous constitutive promoters are known to those skilled in the art.

In one embodiment the constitutive promoter is a cauliflower mosaic virus (CaMV) promoter.

In one embodiment the CaMV promoter is a CaMV 35S promoter.

In a further embodiment the constitutive promoter is a ubiquitin promoter.

In a further embodiment the constitutive promoter is an actin promoter.

### Green Tissue-Preferred Promoters

In one embodiment the promoter operably linked to the polynucleotide, or in the construct, to drive expression of either, or both, of the oil synthesising enzyme and the oil-encapsulating protein is a green tissue-preferred promoter.

Numerous green tissue-preferred promoters are known to those skilled in the art.

### Cab Promoters

In one embodiment the green tissue-preferred promoter is a chlorophyll a/b (Cab) binding protein promoter, also know as a cab promoter.

### RbcS Promoters

In a further embodiment the green tissue-preferred promoter is a promoter from a small subunit of ribulose-bisphosphate carboxylase (Rubisco) gene, also know as an rbcS promoter.

### GSE Promoters

In a further embodiment the green tissue-preferred promoter is a promoter from a green special express (GSE) gene, also know as a GSE promoter (Xue M et al., 2018, Int. J. Mol. Sci. 2018).

### C4 PEPC Promoters

In a further embodiment the green tissue-preferred promoter is a promoter from a phosphoenol pyruvate carboxylase (C4 PEPC) gene, also know as a C4 PEPC promoter.

### C4 PPDK Promoters

In a further embodiment the green tissue-preferred promoter is a promoter from a pyruvate phosphate dikinase (C4 PPDK) gene, also know as a C4 PPDK promoter.

The term green tissue-preferred also encompassed green tissue-specific promoters.

In one embodiment the green tissue-preferred promoter is a green tissue-specific promoter.

### Light-Induced Promoters

In one embodiment the promoter operably linked to the polynucleotide, or in the construct, to drive expression of either, or both, of the oil synthesising enzyme and the oil-encapsulating protein is a light-induced promoter.

Light-induced promoters are known to those skilled in the art and include but are not limited to the green-tissue-preferred promoters described above.

### Plant Parts, Propagule or Progeny

In a further aspect the invention provides a part, propagule or progeny of a plant of the invention.

In one embodiment the part, propagule or progeny comprises at least one of the polynucleotides and constructs as herein described.

In a further embodiment the part, propagule or progeny is transgenic for a polynucleotide or construct encoding an oil synthesising enzyme as herein described.

In a further embodiment the part, propagule or progeny is transgenic for a polynucleotide or construct encoding an oil encapsulating protein as herein described.

In a further embodiment the part, propagule or progeny is transgenic for a polynucleotide or construct encoding an oil synthesising enzyme as herein described, and a polynucleotide or construct encoding an oil encapsulating protein as herein described.

In one embodiment the progeny has increased oil content in its seeds, relative to that in a control plant, without reduced protein content of the seeds, and ectopically expresses an oil-synthesising enzyme, and wherein expression of the oil-synthesising enzyme is not seed-preferred or seed-specific expression.

### Seed

In one embodiment the plant part is a seed with increased oil content, without reduced protein content, relative to that in the seed of a control plant.

In one embodiment the increased oil content is as herein described.

In a further embodiment the lack of reduced protein content is as herein described.

In a further embodiment the protein content of the seed is increased relative to that in the seed of a control plant.

In a further embodiment the increased protein content is as herein described.

### Method of Testing the Oil Content of the Seed, and Selecting Seed

In a further aspect the invention provides a method for testing the oil content of the seed of the invention, or the seed of a plant of the invention.

In a further aspect the invention provides a method selecting seed based in measuring the oil content of the seed of the invention, or the seed of a plant of the invention, or the seed of a plant produced by a method of the invention.

In one embodiment the oil content is as herein described.

### Method of Testing the Protein Content of the Seed, and Selecting Seed

In a further aspect the invention provides a method for testing the protein content of the seed of the invention, or the seed of a plant of the invention.

In a further aspect the invention provides a method selecting seed based in measuring the protein content of the seed of the invention, or the seed of a plant of the invention, or the seed of a plant produced by a method of the invention.

In one embodiment the protein content is as herein described.

### Method of Testing the Oil and Protein Content of the Seed, and Selecting Seed

In a further aspect the invention provides a method for testing the oil and protein content of the seed of the invention, or the seed of a plant of the invention, or the seed of a plant produced by a method of the invention.

In a further aspect the invention provides a method selecting seed based in measuring the oil and protein content of the seed of the invention, or the seed of a plant of the invention.

In one embodiment the oil content is as herein described.

In one embodiment the protein content is as herein described.

In a further aspect the invention provides a method for producing oil, the method comprising extracting oil from the seeds of a plant of the invention, or a seed of the invention.

In a further aspect the invention provides a method for producing oil the method comprising producing a plant or seed according to the invention, extracting oil from the seeds of the plant, or the seed.

In one embodiment the oil extraction is by at least one of:
a) solvent extraction,
b) crushing, and
c) critical point extraction

In a further embodiment the oil is processed into at least one of:
a) a fuel,
b) an oleochemical,
c) a nutritional oil,
d) a cosmetic oil,
e) a polyunsaturated fatty acid (PUFA), and
f) a combination of any of a) to e).

In a further aspect the invention provides a method for producing a protein-enriched co-product, the method comprising extracting oil from the seeds of a plant of the invention, or a seed of the invention, and collecting the remaining protein-enriched co-product.

In a further aspect the invention provides a method for producing a protein-enriched co-product, the method comprising producing a plant or seed according to the invention, extracting oil from the seeds of the plant, or the seed, and collecting the remaining protein-enriched co-product.

In one embodiment the extraction is by at least one of:
a) solvent extraction,
b) crushing, and
c) critical point extraction.

In one embodiment the protein-enriched co-product has a higher protein content than that produced from the seeds of a control plant, or from control seeds.

In one embodiment protein content of the protein-enriched co-product is increased by at least 0.1%, preferably at least 0.2%, more preferably at least 0.3%, more preferably at least 0.4%, more preferably at least 0.5%, more preferably at least 0.6%, more preferably at least 0.7%, more preferably at least 0.8%, more preferably at least 0.9%, more preferably at least 1%, more preferably at least 1.1%, more preferably at least 1.2%, more preferably at least 1.3%, more preferably at least 1.4%, more preferably at least 1.5%, more preferably at least 1.6%, more preferably at least 1.7%, more preferably at least 1.8%, more preferably at least 1.9%, more preferably at least 2%, more preferably at least 2.2%, preferably at least 2.4%, preferably at least 2.6%, preferably at least 2.8%, more preferably at least 3%, more preferably at least 3.5%, more preferably at least 4%, more preferably at least 5%, more preferably at least 6%, more preferably at least 7%, more preferably at least 8%, more preferably at least 9%, more preferably at least 10%, more preferably at least 11%, more preferably at least 12%, more preferably at least 13%, more preferably at least 14%, more preferably at least 15%, more preferably at least 16%, more preferably at least 17%, more preferably at least 18%, more preferably at least 19%, more preferably at least 20%, relative to that in corresponding co-product produced from the seeds of a control plant, or from control seed.

In one embodiment the protein-enriched co-product may contain fibre, carbohydrate and residual oil.

In a further embodiment the protein-enriched co-product is enriched for protein, by removal of carbohydrate.

In a further embodiment the protein-enriched co-product may be, or may be processed into, a protein meal, protein concentrate, protein isolate as known in the art.

In a further aspect the invention provides a protein enriched co-product produced by a method of the invention.

In one embodiment, the protein-enriched co-product contains a polynucleotide or construct encoding an oil synthesising enzyme as herein described, and a polynucleotide or construct encoding an oil encapsulating protein as herein described.

In a further embodiment the invention provides an animal feedstock, or food ingredient comprising a protein-enriched co-product of the invention, or produced by a method of the invention.

### DETAILED DESCRIPTION OF THE INVENTION

The applicant's invention involves increasing the production of oil in the seeds of a plant without the usual reduction of protein content seen when oil content is increased, surprisingly achieved by ectopically expressing in a non seed-preferred or seed specific manner, an oil-synthesising enzyme, and optionally an oil encapsulating protein, in the plant.

The nutritional value of oilseeds (for example soybeans) is largely determined based on protein and oil content. For the major oilseed processers, oil is the primary commercial driver as the oil is worth significantly more than the resulting protein co-product; for example “protein meal, protein concentrate, protein isolate”. As the demand for renewable liquid fuels continues to increase, plants oils has increased significantly in comparison with the other product components derived from the seed. For downstream users of the protein meal, the protein level is critical as it impacts feed conversion and overall animal health. Improved protein content means nutrionists can achieve the same productivity but with less volume of feed not only increasing the profitability but also the sustainability of their operations when factoring in reduced energy costs of transport and storage. Consequently, the traditional trade off, increased oil at the expense of protein and vice versa, meant the cost benefit had to be weighed against each other depending on market prices for the two different products. Breaking the inverse relationship between increased oil or increased protein means the seeds are more attractive to multiple users, e.g., consumers of vegetable oils and livestock producters and aligns incentives for all players along the supply chain.

In the first aspect the invention thus provides a method for increasing the production of oil in the seeds of a plant, relative to that in a control plant, without reducing the protein content of the seeds of the plant, wherein the method comprises the step of ectopically expressing an oil-synthesising enzyme in the plant, wherein expression of the oil-synthesising enzyme is not seed-preferred or seed-specific expression.

In one embodiment the method also includes the step of ectopically expressing an oil-encapsulating protein in the plant. Preferably expression of the oil-encapsulating protein is not seed-preferred or seed-specific expression.

### Oil Production

The term “oil production” encompasses the combination of processes resulting in a given oil content. Such processes include oil biosynthesis, oil degradation/utilisation and oil storage, resulting in the oil content. Increasing oil production therefore increases oil content. “Oil production” as used herein is also synonymous with oil accumulation.

### Oil

In one embodiment the oil is triacylglycerol (TAG)

### Oil Synthesising Enzyme

Oil synthesising enzymes for use in the invention are well-known to those skilled in the art and include for example DGAT1 (Liu Q, Siloto R M, Lehner R, Stone S J, Weselake R J 2012. Acyl-CoA:diacylglycerol acyltransferase: molecular biology, biochemistry and biotechnology. Prog. Lipid Res. 51:350-377), DGAT2 (Liu Q, Siloto R M, Lehner R, Stone S J, Weselake R J 2012. Acyl-CoA:diacylglycerol acyltransferase: molecular biology, biochemistry and biotechnology. Prog. Lipid Res. 51:350-377), DGAT3 (Chi X, Hu R, Zhang X, Chen M, Chen N, Pan L, Want T, Wang M, Yang Z, Want Q, Yu S 2014. Cloning and functional analysis of three diacylglycerol acyltransferase genes from peanut (Arachis hypogaea L.) Plos One https://doi.org/10.1371/journal.pone.0105834), PDAT (Lager I, Jeppson S, Gippert A-L, Feussner I, Stymne S, Marmon S 2020. Acyltransferases regulate oil quality in Camelina sativa through both acyl donor and acyl acceptor specificities. Front. Plant Sci. https://doi.org/10.3389/fpls.2020.01144), MGAT (Liu Q, Siloto R M, Lehner R, Stone S J, Weselake R J 2012. Acyl-CoA:diacylglycerol acyltransferase: molecular biology, biochemistry and biotechnology. Prog. Lipid Res. 51:350-377), and triacylglycerol (TAG) synthesising enzymes.

In a preferred embodiment the oil synthesising enzymes is a TAG synthesising enzyme.

### Triacylglycerol (TAG) Biosynthesis

The only committed step in TAG biosynthesis is the last one, i.e. the addition of a third fatty acid to an existing diacylglycerol, thus generating TAG. In plants this step is predominantly (but not exclusively) performed by one of five (predominantly ER localised) TAG synthesising enzymes including: acyl CoA: diacylglycerol acyltransferase (DGAT1); an unrelated acyl CoA: diacylglycerol acyl transferase (DGAT2); a soluble DGAT (DGAT3) which has less than 10% identity with DGAT1 or DGAT2 (Saha et al., 2006); phosphatidylcholine-sterol O-acyltransferase (PDAT); and a wax synthase (WSD1, Li et al., 2008). The DGAT1 and DGAT2 proteins are eoncoded by two distinct gene families, with DGAT1 containing approximately 500 amino acids and 10 predicted transmembrane domains and DGAT2 has only 320 amino acids and two transmembrane domains (Shockey et al., 2006).

The term “triacylglycerol synthesising enzyme” or “TAG synthesising enzyme” as used herein means an enzyme capable of catalysing the addition of a third fatty acid to an existing diacylglycerol, thus generating TAG.

Preferred TAG synthesising enzymes include but are not limited to: acyl CoA: diacylglycerol acyltransferase1 (DGAT1); diacylglycerol acyl transferase2 (DGAT2); phosphatidylcholine-sterol O-acyltransferase (PDAT) and cytosolic soluble form of DGAT (soluble DGAT or DGAT3).

Examples of these TAG synthesising enzymes, suitable for use in the methods and compositions of the invention, from members of several plant species are provided in Table 1 below. The sequences (both polynucleotide and polypeptide are provided in the Sequence Listing).

TABLE 1
TAG synthesising enzyme sequences

| TAG | | | SEQ | | SEQ |
|---|---|---|---|---|---|
| synthesising | | Protein | ID | cDNA | ID |
| enzyme | Species | accession no. | NO: | accession no. | NO: |
| DGAT1 | A. thaliana | NP_179535 | 116 | NM_127503 | 124 |
| DGAT1 | T. majus | AAM03340 | 117 | AY084052 | 125 |
| DGAT1 | Z. mays | ABV91586 | 118 | EU039830 | 126 |
| DGAT2 | A. thaliana | NP_566952 | 119 | NM_115011 | 127 |
| DGAT2 | B. napus | AC090187 | 120 | FJ858270 | 128 |
| DGAT3 | A. hypogaea | AAX62735 | 121 | AY875644 | 129 |
| (soluble | | | | | |
| DGAT) | | | | | |
| PDAT | A. thaliana | NP_196868 | 122 | NM_121367 | 130 |
| PDAT | R. communis | XP_002521350 | 123 | XM_002521304 | 131 |
| MGAT | M. musculus | AAK84177.1 | 156 | AF384162.1 | 157 |

The invention also contemplates use of modified TAG synthesizing enzymes, that are modified (for example in their sequence by substitutions, insertions or additions and the like) to alter their specificity and or activity.

In one embodiment the TAG synthesizing enzymes is DGAT1.

### DGAT1

The term “DGAT1” as used herein means acyl CoA: diacylglycerol acyltransferase (EC 2.3.1.20)

DGAT1 is typically the major TAG synthesising enzyme in both the seed and senescing leaf (Kaup et al., 2002, Plant Physiol. 129(4):1616-26; for reviews see Lung and Weselake 2006, Lipids. December 2006; 41(12):1073-88; Cahoon et al., 2007, Current Opinion in Plant Biology. 10:236-244; and Li et al., 2010, Lipids. 45:145-157).

DGAT1 contains approximately 500 amino acids and has been reported to have up to 10 predicted transmembrane domains whereas DGAT2 has only 320 amino acids and is predicted to contain only two transmembrane domains; both proteins were also predicted to have their N- and C-termini located in the cytoplasm (Shockey et al., 2006, Plant Cell 18:2294-2313). Both DGAT1 and DGAT2 have orthologues in animals and fungi and are transmembrane proteins located in the ER.

In most dicotyledonous plants DGAT1 & DGAT2 appear to be single copy genes whereas there are typically two versions of each in the grasses which presumably arose during the duplication of the grass genome (Salse et al., 2008, Plant Cell, 20:11-24).

Examples of these DGAT1 sequences, suitable for use in the methods and compositions of the invention, from members of several plant species are provided in Table 2 below. The sequences (both polynucleotide and polypeptide are provided in the Sequence Listing)

TABLE 2
DGAT1 sequences from multiple species

| DGAT1 | PROTEIN | SEQ | DNA accession #s | SEQ |
|---|---|---|---|---|
| Species | accession #s | ID | & | ID |
| Source | & BAC # | NO: | BAC # | NO: |
| A. thaliana | NP_179535 | 58 | NM_127503 | 87 |
| B. juncea | AAY40784 | 59 | AF164434 | 88 |
| B. napus | AAD45536.1 | 60 | AF164434_1 | 89 |
| B. juncea | AAY40785 | 61 | DQ016107 | 90 |
| T. majus | AAM03340 | 62 | AY084052 | 91 |
| E. pitardii | ACO55635 | 63 | FJ226588 | 92 |
| V. galamensis | ABV21945 | 64 | EF653276 | 93 |
| N. tabacum | AAF19345.1 | 65 | AF129003_1 | 94 |
| P. frutescens | AAG23696.1 | 66 | AF298815_1 | 95 |
| Z. mays | From: CHORI-201 | 67 | From: CHORI-201 Maize | 96 |
| | Maize B73 BAC | | B73 BAC | |
| S. bicolor | XP_002439419 | 68 | XM_002439374 | 97 |
| O. sativa | NP_001054869 | 69 | Os05g0196800 | 98 |
| O. sativa | From: AP003714.1 | 70 | From: AP003714.1 | 99 |
| S. bicolor | XP_002437165 | 71 | XM_002437120.1 | 100 |
| Z. mays | ABV91586 | 72 | EU039830 | 101 |
| P. patens | XP_001770929 | 73 | XM_001770877.1 | 102 |
| S. | XP_002964165 | 74 | XM_002964119 | 103 |
| moellendorffii | | | | |
| E. alatus | AAV31083 | 75 | AY751297 | 104 |
| V. vinifera | XP_002279345 | 76 | XM_002279309 | 105 |
| G. max | AAS78662 | 77 | AY496439 | 106 |
| G. max | BAE93461 | 78 | AB257590 | 107 |
| L. japonicus | AAW51456 | 79 | AY859489 | 108 |
| M. truncatula | ABN09107 | 80 | AC174465.2 | 109 |
| J. curcas | ABB84383 | 81 | DQ278448.1 | 110 |
| V. fordii | ABC94472 | 82 | DQ356680.1 | 11 |
| V. galamensis | ABV21945 | 83 | EF653276.1 | 112 |
| R. communis | XP_002514132 | 84 | XM_002514086.1 | 113 |
| P. trichocarpa | XP_002308278 | 85 | XM_002308242.1 | 114 |
| P. trichocarpa | XP_002330510 | 86 | XM_002330474.1 | 115 |

In one embodiment the DGAT1 has the amino acid sequence of any one of SEQ ID NO: 58 to 86 (Table 2), 116 to 118 (Table 1), 8, and 12 (Table 4), or a variant thereof. Preferably the variant has at least 70% identity to any one of SEQ ID NO: 58 to 86, 116 to 118, 8 and 12. In a further embodiment the DGAT1 has the amino acid sequence of any one of SEQ ID NO: 58 to 86, 116 to 118, 8 and 12.

In one embodiment the DGAT1 is encoded by the polynucleotide sequence of any one of SEQ ID NO: 87 to 115, 124 to 126, 7 and 11, or a variant thereof. Preferably the variant has at least 70% identity to any one of SEQ ID NO: 87 to 115, 124 to 126, 7 and 11. In a further embodiment the DGAT1 is encoded by the polynucleotide sequence of any one of SEQ ID NO: 87 to 115, 124 to 126, 7 and 11.

### Oil Encapsulating Protein

Oil encapsulating proteins for use in the invention are well-known to those skilled in the art and include for example oleosins (Shao et al., 2019, New insights into the role of seed oil body proteins in metabolism and plant development, Front. Plant Sci., https://doi.org/10.3389/fpls.2019.01568), steroleosins (Lin et al., 2002, Steroleosin, a sterol-binding dehydrogenase in seed oil bodies. Plant Physiol. 128:1200-1211), caoleosins (Hsieh and Huan, 2004, Endoplasmic reticulum, oleosins, and oil seeds in tapetum cells. Plant Physiology, 136:3427-3434), polyoleosins, (WO2007045019), oleosin including at least one artificially introduced cysteine (WO2011/053169), oleosin where the lysine residues in the amphipathic arms have been replaced with arginine (US02021/0261632 A1).

In one embodiment the oil encapsulating protein is an oleosin.

### Oleosins

Oleosins are comparatively small (15 to 24 kDa) proteins which allow the OBs to become tightly packed discrete organelles without coalescing as the cells desiccate or undergo freezing conditions (Leprince et al., 1998; Siloto et al., 2006; Slack et al., 1980; Shimada et al.2008).

Oleosins have three functional domains consisting of an amphipathic N-terminal arm, a highly conserved central hydrophobic core (˜72 residues) and a C-terminal amphipathic arm. The accepted topological model is one in which the N- and C-terminal amphipathic arms are located on the outside of the OBs and the central hydrophobic core is located inside the OB (Huang, 1992; Loer and Herman, 1993; Murphy 1993). The negatively charged residues of the N- and C-terminal amphipathic arms are exposed to the aqueous exterior whereas the positively charged residues are exposed to the OB interior and face the negatively charged lipids. Thus, the amphipathic arms with their outward facing negative charge are responsible for maintaining the OBs as individual entities via steric hinderance and electrostatic repulsion both in vivo and in isolated preparation (Tzen et al., 1992). The N-terminal amphipathic arm is highly variable and as such no specific secondary structure can describe all examples. In comparison the C-terminal arm contains a α-helical domain of 30-40 residues (Tzen et al., 2003). The central core is highly conserved and thought to be the longest hydrophobic region known to occur in nature; at the centre is a conserved 12 residue proline knot motif which includes three spaced proline residues (for reviews see Frandsen et al., 2001; Tzen et al., 2003). The secondary, tertiary and quaternary structure of the central domain is still unclear. Modelling, Fourier Transformation-Infra Red (FT-IR) and Circular Dichromism (CD) evidence exists for a number of different arrangements (for review see Roberts et al., 2008).

The properties of the major oleosins is relatively conserved between plants and is characterised by the following:
15-25 kDa protein corresponding to approximately 140-230 amino acid residues.
The protein sequence can be divided almost equally along its length into 4 parts which correspond to a N-terminal hydrophilic region, two centre hydrophobic regions (joined by a proline knot or knob) and a C-terminal hydrophilic region.
The topology of oleosin is attributed to its physical properties which includes a folded hydrophobic core flanked by hydrophilic domains. This arrangement confers an amphipathic nature to oleosin resulting in the hydrophobic domain being embedded in the phospholipid monolayer (Tzen et al., 1992) while the flanking hydrophilic domains are exposed to the aqueous environment of the cytoplasm.
Typically oleosins do not contain cysteines.

Preferred oleosins for use in the invention are those which contain a central domain of approximately 70 non-polar amino acid residues (including a proline knot) uninterrupted by any charged residues, flanked by two hydrophilic arms.

Examples of oleosin sequences suitable for use in the invention in their native form, or suitable to be modified for use in the invention, and modified oleosins, are shown in Table 3 below. The sequences (both polynucleotide and polypeptide are provided in the Sequence Listing).

TABLE 3
Oleosin sequences from multiple species

| | | | SEQ | | SEQ |
|---|---|---|---|---|---|
| | | Protein | ID | | ID |
| Sequences | Species | accession no. | NO: | cDNA accession no. | NO: |
| Oleosin | S. indicum | AAG23840 | 132 | AF302907 | 144 |
| Oleosin | S. indicum | AAB58402 | 133 | U97700 | 145 |
| Oleosin | A. thaliana | CAA44225 | 134 | X62353 | 146 |
| Oleosin | A. thaliana | AAZ23930 | 135 | BT023738 | 147 |
| Oleosin | H. annuus | CAA44224.1 | 136 | X62352.1 | 148 |
| Oleosin | B. napus | CAA57545.1 | 137 | X82020.1 | 149 |
| Oleosin | Z. mays | NP_001147032.1 | 138 | NM_001153560.1 | 150 |
| Oleosin | O. sativa | AAL40177.1 | 139 | AAL40177.1 | 151 |
| Oleosin | B. oleracea | AAD24547.1 | 140 | AF117126.1 | 152 |
| Oleosin | C. arabica | AAY14574.1 | 141 | AY928084.1 | 153 |
| Oleosin | B. oleraceae | CAA65272.1 | 142 | X96409 | 154 |
| Oleosin | S. indicum | AAD42942 | 143 | AF091840 | 155 |
| Oleosin | G. max | NP_001358853 | 14 | NP_001358853 | 11 |
| Oleosin | G. max | NP_001347234.1 | 173 | NM_001360305.1 | 177* |
| Oleosin | G. max | NP_001238518.1 | 174 | NM_001251589.2 | 178 |
| Oleosin | G. soja | XP_028228155.1 | 175 | XM_028372354.1 | 179 |
| Oleosin | G. soja | KHN18226.1 | 176 | KN659929.1 | 180 |

*The polynucleotide sequence of SEQ ID NO: 177 encodes a polypeptide sequence that is slightly different from that of SEQ ID NO: 173, but in the National Center for Biotechnology Information (NCBI) database (available at https://www.ncbi.nlm.nih.gov/), NM_001360305.1 is the reference polynucleotide for the polypeptide sequence of NP_001347234.1 (reproduced as SEQ ID NO: 73).

In one embodiment the oleosin has the amino acid sequence of any one of SEQ ID NO: 132 to 143 (Table 3), and 10, 14, 16, 18, 20, 22, 24, 26, 28, 30, 32, 34, and 36 (Table 4), or a variant thereof. Preferably the variant has at least 70% identity to any one of SEQ ID NO: 132 to 143, and 10, 14, 16, 18, 20, 22, 24, 26, 28, 30, 32, 34, and 36. In a further embodiment the oleosin has the amino acid sequence of any one of SEQ ID NO: 14 and 132 to 143.

In one embodiment the oleosin is encoded by the polynucleotide sequence of any one of SEQ ID NO: 144 to 155 (Table 3) and 9, 13, 15, 17, 19, 21, 23, 25, 27, 29, 31, 33, and 35 (Table 4), or a variant thereof. Preferably the variant has at least 70% identity to any one of SEQ ID NO: 144 to 155 and 9, 13, 15, 17, 19, 21, 23, 25, 27, 29, 31, 33, and 35. In a further embodiment the oleosin is encoded by the polynucleotide sequence of any one of SEQ ID NO: 13, 144 to 155 and 177 to 180.

Oleosin are well known to those skilled in the art. Further sequences from many different species can be readily identified by methods well-known to those skilled in the art. For example, further sequences can be easily identified by an NCBI Entrez Cross-Database Search (available at http://www.ncbi.nlm.nih.gov/sites/gquery) using oleosin as a search term.

### Modified Oleosins

The invention also contemplates the use of modified oleosins including polyoleosins (WO/2007/045019), cysteine oleosins (WO/2011/053169) and lysine modified oleosins (US02021/0261632 A1).

### Cysteine Oleosins

Cysteine oleosins for use in the methods of the invention, are modified to contain at least one artificially introduced cysteine residue. Preferably the engineered oleosins contain at least two cysteines.

Various methods well-known to those skilled in the art may be used in production of the modified oleosins with artificially introduced cysteines.

Such methods include site directed mutagenesis (U.S. Pat. No. 6,448,048) in which the polynucleotide encoding an oleosin is modified to introduce a cysteine into the encoded oleosin protein.

Alternatively, the polynucleotide encoding the modified oleosins, may be synthesised in its entirety.

Further methodology for producing modified oleosins and for use in the methods of the invention are described in WO/2011/053169, U.S. Pat. No. 8,987,551, and WO/2013/022353.

The introduced cysteine may be an additional amino acid (i.e. an insertion) or may replace an existing amino acid (i.e. a replacement). Preferably the introduced cysteine replaces an existing amino acid. In a preferred embodiment the replaced amino acid is a charged residue. Preferably the charged residue is predicted to be in the hydrophilic domains and therefore likely to be located on the surface of the oil body.

The hydrophilic, and hydrophobic regions/arms of the oleosin can be easily identified by those skilled in the art using standard methodology (for example: Kyte and Doolitle (1982).

The modified oleosins for use in the methods of the invention are preferably range in molecular weight from 5 to 50 kDa, more preferably, 10 to 40 kDa, more preferably 15 to 25 kDa.

The modified oleosins for use in the methods of the invention are preferably in the size range 100 to 300 amino acids, more preferably 110 to 260 amino acids, more preferably 120 to 250 amino acids, more preferably 130 to 240 amino acids, more preferably 140 to 230 amino acids.

Preferably the modified oleosins comprise an N-terminal hydrophilic region, two centre hydrophobic regions (joined by a proline knot or knob) and a C-terminal hydrophilic region.

Preferably the modified oleosins can be divided almost equally their length into four parts which correspond to the N-terminal hydrophilic region (or arm), the two centre hydrophobic regions (joined by a proline knot or knob) and a C-terminal hydrophilic region (or arm).

Preferably the topology of modified oleosin is attributed to its physical properties which include a folded hydrophobic core flanked by hydrophilic domains.

Preferably the modified oleosins can be formed into oil bodies when combined with triacylglycerol (TAG) and phospholipid.

Preferably topology confers an amphipathic nature to modified oleosin resulting in the hydrophobic domain being embedded in the phospholipid monolayer of the oil body while the flanking hydrophilic domains are exposed to the aqueous environment outside the oil body, such as in the cytoplasm.

Preferably the modified oleosin includes at least one artificially introduced cysteine, wherein the cysteine is introduced into at least one of:
a) in the N-terminal hydrophilic region of the oleosin, and
b) in the C-terminal hydrophilic region of the oleosin.

In one embodiment the modified oleosin for use in the method of the invention, comprises a sequence with at least 70% identity to the hydrophobic domain of any of the oleosin protein sequences referred to in Table 3 above.

In one embodiment the modified oleosin has the amino acid sequence with 70% identity to any one of SEQ ID NO: 132 to 143 and 173 to 176 (Table 3), and 10, 14, 16, 18, 20, 22, 24, 26, 28, 30, 32, 34, and 36 (Table 4).

In a further embodiment the modified oleosin has the amino acid sequence of any one of SEQ ID NO: 10, 16, 18, 20, 22, 24, 26, 28, 30, 32, 34, and 36.

In one embodiment the modified oleosin is encoded by the polynucleotide sequence with 70% identity to of any one of SEQ ID NO: 144 to 155 and 177 to 180 (Table 3) and 9, 13, 15, 17, 19, 21, 23, 25, 27, 29, 31, 33, and 35 (Table 4).

In a further embodiment the modified oleosin is encoded by the polynucleotide sequence of any one of SEQ ID NO: 9, 15, 17, 19, 21, 23, 25, 27, 29, 31, 33, and 35.

In one embodiment the modified oleosin for use in the method of the invention, comprises a sequence with at least 70% identity to the hydrophobic domain of any of the amino acid sequences of SEQ ID NO: 132 to 143, and 10, 14, 16, 18, 20, 22, 24, 26, 28, 30, 32, 34, and 36.

In one embodiment the modified oleosin for use in the method of the invention, comprises a sequence with at least 70% identity to of any of the oleosin amino acid sequences referred to in Table 3 above.

In further embodiment the modified oleosin is essentially the same as any of the unmodied oleosins referred above, apart from the additional artificially introduced cysteine or cysteines.

### Ectopic Expression

The term “ectopic expression”, and grammatical equivalents thereof, as used here in means expression of a polynucleotide, gene or protein in a cell type, tissue type, or developmental stage, or an expression level, in/at which the polynucleotide, gene or protein is not usually endogenously expressed. Ectopic expression as used herein also encompassed transgenic expression, including over-expression.

### Seed-Preferred Expression

The term “seed-preferred expression”, and grammatical equivalents thereof, as used herein means expression predominantly in the seed of a plant relative to other tissues and parts of the plant. This term does not exclude some, albeit relatively low, expression in the seed of the plant.

For example FIG. 1 shows the combined expression profile of the 90 most highly expressed seed-preferred genes in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max. Data is from Soybean Expression Atlas (available at https://soybase.org/soyseq/). As shown expression is predominantly in the seed with only background found in the young leaf, flower, root and nodule. The low level of expression seen in some pod samples is likey contamination with seed material.

In one embodiment seed-preferred expression means an expression pattern substantially the same as that shown in FIG. 1.

By way of further example FIG. 2 shows expression of seed-preferred glycinin genes (Gly 2, Gly G2, Gly 4, Gly 5 and Gly 3) and most abundant oleosin gene (P24) from Glycine max in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max. Data is from Soybase.org. As shown expression is predominantly in the maturing seed.

In one embodiment seed-preferred expression means an expression pattern substantially the same as that shown in FIG. 2.

The term “seed-preferred expression”, and grammatical equivalents thereof, as used herein means also encompasses “seed-specific expression”, and grammatical equivalents thereof.

In one embodiment “seed-preferred expression” is “seed-specific expression”.

### Seed-Specific Expression

The term “seed-specific expression”, and grammatical equivalents thereof, as used herein means expression exclusively in the seed of a plant, and not in other tissues or parts of the plant.

### Non-Seed-Preferred Expression

By definition, non-seed-preferred expression means an expression pattern different from that of seed-preferred expression as described above.

In one embodiment not seed-preferred expression means an expression pattern substantially different from that shown in FIG. 1.

In a further embodiment not seed-preferred expression means an expression pattern substantially different from that shown in FIG. 2.

### Constitutive Expression

The term “constitutive expression”, and grammatical equivalents thereof, as used herein means expression in essentially all tissues of a plant.

By way of example For example FIGS. 3 and 4 show the constitutive expression of ubiquitin genes UBQ (the highest expressing two UBQ genes in FIG. 3 and the next highest expressing 5 UBQ genes in FIG. 4) and UBQ in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max. Data is from Soybean Expression Atlas (also available at https://soybase.org/soyseq/) and from Soybase.org. As shown all 7 UBQ genes are expressed in all of these tissues.

In one embodiment constitutive expression means an expression pattern substantially the same as that shown in one of FIGS. 3 and 4.

### Green Tissue-Preferred

The term “green tissue-preferred expression”, and grammatical equivalents thereof, as used herein means expression predominantly in the green tissues of a plant relative to other tissues of the plant. This term does not exclude some, albeit relatively low, expression in non-green tissues of the plant.

By way of example FIG. 5 shows the green tissue-preferred expression of CAB3, CAB6 and two RBCS genes in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max.

As shown expression is predominantly in the leaf and tissues that would be green at the time of sampling (flower and young pod).

The term “green tissue-preferred expression”, and grammatical equivalents thereof, as used herein also encompassed “green tissue-specific expression”, and grammatical equivalents thereof.

In one embodiment “green tissue-preferred expression” is “green tissue-specific expression”.

### Green Tissue-Specific

The term “green tissue-specific expression”, and grammatical equivalents thereof, as used herein means expression exclusively in the green tissues of a plant, and not in other non-green tissues of the plant.

### Light-Induced Expression

The term “Light-induced expression”, and grammatical equivalents thereof, as used herein means expression induced by light, in the plant.

### Promoters

### Seed-Preferred Promoters

Seed-preferred promoters drive expression of operably linked polynucleotides predominantly in the seeds of plants. This term does not exclude some, albeit relatively low, expression in other non-seed tissues of the plant.

Numerous examples of seed-preferred promoters are known by those skilled in the art and include, by way of example, but are not limited to: seed-preferred promoters found in U.S. Pat. Nos. 6,342,657; and 7,081,565; and 7,405,345; and 7,642,346; and 7,371,928, and napin promoters, legumin B4, 7S globulin, and 11S globulin (Zakharov et al., 2004, J. Exp. Bot., 55:1463-1471), dlec2, Arc5-1, lectin, and usp (Stoger et al., 2005, Current Opinion in Biotechnology, 16:167-173).

The term “seed-preferred promoter”, and grammatical equivalents thereof, as used herein also encompasses “seed-specific promoter”, and grammatical equivalents thereof.

In one embodiment “seed-preferred promoter” is a “seed-specific promoter”.

### Seed Specific Promoters

Seed-specific promoters drive expression of operably linked polynucleotides exclusively in the seeds of plants.

### Non-Seed Preferred Promoters

By definition, a non-seed preferred promoter includes any promoter that is not a seed-preferred or seed-specific promoter.

Preferably, the non-seed preferred promoter is capable of driving expression of an operably linked polynucleotide in a plant.

### Constitutive Promoters

Numerous constitutive promoters are known to those skilled in the art, and include for example CaMV35s (Hoshino 2007. Isolamento e caracterizacao de promotores tecido especificos a partir das informacoes do Sucest (Sugarcane expressed sequence tags). Ph.D. Thesis, Instituto de Biociencias da Universidade Estadual Paulista, Botucatu-SP., Ubiqutin (Christensen and Quail, 1996, Ubiqutin promoter-based vectors for high-level expression of selectable and/or screenable marker genes in monocotyledonous plants. Transgenic Research 5:213-218), Actin (McElroy et al., 1990, Isolation of an efficient actin promoter for use in rice transformation. Plant Cell 2:163-171), and Cassava Vein Mosaic Virus (Verdaguer et al., 1998, Plant Mol Biol., 37(6):1055-67).

### CaMV Promoters

In one embodiment the constitutive promoter is a cauliflower mosaic virus (CaMV) promoter.

In one embodiment the CaMV promoter is a CaMV 35S promoter.

In one embodiment the CaMV 35S promoter has at least 70% identity to the polynucleotide sequence of SEQ ID NO: 1.

In a further embodiment the CaMV 35S promoter has the polynucleotide sequence of SEQ ID NO:1.

Use of double CaMV 35S promoters is also contemplated.

### Ubiquitin Promoters

In a further embodiment the constitutive promoter is a ubiquitin promoter.

In one embodiment the ubiquitin promoter has at least 70% identity to the polynucleotdies sequence of any one of SEQ ID NO: 164-165.

In a further embodiment the ubiquitin promoter has the polynucleotdies sequence of any one of SEQ ID NO: 164-165.

Use of double Ubiquitin promoters is also contemplated.

### Green Tissue-Preferred and Green Tissue-Specific Promoters

### Cab Promoters

In a further embodiment the green tissue-preferred or green tissue-specific promoter is a chlorophyll a/b (Cab) binding promoter.

In one embodiment the cab promoter has at least 70% identity to the polynucleotdies sequence of any one of SEQ ID NO: 2, 5, 6, and 158-161.

In a further embodiment the cab promoter has the polynucleotide sequence of any one of SEQ ID NO: 2, 5, 6, and 158-161.

### Rbcs Promoters

In a further embodiment the green tissue-preferred or green tissue-specific promoter is a promoter from a small subunit of ribulose-bisphosphate carboxylase (Rubisco) promoter, also know as an rbcS promoter.

In one embodiment the rbcS promoter has at least 70% identity to the polynucleotide sequence of any one of SEQ ID NO: 3, 4, 162 and 163.

In a further embodiment the rbcS promoter has the polynucleotide sequence of any one of SEQ ID NO: 3, 4, 162 and 163.

### Source of Plant Sequences and Plants Used in the Invention.

The plant-derived oil synthesising enzymes, the oil encapsulating proteins, promoters and plants used in the invention, may be from any plant species.

In one embodiment the plant is derived from a gymnosperm plant species.

In a further embodiment the plant is derived from an angiosperm plant species.

In a further embodiment the plant is derived from a from dicotyledonous plant species.

In a further embodiment the plant is derived from a monocotyledonous plant species.

Other preferred plants are forage plant species from a group comprising but not limited to the following genera: Zea, Lolium, Hordium, Miscanthus, Saccharum, Festuca, Dactylis, Bromus, Thinopyrum, Trifolium, Medicago, Pheleum, Phalaris, Holcus, Glycine, Lotus, Plantago and Cichorium.

Other preferred plants are leguminous plants. The leguminous plant or part thereof may encompass any plant in the plant family Leguminosae or Fabaceae. For example, the plants may be selected from forage legumes including, alfalfa, clover; leucaena; grain legumes including, beans, lentils, lupins, peas, peanuts, soy bean; bloom legumes including lupin, pharmaceutical or industrial legumes; and fallow or green manure legume species.

A further contemplated genus is Trifolium. Preferred Trifolium species include Trifolium repens; Trifolium arvense; Trifolium affine; and Trifolium occidentale. A particularly preferred Trifolium species is Trifolium repens.

Another preferred genus is Medicago. Preferred Medicago species include Medicago sativa and Medicago truncatula. A particularly preferred Medicago species is Medicago sativa, commonly known as alfalfa.

Another preferred genus is Glycine. Preferred Glycine species include Glycine max, Glycine wightii (also known as Neonotonia wightii) and Glycine soja. A particularly preferred Glycine species is Glycine max, commonly known as soy bean. A particularly preferred Glycine species is Glycine wightii, commonly known as perennial soybean.

Another preferred genus is Vigna. A particularly preferred Vigna species is Vigna unguiculata commonly known as cowpea.

Another preferred genus is Mucana. Preferred Mucana species include Mucana pruniens. A particularly preferred Mucana species is Mucana pruniens commonly known as velvet bean.

Another preferred genus is Arachis. A particularly preferred Arachis species is Arachis glabrata commonly known as perennial peanut.

Another preferred genus is Pisum. A preferred Pisum species is Pisum sativum commonly known as pea.

Another preferred genus is Lotus. Preferred Lotus species include Lotus corniculatus, Lotus pedunculatus, Lotus glabar, Lotus tenuis and Lotus uliginosus. A preferred Lotus species is Lotus corniculatus commonly known as Birdsfoot Trefoil. Another preferred Lotus species is Lotus glabar commonly known as Narrow-leaf Birdsfoot Trefoil. Another preferred preferred Lotus species is Lotus pedunculatus commonly known as Big trefoil. Another preferred Lotus species is Lotus tenuis commonly known as Slender trefoil.

Another preferred genus is Brassica. A preferred Brassica species is Brassica oleracea, commonly known as forage kale and cabbage.

Other preferred species are oil seed crops.

Preferably oil seed crops include but are not limited to the following genera: Brassica, Carthumus, Helianthus, Zea and Sesamum.

A preferred oil seed genera is Glycine. Preferred Glycine species include Glycine max and Glycine soja. Preferred Glycine species is Glycine max.

A preferred oil seed genera is Brassica. A preferred oil seed species is Brassica napus.

A preferred oil seed genera is Brassica. A preferred oil seed species is Brassica oleraceae.

A preferred oil seed genera is Carthamus. A preferred oil seed species is Carthamus tinctorius.

A preferred oil seed genera is Helianthus. A preferred oil seed species is Helianthus annuus.

A preferred oil seed genera is Zea. A preferred oil seed species is Zea mays.

A preferred oil seed genera is Sesamum. A preferred oil seed species is Sesamum indicum.

A preferred silage genera is Zea. A preferred silage species is Zea mays.

A preferred grain producing genera is Hordeum. A preferred grain producing species is Hordeum vulgare.

A preferred grazing genera is Lolium. A preferred grazing species is Lolium perenne.

A preferred grazing genera is Lolium. A preferred grazing species is Lolium arundinaceum.

A preferred grazing genera is Trifolium. A preferred grazing species is Trifolium repens.

A preferred grazing genera is Hordeum. A preferred grazing species is Hordeum vulgare.

Preferred plants also include forage, or animal feedstock plants. Such plants include but are not limited to the following genera: Miscanthus, Saccharum, Panicum.

A preferred biofuel genera is Miscanthus. A preferred biofuel species is Miscanthus giganteus.

A preferred biofuel genera is Saccharum. A preferred biofuel species is Saccharum officinarum.

A preferred biofuel genera is Panicum. A preferred biofuel species is Panicum virgatum.

A particularly preferred genus of plant in which to increase seed oil content, without decreasing seed protein content, in accordance with the invention, is Glycine. A particularly preferred Glycine species in Glycine max.

Particularly preferred genera as sources of the oil synthesizing enzyme for use in the invention are Tropaeolum and Glycine. A particularly preferred Tropaeolum species is Tropaeolum majus. A particularly preferred Glycine species in Glycine max.

Particularly preferred genera as sources of the oil encapsulating for use in the invention are Sesamum and Glycine. A particularly preferred Sesamum species is Sesamum indicum. A particularly preferred Glycine species in Glycine max.

Particularly preferred genera as sources of the constitutive promoters for use in the invention are Arabidopsis and Glycine. A particularly preferred Arabidopsis species is Arabidopsis thaliana. A particularly preferred Glycine species in Glycine max.

Particularly preferred genera as sources of the green tissue-preferred and green tissue specific promoters for use in the invention are Pisum and Glycine. A particularly preferred Pisum species is Pisum sativa. A particularly preferred Glycine species in Glycine max.

### Plant Parts, Propagues and Progeny

The term “plant” is intended to include a whole plant, any part of a plant, a seed, a fruit, propagules and progeny of a plant.

The term ‘propagule’ means any part of a plant that may be used in reproduction or propagation, either sexual or asexual, including seeds and cuttings.

The plants of the invention may be grown and either self-ed or crossed with a different plant strain and the resulting progeny, comprising the polynucleotides or constructs of the invention, also form a part of the present invention.

Preferably the plants, plant parts, propagules and progeny comprise a polynucleotide or construct according to the invention, and/or express a sequence according to the invention.

### Control Plant

Those skilled in the art will know how to choose a suitable control plant.

In one embodiment the control plant is of the same type, and age or developmental stage, but does not ectopically express the oil synthesising enzyme in accordance with the invention.

In one embodiment the control plant is of the same type, and age or developmental stage, but does not ectopically express the oil encapsulating protein in accordance with the invention.

In a further embodiment the control plant is of the same type, and age or developmental stage, but does not ectopically express the oil synthesising enzyme in accordance with the invention, or the oil encapsulating protein in accordance with the invention.

In a further embodiment the control plant is not transformed with the polynucleotide, or construct, encoding the oil synthesising enzyme in accordance with the invention.

In a further embodiment the control plant is not transformed with the polynucleotide, or construct, encoding the oil encapsulating protein in accordance with the invention.

In a further embodiment the control plant is not transformed with the polynucleotide, or construct, encoding the oil synthesising enzyme in accordance with the invention, or with the polynucleotide, or construct, encoding the oil encapsulating protein in accordance with the invention.

In one embodiment the control plant is an untransformed plant.

In a further embodiment the control plant is transformed with a control construct. In one embodiment the control construct is an “empty vector” construct.

In a further embodiment the control plant is a null segregant.

In a further embodiment the control plant is a plant that has not been modified, by a gene-editing technique to express the protein according to the invention.

Preferably the control part, propagule or progeny is from a control plant as described above.

### Plant Parts

In one embodiment the part is from a reproductive tissue. In a further embodiment the part is a seed.

### Protein-Enriched Co-Product

In a further aspect the invention provides a protein-enriched co-product.

In one embodiment the protein-enriched co-product is what is left after extraction of oil from a seed of the invention or a seed produced by a method of the invention.

In one embodiment the protein-enriched co-product has no less protein relative to an equivalent protein co-product produced from a control seed, or seed from a control plant.

In a further embodiment the protein-enriched co-product has increased protein relative to an equivalent protein co-product produced from a control seed, or seed from a control plant.

### Animal Feedstock

In a further aspect the invention provides an animal feedstock comprising a protein-enriched co-product of the invention, or produced by a method of the invention.

In one embodiment the animal feedstock has no less protein relative to an equivalent animal feedstock produced from a control seed, or seed from a control plant.

In a further embodiment the animal feedstock has increased protein relative to an equivalent animal feedstock produced from a control seed, or seed from a control plant.

### Food Ingredient

In a further aspect the invention provides a food ingredient comprising a protein-enriched co-product of the invention, or produced by a method of the invention.

In one embodiment the food ingredient has no less protein relative to an equivalent food ingredient produced from a control seed, or seed from a control plant.

In a further embodiment the food ingredient has increased protein relative to an equivalent food ingredient produced from a control seed, or seed from a control plant.

### Method for Producing Oil

In a further aspect the invention provides a method for producing oil, the method comprising extracting lipid from at least one of a plant, plant part, propagule and progeny of the invention, or produced by a method of the invention.

In a preferred embodiment the plant part is a seed.

In one embodiment the method includes the step of extracting lipid via crushing.

In one embodiment the crushing is expeller crushing.

In a further embodiment the method includes the step of extracting lipid via solvent extraction.

In one embodiment the solvent is hexane.

In a further embodiment the method includes the step of extracting lipid via critical point extraction.

In a further embodiment the lipid is processed into at least one of
a) a fuel,
b) an oleochemical,
c) a nutritional oil,
d) a cosmetic oil,
e) a polyunsaturated fatty acid (PUFA), and
f) a combination of any of a) to e).

### Lipid Production

In certain embodiments the cell, tissues, plants and plant parts of the invention produces more lipid than control cells, tissues, plants and plant parts.

Those skilled in the art are well aware of methods for measuring lipid production. This may typically be done by quantitative fatty acid methyl ester gas chromatography mass spectral analysis (FAMES GC-MS). Suitable methods are also described in the examples section of this specification.

### Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of agene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides.

### Proteins Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides or proteins of the present invention, or used in the methods of the invention, may be purified natural products, or may be produced partially or wholly using recombinant or synthetic techniques. The modified DGAT1 proteins may also be expressed from endogenous polynucleotides that have been modified using gene editing approaches.

A “fragment” of a polypeptide is a subsequence of the polypeptide that preferably performs a function of and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides of the invention being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

### Variants

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polypeptides possess biological activities that are the same or similar to those of the inventive polypeptides or polypeptides. The term “variant” with reference to polypeptides and polypeptides encompasses all forms of polypeptides and polypeptides as defined herein.

### Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present invention. Identity is found over a comparison window of at least 20 nucleotide positions, preferably at least 50 nucleotide positions, more preferably at least 100 nucleotide positions, and most preferably over the entire length of a polynucleotide of the invention.

Polynucleotide sequence identity can be determined in the following manner. The subject polynucleotide sequence is compared to a candidate polynucleotide sequence using BLASTN (from the BLAST suite of programs, version 2.2.5 [November 2002]) in b12seq (Tatiana A. Tatusova, Thomas L. Madden (1999), “Blast 2 sequences—a new tool for comparing protein and nucleotide sequences”, FEMS Microbiol Lett. 174:247-250), which is publicly available from the NCBI website on the World Wide Web at ftp://ftp.ncbi.nih.gov/blast/. The default parameters of b12seq are utilized except that filtering of low complexity parts should be turned off.

The identity of polynucleotide sequences may be examined using the following unix command line parameters:
b12seq -i nucleotideseq1 -j nucleotideseq2 -F F -p blastn

The parameter-F F turns off filtering of low complexity sections. The parameter-p selects the appropriate algorithm for the pair of sequences. The b12seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from the world wide web at http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

A preferred method for calculating polynucleotide % sequence identity is based on aligning sequences to be compared using Clustal X (Jeanmougin et al., 1998, Trends Biochem. Sci. 23, 403-5.).

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available b12seq program from the BLAST suite of programs (version 2.2.5 [November 2002]) from the NCBI website on the World Wide Web at ftp://ftp.ncbi.nih.gov/blast/.

The similarity of polynucleotide sequences may be examined using the following unix command line parameters:
b12seq-i nucleotideseq1-j nucleotideseq2-F F-p tblastx

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the b12seq program. For small E values, much less than one, the E value is approximately the probability of such a random match.

Variant polynucleotide sequences preferably exhibit an E value of less than 1×10-6 more preferably less than 1×10-9, more preferably less than 1×10-12, more preferably less than 1×10-15, more preferably less than 1×10-18, more preferably less than 1×10-21, more preferably less than 1×10-30, more preferably less than 1×10-40, more preferably less than 1×10-50, more preferably less than 1×10-60, more preferably less than 1×10-70, more preferably less than 1×10-80, more preferably less than 1×10-90 and most preferably less than 1×10-100 when compared with any one of the specifically identified sequences.

Alternatively, variant polynucleotides of the present invention, or used in the methods of the invention, hybridize to the specified polynucleotide sequences, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C-log(Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length) ° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides of the present invention, or used in the methods of the invention, also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available b12seq program from the BLAST suite of programs (version 2.2.5 [November 2002]) from the NCBI website on the World Wide Web at ftp://ftp.ncbi.nih.gov/blast/via the tblastx algorithm as previously described.

### Polypeptide Variants

The term “variant” with reference to polypeptides encompasses naturally occurring, recombinantly and synthetically produced polypeptides. Variant polypeptide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.5 [November 2002]) in b12seq, which is publicly available from the NCBI website on the World Wide Web at ftp://ftp.ncbi.nih.gov/blast/. The default parameters of b12seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

A preferred method for calculating polypeptide % sequence identity is based on aligning sequences to be compared using Clustal X (Jeanmougin et al., 1998, Trends Biochem. Sci. 23, 403-5.).

Polypeptide variants of the present invention, or used in the methods of the invention, also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available b12seq program from the BLAST suite of programs (version 2.2.5 [November 2002]) from the NCBI website on the World Wide Web at ftp://ftp.ncbi.nih.gov/blast/. The similarity of polypeptide sequences may be examined using the following unix command line parameters:
b12seq -i peptideseq1 -j peptideseq2 -F F -p blastp

Variant polypeptide sequences preferably exhibit an E value of less than 1×10-6 more preferably less than 1×10-9, more preferably less than 1×10-12, more preferably less than 1×10-15, more preferably less than 1×10-18, more preferably less than 1×10-21, more preferably less than 1×10-30, more preferably less than 1×10-40, more preferably less than 1×10-50, more preferably less than 1×10-60, more preferably less than 1×10-70, more preferably less than 1×10-80, more preferably less than 1×10-90 and most preferably 1×10-100 when compared with any one of the specifically identified sequences.

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

### Constructs, Vectors and Components Thereof

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence may, in some cases, identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination, mRNA stability, and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors. Introns within coding sequences can also regulate transcription and influence post-transcriptional processing (including splicing, capping and polyadenylation).

A promoter may be homologous with respect to the polynucleotide to be expressed. This means that the promoter and polynucleotide are found operably linked in nature.

Alternatively the promoter may be heterologous with respect to the polynucleotide to be expressed. This means that the promoter and the polynucleotide are not found operably linked in nature.

In certain embodiments the polynucleotides/polypeptides of the invention may be advantageously expressed under the control of selected promoter sequences as described below.

### Vegetative Tissue Specific Promoters

An example of a vegetative specific promoter is found in U.S. Pat. Nos. 6,229,067; and 7,629,454; and 7,153,953; and 6,228,643.

### Photosynthetic Tissue Preferred Promoters

Photosynthetic tissue preferred promoters include those that are preferentially expressed in photosynthetic tissues of the plants. Photosynthetic tissues of the plant include leaves, stems, shoots and above ground parts of the plant. Photosynthetic tissue preferred promoters include light regulated promoters.

### Light Regulated Promoters

Numerous light regulated promoters are known to those skilled in the art and include for example chlorophyll a/b (Cab) binding protein promoters and Rubisco Small Subunit (SSU) promoters. An example of a light regulated promoter is found in U.S. Pat. No. 5,750,385. Light regulated in this context means light inducible or light induced.

A “transgene” is a polynucleotide that is taken from one organism and introduced into a different organism by transformation. The transgene may be derived from the same species or from a different species as the species of the organism into which the transgene is introduced.

### Transgenic Plant

A “transgenic plant” refers to a plant which contains new genetic material as a result of genetic manipulation or transformation. The new genetic material may be derived from a plant of the same species as the resulting transgenic plant or from a different species.

### Methods for Isolating or Producing Polynucleotides

The polynucleotide molecules of the invention can be isolated by using a variety of techniques known to those of ordinary skill in the art. By way of example, such polypeptides can be isolated through use of the polymerase chain reaction (PCR) described in Mullis et al., Eds. 1994 The Polymerase Chain Reaction, Birkhauser, incorporated herein by reference. The polypeptides of the invention can be amplified using primers, as defined herein, derived from the polynucleotide sequences of the invention.

Further methods for isolating polynucleotides of the invention include use of all, or portions of, the polypeptides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the invention may be produced by techniques well-known in the art such as restriction endonuclease digestion, oligonucleotide synthesis and PCR amplification.

A partial polynucleotide sequence may be used, in methods well-known in the art to identify the corresponding full length polynucleotide sequence. Such methods include PCR-based methods, 5′RACE (Frohman M A, 1993, Methods Enzymol. 218: 340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. For these reasons among others, it is desirable to be able to identify and isolate orthologues of a particular gene in several different plant species.

Variants (including orthologues) may be identified by the methods described.

### Methods for Identifying Variants

### Physical Methods

Variant polypeptides may be identified using PCR-based methods (Mullis et al., Eds. 1994 The Polymerase Chain Reaction, Birkhauser). Typically, the polynucleotide sequence of a primer, useful to amplify variants of polynucleotide molecules of the invention by PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides of the invention (Sambrook et al., Molecular Cloning: A Laboratory Manual, 2nd Ed. Cold Spring Harbor Press, 1987) or by identifying polypeptides from natural sources with the aid of such antibodies.

### Computer Based Methods

The variant sequences of the invention, including both polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, MD 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/Biolnfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

### Methods for Isolating Polypeptides

The polypeptides of the invention, or used in the methods of the invention, including variant polypeptides, may be prepared using peptide synthesis methods well known in the art such as direct peptide synthesis using solid phase techniques (e.g. Stewart et al., 1969, in Solid-Phase Peptide Synthesis, WH Freeman Co, San Francisco California, or automated synthesis, for example using an Applied Biosystems 431A Peptide Synthesizer (Foster City, California). Mutated forms of the polypeptides may also be produced during such syntheses.

The polypeptides and variant polypeptides of the invention, or used in the methods of the invention, may also be purified from natural sources using a variety of techniques that are well known in the art (e.g. Deutscher, 1990, Ed, Methods in Enzymology, Vol. 182, Guide to Protein Purification,).

Alternatively the polypeptides and variant polypeptides of the invention, or used in the methods of the invention, may be expressed recombinantly in suitable host cells and separated from the cells as discussed below.

### Methods for Producing Constructs and Vectors

The genetic constructs of the present invention comprise one or more polynucleotide sequences of the invention and/or polynucleotides encoding polypeptides of the invention, and may be useful for transforming, for example, bacterial, fungal, insect, mammalian or plant organisms. The genetic constructs of the invention are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987.

### Methods for Producing Host Cells Comprising Polynucleotides, Constructs or Vectors

The invention provides a host cell which comprises a genetic construct or vector of the invention.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

### Methods for Producing Plant Cells and Plants Comprising Constructs and Vectors

The invention further provides plant cells which comprise a genetic construct of the invention, and plant cells modified to alter expression of a polynucleotide or polypeptide of the invention, or used in the methods of the invention. Plants comprising such cells also form an aspect of the invention.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

### Methods for Genetic Manipulation of Plants

A number of plant transformation strategies are available (e.g. Birch, 1997, Ann Rev Plant Phys Plant Mol Biol, 48, 297; Hellens et al., 2000, Plant Mol Biol 42: 819-32; Hellens et al., Plant Meth 1: 13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Genetic constructs for expression of genes in transgenic plants typically include promoters for driving the expression of one or more cloned polynucleotide, terminators and selectable marker sequences to detect presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues respond to internal developmental signals or external abiotic or biotic stresses are described in the scientific literature. Exemplary promoters are described, e.g., in WO 02/00894 and WO2011/053169, which is herein incorporated by reference.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

Numerous binary vectors that replicate in Agrobacterium and can be used for delivery of constructs into plant cells are known to those skilled in the art and include for example: pBIN19 (Bevan M, 1984, Nucleic Acids Research. 12 (22): 8711-21), pGreen0000 (Hellens et al., Plant Molecular Biology. 42 (6): 819-32.) and pLX-B2 (Pasin et al., 2017, ACS Synthetic Biology. 6 (10): 1962-1968). Binary vectors for Ochrobactrum-mediated transformation are also well-known in the art as described for example in EP3341483B1, US20180216123A1, PCT/US2016/049135, and Cho et al., 2022, Plant Biotechnol J., https://doi.org/10.1111/pbi.13777.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci.104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,045 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24(1):45-51); Prunus (Ramesh et al., 2006 Plant Cell Rep. 25(8):821-8; Song and Sink 2005 Plant Cell Rep. 2006; 25(2):117-23; Gonzalez Padilla et al., 2003 Plant Cell Rep. 22(1):38-45); strawberry (Oosumi et al., 2006 Planta. 223(6):1219-30; Folta et al., 2006 Planta Apr 14; PMID: 16614818), rose (Li et al., 2003), Rubus (Graham et al., 1995 Methods Mol Biol. 1995; 44:129-33), tomato (Dan et al., 2006, Plant Cell Reports V25:432-441), apple (Yao et al., 1995, Plant Cell Rep. 14, 407-412), Canola (Brassica napus L.).(Cardoza and Stewart, 2006 Methods Mol Biol. 343:257-66), safflower (Orlikowska et al, 1995, Plant Cell Tissue and Organ Culture 40:85-91), ryegrass (Altpeter et al., 2004 Developments in Plant Breeding 11(7):255-250), rice (Christou et al., 1991 Nature Biotech. 9:957-962), maize (Wang et al., 2009 In: Handbook of Maize pp. 609-639) and Actinidia eriantha (Wang et al., 2006, Plant Cell Rep. 25, 5:425-31). Transformation of other species is also contemplated by the invention. Suitable methods and protocols are available in the scientific literature.

### Modification of Endogenous Genomes

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of cell types and in organisms that have traditionally been challenging to manipulate genetically. A modified version of the CRISPR-Cas9 system has been developed to recruit heterologous domains that can regulate endogenous gene expression or label specific genomic loci in living cells (Nature Biotechnology 32, 347-355 (2014). The system is applicable to plants, and can be used to regulate expression of target genes. (Bortesi and Fischer, Biotechnology Advances Volume 33, Issue 1, January-February 2015, Pages 41-52). Use of CRISPR technology in plants is also reviewed in Zhang et al., 2019, Nature Plants, Volume 5, pages778-794.

### BRIEF DESCRIPTION OF THE FIGURES

FIG. 1 shows the combined expression profile of the 90 most highly expressed seed-preferred genes in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max.

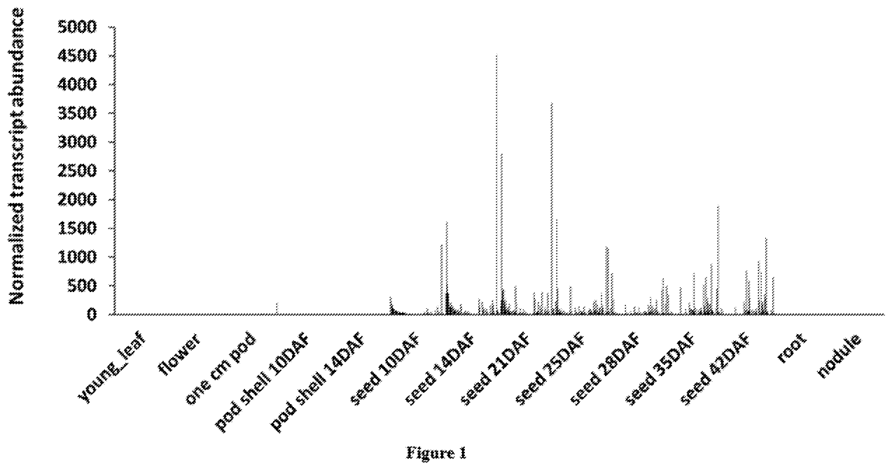

FIG. 2 shows expression of seed-preferred glycinin genes (Gly 2, Gly G2, Gly 4, Gly 5 and Gly 3) and most abundant oleosin gene (P24) from Glycine max in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max.

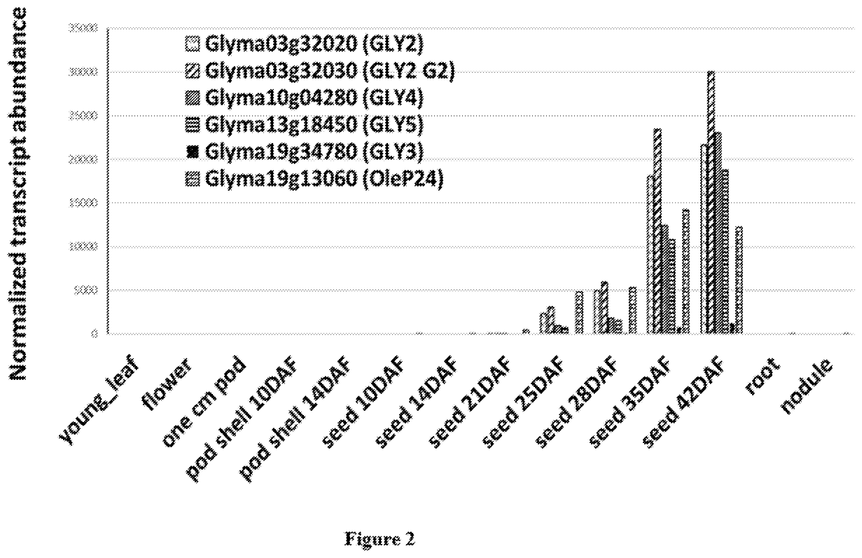

FIG. 3 shows the two highest expressing ubiquitin genes (UBQ) in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max.

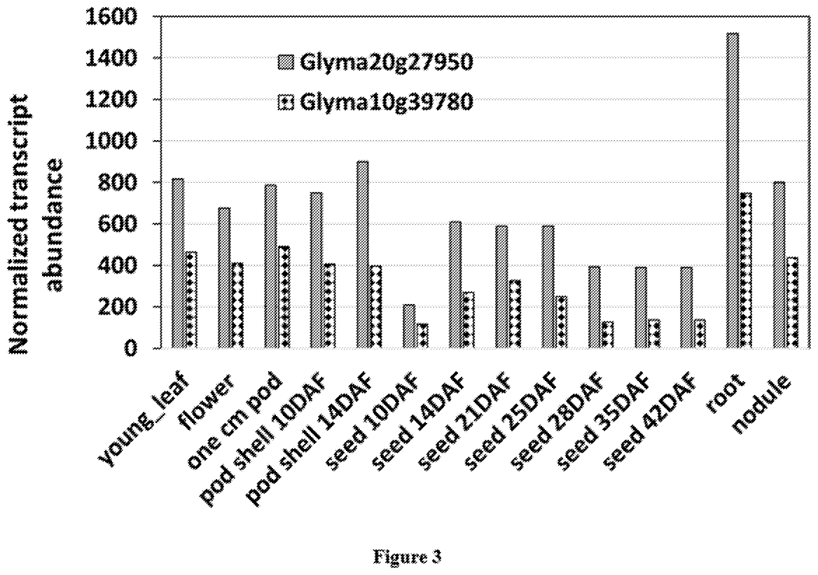

FIG. 4 shows the 3^{rd}, 4^{th}, 5^{th}, 6^{th} and 7^{th} highest expressing ubiquitin genes (UBQ) in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max.

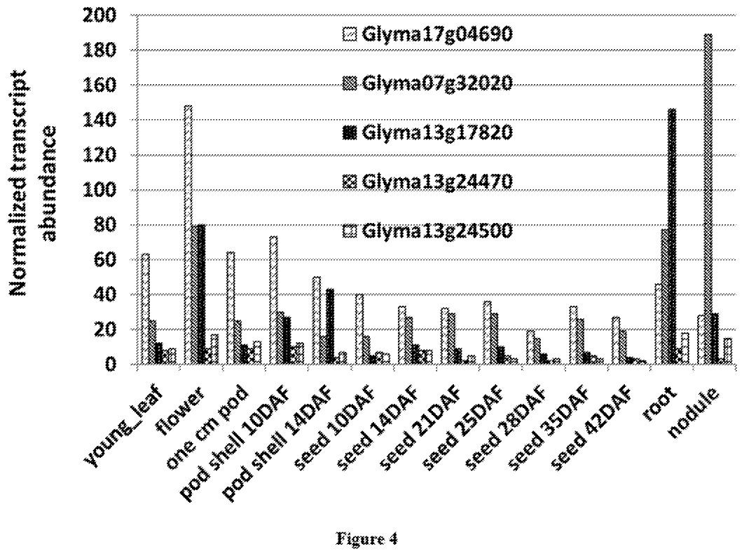

FIG. 5 shows the green tissue-preferred expression of CAB3, CAB6 and two RBCS genes in various tissues (young, leaf, flower, one cm pod, pod shell 10 days after flowering [DAF], pod shell 14 DAF, seed 10 DAF, seed 14 DAF, seed 21 DAF, seed 25 DAF, seed 28 DAF, seed 35 DAF, seed 42 DAF, root and nodule) in Glycine max.

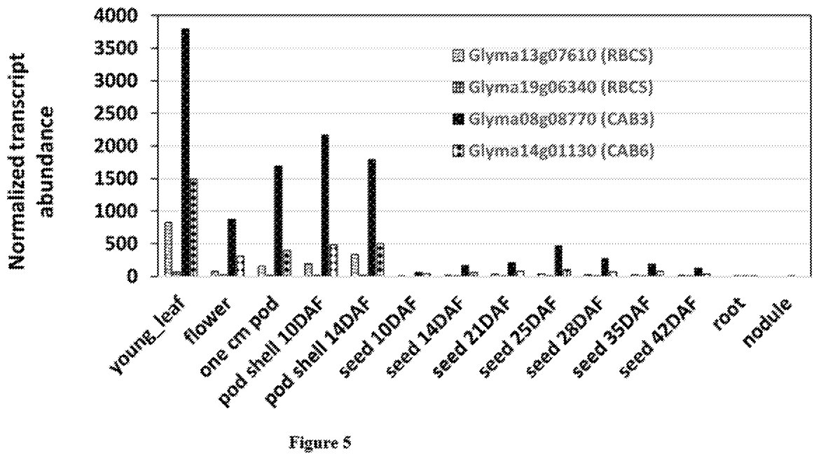

### EXAMPLES

The invention will now be described with reference to the following non-limiting examples.

### Example 1

Generation of construct (C1) containing a CaMV35S (Table 4, SEQ ID NO: 1) promoter driving T. majus DGAT1-V5 (Table 4, SEQ ID NO: 7+V5) and in a tandem orientation a second CaMV35S promoter driving S. indicum Cys-Ole (Table 4, SEQ ID NO: 9) for transformation into soybean.

The open reading frames of T. majus DGAT1-V5 and S. indicum Cys-Ole were optimized for expression in G. max; this included the addition of the S. tuberosum LS1 intron 2 into both open reading frames (Table 4, SEQ ID NO: 37). The complete T-DNA was contained within the binary vector pZY101 (Zeng et al., 2004).

### Example 2

Generation of construct (C2) containing P. sativum CAB (Table 4, SEQ ID NO: 2) and rbcS (Table 4, SEQ ID NO: 3) promoters in a back to back orientation driving T. majus DGAT1-V5 (Table 4, SEQ ID NO: 7+V5) and S. indicum Cys-Ole (Table 4, SEQ ID NO: 9) respectively, for transformation into soybean.

The open reading frames of T. majus DGAT1-V5 and S. indicum Cys-Ole were optimized for expression in G. max; this included the addition of introns into the Tm DGAT1 ORF and the Si Cys-Ole ORF (Table 4, SEQ ID NO: 38). The complete T-DNA was contained within the binary vector pZY101 (Zeng et al., 2004).

### Example 3

Generation of construct (C3) containing P. sativum CAB (Table 4, SEQ ID NO: 2) and rbcS promoters (Table 4, SEQ ID NO: 3) in a tandem orientation driving TmDGAT1-V5 (Table 4, SEQ ID NO: 7+V5) and SiCys-Ole (Table 4, SEQ ID NO: 9) respectively, for transformation into soybean.

The open reading frames of T. majus DGAT1-V5 and S. indicum Cys-Ole were optimized for expression in G. max; this included the addition of introns into the Tm DGAT1 ORF and the Si Cys-Ole ORF (Table 4, SEQ ID NO: 39). The complete T-DNA was contained within the binary vector pZY101 (Zeng et al., 2004).

### Example 4

Generation of construct (C4) containing the P. sativum rbcS promoter (Table 4, SEQ ID NO: 3) driving SiCys-Ole (Table 4, SEQ ID NO: 9) and P. sativum rbcS promoter (Table 4, SEQ ID NO: 3) driving TmDGAT1 (Table 4, SEQ ID NO: 7) in a tandem orientation for transformation into soybean.

The open reading frames of T. majus DGAT1 and S. indicum Cys-Ole were optimized for expression in G. max; this included the addition of introns into the Tm DGAT1 ORF and the Si Cys-Ole ORF (Table 4, SEQ ID NO: 40). The complete T-DNA was contained within the binary vector pZY101 (Zeng et al., 2004).

### Example 5

Generation of construct (C5) containing the P. sativum rbcS promoter (Table 4, SEQ ID NO: 3) driving SiCys-Ole (Table 4, SEQ ID NO: 9) and P. sativum rbcS promoter (Table 4, SEQ ID NO: 3) driving TmDGAT1-V5 (Table 4, SEQ ID NO: 7+V5) in a tandem orientation for transformation into soybean.

The open reading frames of T. majus DGAT1-V5 and S. indicum Cys-Ole were optimized for expression in G. max; this included the addition of introns into the Tm DGAT1 ORF and the Si Cys-Ole ORF (Table 4, SEQ ID NO: 41). The complete T-DNA was contained within the binary vector pZY101 (Zeng et al., 2004).

### Example 6

Generation of a construct (C6) containing a G. max rbcS promoter (Table 4, SEQ ID NO: 4) and a P. sativum rbcS promoter (Table 4, SEQ ID NO: 3) driving SiCys-Ole (Table 4, SEQ ID NO: 9) and TmDGAT1 (Table 4, SEQ ID NO: 7) respectively, in a tandem orientation for transformation into soybean.

The open reading frames of T. majus DGAT1-V5 and S. indicum Cys-Ole were optimized for expression in G. max; this included the addition of introns into the Tm DGAT1 ORF and the Si Cys-Ole ORF (Table 4, SEQ ID NO: 42). The complete T-DNA was contained within the binary vector pZY101 (Zeng et al., 2004).

### Example 7

Generation of a construct (C7) containing a P. sativum rbcS promoter (Table 4, SEQ ID NO: 3) and a G. max rbcS promoter (Table 4, SEQ ID NO: 4) driving SiCys-Ole (Table 4, SEQ ID NO: 9) and TmDGAT1 (Table 4, SEQ ID NO: 7) respectively, in a tandem orientation for transformation into soybean.

The open reading frames of T. majus DGAT1 and S. indicum Cys-Ole were optimized for expression in G. max; this included the addition of introns into the Tm DGAT1 ORF and the Si Cys-Ole ORF (Table 4, SEQ ID NO: 43). The complete T-DNA was contained within the binary vector pZY101 (Zeng et al., 2004).

### Example 8

Generation of a construct (C8) containing G. max CAB3 (Table 4, SEQ ID NO: 5) and CAB6 (Table 4, SEQ ID NO: 6) promoters driving GmOle1 (Table 4, SEQ ID NO: 13) and GmDGAT1 (Table 4, SEQ ID NO: 11) respectively, for transformation into soybean.

The open reading frames of G. max DGAT1 (Roesler et al., 2016) and G. maxOle1 were optimized for expression in G. max (Table 4, SEQ ID NO: 44). The complete T-DNA was contained within a binary vector.

### Example 9

Generation of a construct (C9) containing G. max CAB3 (Table 4, SEQ ID NO: 5) and CAB6 (Table 4, SEQ ID NO: 6) promoters driving Soy4 (Table 4, SEQ ID NO: 15) and GmDGAT1 (Table 4, SEQ ID NO: 11) respectively, for transformation into soybean.

The open reading frames of G. max DGAT1 (Roesler et al., 2016) and Soy4 were optimized for expression in G. max (Table 4, SEQ ID NO: 45). The complete T-DNA was contained within a binary vector.

### Example 10

Generation of a construct (C10) containing G. max CAB3 (Table 4, SEQ ID NO: 5) and CAB6 (Table 4, SEQ ID NO: 6) promoters driving Soy5 (Table 4, SEQ ID NO: 17) and GmDGAT1 (Table 4, SEQ ID NO: 11) respectively, for transformation into soybean.

The open reading frames of G. max DGAT1 (Roesler et al., 2016) and Soy5 were optimized for expression in G. max (Table 4, SEQ ID NO: 46). The complete T-DNA was contained within a binary vector.

### Example 11

Generation of a construct (C11) containing G. max CAB3 (Table 4, SEQ ID NO: 5) and CAB6 (Table 4, SEQ ID NO: 6) promoters driving Soy6 (Table 4, SEQ ID NO: 19) and GmDGAT1 (Table 4, SEQ ID NO: 11) respectively, for transformation into soybean.

The open reading frames of G. max DGAT1 (Roesler et al., 2016) and Soy6 were optimized for expression in G. max (Table 4, SEQ ID NO: 47). The complete T-DNA was contained within a binary vector.

### Example 12

Generation of a construct (C12) containing G. max CAB3 (Table 4, SEQ ID NO: 5) and CAB6 (Table 4, SEQ ID NO: 6) promoters driving Soy23 (Table 4, SEQ ID NO: 21) and GmDGAT1 (Table 4, SEQ ID NO: 11) respectively, for transformation into soybean.

The open reading frames of G. max DGAT1 (Roesler et al., 2016) and Soy23 were optimized for expression in G. max (Table 4, SEQ ID NO: 48). The complete T-DNA was contained within a binary vector.

### Example 13

Generation of a construct (C13) containing G. max CAB3 (Table 4, SEQ ID NO: 5) and CAB6 (Table 4, SEQ ID NO: 6) promoters driving Soy24 (Table 4, SEQ ID NO: 23) and GmDGAT1 (Table 4, SEQ ID NO: 11) respectively, for transformation into soybean.

The open reading frames of G. max DGAT1 (Roesler et al., 2016) and Soy24 were optimized for expression in G. max (Table 4, SEQ ID NO: 49). The complete T-DNA was contained within a binary vector.

### Example 14

Generation of a construct (C14) containing G. max CAB3 (Table 4, SEQ ID NO: 5) and CAB6 (Table 4, SEQ ID NO: 6) promoters driving Soy7 (Table 4, SEQ ID NO: 25) and GmDGAT1 (Table 4, SEQ ID NO: 11) respectively, for transformation into soybean.

The open reading frames of G. max DGAT1 (Roesler et al., 2016) and Soy7 were optimized for expression in G. max (Table 4, SEQ ID NO: 50). The complete T-DNA was contained within a binary vector.

### Example 15

Generation of a construct (C15) containing G. max CAB3 (Table 4, SEQ ID NO: 5) and CAB6 (Table 4, SEQ ID NO: 6) promoters driving Soy25 (TABLE 1, SEQ ID NO: 27) and GmDGAT1 (Table 4, SEQ ID NO: 11) respectively, for transformation into soybean.

The open reading frames of G. max DGAT1 (Roesler et al., 2016) and Soy25 were optimized for expression in G. max (Table 4, SEQ ID NO: 51). The complete T-DNA was contained within a binary vector.

### Example 16

Generation of a construct (C16) containing G. max CAB3 (Table 4, SEQ ID NO: 5) and CAB6 (Table 4, SEQ ID NO: 6) promoters driving Soy17 (Table 4, SEQ ID NO: 29) and GmDGAT1 (Table 4, SEQ ID NO: 11) respectively, for transformation into soybean.

The open reading frames of G. max DGAT1 (Roesler et al., 2016) and Soy17 were optimized for expression in G. max (Table 4, SEQ ID NO: 52). The complete T-DNA was contained within a binary vector.

### Example 17

Generation of a construct (C17) containing G. max CAB3 (Table 4, SEQ ID NO: 5) and CAB6 (Table 4, SEQ ID NO: 6) promoters driving Soy18 (Table 4, SEQ ID NO: 31) and GmDGAT1 (Table 4, SEQ ID NO: 11) respectively, for transformation into soybean.

The open reading frames of G. max DGAT1 (Roesler et al., 2016) and Soy18 were optimized for expression in G. max (Table 4, SEQ ID NO: 53). The complete T-DNA was contained within a binary vector.

### Example 18

Generation of a construct (C18) containing G. max CAB3 (Table 4, SEQ ID NO: 5) and CAB6 (Table 4, SEQ ID NO: 6) promoters driving Soy19 (Table 4, SEQ ID NO: 33) and GmDGAT1 (Table 4, SEQ ID NO: 11) respectively, for transformation into soybean.

The open reading frames of G. max DGAT1 (Roesler et al., 2016) and Soy19 were optimized for expression in G. max (Table 4, SEQ ID NO: 54). The complete T-DNA was contained within a binary vector.

### Example 19

Generation of a construct (C19) containing G. max CAB3 (Table 4, SEQ ID NO: 5) and CAB6 (Table 4, SEQ ID NO: 6) promoters driving Soy20 (Table 4, SEQ ID NO: 35) and GmDGAT1 (Table 4, SEQ ID NO: 11) respectively, for transformation into soybean.

The open reading frames of G. max DGAT1 (Roesler et al., 2016) and Soy20 were optimized for expression in G. max (Table 4, SEQ ID NO: 55). The complete T-DNA was contained within a binary vector.

### Example 20

Promoter selection for regulation of DGAT and oleosins in Glycine max. The first report of expressing DGAT1 and cysteine oleosin in planta utilized CaMV35s promoters to regulate expression; this resulted in accumulation of lipids in the roots and leaves of Arabidopsis as well as elevated CO₂ assimilation by the leaves (Winichayakul et al., 2013). More recently, monocotyledonous green tissue preferred promoters were used to regulate the expression of DGAT and cysteine oleosins in Lolium perenne (perennial ryegrass). Ryegrass accumulated additional lipids in the leaves but not the roots, and when grown as individual plants the rate of CO₂ assimilation was elevated compared to control plants (Beechey-Gradwell et al., 2020; Cooney et al., 2021).

Here, we have compared a constitutive CaMV35S promoter with a variety of leguminous green tissue preferred promoters for expression of DGAT1 and both native and cysteine oleosins. The predicted expression patterns of the selected green tissue preferred promoters were generated by initially BLAST searching the promoter regions in Phytozome 13 using Glycine max Wm82.a2.v1 as the target. The function of the predicted downstream transcript was used as a confirmation and the specific gene name was used to recover the expression data in two independent soybean expression data bases, i.e., Soybean Expression Atlas (Machado et al., 2020) and Soybase.Org (Severin et al., 2010). The Phyotozome gene name corresponded to the Wn82.a2.v1 format which had to be converted to the Wm82.al.v1 format for searching Soybase.org. The appropriate gene names for the different formats are shown in Table 5.

Soybase.org noted that for the tissue-specific analyses, raw digital gene expression counts were normalized using a variation of the reads/Kb/Million (RPKM) method. Where the RPKM method corrects for biases in total gene exon size and normalizes for the total short read sequences obtained in each tissue library. In comparison, Soybean Expression Atlas normalized based on Transcripts per Million (TPM). Both RPKM and TPM are reported to take into account the number of reads from a gene depends on its length (more reads from longer length) and that the number of reads from a gene depends on the sequencing depth (total number of reads that were sequenced). Again, the more reads would be expected from a greater depth.

The tissues/organs analysed in Soybase.org are shown in Table 6; and the tissues analysed in Soybean Expression Atlas are shown in Table 7. The predicted expression patterns from Soybase.org and Soybean Expression Atlas for the promoters used here are shown in Tables 8 and 9, respectively. As expected, the highest expression for the green tissue promoters was seen in the young leaves (Table 8) and in the seedling and leaves (Table 9). Both expression data bases showed that the predicted expression from these promoters was also relatively high in two additional organs, flowers and the pod (in particular the shell).

### Example 21

### Transformation of Glycine max.

The constructs C1, C2, C3, C4, C5, C6, C7, C20 and C21 were transformed into Glycine max using Agrobacterium-mediated transformation according to the protocols outlined in Zhang et al., (1999).

The constructs C8, C9, C10, C11, C12, C13, C14, C15, C16, C17, C18, and C19 were transformed into Glycine max using Ochrobactrum-mediated transformation according to the protocols outlined in Anand et al., (2016, patent EP3341483B1).

The constructs C22, C23, C24, C25, C26, C27 and C28 were transformed into Glycine max using Agrobacterium-mediated transformation (U.S. Pat. No. 6,384,301 B1).

Lines containing a single locus for the T-DNA were selected for field trials based on one or several of the following changes measured in the leaf (as per Winchayakul et al, 2013): accumulation of cysteine oleosin; increase in the C18:2 fat content relative to the C18:3 fat content; increase in the overall fat content of the leaf. Lines were allowed to self in the glasshouse, the segregation ratio was noted and for each of the lines selected T₁ homozygous and T₁ null seed were selfed to produce sufficient T₂ seed for short row field trials.

### Example 22

### Field Trials.

Field evaluation was conducted at in Missouri. The soil series was a Putnam silt loam (fine, smectitic, mesic Vertic Albaqualfs). Soil was collected, air-dried, ground using a hammer mill to pass through a stainless steel sieve with 2 mm openings, and was analysed by the University of Missouri's Soil and Plant Testing Laboratory for soil characterization using standardized soil test procedures (Nathan et al., 2006).

The field was maintained weed-free through chemical and mechanical (hand-weeding) removal.

The majority of comparisons were short row field trials using a split-plot (paired comparison) arrangement (transgenic vs null) with the paired comparisons arranged in a complete randomised block within each plot. Replication of the complete plots varied between 2-5 depending on seed availability. Also dependent on seed availability was the lengths of the short rows which consisted of two 244-457 cm rows, 76 cm apart. Rows were planted at a seeding rate of approximately 346,00 seeds/hectare.

Seeds were counted and packaged for planting (John Deere planter with Almaco cone units). Soybean were treated with appropriate crop protection chemicals (seed treatment and in-season fungicide, insecticide, herbicides, etc.) for a high yielding environment. Supplemental irrigation was provided based on the Woodruff irrigation scheduling chart (Henggeler, 2008). In 2016 nitrogen was supplied at approximately 45 kg ha⁻¹ in the form of anhydrous ammonium; in 2017 nitrogen was supplied at approximately 90 kg ha⁻¹ as a polymer coated urea. From 2018 and onwards no additional nitrogen was applied in the field except what was present in the phosphorus formulation (monoammonium phosphate), i.e., approximately 12 kg N ha⁻¹.

Plots were harvested with a small plot combine (Wintersteiger Delta, 4 910 Ried, Austria, Dimmelstrasse 9) and samples were collected from the entire plot. Samples were cleaned using an Almaco air blast seed cleaner (Nevada, IA) and analyzed for protein and oil concentration using near-infrared spectroscopy (Foss Infratec 1241 Grain Analyzer, Eden Prairie, MN).

The constructs C1, C2, C3, C4, C5, C6 and C7 were compared against their nulls within separate plantings and as such the significant differences are indicated (where appropriate) for each line (Table 10). Where * indicates a significant difference between the transgenic line and the Null at the 10% probability level, while ns indicates a non-significant difference between the transgenic line and the Null at the 10% probability level.

The constructs C8, C9, C10, C11, C12, C13, C14, C15, C16, C17, C18, and C19 were compared against their nulls but as a grouped planting and as such the significant differences are indicated (where appropriate) for the averages (Table 11). Where * indicates a significant difference between the average of the transgenic lines and the average of the nulls at the 10% probability level, while ns indicates a non-significant difference between the average of the transgenic lines and the averages of the Nulls at the 10% probability level (Table 11).

Data were subjected to either ANOVA (SAS Institute, 2016) and means separated using Fisher's Protected LSD at P=0.1, or compared by Student's T-test (Microsoft Excel V2108) and the means separated by Fishers Least Significant Difference Test at P=0.05.

### Example 23

### Seed Analysis Results

Seeds from field grown plants expressing constructs from Examples 1-19 were analysed for oil content, protein content (Tables 10 and 11).

Table 10 shows that seeds from plants expressing any of the constructs C1, C2, C3, C4, C5, C6 and C7 (Examples 1, 2, 3, 4, 5, 6, and 7), all contained significantly higher oil content than the corresponding null while the protein contents were not significantly different compared to the corresponding null.

Table 11 shows that seeds from transgenic plants expressing any of the constructs C8, C9, C10, C11, C12, C13, C14, C15, C16, C17, C18, and C19 (Examples 8, 9, 10, 11, 12, 13, 14, 15, 16, 17, 18, and 19) all contained more oil and protein than the relevant null controls. Further, student's T-test across all lines expressing constructs C8-C19 shows the % oil and % protein were significantly higher in the seeds of the transgenic lines than in the seeds of the null lines. It should be noted that C8 contains a native oleosin and not a cysteine oleosin.

The C1, C2, C3, C4, C5, C6 and C7 constructs used combinations of the Glycine max RuBisCo, Pisum sativum CAB, and Pisum sativum RuBisCo promoters (lower predicted transcript levels, Table 8). In comparison, the C8, C9, C10, C11, C12, C13, C14, C15, C16, C17, C18, and C19 constructs used the Glycine max CAB3 and Glycine max CAB6 promoters (higher predicted transcript levels, Table 8). On average, the percentage increase in oil and protein content in the seed compared to null siblings for the constructs C2, C3, C4, C5, C6 and C7 (Table 10) was 6.4 and 0.7% respectively. In comparison, the average increases in seed oil and protein for the constructs C8, C9, C10, C11, C12, C13, C14, C15, C16, C17, C18, and C19 (Table 11) were 7.8 and 2.3% respectively.

### Example 24

Identification of Vigna angularis and Vigna radiata orthologous sequences to Glycine max CAB3, CAB6 and RBCS promoters.

Vigna orthologues to Glycine max CAB3 Promoter

NCBI BLAST search using the GmCAB3 promoter (Table 4, SEQ ID NO: 5) matched the 3′ 251 bases to Glycine max CAB3 5′ upstream region for PSII LHCII chlorophyll a/b binding protein (accession X12981.1). Phytozome12 (https://phytozome.jgi.doe.gov/pz/portal.html) BLAST search found 100% match for complete the promoter sequence as Glyma.08G082900|Chr08:6276579..6277935 (forward direction); this sequence was first published as the assembled genome in 2010. CAB3 was first reported by Walling et a., 1988 and was the most abundant of the CAB proteins they reported on.

The Vigna angularis genome (https://plants.ensembl.org/) was searched with the Glycine max CAB3 promoter sequence. The closest sequence found was a 58% match (Table 4, SEQ ID NO: 158). As per the Glycine max genome, immediately downstream of the promoter was the complete CAB3 ORF (no introns). Alignment of the translated peptide sequence was 98% match to G. max CAB3.

Similarly, the Vigna radiaia genome (https://plants.ensembl.org/) was searched with the Glycine max CAB3 promoter sequence. The closest sequence found was a 58% match (Table 4, SEQ ID NO: 159). As per the Glycine max genome, immediately downstream of the promoter was the complete CAB3 ORF (no introns). Alignment of the translated peptide sequence was 97% match to G. max CAB3.

Vigna orthologues to Glycine max CAB6 Promoter

NCBI BLAST search using the GmCAB6 promoter (Table 4, SEQ ID NO: 6) found only partial matches to Vigna angularis accession AP015036.1 and Vigna unguiculata accession CP039351.1; both were matched to the 3′ end of the promoter. Phytozome12 (https://phytozome.jgi.doe.gov/pz/portal.html) BLAST search found a match for complete the promoter sequence Glycine max Chr14:618008 . . . 619460 (-strand). This sequence was first published as the assembled genome in 2010. Translation of the sequence downstream of the Photozome12 sequence and BLAST searching the peptide sequence confirms that the protein is CAB6 identified by Walling et al., (2001) Accession M97171.1.

Potential alternatives to G. max CAB6 promoter were investigated in both Vigna angularis and Vigna radiata genomes which are searchable at https://plants.ensembl.org/. BLAST searches using ˜200 bp of the 3′ end of the GmCAB6 promoter found matches in both genomes, translation of the downstream regions found proteins that matched CAB6.

Aligning the 1453 bp length of the CAB6 promoter over the same region in the V. angularis and V. radiata genomes gave 58% and 59% identity, respectively. The promoter sequences and the 5′UTR sequences for CAB6 from both V. angularis and V. radiata are shown in Table 4, sequence 160 and 161, respectively.

Vigna orthologues to Glycine max RBCS Promoter

Used Glycine max rbcS promoter sequence (Table 4, SEQ ID NO: 4) in two BLAST searches against the Vigna angularis and Vigna radiata genomes at https://plants.ensembl.org/. Confirmed the appropriate sequences were selected by translating the peptide sequence immediately downstream of the 3′ end of the Vigna promoters to obtain the first exon translation; then used this exon to find the full-length peptides which were then aligned with the same peptide from Glycine max for confirmation.

From this analysis found the Vigna angularis RBCS promoter (Table 4, SEQ ID NO: 162) and the Vigna radiata RBCS promoter (Table 4, SEQ ID NO: 163).

### Example 25

Generation of a construct (C20) containing A. thaliana UBQ10 (Table 4, SEQ ID NO: 164) and G. max UBQ promoters (Table 4, SEQ ID NO: 165) driving S. indicum CYSTEINE-OLEOSIN (Table 4, SEQ ID NO: 9) and T. majus DGAT1 (Table 4, SEQ ID NO: 7+V5) respectively, for transformation into soybean.

The open reading frames of T. majus DGAT1 and S. indicum CYSTEINE-OLEOSIN were optimized for expression in G. max; this included the addition of introns into the Tm DGAT1 ORF and the S. indicum CYSTEINE-OLEOSIN ORF (Table 4, SEQ ID NO: 56).

### Example 26

Generation of a construct (C21) containing A. thaliana UBQ10 (Table 4, SEQ ID NO: 164) and G. max UBQ promoters (Table 4, SEQ ID NO: 165) driving T. majus DGAT1 (Table 4, SEQ ID NO: 7+V5) and S. indicum CYSTEINE-OLEOSIN (Table 4, SEQ ID NO: 9) respectively, for transformation into soybean.

The open reading frames of T. majus DGAT1 and S. indicum CYSTEINE-OLEOSIN were optimized for expression in G. max; this included the addition of introns into the Tm DGAT1 ORF and the S. indicum CYSTEINE-OLEOSIN ORF (Table 4, SEQ ID NO: 57).

### Example 27

Generation of a construct (C22) containing V. angularis RBCS (Table 4, SEQ ID NO: 162) and G. max RBCS (Table 4, SEQ ID NO: 4) promoters driving T. majus DGAT1 (Table 4, SEQ ID NO: 7) and Soy4 (Table 4, SEQ ID NO: 15) respectively, for transformation into soybean.

The open reading frames of T. majus DGAT1 and Soy4 were optimized for expression in G. max; this included the addition of introns into the Tm DGAT1 ORF and Soy4 ORF (Table 4, SEQ ID NO: 166).

### Example 28

Generation of a construct (C23) containing V. angularis RBCS (Table 4, SEQ ID NO: 162) and G. max RBCS (Table 4, SEQ ID NO: 4) promoters driving T. majus DGAT1 (Table 4, SEQ ID NO: 7) and Soy17 (Table 4, SEQ ID NO: 29) respectively, for transformation into soybean.

The open reading frames of T. majus DGAT1 and Soy17 were optimized for expression in G. max; this included the addition of introns into the Tm DGAT1 ORF and Soy17 ORF (Table 4, SEQ ID NO: 167).

### Example 29

Generation of a construct (C24) containing V. angularis RBCS (Table 4, SEQ ID NO: 162) and G. max RBCS (Table 4, SEQ ID NO: 4) promoters driving T. majus DGAT1 (Table 4, SEQ ID NO: 7) and Soy25 (Table 4, SEQ ID NO: 27) respectively, for transformation into soybean.

The open reading frames of T. majus DGAT1 and Soy25 were optimized for expression in G. max; this included the addition of introns into the Tm DGAT1 ORF and Soy25 ORF (Table 4, SEQ ID NO: 168).

### Example 30

Generation of a construct (C25) containing P. sativum RBCS (Table 4, SEQ ID NO: 3) and G. max RBCS (Table 4, SEQ ID NO: 4) promoters driving T. majus DGAT1 (Table 4, SEQ ID NO: 7) and Soy25 (Table 4, SEQ ID NO: 27) respectively, for transformation into soybean.

The open reading frames of T. majus DGAT1 and Soy25 were optimized for expression in G. max; this included the addition of introns into the Tm DGAT1 ORF and Soy25 ORF (Table 4, SEQ ID NO: 169).

### Example 31

Generation of a construct (C26) containing V. angularis RBCS (Table 4, SEQ ID NO: 162) and G. max RBCS (Table 4, SEQ ID NO: 4) promoters driving T. majus DGAT1 (Table 4, SEQ ID NO: 7) and Soy20 (Table 4, SEQ ID NO: 35) respectively, for transformation into soybean.

The open reading frames of T. majus DGAT1 and Soy20 were optimized for expression in G. max; this included the addition of introns into the Tm DGAT1 ORF and Soy20 ORF (Table 4, SEQ ID NO: 170).

### Example 32

Generation of a construct (C27) containing V. angularis CAB6 and CAB3 promoters (Table 4, SEQ ID NOs: 160 and 158 respectively) driving Soy20 (Table 4, SEQ ID NO: 35) and T. majus DGAT1 (Table 4, SEQ ID NO: 7) respectively, for transformation into soybean.

The open reading frames of T. majus DGAT1 and Soy20 were optimized for expression in G. max; this included the addition of introns into the Tm DGAT1 ORF and Soy20 ORF (Table 4, SEQ ID NO: 171).

### Example 33

Generation of a construct (C28) containing V. angularis CAB6 and CAB3 promoters (Table 4, SEQ ID NOs: 160 and 158 respectively) driving T. majus DGAT1 (Table 4, SEQ ID NO: 7) and Soy20 (Table 4, SEQ ID NO: 35), for transformation into soybean.

The open reading frames of T. majus DGAT1 and Soy20 were optimized for expression in G. max; this included the addition of introns into the Tm DGAT1 ORF and Soy20 ORF (Table 4, SEQ ID NO: 172).

### Example 34

Field trials with plants transformed with constructs containing Ubiquitin promoters as well as plants transformed with constructs containing Vigna RBCS promoters.

Field evaluation was conducted at in Missouri. The soil series was a Putnam silt loam (fine, smectitic, mesic Vertic Albaqualfs). Soil was collected, air-dried, ground using a hammer mill to pass through a stainless steel sieve with 2 mm openings, and was analysed by the University of Missouri's Soil and Plant Testing Laboratory for soil characterization using standardized soil test procedures (Nathan et al., 2006).

The field was maintained weed-free through chemical and mechanical (hand-weeding) removal.

The majority of comparisons were short row field trials using a split-plot (paired comparison) arrangement (transgenic vs null) with the paired comparisons arranged in a complete randomised block within each plot. Replication of the complete plots varied between 2-5 depending on seed availability. Also dependent on seed availability was the lengths of the short rows which consisted of two 244-457 cm rows, 76 cm apart. Rows were planted at a seeding rate of approximately 346,00 seeds/hectare.

Seeds were counted and packaged for planting (John Deere planter with Almaco cone units). Soybean were treated with appropriate crop protection chemicals (seed treatment and in-season fungicide, insecticide, herbicides, etc.) for a high yielding environment. Supplemental irrigation was provided based on the Woodruff irrigation scheduling chart (Henggeler, 2008).

No additional nitrogen was applied in the field except what was present in the phosphorus formulation (monoammonium phosphate), i.e., approximately 12 kg N ha⁻¹.

Plots were harvested with a small plot combine (Wintersteiger Delta, 4 910 Ried, Austria, Dimmelstrasse 9) and samples were collected from the entire plot. Samples were cleaned using an Almaco air blast seed cleaner (Nevada, IA) and analyzed for protein and oil concentration using near-infrared spectroscopy (Foss Infratec 1241 Grain Analyzer, Eden Prairie, MN).

Constructs can be compared against their nulls for % oil and % protein. Data can be subjected to either ANOVA (SAS Institute, 2016) and means separated using Fisher's Protected LSD at P=0.1, or compared by Student's T-test (Microsoft Excel V2108) and the means separated by Fishers Least Significant Difference Test at P=0.05.

### Example 35

Seed Analysis Results from plants transformed with constructs containing Ubiqutin promoters as well as plants transformed with constructs containing Vigna RBCS promoters.

Seeds from field grown plants expressing constructs from Examples 25, 26, 27, 28, 29, 30 and 31 can be analysed for oil content and protein content as discussed above.

Tables 12 and 13 show that seeds from plants expressing any of the constructs C21, C22, C23 and C26 (Examples 25, 26, 27, 28, 29, 30 and 31), all contained a higher oil content than the corresponding null while the protein contents were not significantly different or greater than the corresponding null.

Constructs C20, C24, C25, and C26 (Examples 25, 26, 27, 28, 29, 30 and 31) can analysed in the same way. Tables 12 and 13 show the expected range for % oil and % protein for these constructs and their nulls.

### Example 36

Seed Analysis Results from plants transformed with constructs containing Vigna angularis CAB3 and CAB6 promoters

Seeds from glasshouse grown plants expressing constructs from Examples 32, and 33 can be analysed for oil and protein content, as discussed above. Table 14 shows the expected ranges for % oil and % protein constructs C27 and C28 and their nulls.

TABLE 4
promoter, DGAT and oleosin sequences used for expression

| | | Sequence | SEQ |
|---|---|---|---|
| Abbreviation | Description | type | ID |
| CaMV35s | CaMV35s promoter | nucleotide | 1 |
| PsCAB | Pisum sativum CAB promoter | nucleotide | 2 |
| PsrbcS | Pisum sativum Rbcs-3A promoter | nucleotide | 3 |
| GmrbcS | Glycine max RuBisCO promoter | nucleotide | 4 |
| GmCAB3 | Glycine max CAB3 promoter | nucleotide | 5 |
| GmCAB6 | Glycine max CAB6 promoter | nucleotide | 6 |
| TmDGAT | DGAT1 from Tropaeolum majus with S197A | nucleotide | 7 |
| | mutation | | |
| TmDGAT | DGAT1 from Tropaeolum majus with S197A | peptide | 8 |
| | mutation | | |
| SiOLE | Sesamum indicum cysteine oleosin | nucleotide | 9 |
| SiOLE | Sesamum indicum cysteine oleosin | peptide | 10 |
| GmDGAT | Glycine max DGAT1 with 14 residue changes | nucleotide | 11 |
| GmDGAT | Glycine max DGAT1 with 14 residue changes | peptide | 12 |
| GmOle1 | Glycine max native oleosin (accession | nucleotide | 13 |
| | NP_001358853) | | |
| GmOle1 | Glycine max native oleosin (accession | peptide | 14 |
| | NP_001358853) | | |
| Soy4 | Glycine max cysteine oleosin - variant 4 | nucleotide | 15 |
| Soy4 | Glycine max cysteine oleosin - variant 4 | peptide | 16 |
| Soy5 | Glycine max cysteine oleosin - variant 5 | nucleotide | 17 |
| Soy5 | Glycine max cysteine oleosin - variant 5 | peptide | 18 |
| Soy6 | Glycine max cysteine oleosin - variant 6 | nucleotide | 19 |
| Soy6 | Glycine max cysteine oleosin - variant 6 | peptide | 20 |
| Soy23 | Glycine max cysteine oleosin - variant 23 | nucleotide | 21 |
| Soy23 | Glycine max cysteine oleosin - variant 23 | peptide | 22 |
| Soy24 | Glycine max cysteine oleosin - variant 24 | nucleotide | 23 |
| Soy24 | Glycine max cysteine oleosin - variant 24 | peptide | 24 |
| Soy7 | Glycine max cysteine oleosin - variant 7 | nucleotide | 25 |
| Soy7 | Glycine max cysteine oleosin - variant 7 | peptide | 26 |
| Soy25 | Glycine max cysteine oleosin - variant 25 | nucleotide | 27 |
| Soy25 | Glycine max cysteine oleosin - variant 25 | peptide | 28 |
| Soy17 | Glycine max cysteine oleosin - variant 17 | nucleotide | 29 |
| Soy17 | Glycine max cysteine oleosin - variant 17 | peptide | 30 |
| Soy18 | Glycine max cysteine oleosin - variant 18 | nucleotide | 31 |
| Soy18 | Glycine max cysteine oleosin - variant 18 | peptide | 32 |
| Soy19 | Glycine max cysteine oleosin - variant 19 | nucleotide | 33 |
| Soy19 | Glycine max cysteine oleosin - variant 19 | peptide | 34 |
| Soy20 | Glycine max cysteine oleosin - variant 20 | nucleotide | 35 |
| Soy20 | Glycine max cysteine oleosin - variant 20 | peptide | 36 |
| C1 | 2 CaMV35s promoters driving Tm DGAT1 and | nucleotide | 37 |
| | SiOle | | |
| | RIGHT BORDER 116-140 | | |
| | CaMV35s -PROMOTER 426-873 | | |
| | 5′UTR 874-937 | | |
| | SiOLE 938-1567 | | |
| | S. tuberosum ST-LS1 INTRON 2 964-1152 | | |
| | 35S/NOS TERMINATOR 1568-2099 | | |
| | CaMV35s -PROMOTER 2100-2547 | | |
| | 5′UTR 2548-2611 | | |
| | TmDGAT1 (S197A) 2612-4462 | | |
| | S. tuberosum ST-LS1 INTRON 2 2636-2824 | | |
| | 35S/NOS TERMINATOR 4463-4994 | | |
| | MCS 5001-5030 | | |
| | CaMV35s PROMOTER 5031-5470 | | |
| | BAR RESISTANCE GENE 5622-6191 | | |
| | LEFT BORDER 6973-6997 | | |
| C2 | P. sativum CAB and rbcS promoters in a back to | nucleotide | 38 |
| | back orientation driving T. majus DGAT1-V5 | | |
| | (+intron) and S. indicum Cys-Ole (+intron) | | |
| | (respectively) | | |
| | RIGHT BORDER 116-140 | | |
| | 35s-NOS TERMINATOR 426-896 | | |
| | TmDGAT1 (S197A) 957-2782 | | |
| | Gm vspB INTRON 2 2596-2758 | | |
| | PsCAB PROMOTER 2847-3245 | | |
| | PsrbcS PROMOTER 3252-3683 | | |
| | SiOLE 3694-4324 | | |
| | S. tuberosum ST-LS1 INTRON 2 3741-3929 | | |
| | Gm vspB TERMINATOR 4330-4902 | | |
| | MULTIPLE CLONING SITE 4908-4937 | | |
| | CaMV35s PROMOTER 4938-5377 | | |
| | BAR RESISTANCE GENE 5529-6098 | | |
| | LEFT BORDER 6880-6904 | | |
| C3 | P. sativum CAB and rbcS promoters in a tandem | nucleotide | 39 |
| | orientation driving T. majus DGAT1-V5 (+intron) | | |
| | and S. indicum Cys-Ole (+intron) (respectively) | | |
| | RIGHT BORDER 116-140 | | |
| | PsCAB PROMOTER 426-824 | | |
| | TmDGAT1 (S197A) 889-2714 | | |
| | Gm vspB INTRON 2 913-1075 | | |
| | 35s/NOS TERMINATOR 2775-3245 | | |
| | PsrbcS PROMOTER 3252-3683 | | |
| | SiOLE 3694-4184 | | |
| | S. tuberosum ST-LS1 INTRON 2 3741-3929 | | |
| | Gm vspB TERMINATOR 4190-4762 | | |
| | MULTIPLE CLONING SITE 4768-4797 | | |
| | CaMV35s PROMOTER 4798-5237 | | |
| | BAR RESISTANCE GENE 5389-5958 | | |
| | LEFT BORDER 6740-6764 | | |
| C4 | 2 P. sativum Rbcs promoters driving S. indicum | | 40 |
| | Cys-Ole (+intron) and T. majus DGAT1 (+intron) | | |
| | RIGHT BORDER 116-140 | | |
| | PsrbcS PROMOTER 427-858 | | |
| | PsrbcS 5′UTR 838-858 | | |
| | 5′ UTR 867-929 | | |
| | SiOLE 930-1559 | | |
| | S. tuberosum ST-LS1 INTRON 2 984-1172 | | |
| | Gm vspB TERMINATOR 1569-2141 | | |
| | PsrbcS PROMOTER 2153-2584 | | |
| | PsrbcS 5′ UTR 2564-2584 | | |
| | Tm DGAT1-V5 (S197A) 2601-4323 | | |
| | Gm vspB INTRON 2 2625-2787 | | |
| | 35S/NOS TERMINATOR 4391-4861 | | |
| | MCS 4867-4901 | | |
| | CaMV35s PROMOTER 4902-5341 | | |
| | BAR RESISTANCE GENE 5493-6062 | | |
| | LEFT BORDER 6844-6868 | | |
| C5 | 2 P. sativum Rbcs promoters driving S. indicum | nucleotide | 41 |
| | Cys-Ole (+intron) and T. majusDGAT1-V5 | | |
| | (+intron) | | |
| | RIGHT BORDER 116-140 | | |
| | PsrbcS PROMOTER 427-858 | | |
| | PsrbcS 5′UTR 838-858 | | |
| | 5′ UTR 867-929 | | |
| | SiOLE 930-1559 | | |
| | S. tuberosum ST-LS1 INTRON 2 984-1172 | | |
| | Gm vspB TERMINATOR 1569-2141 | | |
| | PsrbcS PROMOTER 2153-2584 | | |
| | PsrbcS 5′ UTR 2564-2584 | | |
| | Tm DGAT1-V5 (S197A) 2601-4426 | | |
| | Gm vspB INTRON 2 2625-2787 | | |
| | 35S/NOS TERMINATOR 4493-4963 | | |
| | MCS 4969-5003 | | |
| | CaMV35s PROMOTER 5004-5443 | | |
| | BAR RESISTANCE GENE 5595-6164 | | |
| | LEFT BORDER 6946-6970 | | |
| C6 | G. max rbcS promoter and a P. sativum rbcs | nucleotide | 42 |
| | promoter driving S. indicum Cys-Ole (+intron) and | | |
| | T. majus DGAT1 (+intron) (respectively) | | |
| | RIGHT BORDER 116-140 | | |
| | Gm RBCS SR1 PROMOTER 428-1891 | | |
| | 5′UTR 1900-1962 | | |
| | SiOLE 1963-2592 | | |
| | S. tuberosum ST-LS1 INTRON 2 2017-2205 | | |
| | Gm vspB TERMINATOR 2602-3174 | | |
| | PsrbcS PROMOTER 3186-3617 | | |
| | PsrbcS 5′ UTR 2597-3617 | | |
| | TmDGAT1-V5 (S197A) 3634-5459 | | |
| | Gm vspB INTRON 2 3658-3820 | | |
| | 35S/NOS TERMINATOR 5526-5996 | | |
| | MCS 6002-6036 | | |
| | CaMV35s PROMOTER 6037-6476 | | |
| | BAR RESISTANCE GENE 6628-7197 | | |
| | LEFT BORDER 7979-8003 | | |
| C7 | G. max rbcS promoter and a P. sativum rbcS | nucleotide | 43 |
| | promoter driving T. majus DGAT1 (+intron) and S. | | |
| | indicum Cys-Ole (+intron) (respectively) | | |
| | RIGHT BORDER 116-140 | | |
| | PsrbcS PROMOTER 427-858 | | |
| | PsrbcS 5′ UTR 838-858 | | |
| | 5′ UTR 867-929 | | |
| | SiOLE 930-1559 | | |
| | S. tuberosum ST-LS1 INTRON 2 984-1172 | | |
| | Gm vspB TERMINATOR 1569-2141 | | |
| | GmrbcS PROMOTER 2143-3606 | | |
| | TmDGAT1-V5 (S197A) 3624-5449 | | |
| | Gm vspB INTRON 2 3648-3810 | | |
| | 35S/NOS TERMINATOR 5516-5986 | | |
| | MCS 5992-6026 | | |
| | CaMV35s PROMOTER 6027-6466 | | |
| | BAR RESISTANCE GENE 6618-7187 | | |
| | LEFT BORDER 7969-7993 | | |
| C8 | G. max CAB3 promoter driving GmDGAT1 G. max | | 44 |
| | CAB6 promoter driving GmOle1 | | |
| | LEFT BORDER 1-25 | | |
| | Gm CAB3 PROMOTER 1109-2141 | | |
| | Gm DGAT1 2153-3667 | | |
| | UBQ14 TERMINATOR 3688-4589 | | |
| | Gm CAB6 PROMOTER 4765-6217 | | |
| | Gm CAB6 5′ UTR 6218-6341 | | |
| | GmOle1 6358-6801 | | |
| | UBQ10 TERMINATOR 6822-7701 | | |
| | RIGHT BORDER 9197-9221 | | |
| C9 | G. max CAB3 promoter driving GmDGAT1 G. max | | 45 |
| | CAB6 promoter driving Soy4 | | |
| | LEFT BORDER 1-25 | | |
| | Gm CAB3 PROMOTER 1109-2141 | | |
| | Gm DGAT1 2153-3667 | | |
| | UBQ14 TERMINATOR 3688-4589 | | |
| | Gm CAB6 PROMOTER 4765-6217 | | |
| | Gm CAB6 5′ UTR 6218-6341 | | |
| | Soy4 4546-51826358-6801 | | |
| | UBQ10 TERMINATOR 6822-7701 | | |
| | RIGHT BORDER 9197-9221 | | |
| C10 | G. max CAB3 promoter driving GmDGAT1 G. max | | 46 |
| | CAB6 promoter driving Soy5 | | |
| | LEFT BORDER 1-25 | | |
| | Gm CAB3 PROMOTER 1109-2141 | | |
| | Gm DGAT1 2153-3667 | | |
| | UBQ14 TERMINATOR 3688-4589 | | |
| | Gm CAB6 PROMOTER 4765-6217 | | |
| | Gm CAB6 5′ UTR 6218-6341 | | |
| | Soy5 6358-6801 | | |
| | UBQ10 TERMINATOR 6822-7701 | | |
| | RIGHT BORDER 9197-9221 | | |
| C11 | G. max CAB3 promoter driving GmDGAT1 G. max | | 47 |
| | CAB6 promoter driving Soy6 | | |
| | LEFT BORDER 1-25 | | |
| | Gm CAB3 PROMOTER 1109-2141 | | |
| | Gm DGAT1 2153-3667 | | |
| | UBQ14 TERMINATOR 3688-4589 | | |
| | Gm CAB6 PROMOTER 4765-6217 | | |
| | Gm CAB6 5′ UTR 6218-6341 | | |
| | Soy6 6358-6801 | | |
| | UBQ10 TERMINATOR 6822-7701 | | |
| | RIGHT BORDER 9197-9221 | | |
| C12 | G. max CAB3 promoter driving GmDGAT1 G. max | | 48 |
| | CAB6 promoter driving Soy23 | | |
| | LEFT BORDER 1-25 | | |
| | Gm CAB3 PROMOTER 1109-2141 | | |
| | Gm DGAT1 2153-3667 | | |
| | UBQ14 TERMINATOR 3688-4589 | | |
| | Gm CAB6 PROMOTER 4765-6217 | | |
| | Gm CAB6 5′ UTR 6218-6341 | | |
| | Soy23 6358-6801 | | |
| | UBQ10 TERMINATOR 6822-7701 | | |
| | RIGHT BORDER 9197-9221 | | |
| C13 | G. max CAB3 promoter driving GmDGAT1 G. max | | 49 |
| | CAB6 promoter driving Soy24 | | |
| | LEFT BORDER 1-25 | | |
| | Gm CAB3 PROMOTER 1109-2141 | | |
| | Gm DGAT1 2153-3667 | | |
| | UBQ14 TERMINATOR 3688-4589 | | |
| | Gm CAB6 PROMOTER 4765-6217 | | |
| | Gm CAB6 5′ UTR 6218-6341 | | |
| | Soy24 6358-6801 | | |
| | UBQ10 TERMINATOR 6822-7701 | | |
| | RIGHT BORDER 9197-9221 | | |
| C14 | G. max CAB6 promoter driving GmDGAT1 G. max | nucleotide | 50 |
| | CAB3 promoter driving Soy7 | | |
| | LEFT BORDER 1-25 | | |
| | Gm CAB6 PROMOTER 1109-2561 | | |
| | Gm CAB6 5′ UTR 2562-2685 | | |
| | Gm DGAT1 2702-4216 | | |
| | UBQ14 TERMINATOR 4237-5138 | | |
| | Gm CAB3 PROMOTER 5314-6346 | | |
| | Soy7 6358-6801 | | |
| | UBQ10 TERMINATOR 6822-7701 | | |
| | RIGHT BORDER 9197-9221 | | |
| C15 | G. max CAB6 promoter driving GmDGAT1 G. max | nucleotide | 51 |
| | CAB3 promoter driving Soy25 | | |
| | LEFT BORDER 1-25 | | |
| | Gm CAB6 PROMOTER 1109-2561 | | |
| | Gm CAB6 5′ UTR 2562-2685 | | |
| | Gm DGAT1 2702-4216 | | |
| | UBQ14 TERMINATOR 4237-5138 | | |
| | Gm CAB3 PROMOTER 5314-6346 | | |
| | Soy25 6358-6801 | | |
| | UBQ10 TERMINATOR 6822-7701 | | |
| | RIGHT BORDER 9197-9221 | | |
| C16 | G. max CAB6 promoter driving GmDGAT1 G. max | nucleotide | 52 |
| | CAB3 promoter driving Soy17 | | |
| | LEFT BORDER 1-25 | | |
| | Gm CAB6 PROMOTER 1109-2561 | | |
| | Gm CAB6 5′ UTR 2562-2685 | | |
| | Gm DGAT1 2702-4216 | | |
| | UBQ14 TERMINATOR 4237-5138 | | |
| | Gm CAB3 PROMOTER 5314-6346 | | |
| | Soy17 6358-6801 | | |
| | UBQ10 TERMINATOR 6822-7701 | | |
| | RIGHT BORDER 9197-9221 | | |
| C17 | G. max CAB6 promoter driving GmDGAT1 G. max | nucleotide | 53 |
| | CAB3 promoter driving Soy18 | | |
| | LEFT BORDER 1-25 | | |
| | Gm CAB6 PROMOTER 1109-2561 | | |
| | Gm CAB6 5′ UTR 2562-2685 | | |
| | Gm DGAT1 2702-4216 | | |
| | UBQ14 TERMINATOR 4237-5138 | | |
| | Gm CAB3 PROMOTER 5314-6346 | | |
| | Soy18 6358-6801 | | |
| | UBQ10 TERMINATOR 6822-7701 | | |
| | RIGHT BORDER 9197-9221 | | |
| C18 | G. max CAB6 promoter driving GmDGAT1 G. max | nucleotide | 54 |
| | CAB3 promoter driving Soy19 | | |
| | LEFT BORDER 1-25 | | |
| | Gm CAB6 PROMOTER 1109-2561 | | |
| | Gm CAB6 5′ UTR 2562-2685 | | |
| | Gm DGAT1 2702-4216 | | |
| | UBQ14 TERMINATOR 4237-5138 | | |
| | Gm CAB3 PROMOTER 5314-6346 | | |
| | Soy19 6358-6801 | | |
| | UBQ10 TERMINATOR 6822-7701 | | |
| | RIGHT BORDER 9197-9221 | | |
| C19 | G. max CAB6 promoter driving GmDGAT1 G. max | nucleotide | 55 |
| | CAB3 promoter driving Soy20 | | |
| | LEFT BORDER 1-25 | | |
| | Gm CAB6 PROMOTER 1109-2561 | | |
| | Gm CAB6 5′ UTR 2562-2685 | | |
| | Gm DGAT1 2702-4216 | | |
| | UBQ14 TERMINATOR 4237-5138 | | |
| | Gm CAB3 PROMOTER 5314-6346 | | |
| | Soy20 6358-6801 | | |
| | UBQ10 TERMINATOR 6822-7701 | | |
| | RIGHT BORDER 9197-9221 | | |
| C20 | A thaliana UBQ10 promoter driving S. indicum | nucleotide | 56 |
| | CYSTEINE OLEOSIN, G. max UBQ promoter | | |
| | driving T. majus DGAT1. | | |
| | RIGHT BORDER 116-140 | | |
| | At UBQ10 PROMOTER 426-1759 | | |
| | 5′ UTR 1768-1830 | | |
| | SiOLE 1831-2460 | | |
| | S. tuberosum ST-LS1 INTRON 2 1885-2073 | | |
| | Gm vspB TERMINATOR 2469-3041 | | |
| | Gm UBQ PROMOTER 3048-4991 | | |
| | Tm DGAT1-V5 (S197A) 5008-6833 | | |
| | Gm vspB INTRON2 5032-5194 | | |
| | UBQ14 TERMINATOR 6840-7741 | | |
| | MULTIPLE CLONING SITE 7743-7777 | | |
| | CaMV35s PROMOTER 7778-8217 | | |
| | BAR RESISTANCE GENE 8369-8938 | | |
| | LEFT BORDER 9720-9744 | | |
| C21 | A thaliana UBQ10 promoter driving T. majus | nucleotide | 57 |
| | DGAT1, G. max UBQ promoter driving S. indicum | | |
| | CYSTEINE OLEOSIN. | | |
| | RIGHT BORDER 116-140 | | |
| | At UBQ10 PROMOTER 426-1759 | | |
| | Tm DGAT1-V5 (S197A) 1776-3601 | | |
| | Gm vspB INTRON2 1800-1962 | | |
| | Gm vspB TERMINATOR 3608-4180 | | |
| | Gm UBQ PROMOTER 4187-6130 | | |
| | 5′UTR 6139-6201 | | |
| | SiOLE 6202-6831 | | |
| | S. tuberosum ST-LS1 INTRON 2 6256-6444 | | |
| | At UBQ14 TERMINATOR 6839-7740 | | |
| | MULTIPLE CLONING SITE 7742-7776 | | |
| | CaMV35s PROMOTER 7777-8216 | | |
| | BAR RESISTANCE GENE 8368-8937 | | |
| | LEFT BORDER 9719-9743 | | |
| VaCAB3 | Vigna angularis CAB3 promoter | nucleotide | 158 |
| VrCAB3 | Vigna radiata CAB3 promoter | nucleotide | 159 |
| VaCAB6 | Vigna angularis CAB6 promoter | nucleotide | 160 |
| VrCAB6 | Vigna radiata CAB6 promoter | nucleotide | 161 |
| VaRBCS | Vigna angularis RBCS promoter | nucleotide | 162 |
| VrRBCS | Vigna radiata RBCS promoter | nucleotide | 163 |
| AtUBQ | Arabidopsis thaliana UBQ10 promoter | nucleotide | 164 |
| GmUBQ | Glycine max UBQ promoter | nucleotide | 165 |
| C22 | V. angularis RBCS PROMOTER DRIVING T. | nucleotide | 166 |
| | majus DGAT1(+intron); G. max RBCS | | |
| | PROMOTER DRIVING Soy4 (+intron). | | |
| | LEFT BORDER 1-25 | | |
| | A. thaliana ACT7\3U TERMINATOR 114-507 | | |
| | ADENYLTRANSFERASE (aadA1) 512-1304 | | |
| | A. thaliana transit peptide 1305-1499 | | |
| | G. max UBI3XLP5U PROMOTER 1504-3926 | | |
| | G. max vspB TERMINATOR 3967-4539 | | |
| | Soy4 4546-5182 | | |
| | S. tuberosum ST-LS1 INTRON 2 4940-5182 | | |
| | 5′ UTR 5183-5245 | | |
| | GmrbcS PROMOTER 5254-6717 | | |
| | UBQ14 TERMINATOR 6724-7625 | | |
| | Tm DGAT1 (S197A) 7635-9357 | | |
| | Gm vspB INTRON 2 9171-9333 | | |
| | VarbcS PROMOTER 9374-10850 | | |
| | RIGHT BORDER 10912-10936 | | |
| C23 | V. angularis RBCS PROMOTER DRIVING T. | nucleotide | 167 |
| | majus DGAT1(+intron); G. max RBCS | | |
| | PROMOTER DRIVING Soy17 (+intron). | | |
| | LEFT BORDER 1-25 | | |
| | A. thaliana ACT7\3U TERMINATOR 114-507 | | |
| | ADENYLTRANSFERASE (aadA1) 512-1304 | | |
| | A. thaliana transit peptide 1305-1499 | | |
| | G. max UBI3XLP5U PROMOTER 1504-3926 | | |
| | G. max vspB TERMINATOR 3967-4539 | | |
| | Soy17 4546-5182 | | |
| | S. tuberosum ST-LS1 INTRON 2 4940-5182 | | |
| | 5′ UTR 5183-5245 | | |
| | GmrbcS PROMOTER 5254-6717 | | |
| | A. thaliana UBQ14 TERMINATOR 6724-7625 | | |
| | Tm DGAT1 (S197A) 7635-9357 | | |
| | Gm vspB INTRON 2 9171-9333 | | |
| | Va rbcS PROMOTER 9374-10850 | | |
| | RIGHT BORDER 10912-10936 | | |
| C24 | V. angularis RBCS PROMOTER DRIVING T. | nucleotide | 168 |
| | majus DGAT1(+intron); G. max RBCS | | |
| | PROMOTER DRIVING Soy25 (+intron). | | |
| | LEFT BORDER 1-25 | | |
| | A. thaliana ACT7\3U TERMINATOR 114-507 | | |
| | ADENYLTRANSFERASE (aadA1) 512-1304 | | |
| | A. thaliana transit peptide 1305-1499 | | |
| | G. max UBI3XLP5U PROMOTER 1504-3926 | | |
| | Gm vspB TERMINATOR 3967-4539 | | |
| | Soy25 4546-5182 | | |
| | S. tuberosum ST-LS1 INTRON 2 4940-5182 | | |
| | 5′ UTR 5183-5245 | | |
| | Gm rbcS PROMOTER 5254-6717 | | |
| | A. thaliana UBQ14 TERMINATOR 6724-7625 | | |
| | Tm DGAT1 (S197A) 7635-9357 | | |
| | Gm vspB INTRON 2 9171-9333 | | |
| | Va rbcS PROMOTER 9374-10850 | | |
| | RIGHT BORDER 10912-10936 | | |
| C25 | P. sativum RBCS PROMOTER DRIVING T. | nucleotide | 169 |
| | majus DGAT1(+intron); G. max RBCS | | |
| | PROMOTER DRIVING Soy25 (+intron). | | |
| | LEFT BORDER 1-25 | | |
| | At ACT7\3U TERMINATOR 114-507 | | |
| | ADENYLTRANSFERASE (aadA1) 512-1304 | | |
| | At transit peptide 1305-1499 | | |
| | Gm UBI3 PROMOTER 1504-3926 | | |
| | Gm vspB TERMINATOR 3967-4539 | | |
| | Soy25 4546-5182 | | |
| | S. tuberosum ST-LS1 INTRON 2 4940-5182 | | |
| | 5′ UTR 5183-5245 | | |
| | GmrbcS PROMOTER 5254-6717 | | |
| | UBQ14 TERMINATOR 6724-7625 | | |
| | Tm DGAT1 (S197A) 7635-9357 | | |
| | Gm vspB INTRON 2 9171-9333 | | |
| | 5′ UTR 9374-9394 | | |
| | Ps rbcS PROMOTER 9374-9805 | | |
| | RIGHT BORDER 9867-9891 | | |
| C26 | V. angularis RBCS PROMOTER DRIVING T. | nucleotide | 170 |
| | majus DGAT1(+intron); G. max RBCS | | |
| | PROMOTER DRIVING Soy20(+intron). | | |
| | LEFT BORDER 1-25 | | |
| | A. thaliana ACT7\3U TERMINATOR 114-507 | | |
| | ADENYLTRANSFERASE (aadA1) 512-1304 | | |
| | A. thaliana transit peptide 1305-1499 | | |
| | G. max UBI3XLP5U PROMOTER 1504-3926 | | |
| | Gm vspB TERMINATOR 3967-4539 | | |
| | Soy20 4546-5182 | | |
| | S. tuberosum ST-LS1 INTRON 2 4940-5182 | | |
| | 5′ UTR 5183-5245 | | |
| | Gm rbcS PROMOTER 5254-6717 | | |
| | A. thaliana UBQ14 TERMINATOR 6724-7625 | | |
| | Tm DGAT1 (S197A) 7635-9357 | | |
| | Gm vspB INTRON 2 9171-9333 | | |
| | Va rbcS PROMOTER 9374-10850 | | |
| | RIGHT BORDER 10912-10936 | | |
| C27 | V. angularis CAB6 PROMOTER DRIVING | nucleotide | 171 |
| | Soy20; V. angularis CAB3 PROMOTER | | |
| | DRIVING T. majus DGAT1. | | |
| | LEFT BORDER 1-25 | | |
| | At ACT7 TERMINATOR 114-507 | | |
| | ADENYLTRANSFERASE (aadA1) 512-1303 | | |
| | At transit peptide 1305-1499 | | |
| | GmUBI3 PROMOTER 1504-3926 | | |
| | UBQ10 TERMINATOR 4012-4891 | | |
| | Soy20 4892-5528 | | |
| | S. tuberosum ST-LS1 INTRON 2 5286-5474 | | |
| | Va CAB6 PROMOTER 5529-7107 | | |
| | 5′ UTR 5529-5645 | | |
| | UBQ14 TERMINATOR 7112-8013 | | |
| | Tm DGAT1 (S197A) 8014-9736 | | |
| | Gm vspB INTRON 2 9550-9712 | | |
| | Va CAB3 PROMOTER 9737-10786 | | |
| | RIGHT BORDER 10850-10874 | | |
| C28 | V. angularis CAB3 PROMOTER DRIVING | nucleotide | 172 |
| | Soy20; V. angularis CAB6 PROMOTER | | |
| | DRIVING T. majus DGAT1. | | |
| | LEFT BORDER 1-25 | | |
| | At ACT7\3U TERMINATOR 114-507 | | |
| | ADENYLTRANSFERASE (aadA1) 512-1303 | | |
| | At transit peptide 1305-1499 | | |
| | Gm UBI3 PROMOTER 1504-3926 | | |
| | UBQ10 TERMINATOR 4012-4891 | | |
| | Soy20 4892-5528 | | |
| | S. tuberosum ST-LS1 INTRON 2 5286-5474 | | |
| | Va CAB3 PROMOTER 5529-6578 | | |
| | UBQ14 TERMINATOR 6583-7484 | | |
| | Tm DGAT1 (S197A) 7485-9207 | | |
| | Gm vspB INTRON 2 9021-9183 | | |
| | VaCAB6 PROMOTER 9208-10786 | | |
| | 5′UTR 9208-9333 | | |
| | RIGHT BORDER 10850-10874 | | |

TABLE 5
Glycine max and orthologous Pisum sativum CAB and RuBisCO gene identification numbers
used to search public domain Glycine max data bases (Soybase.org and Phytozome).

| Gene Description and species | | | | |
|---|---|---|---|---|
| source | Wm82.a1.v1 | Wm82.a1.v1.1 | Wm82.a2.v1 | Wm82.a4.v1 |
| CAB3 G. max | Glyma08g08770 | Glyma08g08770 | Glyma.08g082900 | Glyma.08g082900 |
| CAB6 G. max | Glyma12g01130 | Glyma12g01130 | Glyma.14G008000 | Glyma.12g008700 |
| RuBisCO G. max | Glyma13g07610 | Glyma13g07610 | Glyma.13g046200 | Glyma.13g046200 |
| CAB P. sativum | Glyma16g28070 | Glyma16g28070 | Glyma.16g165800 | Glyma.16g165800 |
| RuBisCO P. sativum | Glyma18g53430 | Glyma18g53430 | Glyma.18g296900 | Glyma.18g296900 |

TABLE 6
Tissues and organs analysed by RNA-SEQ at Soybase.org

| | | | | | | | | | | | | | |
|---|---|---|---|---|---|---|---|---|---|---|---|---|---|
| young | flower | one | pod | pod | seed | seed | seed | seed | seed | seed | seed | root | nodule |
| leaf | | cm | shell | shell | 10DAF | 14DAF | 21DAF | 25DAF | 28DAF | 35DAF | 42DAF | | |
| | | pod | 10DAF | 14DAF | | | | | | | | | |

TABLE 7
Tissues and organs analysed by RNA-SEQ at Soybean Expression Atlas

| | | | | | | | |
|---|---|---|---|---|---|---|---|
| CALLUS | SEEDLING | LEAVES | FLOWER | POD | SUSPENSOR | EMBRYO | COTYLEDON |

| | | | | | | |
|---|---|---|---|---|---|---|
| ENDOSPERM | HYPOCOTYL | SEED | SEED | SHOOT | ROOT | NODULE |
| | | | COAT | | | |

TABLE 8
RNA-SEQ results from Soybase.org for CAB and RuBisCo transcripts

| | | PROMOTER | | | | | | | | |
|---|---|---|---|---|---|---|---|---|---|---|
| | | AND/OR | | TISSUE/ORGAN | | | | | | |
| | | TRANSCRIPT | | | | one | pod | pod | | |
| PROMOTER | | NCBI | EQUIVALENT | young | | cm | shell | shell | seed | seed |
| SPECIES | GENE | ACCESSION | Wm82.a1 | leaf | flower | pod | 10DAF | 14DAF | 10DAF | 14DAF |
| SOURCE | DESCRIPTION | NUMBER | GENE NAME | RELATIVE TRANSCRIPT EXPRESSION (soybase.org) | | | | | | |
| Glycine max | CAB 3 | NM 001254348.3 | Glyma08g08770 | 3789 | 873 | 1693 | 2167 | 1791 | 59 | 166 |
| Glycine max | CAB 6 | NM 001253254.2 | Glyma14g01130 | 1490 | 303 | 394 | 436 | 500 | 45 | 81 |
| Glycine max | RuBisCO | NM 001248385.2 | Glyma13g07610 | 828 | 80 | 158 | 193 | 332 | 6 | 13 |
| Pisum sativum | CAB | M64619.1 | Glyma16g28070 | 295 | 43 | 103 | 105 | 114 | 2 | 2 |
| Pisum sativum | RuBisCO | M21356.1 | Glyma18g53430 | 0 | 7 | 0 | 0 | 0 | 0 | 0 |

| | | PROMOTER | | | | | | | | |
|---|---|---|---|---|---|---|---|---|---|---|
| | | AND/OR | | | | | | | | |
| | | TRANSCRIPT | | TISSUE/ORGAN | | | | | | |
| PROMOTER | | NCBI | EQUIVALENT | seed | seed | seed | seed | seed | | |
| SPECIES | GENE | ACCESSION | Wm82.a1 | 21DAF | 25DAF | 28DAF | 35DAF | 42DAF | root | nodule |
| SOURCE | DESCRIPTION | NUMBER | GENE NAME | RELATIVE TRANSCRIPT EXPRESSION (soybase.org) | | | | | | |
| Glycine max | CAB 3 | NM 001254348.3 | Glyma08g08770 | 211 | 462 | 274 | 179 | 121 | 1 | 1 |
| Glycine max | CAB 6 | NM 001253254.2 | Glyma14g01130 | 72 | 106 | 64 | 78 | 35 | 2 | 0 |
| Glycine max | RuBisCO | NM 001248385.2 | Glyma13g07610 | 30 | 29 | 21 | 22 | 12 | 0 | 0 |
| Pisum sativum | CAB | M64619.1 | Glyma16g28070 | 5 | 7 | 9 | 2 | 1 | 0 | 0 |
| Pisum sativum | RuBisCO | M21356.1 | Glyma18g53430 | 0 | 0 | 0 | 0 | 0 | 0 | 1 |

TABLE 9
RNA-SEQ results from Soybean Expression Atlas for CAB and RuBisCo transcripts

| | | PROMOTER AND OR | | | | | | |
|---|---|---|---|---|---|---|---|---|
| | | TRANSCRIPT NCBI | EQUIVALENT | | | | | |
| PROMOTER | GENE | ACCESSION | Wm82.a1.v1GENE | | | | | |
| SPECIES SOURCE | DESCRIPTION | NUMBER | NAME | CALLUS | SEEDLING | LEAVES | FLOWER | POD |
| Glycine max | CAB 3 | NM_001254348.3 | Glyma.08g082900 | 94 | 11.8 | 10.9 | 11.0 | 11.4 |
| Glycine max | CAB 6 | NM_001253254.2 | Glyma.14G008000 | 3.9 | 10.3 | 9.5 | 8.5 | 9.2 |
| Glycine max | RuBisCO | NM_001248385.2 | Glyma.13g046200 | 4.8 | 12.6 | 12.4 | 9.5 | 10.3 |
| Pisum sativum | CAB | M64619.1 | Glyma.16g165800 | 4.2 | 8.6 | 7.6 | 7.1 | 8.3 |
| Pisum sativum | RuBisCO | M21356.1 | Glyma.18g296900 | 2.1 | 3.9 | 3.5 | 4.0 | 3.8 |

| | | PROMOTER AND OR | | | | | | |
|---|---|---|---|---|---|---|---|---|
| | | TRANSCRIPT NCBI | EQUIVALENT | | | | | |
| PROMOTER | GENE | ACCESSION | Wm82.a1.v1GENE | SUSPEN- | | | ENDO- | HYPO- |
| SPECIES SOURCE | DESCRIPTION | NUMBER | NAME | SOR | EMBRYO | COTYLEDON | SPERM | COTYL |
| Glycine max | CAB 3 | NM_001254348.3 | Glyma.08g082900 | 3.2 | 6.0 | 9.0 | 1.1 | 10.2 |
| Glycine max | CAB 6 | NM_001253254.2 | Glyma.14G008000 | 3.3 | 4.6 | 6.7 | 0.8 | 7.6 |
| Glycine max | RuBisCO | NM_001248385.2 | Glyma.13g046200 | 0.0 | 4.5 | 8.4 | 0.2 | 9.7 |
| Pisum sativum | CAB | M64619.1 | Glyma.16g165800 | 0.0 | 1.4 | 4.0 | 0.0 | 5.3 |
| Pisum sativum | RuBisCO | M21356.1 | Glyma.18g296900 | 3.8 | 2.8 | 2.1 | 3.5 | 2.6 |

| | | PROMOTER AND OR | | | | | | |
|---|---|---|---|---|---|---|---|---|
| | | TRANSCRIPT NCBI | EQUIVALENT | | | | | |
| PROMOTER | GENE | ACCESSION | Wm82.a1.v1GENE | | SEED- | | | |
| SPECIES SOURCE | DESCRIPTION | NUMBER | NAME | SEED | COAT | SHOOT | ROOT | NODULE |
| Glycine max | CAB 3 | NM_001254348.3 | Glyma.08g082900 | 6.2 | 6.9 | 8.7 | 5.4 | 2.7 |
| Glycine max | CAB 6 | NM_001253254.2 | Glyma.14G008000 | 3.9 | 4.0 | 2.1 | 2.3 | 0.0 |
| Glycine max | RuBisCO | NM_001248385.2 | Glyma.13g046200 | 5.1 | 4.4 | 9.0 | 3.8 | 0.6 |
| Pisum sativum | CAB | M64619.1 | Glyma.16g165800 | 2.1 | 2.3 | 4.5 | 0.9 | 0.0 |
| Pisum sativum | RuBisCO | M21356.1 | Glyma.18g296900 | 1.8 | 3.6 | 2.2 | 2.3 | 0.6 |

TABLE 10
Average seed oil content and seed protein content
for constructs described in Examples 1-7.

| | | | % OIL | | % OIL cf | % PROTEIN | | % Prot cf |
|---|---|---|---|---|---|---|---|---|
| CONSTRUCT | LINE | YEAR | Trans | Null | Null | Trans | Null | Null |
| C1 | C1-1 | 2016 | 21.2 * | 19.0 | 11.6 | 36.5 ns | 36.3 | 0.8 |
| C2 | C2-1 | 2017 | 20.6 * | 19.3 | 6.8 | 35.6 ns | 35.1 | 1.4 |
| C2 | C2-1 | 2018a | 22.3 * | 20.2 | 10.4 | 34.4 ns | 33.7 | 2.1 |
| C3 | C3-1 | 2017 | 20.1 * | 19.0 | 6.0 | 35.6 ns | 35.4 | 0.6 |
| C3 | C3-2 | 2017 | 20.1 * | 18.6 | 8.1 | 35.6 ns | 35.5 | 0.1 |
| C3 | C3-2 | 2018a | 21.4 * | 20.1 | 6.5 | 33.4 ns | 33.5 | −0.3 |
| C3 | C3-2 | 2018b | 21.6 * | 20.2 | 6.9 | 33.8 ns | 33.8 | 0.0 |
| C4 | C4-1 | 2019 | 19.3 * | 19.0 | 1.6 | 36.4 ns | 35.9 | 1.4 |
| C5 | C5-1 | 2019 | 20.3 * | 19.1 | 6.3 | 36.0 ns | 35.3 | 2.0 |
| C6 | C6-1 | 2020 | 19.4 * | 18.7 | 3.2 | 34.9 ns | 34.6 | 0.9 |
| C7 | C7-1 | 2020 | 19.9 * | 18.5 | 7.6 | 34.9 ns | 35.4 | −1.4 |

* indicates significant difference to null via ANOVA

TABLE 11
Average seed oil content and seed protein content
for constructs described in Examples 8-19.

| | | | | | % OIL | % PROTEIN | | % Prot cf |
|---|---|---|---|---|---|---|---|---|
| CONSTRUCT | LINE | YEAR | Trans | Null | cf Null | Trans | Null | Null |
| C8 | C8-14 | 2020 | 19.9 | 18.7 | 6.4 | 35.6 | 35.3 | 0.8 |
| C9 | C9-35 | 2020 | 20.7 | 18.4 | 12.5 | 35.1 | 34.6 | 1.4 |
| C9 | C9-10 | 2020 | 20.4 | 19.0 | 7.4 | 36.0 | 35.0 | 2.9 |
| C10 | C10-17 | 2020 | 20.6 | 19.3 | 6.7 | 35.8 | 35.0 | 2.3 |
| C10 | C10-23 | 2020 | 20.7 | 19.1 | 8.4 | 36.3 | 35.5 | 2.3 |
| C11 | C11-19 | 2020 | 20.6 | 19.2 | 7.3 | 35.9 | 35.0 | 2.6 |
| C11 | C11-17 | 2020 | 20.7 | 19.7 | 5.1 | 35.1 | 34.5 | 1.7 |
| C12 | C12-21 | 2020 | 21.2 | 19.7 | 7.6 | 35.2 | 34.6 | 1.7 |
| C12 | C12-10 | 2020 | 19.7 | 18.9 | 4.2 | 36.0 | 35.1 | 2.6 |
| C13 | C12-8 | 2020 | 20.8 | 19.3 | 7.8 | 35.4 | 35.0 | 1.1 |
| C13 | C12-30 | 2020 | 20.2 | 19.3 | 4.7 | 36.0 | 35.0 | 2.9 |
| C14 | C14-13 | 2020 | 20.2 | 18.6 | 8.6 | 36.7 | 35.3 | 4.0 |
| C15 | C15-49 | 2020 | 21.1 | 19.4 | 8.8 | 35.4 | 34.7 | 2.0 |
| C15 | C15-34 | 2020 | 21.1 | 19.0 | 11.1 | 35.7 | 34.9 | 2.3 |
| C16 | C16-63 | 2020 | 21.0 | 19.3 | 8.8 | 35.3 | 34.8 | 1.4 |
| C16 | C16-15 | 2020 | 20.5 | 19.3 | 6.2 | 35.7 | 34.7 | 2.9 |
| C17 | C17-1 | 2020 | 20.4 | 18.6 | 9.7 | 35.8 | 35.5 | 0.8 |
| C18 | C18-1 | 2020 | 20.2 | 19.4 | 4.1 | 36.8 | 35.2 | 4.5 |
| C18 | C18-2 | 2020 | 20.8 | 19.0 | 9.5 | 35.9 | 34.7 | 3.5 |
| C19 | C19-21 | 2020 | 21.7 | 19.8 | 9.6 | 35.4 | 34.5 | 2.6 |
| C19 | C19-25 | 2020 | 20.9 | 19.2 | 8.9 | 35.8 | 35.2 | 1.7 |
| Average all | | | 20.6* | 19.2 | 2.8 | 35.8* | 35.0 | 2.3 |
| lines | | | | | | | | |

*indicates significantly different than null via student's t-test

TABLE 12
Average seed oil and protein content for constructs described in Examples 25-26.

| | | | % OIL | | % OIL cf | % PROTEIN | | % Prot cf |
|---|---|---|---|---|---|---|---|---|
| CONSTRUCT | | YEAR | Trans | Null | Null | Trans | Null | Null |
| C20** | LINE | 2022 | 19.0-30 | 18.0-24.0 | 4-50 | 32.0-40.0 | 33.0-38.0 | 0.2-20.0 |

| | | | | | | | |
|---|---|---|---|---|---|---|---|
| C21 | 2022 | 21.6 | 20.6 | 5.1% | 34.5 | 34.4 | 0.2 |

**indicates expected range

TABLE 13
Average seed oil and protein content for constructs described in Examples 27-31.

| | | | % OIL | | % OIL cf | % PROTEIN | | % Prot cf |
|---|---|---|---|---|---|---|---|---|
| CONSTRUCT | | YEAR | Trans | Null | Null | Trans | Null | Null |
| C22 | LINE | 2022 | 21.3 | 19.8 | 7.6% | 35.0 | 33.6 | 4.4 |

| | | | | | | | |
|---|---|---|---|---|---|---|---|
| C23 | 2022 | 20.3 | 19.6 | 6.5% | 35.5 | 33.5 | 5.8% |
| C24** | 2022 | 19.0-30 | 18.0-24.0 | 4-50 | 32.0-40.0 | 33.0-38.0 | 0.2-20.0 |
| C25** | 2022 | 19.0-30 | 18.0-24.0 | 4-50 | 32.0-40.0 | 33.0-38.0 | 0.2-20.0 |
| C26 | 2022 | 21.4 | 19.8 | 7.9% | 38.4 | 33.7 | 5.1 |

**indicates expected range

TABLE 14
Average seed oil and protein content for constructs described in Examples 32 and 33.

| | | | % OIL | | % OIL cf | % PROTEIN | | 8% Prot cf |
|---|---|---|---|---|---|---|---|---|
| CONSTRUCT | LINE | YEAR | Trans | Null | Null | Trans | Null | Null |

| | | | | | | | |
|---|---|---|---|---|---|---|---|
| C27** | 2022 | 19.0-30 | 18.0-24.0 | 4.50 | 32.0-40.0 | 33.0-38.0 | 0.2-20.0 |
| C28** | 2022 | 19.0-30 | 18.0-24.0 | 4.50 | 32.0-40.0 | 33.0-38.0 | 0.2-20.0 |

**indicates expected range

### REFERENCES

Anand A, Bass S H, Bertain S M, Cho H-J, Kinney A J, Klein T M, Lassner M, McBride K E, Moy Y, Rosen B A M, Wei, J-Z (2016). Ochrobactrum-mediated transformation of Plants. International Patent PCT/US2016/049135.
Beechey-Gradwell Z, Cooney L, Winichayakul S, Andrews M, Hea S Y, Crowther T, Roberts N (2020). Storing carbon in leaf lipid sinks enhanced perennial ryegrass carbon capture especially under high N and elevated CO2. J Exp Bot. 71, 2351-2361.
Cooney L, Beechey-Gradwell Z, Winichayakul S, Richardson K A, Crowther T, Anderson P, Scott R W, Bryan G, Roberts N J (2021). Changes in leaf-level nitrogen partitioning and mesophyll conductance deliver increased photosynthesis for Lolium perenne leaves engineered to accumulate lipid carbon sinks. Fronters in Plant Science. doi: 10.3389/fpls.2021.641822
de Borja Reis A F, Tamagno S, Rosso L H M, Ortez O A, Naeve S, Clampitti I A (2020). Historical trend on seed amino acid concentration does not follow protein changes in soybeans. Sci Rep. 2020 Oct. 19; 10(1):17707. doi: 10.1038/s41598-020-74734-1.
Cho, H., Moy, Y., Rudnick, N. A., Klein, T. M., Yin, J., Bolar, J., Hendrick, C., Beatty, M., Casta-neda, L., Kinney, A. J., Jones, T. J. and Chilcoat, N. D. (2022) Development of an efficient marker-free soybean transformation method using the novel bacterium Ochrobactrum haywardense H1. Plant Biotechnol J., https://doi.org/10.1111/pbi.13777
Henggeler J (2009). Irrigation Scheduling. University of Missouri Extension. http://agebb.missouri.edu/drought/agriculture/irrigation/13IrrigationScheduling.pdf
Hymowitz T, Collins F I, Panczner J, Walker W M (1972). Relationship between the content of oil, protein, and sugar in soybean seed. Agronomy J. 64: 613-616.
Kambhampati S, Aznar-Moreno J A, Hostetler C, Caso T, Bailey S R, Hubbard A H, Durrett T, Allen D K (2020). On the inverse correlation of protein and oil: examining the effects of altered central carbon metabolism on seed composition using soybean fast neutron mutants. Metabolites 2020, 10, 18; doi:10.3390/metabo10010018
Machado F B, Moharana K C, Almeida-Silva F, Gazara R K, Pedrosa-Silva F, Coelho F S, Grativol C, Venancio T M (2020). Systematic analysis of 1,298 RNA-Seq samples and construction of a comprehensive soybean (Glycine max) expression atlas. The Plant Journal, 2020; doi: 2020: doi: https://doi.org/10.1111/tpj.14850
Mahmoud A A, Natarajan S S, Bennett J O, Mawhinney T P, Wiebold W J, Krishnan H B (2006). Effect of six decades of selective breeding on soybean protein composition and quality: A biochemical and molecular analysis. J. Agric. Food Chem. 54:3916-3922.
Nathan, M., J. Stecker, and Y. Sun. 2006. Soil testing in Missouri: A guide for conducting soil tests in Missouri. EC923. Missouri Cooperative Extension Service, Univ. of Missouri-Lincoln Univ., Columbia, M O. pp. 33.
Roesler K, Shen B, Bermudez E, Li C, Hunt J, Damude H G, Ripp K G, Everard J D, Boot J R, Castaneda L, Feng, Meyer K (2016). An improved variant of soybean type 1 diacyglycerol acyltransferase increases the oil content and decreases the soluble carbohydrate content of soybeans. Plant Phys. 171:878-893.
Severin A J, Woody J L, Bolon Y-T, Joseph B, Diers B W, Farmer A D, Muehlbauer G J, Nelson R T, Grant D, Specht J E, Graham M A, Cannon S B, May G D, Vance C P, Shoemaker R C (2010). RNA-Seq Atlas of Glycine max: A guide to the soybean transcriptome. BMC Plant Biology 10:160 http://www.biomedcentral.com/1471-2229/10/160.
Wilson E W, Rowntree S C, Suhre J J, Weidenbenner N H, Conley S P, Davis V M, Diers B W, Esker P D, Naeve S L, Specht J E, Casteel S N. (2013). Genetic Gain X Management Interactions in Soybean: I I. Nitrogen Utilization. Crop Sci. 54:340-348.
Zeng P, Vadnais D A, Zhang Z, Polacco J C. (2004). Refined glufosinate selection in Agrobacterium-mediated transformation of soybean [Glycine max (L.) Merrill]. Plant Cell Feb; 22(7):478-82. Epub 2003 Sep. 30. 10.1007/s00299-003-0712-8 PubMed 15034747
Zhang Z, Xing A, Staswick P, Clemente T. (1999). The use of glufosinate as a selective agent in Agrobacterium-mediated transformation of soybean. Plant Cell Tiss Org Cult 56: 3

What is claimed is:

1. (canceled)

2. A method for producing seed with increased oil content relative to that in seed of a control plant, or control seed, without significantly reduced protein content relative to that in seed of the control plant, or control seed, the method comprising:
a) ectopically expressing an oil-synthesising enzyme in a plant, wherein expression of the oil-synthesising enzyme is not seed-preferred expression, and
b) ectopically expressing an oil-encapsulating protein in the plant, wherein expression of the oil-encapsulating protein is not seed-preferred expression,
wherein the ectopic expression in a) and b) leads to the production of seed with increased the oil content, without significantly reduced protein content.

3. The method of claim 2 further including at least one of the steps of:
a) measuring the oil production or content in the seed, and/or
b) measuring the protein production or content in the seed.

4. (canceled)

5. The method of claim 2 which includes at least one of the steps of:
a) selecting the plant or seed based on measuring an increase in the oil production or content in the seed, and no reduction in the protein production or content in the seed, and/or
b) selecting the plant or seed based on measuring an increase in the oil production or content in the seed, and an increase in the protein production or content in the seed.

6. (canceled)

7. The method of claim 2, wherein at least one of the following applies:
a) the production or content of oil in the seeds of the plant is increased by at least 1%, and/or
b) the production or content of protein in the seeds of the plant is increased by at least 0.1%.

8. (canceled)

9. (canceled)

10. (canceled)

11. The method of claim 2 in which:
a expression of the oil synthesising enzyme is from a polynucleotide encoding the oil synthesising enzyme,
b) the polynucleotide encoding the oil synthesising enzyme is part of a construct comprising a promoter operably linked to the polynucleotide, and
c) the promoter drives non-seed-preferred expression of the polynucleotide.

12. (canceled)

13. (canceled)

14. (canceled)

15. The method of claim 2 in which:
a) expression of the oil encapsulating protein is from a polynucleotide encoding the oil encapsulating protein,
b) the polynucleotide encoding the oil encapsulating protein is part of a construct comprising a promoter operably linked to the polynucleotide, and
c) the promoter drives non-seed-preferred expression of the polynucleotide.

16. (canceled)

17. (canceled)

18. (canceled)

19. A seed produced by the method of claim 2.

20. (canceled)

21. (canceled)

22. The seed of claim 19 that is transgenic for a polynucleotide or construct encoding the oil-synthesising enzyme, and a polynucleotide or construct encoding the oil-encapsulating protein.

23. The seed of claim 22 that has increased oil content relative to that in seed of a control plant, or control seed, without reduced protein content relative to that in seed of the control plant, or control seed.

24. A plant with increased production or content of oil in its seed relative to that in a control plant, without significantly decreased production or content of protein in its seed relative to that in the control plant, wherein the plant:
a) ectopically expresses an oil-synthesising enzyme in the plant, wherein expression of the oil-synthesising enzyme is not seed-preferred expression, and
b) ectopically expresses an oil-encapsulating protein in the plant, wherein expression of the oil-encapsulating protein is not seed-preferred expression,
wherein the ectopic expression in a) and b) leads to the increased the production or content of oil in the seed, without the significantly decreased production or content of protein in the seed.

25. A method for producing a plant with increased production or content of oil in its seed relative to that in a control plant, without significantly decreased production or content of protein in its seed relative to that in the control plant, the method comprising crossing the plant of claim 24 with another plant.

26. A method for producing a seed with increased oil production or oil content relative to that in a control plant, without significantly decreased oil production or oil content relative to that in the control plant, the method comprising:
a) crossing a plant of claim 24 with another plant, and
b) harvesting the seed produced.

27. The plant of claim 24 in which production or content of oil in the seeds of the plant is increased by at least 1%.

28. The plant of claim 24 in which production or content of protein in the seeds of the plant is increased by at least 0.1%.

29. (canceled)

30. A method for producing oil, the method comprising extracting oil from the seed of claim 19.

31. A method for producing oil, the method comprising producing seed according to the method of claim 2 and extracting oil from the seed.

32. The method of claim 30 in which the oil extraction is by at least one of:
a) solvent extraction,
b) crushing, and
c) critical point extraction.

33. The method claim 32 in which the oil is processed into at least one of:
a) a fuel,
b) an oleochemical,
c) a nutritional oil,
d) a cosmetic oil,
e) a polyunsaturated fatty acid (PUFA), and
f) a combination of any of a) to e).

34. A method for producing a protein enriched co-product, the method comprising extracting oil from a seed of claim 19, and collecting the remaining protein-enriched co-product.

35. A method for producing a protein-enriched co-product, the method comprising producing seed according to the method of claim 2, extracting oil from the seed, and collecting the remaining protein-enriched-co-product.

36. The method of claim 34 in which extraction is by at least one of:
a) solvent extraction,
b) crushing, and
c) critical point extraction.

37. The method of claim 34 in which the protein-enriched co-product has a higher protein content than that produced from the seeds of a control plant, or control seeds.

38. A protein-enriched co-product produced by the method of claim 34.

39. An animal feedstock comprising a protein-enriched co-product of claim 38.

40. A food ingredient comprising a protein-enriched co-product of claim 38.

41. A plant part, propagule, progeny or seed of the plant of claim 24 that is transgenic for a polynucleotide or construct encoding the oil-synthesising enzyme, and a polynucleotide or construct encoding the oil-encapsulating protein.


SEQUENCE LISTING (180 sequences)

SEQ ID NO: 1 - DNA, 456 bp, synthetic construct
  source 1..456: mol_type = other DNA
ttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatcccc

SEQ ID NO: 2 - DNA, 399 bp, Pisum sativum
  source 1..399: mol_type = genomic DNA
cagctgtccataacagtcacaatttttctcaaatcttgtggctctcaaacactgtataaaacatgacaaatgtggacccaaaatagagaaagcatagccccataaattaagccattttatgaaaaaataatattatgttgagtcatatatccataagaatccccacagtcacacatggaagagcagcattggatacaaatgatatgaagattttgatccatgaacggattctagaattgcaaagaaaatctccaactagccatagctttagataacacacgataagagcatctgcattataaatacagactcatattcatcttacaaaatcaccattgataaggatacaattatcaaaagcataacaatcttttcaatttcattgcaatataatacacg

SEQ ID NO: 3 - DNA, 435 bp, Pisum sativum
  source 1..435: mol_type = genomic DNA
gatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaatg

SEQ ID NO: 4 - DNA, 1463 bp, Glycine max
  source 1..1463: mol_type = genomic DNA
ctagtagtggaagtggtgcagaagaggggagggaatgtttggggtacatgatttgaaaaaaggggagaatgattagaagagatagaaaattggcagaccaacattgaaggattcataggaatgttgtgtgttagtaaattggacaaatgttgctcttgatattttcagcatatcattcatcttagaagcccaactgttcttttttatatatattctctgttgccttcctttccttcaaaacacggcttttgaattttgaaccattaactgagttggaatagtggtaaaatcatgtagatttttcttttcgttttgtggggatatgaatacgaaatatccttacaaccagacaaagctgtggctcagaagaaagagaaacatgcctcactgctagaatcactagaactcacatcttgtatacaaactgttataccaaactgagaattttacaatttccaaagatgcaattctcaaccattaaccaatttcatcttaaacactaacagttcttcagtcttgaactttccatgctgaaaatttgttgcctatttcttttataggcagtggcttcttatgagagtcacactctctgaacttcttacgtaatacttgacttttcattttgtgctttataattgttatctcatgcattgcacttttaagataaggttgttgtgcattgtttggttgagtgcaacgatttactgattaccatagctacttgtggatcttatattttcaaaagtgtatgcttggtcattaatcaaccaagcatgataagccccatcatctaccaccccttctataccatatacgcattttgcttatctacattgctactgttacactcaaacaagtctaacagataatacgttaagttaatgaacatttttagtaatattattataaggattggccaatgtaattgtgaagagagaagcatgctttaagctacccaacaaaatggataagagtttcagttgatatggttctcttgtttcttttccaataaaaaaccaactttaaaatataaaatttactgtaacaaaggaacaaagagttttcacttaatccatgaatgagaaaggatggtcacaaaatatgttaggttaatatggaatgagggcactgtgcaaactacacaaataatatcaattccaccaccatcacacatttacgttcttccaaggaagagataagataatggagcctccacgtgtcacctccacatggtacctaacaataaggctaccattcaaaattttcctcactcgtgtggcctatatgctgtaatgtcatcacttattcaatccaacggttgtaacttttcggcaaccaatcctctccatttcacaccattggattagtactacacaaatcacactattatatatagcaagtttgagcagaagcttggatatctggcagcagaaaaacaagtagttgagaactaagaagaagga

SEQ ID NO: 5 - DNA, 1033 bp, Glycine max
  source 1..1033: mol_type = genomic DNA
cttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaa

SEQ ID NO: 6 - DNA, 1453 bp, synthetic construct
  source 1..1453: mol_type = other DNA
gcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgag

SEQ ID NO: 7 - DNA, 1663 bp, synthetic construct
  source 1..1663: mol_type = other DNA
atggccgtggctgagtcatctcaaaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaggctaagggcgagcttaggggacatccattcgagggaaagccaattccaaacccacttctcggactcgattctaccagaaccggacatcatcatcaccaccactaatgaa

SEQ ID NO: 8 - protein, 518 aa, synthetic construct
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEK

SEQ ID NO: 9 - DNA, 441 bp, synthetic construct
  source 1..441: mol_type = other DNA
atggcttgccattacggacaacaacaacaaacctgcgctccacaccttcaacttcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatga

SEQ ID NO: 10 - protein, 145 aa, synthetic construct
MACHYGQQQQTCAPHLQLQPRACRVVKAATAVTAGGSLLVLSGLTLAGTVIALTIATPLLVIFSPVLVPAVITIFLLGAGFLASGGFGVAALSVLSWIYRYLTGKHPPGADCLESAKTKLASCAREMKDRAEQFSCQPVAGSQTS

SEQ ID NO: 11 - DNA, 1515 bp, synthetic construct
  source 1..1515: mol_type = other DNA
atggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactga

SEQ ID NO: 12 - protein, 504 aa, synthetic construct
MAISDEPESVATALNHSSLRRRPSATSTAGLFNSPETTTDSSGDDLAKDSGSDDSINNDDAAVNSQQQNEKQDTDFSVLKFAYRPSVPAHRKVKESPLSSDTIFRQSHAGLFNLCIVVLVAVNSRLIIENLMKYGWLIKSGFWFSAKSLRDWPLFMCCLSLVVFPFAAFMVEKLAQRKCIPEPVVVVLHIIITSTSLFYPVLVILKCDSAFVSGVTLMLFSCVVWLKLVSFAHTNYDMRALTKLVEKGEALLDTLNMEYPYNVTFKSLAYFLLAPTLCYQPSYPRTPYIRKGWLFRQLVKLIVFTGVMGFIIEQYINPIVQNSQHPLKGNLLYATERVLKLSVPNLYVWLCMFYCFFHLWLNIVAELLRFGDREFYKDWWNAKTVEDYWRMWNMPVHKWMIRHLYFPCLRHGLPKAAALLISFLVSALFHELCIAVPCHMFKLWAFGGIMFQVPLVLITNYLQNKFKNSMVGNMIFWFIFSIVGQPMCVLLYYHDLMNRKGKLD

SEQ ID NO: 13 - DNA, 444 bp, Glycine max
  source 1..444: mol_type = other DNA
atggctgagcttcactaccaacaacaacaccaataccctcaccgataccctaatgatccatacgaacaacaaactagctactccactcaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggatcagttggacagcgcgcctcacaagatcatggacaaggcgcgtgagatcaaggactatggacagcagcaaatcagtggggtgcaggcttcttaa

SEQ ID NO: 14 - protein, 147 aa, Glycine max
MAELHYQQQHQYPHRYPNDPYEQQTSYSTQVVKAATAVTAGGSLLILASLILAGTIIALTIVTPPLVIFSPVLVPAVITVALLSLGFLASGGFGVAAITVLAWIYRYVTGKYPPGADQLDSAPHKIMDKAREIKDYGQQQISGVQAS

SEQ ID NO: 15 - DNA, 444 bp, synthetic construct
  source 1..444: mol_type = other DNA
atggctgagcttcactaccaatgccaacaccaataccctcaccgataccctaattgcccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaa

SEQ ID NO: 16 - protein, 147 aa, synthetic construct
MAELHYQCQHQYPHRYPNCPYEQQTSYSCQVVKAATAVTAGGSLLILASLILAGTIIALTIVTPPLVIFSPVLVPAVITVALLSLGFLASGGFGVAAITVLAWIYRYVTGKYPPGADCLDSAPHKIMDCAREIKDYGQQCISGVQAS

SEQ ID NO: 17 - DNA, 444 bp, synthetic construct
  source 1..444: mol_type = other DNA
atggctgagcttcactaccaatgccaacaccaataccctcaccgataccctaattgcccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaa

SEQ ID NO: 18 - protein, 147 aa, synthetic construct
MAELHYQCQHQYPHRYPNCPYEQQTSYSTCVVKAATAVTAGGSLLILASLILAGTIIALTIVTPPLVIFSPVLVPAVITVALLSLGFLASGGFGVAAITVLAWIYRYVTGKYPPGADCLDSAPHKIMDCAREIKDYGQQCISGVQAS

SEQ ID NO: 19 - DNA, 444 bp, synthetic construct
  source 1..444: mol_type = other DNA
atggcttgccttcactaccaacaacaacaccaataccctcactgctaccctaatgatccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggatgccagcaaatcagtggggtgcaggcttcttaa

SEQ ID NO: 20 - protein, 147 aa, synthetic construct
MACLHYQQQHQYPHCYPNDPYEQQTSYSCQVVKAATAVTAGGSLLILASLILAGTIIALTIVTPPLVIFSPVLVPAVITVALLSLGFLASGGFGVAAITVLAWIYRYVTGKYPPGADCLDSAPHKIMDCAREIKDYGCQQISGVQAS

SEQ ID NO: 21 - DNA, 444 bp, synthetic construct
  source 1..444: mol_type = other DNA
atggcttgccttcactaccaacaacaacaccaataccctcactgctaccctaatgatccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggatgccagcaaatcagtggggtgcaggcttcttaa

SEQ ID NO: 22 - protein, 147 aa, synthetic construct
MACLHYQQQHQYPHCYPNDPYEQQTSYSTCVVKAATAVTAGGSLLILASLILAGTIIALTIVTPPLVIFSPVLVPAVITVALLSLGFLASGGFGVAAITVLAWIYRYVTGKYPPGADCLDSAPHKIMDCAREIKDYGCQQISGVQAS

SEQ ID NO: 23 - DNA, 444 bp, synthetic construct
  source 1..444: mol_type = other DNA
atggcttgccttcactaccaacaacaacaccaataccctcactgctaccctaatgatccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaa

SEQ ID NO: 24 - protein, 147 aa, synthetic construct
MACLHYQQQHQYPHCYPNDPYEQQTSYSTCVVKAATAVTAGGSLLILASLILAGTIIALTIVTPPLVIFSPVLVPAVITVALLSLGFLASGGFGVAAITVLAWIYRYVTGKYPPGADCLDSAPHKIMDCAREIKDYGQQCISGVQAS

SEQ ID NO: 25 - DNA, 444 bp, synthetic construct
  source 1..444: mol_type = other DNA
atggcttgccttcactaccaacaacaacactgctaccctcaccgataccctaattgcccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaa

SEQ ID NO: 26 - protein, 147 aa, synthetic construct
MACLHYQQQHCYPHRYPNCPYEQQTSYSCQVVKAATAVTAGGSLLILASLILAGTIIALTIVTPPLVIFSPVLVPAVITVALLSLGFLASGGFGVAAITVLAWIYRYVTGKYPPGADCLDSAPHKIMDCAREIKDYGQQCISGVQAS

SEQ ID NO: 27 - DNA, 444 bp, synthetic construct
  source 1..444: mol_type = other DNA
atggcttgccttcactaccaacaacaacactgctaccctcaccgataccctaattgcccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaa

SEQ ID NO: 28 - protein, 147 aa, unidentified
MACLHYQQQHCYPHRYPNCPYEQQTSYSTCVVKAATAVTAGGSLLILASLILAGTIIALTIVTPPLVIFSPVLVPAVITVALLSLGFLASGGFGVAAITVLAWIYRYVTGKYPPGADCLDSAPHKIMDCAREIKDYGQQCISGVQAS

SEQ ID NO: 29 - DNA, 444 bp, synthetic construct
  source 1..444: mol_type = other DNA
atggcttgccttcactaccaacaacaacactgctaccctcaccgataccctaattgcccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctactgctacgtcaccgggaagtacccacctggcgcggattgcttggacagcgcgcctcacaagatcatggactgcgcgcgtgagatcaaggactatggacagcagtgcatcagtggggtgcaggcttcttaa

SEQ ID NO: 30 - protein, 147 aa, synthetic construct
MACLHYQQQHCYPHRYPNCPYEQQTSYSCQVVKAATAVTAGGSLLILASLILAGTIIALTIVTPPLVIFSPVLVPAVITVALLSLGFLASGGFGVAAITVLAWIYCYVTGKYPPGADCLDSAPHKIMDCAREIKDYGQQCISGVQAS

SEQ ID NO: 31 - DNA, 444 bp, synthetic construct
  source 1..444: mol_type = other DNA
atggcttgccttcactaccaacaacaacactgctaccctcaccgataccctaattgcccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggtgctacccacctggcgcggattgcttggacagcgcgcctcacaagatcatggactgcgcgcgtgagatcaaggactatggacagcagtgcatcagtggggtgcaggcttcttaa

SEQ ID NO: 32 - protein, 147 aa, synthetic construct
MACLHYQQQHCYPHRYPNCPYEQQTSYSCQVVKAATAVTAGGSLLILASLILAGTIIALTIVTPPLVIFSPVLVPAVITVALLSLGFLASGGFGVAAITVLAWIYRYVTGCYPPGADCLDSAPHKIMDCAREIKDYGQQCISGVQAS

SEQ ID NO: 33 - DNA, 444 bp, synthetic construct
  source 1..444: mol_type = other DNA
atggcttgccttcactaccaacaacaacactgctaccctcaccgataccctaattgcccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctactgctacgtcaccgggaagtacccacctggcgcggattgcttggacagcgcgcctcacaagatcatggactgcgcgcgtgagatcaaggactatggacagcagtgcatcagtggggtgcaggcttcttaa

SEQ ID NO: 34 - protein, 147 aa, synthetic construct
MACLHYQQQHCYPHRYPNCPYEQQTSYSTCVVKAATAVTAGGSLLILASLILAGTIIALTIVTPPLVIFSPVLVPAVITVALLSLGFLASGGFGVAAITVLAWIYCYVTGKYPPGADCLDSAPHKIMDCAREIKDYGQQCISGVQAS

SEQ ID NO: 35 - DNA, 444 bp, synthetic construct
  source 1..444: mol_type = other DNA
atggcttgccttcactaccaacaacaacactgctaccctcaccgataccctaattgcccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggtgctacccacctggcgcggattgcttggacagcgcgcctcacaagatcatggactgcgcgcgtgagatcaaggactatggacagcagtgcatcagtggggtgcaggcttcttaa

SEQ ID NO: 36 - protein, 147 aa, synthetic construct
MACLHYQQQHCYPHRYPNCPYEQQTSYSTCVVKAATAVTAGGSLLILASLILAGTIIALTIVTPPLVIFSPVLVPAVITVALLSLGFLASGGFGVAAITVLAWIYRYVTGCYPPGADCLDSAPHKIMDCAREIKDYGQQCISGVQAS

SEQ ID NO: 37 - DNA, 6997 bp, synthetic construct
  source 1..6997: mol_type = other DNA
ttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggtaccccatcacaagtttgtacaaaaaagcaggctgcggccgcttgctcccttaaaaaaaaccatggcttgccattacggacaacaacagtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagacaaacctgcgctccacaccttcaacttcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatgaaatcaacaactctcctggcgcaccatcgtcggctacagcctcgggaattgctaccgagctcggtacccggcgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacccgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggtaccccatcacaagtttgtacaaaaaagcaggctgcggccgcttgctcccttaaaaaaaaccatggccgtggctgagtcatctcaagtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaggctaagggcgagcttaggggacatccattcgagggaaagccaattccaaacccacttctcggactcgattctaccagaaccggacatcatcatcaccaccactaatgaaatcaacaactctcctggcgcaccatcgtcggctacagcctcgggaattgctaccgagctcggtacccggcgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacccgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 38 - DNA, 6904 bp, synthetic construct
  source 1..6904: mol_type = other DNA
ttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgccgggtaccgagctcggtagcaattcccgaggctgtagccgacgatggtgcgccaggagagttgttgatttcattagtggtggtgatgatgatgtccggttctggtagaatcgagtccgagaagtgggtttggaattggctttccctcgaatggatgtcccctaagctcgcccttagccttctctttgaggttgatgaggtcgtggtagtaaagcaacacgcacattggctggccgaggatgcagaagatgaaccagaaaatcatgttgcccaccatggagttggagaacttctcttggaggtagttggtaatgagcacgagtggcacctggaacatgattccgatgaaagcccagagcttgaacacgtggcatggaacagcaatgcacaattcgtggaaggcaccagacacgaggaatgcgataatgatagcgccctcttttggaatgccgttcctcaggcatgggaaataaaggtgcctaaccatccaacggtgcactggcatgttccacatcttccaatactctgccacggtcttagcgttccaccaatccttgtagaactctctgtcgccgaacctaagaagctcagcgaggatattgagccagaggtggaagaatgagtagaacatgcagagccacacgtaaaggtttggcacagagagcttgagcaccctctcaatagcgtagaggaaatcgcccttaagagggtgcttagagttcctcacgatggggttgatgtactgctcgatgatgaagcccatgagtccgatgaacacaatgagcttcacgaactgcctcacaacccatccctttctaatgcaggaggaccttggataagagggctggtagcaaagggttggagccaccataaagtaagcgagggacttaagggacacgtcgtaagagcaagacacgatggtggagtttgggtgagcatcacccttgtatccggacttagcaagggtcctaatatcagaggaggtgtgggcgtaagaaacgagcttgagccacatgatgcagccgaaaagcataagcacaacgccagacatgtacacggagtcgcaggtaagaatcacgatcactgggtaaagcacagcagcggtagaaacgatcacgtgaagaagcacagccacaagctcagcaatgtggttcctctgaacgagcttctcaacgatgaaagcagcaagtgggaaaattgggagggtgaggcagcacatgaaaatggaccagtctccaagggatctagaggagaaccagaatccggtatcgatgagccagccgtacttcatgaggttctcgataatgagcctggagttcacggcaatgagcaccacaatgcagaggttgaagagtccagcgtgagactgcttgaagatagcatcggaggagagaggagactctctcacccttctatgagctgggaaggatggtctgtaggtgaacctgatatcagtctcgcccacttgcttctcttcgtttccgatcaccacaccaccaccgtttccaatgaggttcacagatccggtagcgttttccatagctcccaccctatcctgatccctgttctcagcaacatgtccagtagctggaacaccgttggtagaggtgaagccagaagaagaaggctcaatcacagagctggagggcttacgccttctgaagttgttaagatcggaatcgccgtggccggacatagtggtagtgttctacaccaaccaaaaatagatccaattagtatttcaaactcatcacattatttgaaatggttgaattaagtattattgatatcaatgacttgttaaccaacagcacttgttatatataaaagataaataagttatatctatagagagaaaaaaggaaacatacttgagatgactcagccacggccatggttttttttaagggagcaagcggccgcagcctgcttttttgtacaaacttgtgatggggtacccgtgtattatattgcaatgaaattgaaaagattgttatgcttttgataattgtatccttatcaatggtgattttgtaagatgaatatgagtctgtatttataatgcagatgctcttatcgtgtgttatctaaagctatggctagttggagattttctttgcaattctagaatccgttcatggatcaaaatcttcatatcatttgtatccaatgctgctcttccatgtgtgactgtggggattcttatggatatatgactcaacataatattattttttcataaaatggcttaatttatggggctatgctttctctattttgggtccacatttgtcatgttttatacagtgtttgagagccacaagatttgagaaaaattgtgactgttatggacagctggggcccgatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaaaaaaaaaccatggcttgccattacggacaacaacaacaaacctgcgctccacacctgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagtcaacttcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatgaataccttcacctctctcaacaatctagctagagtttgctcctatctatatgtaataagctatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcaaattgggttggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 39 - DNA, 6764 bp, synthetic construct
  source 1..6764: mol_type = other DNA
ttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattccagctgtccataacagtcacaatttttctcaaatcttgtggctctcaaacactgtataaaacatgacaaatgtggacccaaaatagagaaagcatagccccataaattaagccattttatgaaaaaataatattatgttgagtcatatatccataagaatccccacagtcacacatggaagagcagcattggatacaaatgatatgaagattttgatccatgaacggattctagaattgcaaagaaaatctccaactagccatagctttagataacacacgataagagcatctgcattataaatacagactcatattcatcttacaaaatcaccattgataaggatacaattatcaaaagcataacaatcttttcaatttcattgcaatataatacacgggtaccccatcacaagtttgtacaaaaaagcaggctgcggccgcttgctcccttaaaaaaaaccatggccgtggctgagtcatctcaagtatgtttccttttttctctctatagatataacttatttatcttttatatataacaagtgctgttggttaacaagtcattgatatcaataatacttaattcaaccatttcaaataatgtgatgagtttgaaatactaattggatctatttttggttggtgtagaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaggctaagggcgagcttaggggacatccattcgagggaaagccaattccaaacccacttctcggactcgattctaccagaaccggacatcatcatcaccaccactaatgaaatcaacaactctcctggcgcaccatcgtcggctacagcctcgggaattgctaccgagctcggtacccggcgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacccgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggggcccgatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaaaaaaaaaccatggcttgccattacggacaacaacaacaaacctgcgctccacacctgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagtcaacttcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacaggaataccttcacctctctcaacaatctagctagagtttgctcctatctatatgtaataagctatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcaaattgggttggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 40 - DNA, 6868 bp, synthetic construct
  source 1..6868: mol_type = other DNA
ttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcggatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaggcgcgccgtaccccatcacaagtttgtacaaaaaagcaggcttcggccgcttgctcccttaaaaaaaaccatggcttgccattacggacaacaacaacaaacctgcgctccacaccttcaacttgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatgaagcggccgctcacctctctcaacaatctagctagagtttgctcctatctatatgtaataagctatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcaaattgggttgcctaggctcgagatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaacgcgtaaaaaaaaccatggccgtggctgagtcatctcaagtatgtttccttttttctctctatagatataacttatttatcttttatatataacaagtgctgttggttaacaagtcattgatatcaataatacttaattcaaccatttcaaataatgtgatgagtttgaaatactaattggatctatttttggttggtgtagaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaataatgaagggcccatcaacaactctcctggcgcaccatcgtcggctacagcctcgggaattgctaccgagctcggaacccggcgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacccgttcaaacatttggcaataaagtttcttaagattgaatcctgttgccggtcttgcgatgattatcatataatttctgttgaattacgttaagcatgtaataattaacatataatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactaggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 41 - DNA, 6970 bp, synthetic construct
  source 1..6970: mol_type = other DNA
ttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcggatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaggcgcgccgtaccccatcacaagtttgtacaaaaaagcaggcttcggccgcttgctcccttaaaaaaaaccatggcttgccattacggacaacaacaacaaacctgcgctccacaccttcaacttgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatgaagcggccgctcacctctctcaacaatctagctagagtttgctcctatctatatgtaataagctatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcaaattgggttgcctaggctcgagatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaacgcgtaaaaaaaaccatggccgtggctgagtcatctcaagtatgtttccttttttctctctatagatataacttatttatcttttatatataacaagtgctgttggttaacaagtcattgatatcaataatacttaattcaaccatttcaaataatgtgatgagtttgaaatactaattggatctatttttggttggtgtagaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaggctaagggcgagcttaggggacatccattcgagggaaagccaattccaaacccacttctcggactcgattctaccagaaccggacatcatcatcaccaccactaatgaagggcccatcaacaactctcctggcgcaccatcgtcggctacagcctcgggaattgctaccgagctcggaacccggcgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacccgttcaaacatttggcaataaagtttcttaagattgaatcctgttgccggtcttgcgatgattatcatataatttctgttgaattacgttaagcatgtaataattaacatataatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactaggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 42 - DNA, 8003 bp, synthetic construct
  source 1..8003: mol_type = other DNA
ttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggaattcactagtagtggaagtggtgcagaagaggggagggaatgtttggggtacatgatttgaaaaaaggggagaatgattagaagagatagaaaattggcagaccaacattgaaggattcataggaatgttgtgtgttagtaaattggacaaatgttgctcttgatattttcagcatatcattcatcttagaagcccaactgttcttttttatatatattctctgttgccttcctttccttcaaaacacggcttttgaattttgaaccattaactgagttggaatagtggtaaaatcatgtagatttttcttttcgttttgtggggatatgaatacgaaatatccttacaaccagacaaagctgtggctcagaagaaagagaaacatgcctcactgctagaatcactagaactcacatcttgtatacaaactgttataccaaactgagaattttacaatttccaaagatgcaattctcaaccattaaccaatttcatcttaaacactaacagttcttcagtcttgaactttccatgctgaaaatttgttgcctatttcttttataggcagtggcttcttatgagagtcacactctctgaacttcttacgtaatacttgacttttcattttgtgctttataattgttatctcatgcattgcacttttaagataaggttgttgtgcattgtttggttgagtgcaacgatttactgattaccatagctacttgtggatcttatattttcaaaagtgtatgcttggtcattaatcaaccaagcatgataagccccatcatctaccaccccttctataccatatacgcattttgcttatctacattgctactgttacactcaaacaagtctaacagataatacgttaagttaatgaacatttttagtaatattattataaggattggccaatgtaattgtgaagagagaagcatgctttaagctacccaacaaaatggataagagtttcagttgatatggttctcttgtttcttttccaataaaaaaccaactttaaaatataaaatttactgtaacaaaggaacaaagagttttcacttaatccatgaatgagaaaggatggtcacaaaatatgttaggttaatatggaatgagggcactgtgcaaactacacaaataatatcaattccaccaccatcacacatttacgttcttccaaggaagagataagataatggagcctccacgtgtcacctccacatggtacctaacaataaggctaccattcaaaattttcctcactcgtgtggcctatatgctgtaatgtcatcacttattcaatccaacggttgtaacttttcggcaaccaatcctctccatttcacaccattggattagtactacacaaatcacactattatatatagcaagtttgagcagaagcttggatatctggcagcagaaaaacaagtagttgagaactaagaagaaggaaggcgcgccgtaccccatcacaagtttgtacaaaaaagcaggcttcggccgcttgctcccttaaaaaaaaccatggcttgccattacggacaacaacaacaaacctgcgctccacaccttcaacttgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatgaagcggccgctcacctctctcaacaatctagctagagtttgctcctatctatatgtaataagctatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcaaattgggttgcctaggctcgagatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaacgcgtaaaaaaaaccatggccgtggctgagtcatctcaagtatgtttccttttttctctctatagatataacttatttatcttttatatataacaagtgctgttggttaacaagtcattgatatcaataatacttaattcaaccatttcaaataatgtgatgagtttgaaatactaattggatctatttttggttggtgtagaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaggctaagggcgagcttaggggacatccattcgagggaaagccaattccaaacccacttctcggactcgattctaccagaaccggacatcatcatcaccaccactaatgaagggcccatcaacaactctcctggcgcaccatcgtcggctacagcctcgggaattgctaccgagctcggaacccggcgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacccgttcaaacatttggcaataaagtttcttaagattgaatcctgttgccggtcttgcgatgattatcatataatttctgttgaattacgttaagcatgtaataattaacatataatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactaggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 43 - DNA, 7993 bp, synthetic construct
  source 1..7993: mol_type = other DNA
ttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcggatccaaaagcttggacaggaacaaatgttacccatacataaaagatatttgtgaagtaacagtcacaaaattccatgaggccaacatactacaattgaattttcatggatacaattcttacaaaataaaaatatcgacataaccaccatcacacatttacactcttcacatgaaaagataagatcagtgaggtaatatccacatggcactgtcctattggtggcttatgataaggctagcacacaaaatttcaaatcttgtgtggttaatatggctgcaaactttatcattttcactatctaacaagattggtactaggcagtagctaagtaccacaatattaagaccataatattggaaatagataaataaaaacattatatatagcaagttttagcagaagctttgcaattcatacagaagtgagaaaaggcgcgccgtaccccatcacaagtttgtacaaaaaagcaggcttcggccgcttgctcccttaaaaaaaaccatggcttgccattacggacaacaacaacaaacctgcgctccacaccttcaacttgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatgaagcggccgctcacctctctcaacaatctagctagagtttgctcctatctatatgtaataagctatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcaaattgggttgcctagtagtggaagtggtgcagaagaggggagggaatgtttggggtacatgatttgaaaaaaggggagaatgattagaagagatagaaaattggcagaccaacattgaaggattcataggaatgttgtgtgttagtaaattggacaaatgttgctcttgatattttcagcatatcattcatcttagaagcccaactgttcttttttatatatattctctgttgccttcctttccttcaaaacacggcttttgaattttgaaccattaactgagttggaatagtggtaaaatcatgtagatttttcttttcgttttgtggggatatgaatacgaaatatccttacaaccagacaaagctgtggctcagaagaaagagaaacatgcctcactgctagaatcactagaactcacatcttgtatacaaactgttataccaaactgagaattttacaatttccaaagatgcaattctcaaccattaaccaatttcatcttaaacactaacagttcttcagtcttgaactttccatgctgaaaatttgttgcctatttcttttataggcagtggcttcttatgagagtcacactctctgaacttcttacgtaatacttgacttttcattttgtgctttataattgttatctcatgcattgcacttttaagataaggttgttgtgcattgtttggttgagtgcaacgatttactgattaccatagctacttgtggatcttatattttcaaaagtgtatgcttggtcattaatcaaccaagcatgataagccccatcatctaccaccccttctataccatatacgcattttgcttatctacattgctactgttacactcaaacaagtctaacagataatacgttaagttaatgaacatttttagtaatattattataaggattggccaatgtaattgtgaagagagaagcatgctttaagctacccaacaaaatggataagagtttcagttgatatggttctcttgtttcttttccaataaaaaaccaactttaaaatataaaatttactgtaacaaaggaacaaagagttttcacttaatccatgaatgagaaaggatggtcacaaaatatgttaggttaatatggaatgagggcactgtgcaaactacacaaataatatcaattccaccaccatcacacatttacgttcttccaaggaagagataagataatggagcctccacgtgtcacctccacatggtacctaacaataaggctaccattcaaaattttcctcactcgtgtggcctatatgctgtaatgtcatcacttattcaatccaacggttgtaacttttcggcaaccaatcctctccatttcacaccattggattagtactacacaaatcacactattatatatagcaagtttgagcagaagcttggatatctggcagcagaaaaacaagtagttgagaactaagaagaaggaaggcgcgtaaaaaaaaccatggccgtggctgagtcatctcaagtatgtttccttttttctctctatagatataacttatttatcttttatatataacaagtgctgttggttaacaagtcattgatatcaataatacttaattcaaccatttcaaataatgtgatgagtttgaaatactaattggatctatttttggttggtgtagaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaggctaagggcgagcttaggggacatccattcgagggaaagccaattccaaacccacttctcggactcgattctaccagaaccggacatcatcatcaccaccactaatgaagggcccatcaacaactctcctggcgcaccatcgtcggctacagcctcgggaattgctaccgagctcggaacccggcgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacccgttcaaacatttggcaataaagtttcttaagattgaatcctgttgccggtcttgcgatgattatcatataatttctgttgaattacgttaagcatgtaataattaacatataatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactaggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 44 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggctgagcttcactaccaacaacaacaccaataccctcaccgataccctaatgatccatacgaacaacaaactagctactccactcaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggatcagttggacagcgcgcctcacaagatcatggacaaggcgcgtgagatcaaggactatggacagcagcaaatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 45 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggctgagcttcactaccaatgccaacaccaataccctcaccgataccctaattgcccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 46 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggctgagcttcactaccaatgccaacaccaataccctcaccgataccctaattgcccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 47 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcttgccttcactaccaacaacaacaccaataccctcactgctaccctaatgatccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggatgccagcaaatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 48 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcttgccttcactaccaacaacaacaccaataccctcactgctaccctaatgatccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggatgccagcaaatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 49 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcttgccttcactaccaacaacaacaccaataccctcactgctaccctaatgatccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 50 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcttgccttcactaccaacaacaacactgctaccctcaccgataccctaattgcccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 51 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcttgccttcactaccaacaacaacactgctaccctcaccgataccctaattgcccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggaagtacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 52 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcttgccttcactaccaacaacaacactgctaccctcaccgataccctaattgcccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctactgctacgtcaccgggaagtacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 53 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcttgccttcactaccaacaacaacactgctaccctcaccgataccctaattgcccatacgaacaacaaactagctactcctgccaggtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggtgctacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 54 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcttgccttcactaccaacaacaacactgctaccctcaccgataccctaattgcccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctactgctacgtcaccgggaagtacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 55 - DNA, 9221 bp, synthetic construct
  source 1..9221: mol_type = other DNA
tggcaggatatattcaattgtaaataaattgacgcttagacaacttaataacacattgcggacgtttttaatgtactgaattgtctagtagcgcttgcacgtttggccggccgtttaaacttagctagctagatctgtttatactctgttgcttccacgtctatgcaaattttcaaaggagatgctgtcgaaattttatatatgaaggttagttgtgtagtttagtagcatttcggggtgtttgtggttcccagggtgttacaccataatggtccctatttgccatgtagaacaaagaggcattattctaagcatatttcaatcaagcaattacgaaaagttagcatgattatcgtcgaccaaccataatgtctaaatatattaagctcataaatattgattattgtcttgttaattttgtatttattatgtatatttagtatattagtttggtaaatctagttattatggaatatttatggttagtttttaagctagatagaatgttatatcttactagtaataaatattagtattttaataaaatatggtaatataaaagcacatataatatcaacaggttcacaaaacatatgcgtcacacgtttccaggcatgcctaacatagcacattaatcaaggctatagggtcccacctaacaaagtacacatttttggagattataccatcctcgtcatgtttgtgtgtgttaatgctcaattaaccgagtagcacaccacaacgatgatttatacttcattactgttgatcttagagcaaaataactatcaaccacatttggagactagaagcacataagaactagtcggtcctagatgccgtaatcattagctaatcgtaagtgacgcttggacacgagcggcttgagctaggaacctacgaagtcatcggaatcaagcgtaagtggcgcgcctagaatgatgatttaaataccgcttgtcagcgcgatcgcggcgcgccaagcttggatctcctgcaggatctggccggccggatgggcggatctcgtacctcaactttgtatagaaaagttgggccgaattcgagctcggtacggccagaatggcccggaccgaagcttgcatgcgcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcgcggccgggatccgccatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacaacgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagtgcaaagtcattgagagactggccccttttcatgtgttgtctttctcttgtggtatttcctttcgctgcctttatggtggagaagttggcacaacggaagtgtatacccgaaccagttgttgttgtacttcatataatcattacctcaacttcgcttttctatccagttttagttattctcaagtgtgattctgcttttgtatcaggtgtcacgttaatgctgttttcttgtgttgtatggttaaaattggtgtcttttgcacatacaaactatgatatgagagcacttaccaaattagttgaaaagggagaagcactgctcgatactctgaacatggagtatccttacaacgtaaccttcaagagcttggcatatttcctgcttgcccctacattatgttaccagccaagctatcctcgcacaccttatattcgaaagggttggttgtttcgccaacttgtcaagctgatagtatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcgtggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaatttccttcctggtttctgctttattccatgagctgtgcattgctgttccttgccacatgttcaagttgtgggctttcggtggaattatgtttcaggttcctttggtcttgatcactaattatctgcaaaataaattcaaaaactcaatggttggaaatatgattttttggttcatattcagtatcgttggtcaacctatgtgtgtactgctatactaccatgacttgatgaataggaaaggcaaacttgactgattcgaattggccacggtaccaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggcgcgccggtcacccggtccgggcctagaaggccacccggggtcggaggagactagccgttagttagtgcagaccgtctctaatcagtgactagcgacgtcccgacccacccagctttcttgtacaaagtggggatcggccagaatggcccggaccgggttaccaagcggccgccttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaagataagaacccaaccacttcaaccccatataaataaacccggacacaacttcaccaagtcactcaccacttcaaaacactcataacacaaagcacaaagcaaagctcatccttgagttaaaaaaggatccaaacaatggcttgccttcactaccaacaacaacactgctaccctcaccgataccctaattgcccatacgaacaacaaactagctactccacttgcgtcgtcaaggcggccaccgccgtcaccgcgggcggctccctcttgatcctcgcctcgttgatccttgccggcaccatcatcgccctcaccatcgtcacaccaccgctcgtcatcttcagtccggttctcgtccccgcggtgatcaccgtcgcgctgctgagcctggggttccttgcctccggcgggttcggtgtggcggcgatcacggtgctggcgtggatctacaggtacgtcaccgggtgctacccacctggcgcggattgcttggacagcgcgcctcacaagatcatggactgcgcgcgtgagatcaaggactatggacagcagtgcatcagtggggtgcaggcttcttaattcgaattggccacggtaccctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaattctggcgcgccggtcacccggtccgggcctagaaggccacccggggcacttaggagcgaagactaacggttagctagggctgatacctaaaccttagtcactagcagcgtctactagcccccaactttattatacaaagttgatggtacggatccgtcgacggcgcgccgcgatcgcgaataagtgactagggtcacgtgaccctagtcacttaggtgaccggctgagcataacttcgtataatgtatgctatacgaagttataacctagctaggtaagtccggaactatagctaagtcgtacgctactgccaccaacagtcgtgaagctaaccgtcatagtaaccgtgagcgcttcatgtcacctcatagggtaactagctcgattagcagctagctagtgaaccggtagtaaccgctcttccagttgaagagcgttcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgtttgagctcctgcaggatgcggccgcagatcctgatcctgatcccgatttttttgatgttttggtgttggctgacaggatatataccgttgtaatt

SEQ ID NO: 56 - DNA, 9744 bp, synthetic construct
  source 1..9744: mol_type = other DNA
ttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaacaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagggcgcgccgtaccccatcacaagtttgtacaaaaaagcaggcttcggccgcttgctcccttaaaaaaaaccatggcttgccattacggacaacaacaacaaacctgcgctccacaccttcaacttgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatgaacctaggatcacctctctcaacaatctagctagagtttgctcctatctatatgtaataagctatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcaaattgggttgcccggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggctcgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagacgcgtaaaaaaaaccatggccgtggctgagtcatctcaagtatgtttccttttttctctctatagatataacttatttatcttttatatataacaagtgctgttggttaacaagtcattgatatcaataatacttaattcaaccatttcaaataatgtgatgagtttgaaatactaattggatctatttttggttggtgtagaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaggctaagggcgagcttaggggacatccattcgagggaaagccaattccaaacccacttctcggactcgattctaccagaaccggacatcatcatcaccaccactaatgaaactagtaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 57 - DNA, 9603 bp, synthetic construct
  source 1..9603: mol_type = other DNA
cactgatagtttaaactgaaggcgggaaacgacaatctgatccaagctcaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaacaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagacgcgtaaaaaaaaccatggccgtggctgagtcatctcaagtatgtttccttttttctctctatagatataacttatttatcttttatatataacaagtgctgttggttaacaagtcattgatatcaataatacttaattcaaccatttcaaataatgtgatgagtttgaaatactaattggatctatttttggttggtgtagaacactaccactatgtccggccacggcgattccgatcttaacaacttcagaaggcgtaagccctccagctctgtgattgagccttcttcttctggcttcacctctaccaacggtgttccagctactggacatgttgctgagaacagggatcaggatagggtgggagctatggaaaacgctaccggatctgtgaacctcattggaaacggtggtggtgtggtgatcggaaacgaagagaagcaagtgggcgagactgatatcaggttcacctacagaccatccttcccagctcatagaagggtgagagagtctcctctctcctccgatgctatcttcaagcagtctcacgctggactcttcaacctctgcattgtggtgctcattgccgtgaactccaggctcattatcgagaacctcatgaagtacggctggctcatcgataccggattctggttctcctctagatcccttggagactggtccattttcatgtgctgcctcaccctcccaattttcccacttgctgctttcatcgttgagaagctcgttcagaggaaccacattgctgagcttgtggctgtgcttcttcacgtgatcgtttctaccgctgctgtgctttacccagtgatcgtgattcttacctgcgactccgtgtacatgtctggcgttgtgcttatgcttttcggctgcatcatgtggctcaagctcgtttcttacgcccacacctcctctgatattaggacccttgctaagtccggatacaagggtgatgctcacccaaactccaccatcgtgtcttgctcttacgacgtgtcccttaagtccctcgcttactttatggtggctccaaccctttgctaccagccctcttatccaaggtcctcctgcattagaaagggatgggttgtgaggcagttcgtgaagctcattgtgttcatcggactcatgggcttcatcatcgagcagtacatcaaccccatcgtgaggaactctaagcaccctcttaagggcgatttcctctacgctattgagagggtgctcaagctctctgtgccaaacctttacgtgtggctctgcatgttctactcattcttccacctctggctcaatatcctcgctgagcttcttaggttcggcgacagagagttctacaaggattggtggaacgctaagaccgtggcagagtattggaagatgtggaacatgccagtgcaccgttggatggttaggcacctttatttcccatgcctgaggaacggcattccaaaagagggcgctatcattatcgcattcctcgtgtctggtgccttccacgaattgtgcattgctgttccatgccacgtgttcaagctctgggctttcatcggaatcatgttccaggtgccactcgtgctcattaccaactacctccaagagaagttctccaactccatggtgggcaacatgattttctggttcatcttctgcatcctcggccagccaatgtgcgtgttgctttactaccacgacctcatcaacctcaaagagaaggctaagggcgagcttaggggacatccattcgagggaaagccaattccaaacccacttctcggactcgattctaccagaaccggacatcatcatcaccaccactaatgaaactagttcacctctctcaacaatctagctagagtttgctcctatctatatgtaataagctatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcaaattgggttgcccggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggctcgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagggcgcgccgtaccccatcacaagtttgtacaaaaaagcaggcttcggccgcttgctcccttaaaaaaaaccatggcttgccattacggacaacaacaacaaacctgcgctccacaccttcaacttgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagcaacctagagcttgcagggttgtgaaggctgctactgctgttactgctggtggatctcttcttgtgctttccggacttactcttgctggaaccgtgatcgctcttactattgctaccccactcctcgtgatcttctctcctgttcttgtgccagccgtgattaccattttccttctcggtgctggattcctcgcttctggtggattcggagttgctgctctttctgtgctctcctggatctacagatacctcactggaaagcacccaccaggtgctgattgccttgaatctgctaagaccaagctcgcttcttgcgccagagagatgaaggatagagctgagcaattctcctgccagccagttgctggatctcaaacttcttaatgaacctaggaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggatcctctagagtcgacctgcaggcatgcaagcttgagcttgcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgctctcaacaatctagctagagtttgctcctatctatatgtaataaggtatgctgatatgcactattcaaataggagcattagctatgtttgttaatgtcactttatgttatgtgggtaagtcacctaagacactccacgtacctacttgttgtctcttacgcggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcacaaaaaaacaaacacaaaaagggagtgcattttccagggcatttttgtaataaaaaacagttaaaagggagtgcaatagaaatataggggtgtggaaatagtgatttgagcacgtcttgaagaattagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcaattcggcgttaattcagtacattaaaaacgtccgcaatgtgttattaagttgtctaagcgtcaatttgtttacaccacaatatatcctgccacc

SEQ ID NO: 58 - protein, 520 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMS

SEQ ID NO: 59 - protein, 503 aa, Brassica juncea
MAILDSGGVAVPPTENGVADLDRLHRRKSRSDSSNGLLSDTSPSDDVGAAAAERDRVDSAAEEEAQGTANLAGGDAETRESAGGDVRFTYRPSVPAHRRTRESPLSSDAIFKQSHAGLFNLCVVVLVAVNSRPIIENLMKYGWLIRTDFWFSSTSLRDWPLFMCCLSLSVFPLAAFTVEKMVLQKFISEPVAIILHVIITLTEVLYPVYVTLRCDSAFLSGVTLMLLTCIVWLKLVSYAHTSYDIRTLANSADKVDPEISYYVSLKSLAYFMVAPTLCYQPSYPRSPCIRKGWVARQFAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFMGIMFQVPLVFITNFLQERFGSMVGNMIFWFSFCIFGQPMCVLLYYHDLMNRKGSMS

SEQ ID NO: 60 - protein, 503 aa, Brassica napus
MAILDSGGVAVPPTENGVADLDRLHRRKSSSDSSNGLLSDTSPSDDVGAAAAERDRVDSAAEEEAQGTANLAGGDAETRESAGGDVRFTYRPSVPAHRRTRESPLSSDAIFKQSHAGLFNLCVVVLVAVNSRLIIENLMKYGWLIRTDFWFSSTSLRDWPLFMCCLSLSVFPLAAFTVEKMVLQKFISEPVAIILHVIITMTEVLYPVYVTLRCDSAFLSGVTLMLLTCIVWLKLVSYAHTSYDIRTLANSADKVDPEISYYVSLKSLAYFMVAPTLCYQPSYPRSPCIRKGWVARQLAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFMGIMFQVPLVFITNFLQERFGSMVGNMIFGSASCIFGQPMCGLLYYHDLMNRKGSMS

SEQ ID NO: 61 - protein, 503 aa, Brassica juncea
MAILDSGTVTMATENGVADLDMLRRRKSRSDSSNGLLSETSPSDDAGAPADVEDRVDSAAQGTANLAGDTETRESGGGGGGGNGEVRFTYRPSVPAHRRTRESPLSSDAIFKQSHAGLFNLCVVVLVAVNSRLIIENLMKYGWLIRTDFWFSSTSLRDWPLFMCCLSLSIFPLAAFTVEKLVLQKFISEPVVIILHIIITMTEVLYPVYVTLRCDSAFLSGVTLMLLTCIVWLKLVSYAHTNYDIRTVANSADKVDPEVSYYVSLKSLAYFMVAPTLCYQPSYPRSPGIRKGWVARQFAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFIGIMFQVPLVFITNYLQERFGSMVGNMIFWFSFCIFGQPMCVLLYYHDLMNRKGSMS

SEQ ID NO: 62 - protein, 518 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHISELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEK

SEQ ID NO: 63 - protein, 473 aa, Echium sp.
MTIWESPEIISSDEAAAALRRRGGAKEVAEQRLDSEEEKKKEEENGKLKYTYRASAPAHRRIKESPLSSDAIFKQSHAGLFNLCIVVLVAVNSRLIIENLMKYGWLISSGFWLSSTSLSDWPLLICCLSLPIFPLASFVVEKLSQQEFISEQVVITLHALITTTVIMYPVIVILRCDPAVLSGVILMLFTCIVWLKLVSYAHTNYDMRALAKDCDKLQALSGSSMEDCSFEVNFQALVYFMVAPTLCYQLRYPRTPCIRWGWVTRHLIKLIIFTGLMGFIIEQYINPIVKNSQHPLKGNLLYAIERVLKLSVPNIYVWLCMFYCFFHLWLNILAELLCFGDREFYKDWWNAQTIEEYWRMWNMPVHKWMVRHIYFPCLRNGMPKELAILIAFLISAIFHELCIAVPCHIFKFWAFIGIMFQVPLVLLTNVLVKKFQNSMVGNMIFWCFFCILGQPMSLLLYYHDVLNRKVNAN

SEQ ID NO: 64 - protein, 523 aa, Vernonia galamensis
MALLDTPQIGEITTTATTTIRRRTTVKPDAGIGDGLFDSSSSSKTNSSFEDGDSLNGDFNDKFKEQIGAGDESKDDSKGNGQKIDHGGVKKGRETTVVHYAYRPSSPAHRRIKESPLSSDAIFKQSHAGLFNLCIVVLVAVNGRLIIENLMKYGLLINSNFWFSSRSLRDWPLLMCCLTPSDFPLAAYIVEKLAWKKRISDPVVITLHVIITTTAILYPVFMILRFDSVVLSGVSLMLCACINWLKLVSFVHTNYDMRSLLNSTDKGEVEPMSSNMDYFYDVNFKSLVYFMVAPTLCYQISYPRTAFIRKGWVLRQLIKLVIFTGFMGFIIEQYINPIVKNSRHPLKGDFLYAIERVLKLSVPNLYVWLCMFYCFFHLWLNILAELLCFGDREFYKDWWNAQTIEEYWRLWNMPVHKWIVRHLYFPCLRNGIPKGAAILVAFFMSAVFHELCIAVPCHIFKFWAFIGIMFQVPLVLLTNYLQHKFQNSMVGNMIFWCFFSIFGQPMCVLLYYHDVMNQKGKSK

SEQ ID NO: 65 - protein, 532 aa, Nicotiana tabacum
MVIMELPESVEMTTTTTTSGIENLNSDLNHSVRRRRGSNGFEAASAINSSDANMSEDRRDVCGSGAGLETVNERSKSVGESSDVIRKEDDRNDNVANGEESKSTETTTTPFKFAYRASAPAHRRIKESPLSSDAIFKQSHAGLFNLCVVVLIAVNSRLIIENLMKYGLLIRAGFWFSSKSLRDWPLLMCCLSLQILPLAAFLVEKLAQQRHLTERAVVTLHITITTAAILYPVLVILGCDSAFLFGVILMLVACIVWMKLVSYAHTNHDMRQLAKSTDKDETSDGDFSYDVSFKSLAYFMVAPTLCYQLSYPHTPCIRKGWVARQFIKLVIFTGLMGFIIEQYINPIVQNSQHPLKGNLLYAIERVLKLSVPNLYVWLCMFYCFFHLWLNILAELLCFGDREFYKDWWNAKTIDEYWRMWNMPVHKWMVRHIYFPCLRNGIPKGVAILIAFLVSAVFHELCIAVPCRLFKWWAFMGIMFQVPLVILTNFLQNKFQSSMVGNMMFWCFFCILGQPMCVLLYYHDVMNRKSSAR

SEQ ID NO: 66 - protein, 534 aa, Perilla frutescens
MAILDSPEILDTTSSSADNGAAHHTTLRRRQSARSVPPLLDSDSNSLEAESAINDSENVRNDANLIENLRGGAVESENEKQESYGKEEGAKVKENGETSNGNGTDVMAVKFTFRPAAPAHRKNKESPLSSDAIFKQSHAGLFNLCIVVLVAVNSRLIIENLMKYGWLIKSGFWFSSTSLRDWPLLMCCLSLPVFALASFLVEKLVKLNYIPEWVAVFLHVTITTVEILFPVVVILRCDSAVLSGVTLMLFACTVWLKLVSYAHTNYDLRVLAKSLDKWEAMSRYWNLDYAYDVSFKSLAYFMVAPTLCYQPSYPRTACIRKGWVVRQLIKLVIFTGLMGFIIEQYINPIVQNSQHPLKGNLLYAIERVLKLSVPNLYVWLCMFYCFFHLWLNILAELLCFGDREFYKDWWNARTVEEYWRMWNMPVHKWMVRHIYCPCLQNGIPKIVAVLIAFLVSAIFHELCVAVPCQIFKFWAFSGIMLQVPLVIVTNYLQEKFKNSMVGNMMFWCFFCIFGQPMCVLLYYHDLMNRKASAR

SEQ ID NO: 67 - protein, 512 aa, Zea mays
MADSEDAPPAVHRRPPRPARGAAAAQGFAAALRRRLRSGAAVAARASFAADSGDESGPGEPSSSRRRDNSGGASSAAGGRAGAGDFSAFTFRAAAPVHRKAKESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAK

SEQ ID NO: 68 - protein, 515 aa, Sorghum bicolor
MADTDDAPPAPAVHRRPPRPARGAAAAQGFAAKLRRRLSSGAAAAARASFAADSGDESGPGEPSSSRRRDNGGDASSAADGGRGGAGDFSAFTFRAAAPVHRKAKESPLSSDAIFKQSHAGLFNLCIVVLVAVNSRLIIENLMKYGLLIRSGFWFNATSLRDWPLLMCCLSLPVFPLGAFAVEKLAFNNLITDAAATCFHIFLTTLEIVYPVLVILKCDSAVLSGFVLMFIACIVWLKLVSFAHTNHDIRKLITSGKKVDNELTVADIDNLQAPTLGSLTYFMMAPTLCYQPSYPRTPYVRKGWLVRQVILYLIFTGLQGFIIEQYINPIVVNSQHPLKGGLLNAVETVLKLSLPNVYLWLCMFYCLFHLWLNILAEILRFGDREFYKDWWNAKTIDEYWRKWNMPVHKWMLRHIYFPCIRNGISKEVAAFIAFFVSAVFHELCVAVPCHILKFWAFLGIMLQIPLIILTSYLKNKFNDTMVGNMIFWFFFCIYGQPMCVLLYYHDVMNRTEKTK

SEQ ID NO: 69 - protein, 538 aa, Oryza sativa
MVGSDGDGDGGGGEAHAPAAPAHHHRRPPRPRGGSGAIVEGFAAALRRRIRSGAAAAARASFGGDSGDEAASGEPSSSSSSSPSRRRGGDSNGAEASSAAGGGGGRGGGGDFSAFTFRAAAPVHRKAKESPLSSDAIFKQSHAGLFNLCIVVLVAVNSRLIIENLMKYGLLIRAGFWFNDKSLRDWPLLMCCLSLPAFPLGAFAVEKLAFNNVITDAVATCLHIFLSTTEIVYPVLVILKCDSAVLSGFLLIFIACIVWLKLVSFAHTNHDIRQLTMGGKKVDNELSTVDMDNLQPPTLGNLIYFMMAPTLCYQPSYPRTSCVRKGWLIRQIILYLIFTGLQGFIIEQYINPIVVNSQHPLKGGLLNAVETVLKLSLPNVYLWLCMFYAFFHLWLSILAEILRFGDREFYKDWWNAKTIDEYWRKWNMPVHKWVVRHIYFPCMRNGISKEVAVLISFLVSAVLHEICVAVPCRILKFWAFLGIMLQIPLIVLTAYLKSKFRDTMVGNMIFWFFFCIYGQPMCLLLYYHDVMNRIEKAR

SEQ ID NO: 70 - protein, 477 aa, Oryza sativa
MAPPPSLAPDRGGGEPDDALRLRARAAAAAGDAPAPQQQQEQRHQEQQQQLLWYRASAPAHRRVRESPLSSDAIFRQSHAGLLNLCIVVLVAVNSRLIIENLMKYGLLIRAGFWFSGTSLADWPLLMCCLTLPTFPLAALMVEKLAQRKLISKHVVILLHIVITTSVLVYPVVVILKCDSAVLSGFVLMFLASIIWLKLVSFAHTNYDIRMLSKSIEKGVTHDISIDPENIKWPTFKRLSYFMLAPTLCYQPSYPRTTYIRKGWVVRQLIKCLVFTGLMGFIIEQYINPIVKNSKHPLKGNFLNAIERVLKLSVPTLYVWLCMFYCFFHLWLNILAELLCFGDREFYKDWWNAKTVEEYWRMWNMPVHKWVIRHIYFPCIRNGFSKGVAILISFLVSAAFHELCVAVPCHIFKFWAFIGIMFQIPLVFLTKYLQDKFNNTMVGNMIFWFFFSILGQPMCVLLYYHDVMNRQQAQTNR

SEQ ID NO: 71 - protein, 529 aa, Sorghum bicolor
MAPPPSMAAASDRAVPGADATEASSLRLRRAPSADAGDLADDSSGDRRENGEPQPPQEQQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLIRRKLIGEHVVILLHIIITTSVIVYPVVVTLKCDSAVLSGFLLMFLASIMWMKLVSYAHTNYDIRALSKSTEKGAAYGNYVDPESMKDPTFKSLVYFMLAPTLCYQPTYPRTTCIRKGWVTRQLIKCLVFTGLMGFIIEQYINPIVKNSKHPLKGNFLNAIERVLKLSVPTLYVWLCMFYCFFHLWLNILAELLCFGDREFYKDWWNAKTVEEYWRMWNMPVHKWIIRHIYFPCIRKGFSRGVAILVSFLVSAVFHEICIAVPCHIFKFWAFSGIMFQIPLVFLTRYLQATFKNIMVGNMIFWFFFSIVGQPMCVLLYYHDVMNRQAQQVDNSAETCTLRQVIRSRLERRSRKQQQQQASSPPLPLLPAS

SEQ ID NO: 72 - protein, 494 aa, Zea mays
MAPPPSMPAASDRAGPGRDAGDSSSLRLRRAPSADAGDLAGDSSGGLRENGEPQSPTNPPPQEQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLITRKLIGEHVVILLHIIITTSAIVYPVVVTLKCDSAVLSGFVLMFLASIMWMKLVSYAHTNYDIRVLSKSTEKGAAYGNYVDPENMKDPTFKSLVYFMLAPTLCYQPTYPQTTCIRKGWVTQQLIKCVVFTGLMGFIIEQYINPIVKNSKHPLKGNFLNAIERVLKLSVPTLYVWLCMFYCFFHLWLNIVAELLCFGDREFYKDWWNAKTVEEYWRMWNMPVHKWIIRHIYFPCIRKGFSRGVAILISFLVSAVFHEICIAVPCHIFKFWAFSGIMFQIPLVFLTRYLHATFKHVMVGNMIFWFFFSIVGQPMCVLLYYHDVMNRQAQASR

SEQ ID NO: 73 - protein, 447 aa, Physcomitrella patens
MPVKSSNLAGERAATSHINANTKFDLRGCTPAHRVRRESPLSSDAIFHQSHAGLFNLCIVVLIAVNSRLIIENLMKYGLLIRTGFWFSSKSARDWPLLMCGLSLPTFPFAALLVEKLCWKNENGKWLIFVLHLIISTVGILYPGYVIHRVQSALLPGLVLILIAVTGWMKLISYAHVNKDMRELLRAKEKLPEAPQYADKIEVPDHLTIQNIAYFMLAPTLCYQLSYPRSDTIRKSWVLRQAGKLVVFLGLGGFIIEQYINPTVKNSQHPLRGNYLQALERVLKLSLPVLYVWLCLFYCLFHLWLNIVAELLRFGDREFYKDWWNAQTVEEYWRMWNMPVHKWMVRHIYFPSIRAGLSKKAAVLLVFAISALFHEVIIGVPCHMLRCWAFLGIMMQVPLVYLTNVIKERYHSSMVGNMVFWFFFCIVGQPMCLLLYYHDVFNNFPST

SEQ ID NO: 74 - protein, 425 aa, Selaginella sp.
MRPSLPAHRRSKESPLSSDAIFTQSHAGLFNLCIVVLVAVNSRLIIENLMKYGLLIQAEVLFSSKSLKDWPLLMCGLSLLIFPLAAYVIEKIKARRPATAVAPLHLINLAAALLYPIYVIEMFQSDLLSGLVLMLIAVTGWLKLVSYAHTNADIRAVKKDGGKIELPAEAPAIDYPDNISLKNIAYFMAAPTLCYQLSYPRSPRIRTGWVLRQLGKWIVFNGFMGFIIGQYMNPIIRNSTHPLKGNYLYAIERVLKLSIPTLYVWLGFFYCFFHLWLNIVAEILCFGDREFYKDWWNAKSVDEYWRLWNMPVHRWLVRHVYFPCLRLGLHKQFAILVVFVISGIFHEICIAVPCHMLRGWAFLGIMFQVPLVLVTNVLQRKFQSSMVGNMIFWFFFCIVGQPMCVLLYYHDVVNRQQLQLAGRSK

SEQ ID NO: 75 - protein, 507 aa, Euonymus alatus
MAANLNEASDLNFSLRRRTGGISSTTVPDSSSETSSSEADYLDGGKGAADVKDRGDGAVEFQNSMKNVERIEKHESRVGLDSRFTYRPSVPAHRTIKESPLSSDAIFKQSHAGLFNLCIVVLVAVNSRLIIENLMKYGWLIRSGFWFSSRSLRDWPLFMCCLTLPVFPLAAFLFEKLAQKNLISEPVVVLLHIVNTTAAVLYPVLVILRCDSAFMSGVTLMLFACIVWLKLVSYAHTNYDMRALTKSVEKGDTPLSSQNMDYSFDVNIKSLAYFMVAPTLCYQISYPRTPYVRKGWVVRQFVKLIIFTGLMGFIIEQYINPIVQNSQHPLKGNFLYAIERVLKLSVPNLYVWLCMFYCLFHLWLNILAELLCFGDREFYKDWWNAKTVEEYWRMWNMPVHKWMVRHIYFPCLRNGIPKGVAFVISFLVSAVFHELCIAVPCHIFKLWAFFGIMLQVPLVLITSYLQNKFRSSMVGNMMFWFSFCIFGQPMCLLLYYHDLMNRNGKME

SEQ ID NO: 76 - protein, 518 aa, Vitis vinifera
MAICNSPVSVTTSSSSSHADSDLDFSIRKRFGGKGKAVADSSLETETEAAAAAVLEAEKSVGEVGSGGDRGESGSQVVRNGENGVAEVAAKFAYRPCAPAHRKVKESPLSSDAIFRQSHAGLFNLCIVVLVAVNSRLIIENLMKYGWLIRAGFWFSSKSLRDWPLFMCCLTLPIFPLAAFVVEKLAQQKYISEQVVVSLHIIITTAAVLFPVLVILRCDSAVLSGVTLMLFACIVWLKLVSFAHTNYDMRAVAKLIDKGDDLSTSLNMDYPYDVNFKSLAYFMVAPTLCYQPSYPRSTCIRKGWVFRQFVKLAIFTGVMGFIIEQYINPIVQNSQHPLKGNFFYALERILKLSVPNLYVWLCMFYCFFHLWLNILAELLRFGDREFYKDWWNAKTVEEYWRMWNMPVHKWMVRHLYFPCLRNGISKGVSVVIAFAISAIFHELCIAVPCHMFKLWAFIGIMFQVPLVLVTNYLQNKFRNSMVGNMIFWLFFSILGQPMCVLLYYHDLMNRKETTESSL

SEQ ID NO: 77 - protein, 498 aa, Glycine max SIRE1 virus
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 78 - protein, 504 aa, Glycine max
MAISDEPESVATALNHSSLRRRPSATSTAGLFNSPETTTDSSGDDLAKDSGSDDSINSDDAAVNSQQQNEKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQRKCIPEPVVVVLHIIITSTSLFYPVLVILRCDSAFVSGVTLMLFSCVVWLKLVSYAHTNYDMRALTKLVEKGEALLDTLNMDYPYNVSFKSLAYFLVAPTLCYQPSYPRTPYIRKGWLFRQLVKLIIFTGVMGFIIEQYINPIVQNSQHPLKGNLLYATERVLKLSVPNLYVWLCMFYCFFHLWLNILAELLRFGDREFYKDWWNAKTVEDYWRMWNMPVHKWMIRHLYYPCLRHGLPKAAALLIAFLVSALFHELCIAVPCHIFKLWAFGGIMFQVPLVLITNYLQNKFRNSMVGNMIFWFIFSILGQPMCVLLYYHDLMNRKGKLD

SEQ ID NO: 79 - protein, 511 aa, Lotus sp.
MAISEDSESLFAAAAASSVIQSGSSVRRRPSAISAVATVEDESSSEEPVPVRDSGSDVDDSVSSEQHVSPATANREKNQVHDISATKFAYRPSAPAHRRVKESPLSSDNIFRHHAGLFNLCIVVLVAVNSRLIIENLMKYGWLIRTGFWFSSKSLRDWPLFMCCLSLAIFPFAAFVVEKLVQQKCISEPVVVLHIFISTAAVVYPVLVILRTDSAFPSGVTLMLFACIVWLKLVSYAHTNYDMRELTKSIEKGEALPNTLNMDYSYDVSFKSLAYFMIAPTLCYQPRYPRSPSIRKGWVLRQLVKLIIFTGVMGFIIEQYINPIVQNSQHPLKGNLLYAIERVLKLSVPNLYVWLCMFYCFFHLWLNILAELLRFGDREFYKDWWNAKTFEEYWRMWNMPVHKWMIRHLYFPCLRNGIPKGVAILIAFLVSALFHELCIAVPCHIFKLWAFGGIMFQVPLILITNYLQNKFRNSMVGNMIFWFIFSILGQPMAVLLYYHDLMNRKSKLDQS

SEQ ID NO: 80 - protein, 539 aa, Medicago truncatula
MAISDTPETTATATATVTTIETDTDLKRSSLRRRPSATSTAGGLFDAESAAADAVRDSGSDDSLNGKINNEEEVKDRKTDHAEGIVDDDDDNAVKKNGGNDVINDRENVAVDFKFTYRPSVPAHRRSKESPLSSGNIFRQSHAGLFNLCIVVLVAVNSRLIIENLMKYGWLIRSGFWFSSKSLRDWPLFMCCLSLAIFPLAAFVVEKLAQQKRISEPVIVLLHIVITTVAIIYPVLVILWCDSAFLSGSTLMLLTCIVWLKLVSYAHTTYDMRALAVSNEKGETMPDTFNMEEYPHNVSFQSLAYFMVAPTLCYQPSYPRTPSVRKGWVCRQLLKLVIFTGVMGFIIEQYMNPIVQNSQHPLKGNLLYAIERVLKLSVPNVYVWLCMFYCFFHLWLNILAELLRFGDREFYKDWWNAQTVEEYWRMWNMPVHKWMVRHVYFPCIRFGIPKGAAALTAFLVSAVFHELCIAVPCRMFKLWAFIGIMFQVPLVLITNYLKNKYRNSMVGNMIFWFIFCILGQPMCVLLYYHDLMNRKGEID

SEQ ID NO: 81 - protein, 521 aa, Jatropha curcas
MTILETTTSGGDGVAESSSDLNVSLRRRRKGTSSDGALPELTSNIVELESESGGQVMMDPGMVTEPETEKINGKDCGGDKDKIDNRENRGRSDIKFTYRPSVPAHRALRESPLSSDAIFKQSHAGLFNLCIVVLVAVNSRLIIENLMKYGWLIKTGFWFSSRSLRDWPLLMCCLTLPIFSLAAYLVEKLAYRKYISAPIVIFFHMLITTTAVLYPVSVILSCGSAVLSGVALMLFACIVWLKLVSYAHTNYDMRAIANSADKGDALSDTSGADSSRDVSFKSLVYFMVAPTLCYQPSYPRTDSVRKGWVVRQFVKLIIFTGFMGFIIEQYINPIVQNSQHPLKGDLLYAIERVLKLSVPNLYVWLCMFYCFFHLWLNILAELLRFGDREFYKDWWNARTVEEYWRMWNMPVHKWMVRHIYFPCLRHKIPRGVALLIAFFVSAVFHELCIAVPCHMFKLWAFIGIMFQIPLVGITNYLQNKFRSSMVGNMIFWFIFCILGQPMCVLLYYHDLMNRKGNAELR

SEQ ID NO: 82 - protein, 526 aa, Vernicia fordii
MTIPETPDNSTDATTSGGAESSSDLNLSLRRRRTASNSDGAVAELASKIDELESDAGGGQVIKDPGAEMDSGTLKSNGKDCGTVKDRIENRENRGGSDVKFTYRPSVPAHRALKESPLSSDNIFKQSHAGLFNLCIVVLVAVNSRLIIENIMKYGWLIKTGFWFSSRSLRDWPLLMCCLTLPIFSLAAYLVEKLACRKYISAPTVVFLHILFSSTAVLYPVSVILSCESAVLSGVALMLFACIVWLKLVSYAHTNFDMRAIANSVDKGDALSNASSAESSHDVSFKSLVYFMVAPTLCYQPSYPRTASIRKGWVVRQFVKLIIFTGFMGFIIEQYINPIVQNSQHPLKGDLLYAIERVLKLSVPNLYVWLCMFYCFFHLWLNILAELLRFGDREFYKDWWNARTVEEYWRMWNMPVHKWMVRHIYFPCLRHKIPRGVALLITFFVSAVFHELCIAVPCHIFKLWAFIGIMFQIPLVGITNYLQNKFRSSMVGNMIFWFIFCILGQPMCLLLYYHDLMNRKGTTESR

SEQ ID NO: 83 - protein, 523 aa, Vernonia galamensis; identical to SEQ ID NO: 64

SEQ ID NO: 84 - protein, 521 aa, Ricinus communis
MTILETPETLGVISSSATSDLNLSLRRRRTSNDSDGALADLASKFDDDDDVRSEDSAENIIEDPVAAVTELATAKSNGKDCVANSNKDKIDSHGGSSDFKLAYRPSVPAHRSLKESPLSSDLIFKQSHAGLFNLCIVVLVAVNSRLIIENLMKYGWLIKTGFWFSSRSLRDWPLFMCCLSLPVFPLAAYLVEKAAYRKYISPPIVIFLHVIITSAAVLYPASVILSCESAFLSGVTLMELACMVWLKLVSYAHTNYDMRAIADTIHKEDASNSSSTEYCHDVSFKTLAYFMVAPTLCYQPSYPRTAFIRKGWVFRQFVKLIIFTGFMGFIIEQYINPIVQNSQHPLKGDLLYAIERVLKLSVPNLYVWLCLFYCFFHLWLNIVAELLRFGDREFYKDWWNAKTVEEYWRMWNMPVHKWMVRHIYFPCLRRKIPRGVAIVIAFFVSAVFHELCIAVPCHMFKLWAFFGIMFQIPLVVITNYFQRKFRSSMVGNMIFWFFFCILGQPMCVLLYYHDLMNRDGN

SEQ ID NO: 85 - protein, 446 aa, Physcomitrella patens
MMESEDLKSNGKECDKVTNENRSDIKFNYRPSMPAHRGVRESPLSSDAIFKQSHAGLFNLCIVVLVAINSRLIIENIIKYGWLINGGFWFSSKSLRDWPLFMCCLSLPAFPFAAYLVEKLAYQNYLPQLVVVFLHTIITTGSLLYPVLVILRCDSAFLSGVTLMLFSCIVWLKLVSYAHTNSDLRAIAKSIDREDVPSISPYVGNPYDTYFKSLVYFMVAPTLCYQSSYPRTESVRKGWVVQQFVKLIIFTGFMGFIIEQYINPIVKNSQHPFKGNLLYAIERVLKLSVPNLYVWLCMFYCFFHLWLNILAELLCFGDREFYKDWWNARTVEEYWRMWNMPVHKWMVRHIYFPCLRNKIPKGLAILIAFLVSAVFHELCIAVPCHVFKLWAFIGIMLQVPLVVITKFLQNKFRSSMVGNMIFWLFFSILGQPMCVLLYYHDLMNRK

SEQ ID NO: 86 - protein, 493 aa, Physcomitrella patens
MAESESPENRIAAMESTSSSTSDLNFSIRRRSTVMDSASTEMMGSEGLKSSGKACDKVKIEKQSDMKFNYRPSMPAHSGVRESPLSSDAIFKQSHAGLFNLCIVVLVAVNSRLIIENLIKYGWLINSGFWFSSKSLRDWPLFMCCLSLPAFPLAAYLVEKLAYRNCISELVVVFLHIIITTASLLYPVLVILRCDSALLSGGTLMLFACIVWLKLVSFAHTSSDMRAIAKSIDKENTPSISSKADNSYDANFKSLVYFMVAPTLCYQSSYPRSASVRKGWVVRQFVKLIIFTGFMGFIIEQYINPIVQNSQHPLKGNLLYAIERVLKLSVPNLYVWLCMFYCFFHLWLNILAELLRFGDREFYKDWWNARTVEEYWRMWNMPVHKWMVRHIYFPCLRNKIPKWAALLIAFFVSAVFHELCIAVPCHMFKLWAFIGIMFQVPLVVITKFLQNKFKSSMVGNMIFWLFFSILGQPMCVLLYYHDLMNRKGKTERR

SEQ ID NO: 87 - DNA, 2074 bp, Arabidopsis thaliana
  source 1..2074: mol_type = other DNA
tgaatcctttttcctttcttcttcttcttctcttcagagaaaactttgcttctctttctataaggaaccagacacgaatcccattcccaccgatttcttagcttcttccttcaatccgctctttccctctccattagattctgtttcctctttcaatttcttctgcatgcttctcgattctctctgacgcctcttttctcccgacgctgtttcgtcaaacgcttttcgaaatggcgattttggattctgctggcgttactacggtgacggagaacggtggcggagagttcgtcgatcttgataggcttcgtcgacggaaatcgagatcggattcttctaacggacttcttctctctggttccgataataattctccttcggatgatgttggagctcccgccgacgttagggatcggattgattccgttgttaacgatgacgctcagggaacagccaatttggccggagataataacggtggtggcgataataacggtggtggaagaggcggcggagaaggaagaggaaacgccgatgctacgtttacgtatcgaccgtcggttccagctcatcggagggcgagagagagtccacttagctccgacgcaatcttcaaacagagccatgccggattattcaacctctgtgtagtagttcttattgctgtaaacagtagactcatcatcgaaaatcttatgaagtatggttggttgatcagaacggatttctggtttagttcaagatcgctgcgagattggccgcttttcatgtgttgtatatccctttcgatctttcctttggctgcctttacggttgagaaattggtacttcagaaatacatatcagaacctgttgtcatctttcttcatattattatcaccatgacagaggttttgtatccagtttacgtcaccctaaggtgtgattctgcttttttatcaggtgtcactttgatgctcctcacttgcattgtgtggctaaagttggtttcttatgctcatactagctatgacataagatccctagccaatgcagctgataaggccaatcctgaagtctcctactacgttagcttgaagagcttggcatatttcatggtcgctcccacattgtgttatcagccaagttatccacgttctgcatgtatacggaagggttgggtggctcgtcaatttgcaaaactggtcatattcaccggattcatgggatttataatagaacaatatataaatcctattgtcaggaactcaaagcatcctttgaaaggcgatcttctatatgctattgaaagagtgttgaagctttcagttccaaatttatatgtgtggctctgcatgttctactgcttcttccacctttggttaaacatattggcagagcttctctgcttcggggatcgtgaattctacaaagattggtggaatgcaaaaagtgtgggagattactggagaatgtggaatatgcctgttcataaatggatggttcgacatatatacttcccgtgcttgcgcagcaagataccaaagacactcgccattatcattgctttcctagtctctgcagtctttcatgagctatgcatcgcagttccttgtcgtctcttcaagctatgggcttttcttgggattatgtttcaggtgcctttggtcttcatcacaaactatctacaggaaaggtttggctcaacggtggggaacatgatcttctggttcatcttctgcattttcggacaaccgatgtgtgtgcttctttattaccacgacctgatgaaccgaaaaggatcgatgtcatgaaacaactgttcaaaaaatgactttcttcaaacatctatggcctcgttggatctccgttgatgttgtggtggttctgatgctaaaacgacaaatagtgttataaccattgaagaagaaaagaaaattagagttgttgtatctgcaaaaattttggtagagacacgcgaacccgtttggattttgttatggtgtaaagaaatttcaatcaaaaaactgttgtaataattgttaccaaaaagaaatgcttttctggaaacgaggggaaaaatagtagttttgtt

SEQ ID NO: 88 - DNA, 1512 bp, Brassica juncea
  source 1..1512: mol_type = other DNA
atggcgattttggattctggaggcgtcgctgtaccgccgacggagaacggcgtcgcggatctcgacaggctccaccgtcgtaaatcgagttcggattcttccaacggactcctctccgatacttccccgtcggacgatgttggagctgcggcggccgaaagggatcgggttgattccgctgccgaggaggaggctcagggaacagcgaatttagctggcggagatgccgaaactagggaatccgccggaggcgatgtaaggtttacgtatcgaccgtcggttccagctcatcggaggacgagggagagtcctctcagctccgacgctatcttcaaacaaagccatgcaggattgttcaacctctgtgtagttgttcttgttgctgttaacagtagactcatcatcgaaaacctcatgaagtatggttggttgatcagaactgatttttggtttagttctacatccttacgagactggccgcttttcatgtgttgtctttcactttcggtctttcctttggctgccttcacggtcgagaaaatggtacttcagaaattcatatctgagcctgttgccatcattcttcatgtcattataaccatgacagaggtcttgtatccagtctacgtcacactgaggtgtgattctgccttcttgtcaggtgtcacgttgatgctgctcacttgcattgtgtggctgaagttggtttcttacgctcatactagctacgacataagaaccctggccaattcagctgataaggtcgatcctgaaatctcctactatgttagcttgaagagcttggcgtatttcatggttgctcccacactgtgttatcagccaagctatccacgttctccatgtatccggaagggttgggtggctcgtcaacttgcaaaactggtcatattcactggactcatgggatttataatagagcaatatataaatcctattgttaggaactcaaagcatcctctgaaaggggaccttctatatgctattgaaagagtgttgaagctttcagttccaaatctatatgtgtggctctgcatgttctactgcttcttccacctttggttaaacatattggcagagctcctctgcttcggggaccgtgaattctacaaagattggtggaatgcaaaaagcgttggagattattggagaatgtggaatatgcctgttcacaaatggatggttcgacatgtatactttccgtgcctgcgcatcaagataccaaaagtacccgccattatcattgctttcttagtctctgcagtctttcatgagttatgcatcgcagttccttgccgtctcttcaatctatgggctttcatgggaattatgtttcaggtccctttggtctttatcacaaactttttacaagaaaggtttggctccatggtgggaaacatgatctttggttcagcttcttgcattttcggacaaccgatgtgtgggcttctttattaccatgacctgatgaaccgcaaaggatccatgtcctga

SEQ ID NO: 89 - DNA, 1512 bp, Brassica napus; identical to SEQ ID NO: 88
  source 1..1512: mol_type = other DNA

SEQ ID NO: 90 - DNA, 1510 bp, Brassica juncea
  source 1..1510: mol_type = other DNA
atggcgattttggattctggaactgtcacgatggcgacggagaacggtgtcgcggatctcgatatgcttcgtcgtcgtaaatcgagatcggattcttccaacggacttctctccgagacttccccatcggatgatgctggagctccggccgacgtggaggatcgggttgattcagctgctcagggaacagcgaatttagctggagatacggaaactagggaatccggtggaggaggaggaggaggaaacggcgaggtaaggtttacgtatcgaccgtcggttccagctcatcggaggacgagggagagtccactcagctccgacgctatcttcaaacaaagccatgcaggattgttcaacctctgtgtagttgttcttgttgctgttaacagtagactcatcatcgaaaatctcatgaagtacggttggttgatcagaactgatttctggtttagttctacatccctccgagattggccgcttttcatgtgttgtctttcactttcaatctttcctttggctgcctttaccgtcgagaaattggtactccagaaattcatatctgaacctgttgtcatcattcttcatattattatcaccatgactgaggtcttgtatccagtctacgtcaccctaaggtgtgattcggctttcttatcaggtgtcacattgatgctactcacttgcattgtgtggctgaagttggtttcttacgctcatactaattacgacataagaaccgtagctaattcagctgataaggtcgatcctgaagtctcctactatgttagcttgaagagcttggcgtatttcatggttgctcccacattgtgttatcagccaagctatccacgttctccgggtatccggaagggttgggtggctcgtcaatttgcgaaactggtcatattcactggactcatgggttttataatagagcaatatataaatcctattgtgaggaactcaaagcatcctttgaaaggggatcttctatacgctattgaaagagtgttgaagctttcagttccaaatctatatgtgtggctctgcatgttctactgcttcttccacctttggttaaatatattggcagagctcctttgcttcggggatcgtgaattctacaaagattggtggaatgcaaaaagcgtaggagattattggagaatgtggaatatgcctgttcataaatggatggttcgacatgtatactttccatgcctgcgcataaagataccgaaagtacccgccattatcattgctttcttagtctctgcagttttccatgagctgtgcattgcagttccttgccgtctctttaatttatgggctttcataggaatcatgtttcaggtgcctttggtctttatcacaaactatttacaagaaaggtttggctccatggtgggaaacatgatcttctggttcagcttctgcattttcggacaaccgatgtgtgtgcttctttattaccatgacctcatgaaccgcaaaggatccatgtcct

SEQ ID NO: 91 - DNA, 2090 bp, Tropaeolum majus
  source 1..2090: mol_type = other DNA
acgcggggagttttcaaaatcatattatgctttttcttcactactgcatgaactttctttctacttcttgcaactgatttgtaatccttacacatgtttctagttttctccatataaaaaaaatattctctgagcttctcgattctctagagagagaaggccaaaaaaaaatggcggtggcagagtcgtcacagaacacgacaaccatgagtggtcacggcgactcggatctcaacaatttccgtagaaggaaaccgagttcctccgtgattgaaccttcgtcgtccggttttacatccaccaatggcgtaccggcgactggccacgtggctgagaatcgtgaccaggatcgggtaggggctatggagaacgcaacaggatcggtcaacttaattggaaatggtggaggcgtggttatcgggaatgaagagaaacaggtaggggagactgatatacgattcacttaccggccttcgtttccggctcatcggagggtgagggagagtcctcttagctctgatgcaatcttcaaacagagccatgcgggtttattcaacttgtgtatagtagtgctcattgcagtaaacagtaggcttatcatcgaaaatcttatgaagtatggttggttgatcgatactggtttctggtttagctcaagatcactgggtgattggtccatctttatgtgctgtcttacactcccaattttcccacttgctgcttttattgttgaaaagctggtgcagcgaaatcatatatctgaacttgttgctgttctccttcatgtaatcgtttctaccgctgcagttttatatccagttattgtgatcttaacgtgtgattcggtgtatatgtctggtgtggtattgatgctctttggttgcattatgtggttgaagctggtgtcatatgcacatactagttctgatattagaacactggccaaatctggctataagggggatgcgcaccccaattcaaccattgtgagttgctcatatgatgttagcttgaagagtttggcatacttcatggttgctccgacattatgttaccagcctagctatcctcgttcgtcgtgtatccgcaagggttgggttgttcgtcaatttgtcaaactaatagttttcataggactcatggggttcattatagaacaatatattaatcctatcgttcgaaattccaaacacccattgaaaggagattttttatatgcaatagaaagagttttgaagctttcagttccaaatctatatgtttggctttgcatgttctactcttttttccacctctggttgaacatactggctgagcttcttcgctttggtgatcgtgaattctacaaagattggtggaatgcaaaaactgttgcggagtattggaaaatgtggaatatgcctgttcatagatggatggttcgtcatctatattttccctgtttgaggaatgggatacccaaggaaggtgccattattatcgcgttcttagtttctggtgctttccatgagctctgcattgcagttccttgccacgtattcaagttatgggcctttataggcattatgtttcaggttcccttggtattgattacgaattatctacaagaaaagttcagtaattctatggtgggcaatatgatcttctggttcatcttctgcatacttggccaacctatgtgtgtccttctatattaccatgacctgataaatctaaaggaaaagtgaaaaaatggaagttgcctatgctcagagtattcctatcccaatgcacacattatatggttctgtacaatctgtgcccccttcatcctttacacgtacccatgctggttcctgcacgatgatttgccttttgtttgtaagcaatatttggagagagtccaatttaggaagtgactagtgtggcttatatcttgtatactacctttagtcatgggggggtttttatattactagtaccaaaagtcaagttgtatatgatttacggtttagtttctttcatgttttttgtttttgtgtaaatatacgtttcatatatcactgttttttcaaagtaaaatcaataataccccatagatgttgaaactg

SEQ ID NO: 92 - DNA, 1494 bp, Echium sp.
  source 1..1494: mol_type = other DNA
cacaaatgacaatatgggagtcgccggagatcataagctccgatgaagcagcagcggcgttgaggcggagaggcggcgccaaggaggttgcggaacagagattggatagtgaagaagagaagaagaaggaggaggaaaatggaaaactgaagtatacttatcgagcttccgctccggctcaccggagaatcaaggagagtcctcttagctccgacgccattttcaaacaaagtcatgcaggcctcttcaatctttgcattgtggtgcttgttgctgtaaacagtcggcttatcattgagaatttgatgaagtatggttggctaattagctcagggttttggttaagttcaacatcattaagcgactggccacttctaatatgttgtctcagtctgccgatattccctctggcttcttttgtggttgaaaagttgtctcaacaggaatttatatctgagcaagtggtgatcactcttcatgcactcataacaacgactgtgattatgtatccagtaattgtcatcctcagatgtgatcccgccgttctatcaggtgtgatattgatgcttttcacgtgtattgtgtggttgaagctggtatcttatgcacatacaaactatgatatgcgagcactagcaaaggactgtgataagttacaggcactatcaggctcttcaatggaagattgttcttttgaagtcaacttccaagctttggtatacttcatggttgctccgacattatgttatcagctaaggtatccgcgtaccccctgcattcgttggggttgggttacacgtcatctcatcaagttaatcatatttactggactgatgggattcattattgagcagtatattaatccaattgtgaaaaattcacaacatccattgaaggggaacctgttgtatgctatagagagggtcttgaagctttcagttccaaatatatatgtctggctctgcatgttttattgtctcttccatctttggttaaatatactagccgaacttctgtgttttggggatcgtgagttctacaaagattggtggaatgctcaaacgatagaggagtattggaggatgtggaacatgcctgtacataaatggatggttcgtcatatatatttcccttgcttgcggaatgggatgcctaaggagttggctattttgattgcgttcctaatatctgcaatcttccatgagctgtgcattgctgtgccgtgtcacatctttaagttctgggcttttatcggaataatgtttcaggtccctttggtccttctgacaaatgttttggtaaaaaagttccaaaattcaatggttggcaatatgatattctggtgcttcttctgcattcttggtcaacccatgagtctgctgctctattaccatgatgtcttgaatagaaaagttaatgcaaactgatactacagatatcttgaaaatgtcatcacaaagagtgtgaaggatcgataggtttcgctcaacagga

SEQ ID NO: 93 - DNA, 1828 bp, Vernonia galamensis
  source 1..1828: mol_type = other DNA
tctgagctcaaatcaaatttctgcgactcatacaggattcaactcaatactttcttgatcggttctgctgttcatttacttgtaatttctacttctgctttgctttcatttcaagcttttttccttaataatggcgttattagatacgcctcagattggagaaataacgaccaccgccaccacaactataagacggcggaccactgtcaagcctgatgctggaatcggagatggattgtttgattcttcgtcgtcttccaaaaccaactcatccttcgaggatggtgacagtttgaatggtgatttcaatgacaaatttaaggaacagatcggagctggtgatgaatccaaggacgactccaaggggaacggacagaagatagatcacggaggagttaaaaagggacgtgaaacgactgtggtgcattatgcttatcggccttcttctccggctcatcggagaattaaagaatctccgcttagctctgacgccatcttcaagcagagtcatgcaggcctctttaacctttgcatagtggtgcttgttgcagtaaatggtaggctcatcattgagaatctgatgaagtatggactattgatcaattccaacttttggttcagttcgagatcattgagagactggccacttctgatgtgctgcctcactccttctgactttccacttgctgcctacattgttgagaaattggcatggaaaaaacgtatatccgaccctgttgtaatcacactccatgttataataactacaactgcaattctttatccggtcttcatgattctgaggttcgattcagttgttctatcaggcgtctcgttgatgctgtgtgcttgcattaattggttgaagttggtatcttttgtgcatacaaattatgacatgcggtcgcttttgaactcaactgataagggagaagtggaacccatgtcttcaaatatggattatttttatgatgtcaacttcaaaagcttggtttatttcatggttgctccaactttgtgttaccagataagctatcctcgcactgcatttattcgaaagggttgggtgttacggcaactgatcaagctagtaatatttacagggttcatgggattcatcattgaacaatatatcaatccgattgtcaaaaattctcgtcatccattgaaaggagactttttatatgcgattgagcgggttttaaagctttcagttccgaatttatatgtgtggctctgtatgttctactgcttttttcacctttggttaaatatacttgctgagcttctttgttttggggatcgtgaattttataaagattggtggaatgcacaaactattgaagagtattggaggctatggaatatgcctgttcataaatggattgttaggcacctttattttccatgcttgcgtaatgggatacctaagggtgctgccatattggttgcatttttcatgtctgccgtgttccatgagctttgtattgctgttccctgccacattttcaagttttgggcttttatcgggatcatgtttcaggtcccgttggtcctactcacaaattacttgcagcacaagtttcaaaactcgatggtgggaaatatgatcttctggtgctttttcagcatttttggtcaacccatgtgtgtattactttactaccatgatgtcatgaatcaaaaggggaaaagcaaataaaaagatgtgattgtgttgctccatttgatctcatagcatgactggactaaacaaacccaagggacacattttagtccttaaaggaaaatttttgtaggaaaaaaaaaaaaaaaaaaaaaaaaaaaa

SEQ ID NO: 94 - DNA, 2099 bp, Nicotiana tabacum
  source 1..2099: mol_type = other DNA
ggcacgagcgaaatcttacccaatcctccgttgcttttcttttagatcctctttttctgtcattctctttttcccaataacaacaactcattgcatgtgaaggttgattttgatttttgtgtttattcaaactctctcttcacgattttcttactctttctagaagtatccattactttttagtctgtgattcggcgaaagtaagcaatggtgatcatggaattgccggagagcgtcgaaatgacgacgacgacgacgacttcgggtattgagaacctcaactccgatcttaatcactcggttcggaggagacgtggcagtaatggttttgaggcggctagtgcaattaacagttcggatgcgaatatgagcgaagatagaagagatgtgtgtggcagcggtgctggattggaaacggtgaatgagcggagtaaatcggttggtgagtccagtgatgtaattcgaaaggaggacgacaggaatgataatgttgcgaatggtgaggaaagcaaatcaacggaaacaacaacgacgccgtttaaatttgcttacagggcgtcggcaccagctcaccggcgaatcaaggagagtcctctcagctccgacgccattttcaaacagagtcacgcaggcctgttcaatctctgtgtggtggtgctgattgctgttaacagcaggctgattatcgagaacttgatgaagtatggccttttaattagggctggcttttggtttagctcgaagtcgttgagggattggccgcttctaatgtgctgtctcagtctccaaattttgccgctcgctgcttttcttgtggagaagttggcacagcagaggcatttgactgagcgtgcggtggttactcttcacataactataacaacagctgccattttgtatccagttctggtcattcttgggtgtgattctgcttttctgtttggtgtcatattgatgctggttgcttgcattgtgtggatgaagctggtttcttacgcacatacaaatcatgatatgagacagctcgcaaagtctacggacaaggatgaaacttcagatggggatttctcttatgatgttagcttcaagagtttggcttacttcatggttgcgccaacattatgttatcagcttagctatccccacactccatgcattcgcaaaggttgggtggcacgccaattcatcaagctggtaatatttacaggattgatgggatttatcatagaacagtacattaacccaattgtgcaaaactcacaacatcctttgaaaggaaaccttttatatgccatcgagagggtattgaagctttcggttccaaatttatatgtctggctctgcatgttttactgcttctttcatctttggctaaatatacttgcggaactactatgttttggtgatcgtgagttctacaaggattggtggaatgccaaaacaattgatgagtactggaggatgtggaatatgcctgttcataagtggatggttcgtcacatttatttcccttgcttaagaaacggaattccaaagggggtcgcaatactgattgctttccttgtatctgctgttttccacgagctgtgtattgctgttccatgtcgccttttcaagtggtgggcattcatgggaattatgttccaggttcctttggtcatactcacaaacttcttacaaaacaagttccaaagctcgatggtgggcaatatgatgttctggtgctttttctgcattcttggtcagccaatgtgtgtgcttctgtattaccacgatgtgatgaatagaaaaagcagtgcacgttaagcttcatccagggatgaattgttgtatgagcaagtattttaagttttggatcccaagctctattctactgtttctggcaaggcattcctgctatttccttcatcagttccaacaatattcagatgatacgaaatatctgtttggaatgcacaacacaagccacggccagagatgctgatgtctcacattttattgtgttcttcatgtcggagaaatgtaaaatactatcttgagataactctcatgttagtaaatacctttttgcctctaaaaaaaaaaaaaaaaaaaaaaaa

SEQ ID NO: 95 - DNA, 1964 bp, Physcomitrella patens
  source 1..1964: mol_type = other DNA
tttagaaccaaactattctccgttaagttctgagttcgatttctttcttttctcaaattttccgtgcgatggcgatcttggactcgccggagatcctggacacgacgtcgtccagtgccgacaacggcgccgcacatcacaccactcttcgccggagacaaagtgcgcgctccgttccgcctcttctcgactccgattccaactctctggaggcagagagcgcaatcaatgattccgaaaatgttcgaaacgacgctaatttgatcgaaaatctccgcggcggagccgtggaatccgagaacgaaaaacaggagagttatggtaaggaggagggggcgaaagtgaaggagaatggagaaactagtaatggcaacggaactgatgttatggccgtcaaattcacattcaggccggcggcgcctgctcaccgcaaaaataaggagagtcctcttagctccgacgccatcttcaaacagagccatgcaggcctcttcaacctttgtatagtggtgcttgttgctgtaaatagcagactaataattgagaatttaatgaagtatgggtggctgatcaaatcaggattttggtttagttcaacatcgcttagggattggccactgctaatgtgttgtcttagtcttccagtttttgcactcgcttcatttcttgtcgagaagttggtgaaactaaattatatacctgagtgggtcgcagtctttcttcatgttacaatcacaacagtggaaatcttgtttccagttgttgtcattcttaggtgtgattctgctgttctatcaggtgtcacgctaatgctctttgcttgcactgtatggttgaagctcgtttcctacgcacatacaaactatgatttgagagtacttgcaaaatcacttgataagtgggaagctatgtccaggtactggaacctcgactacgcttatgatgtaagctttaagagtctggcatacttcatggttgctcctacattgtgttaccagccaagctaccctcggacagcttgcattcggaagggttgggtggtaaggcaactaattaagctggtaatattcacaggactcatgggatttattatagaacagtacataaacccgatcgttcaaaattctcaacatcctctgaaaggaaaccttttatatgccattgagagggtcttgaagctttctgttccaaatttatatgtgtggctctgcatgttttattgttttttccacctctggctaaatatacttgctgaacttctgtgctttggggaccgtgagttttataaggattggtggaatgcgaggacagtggaggagtactggagaatgtggaatatgcctgtccataaatggatggttcggcatatatactgtccatgcttacaaaatggaataccaaagatagtggcagttttgatcgcgtttcttgtgtctgcgatttttcatgagctgtgcgttgcagtcccttgccaaatattcaagttttgggcgttctcgggtatcatgcttcaggttcctctcgtaatcgtgactaattacttgcaagaaaagttcaaaaactcaatggtgggcaatatgatgttctggtgcttcttctgtatctttggtcaacctatgtgtgtgttgctgtactaccacgacttgatgaatcgaaaagctagtgcaaggtagggatgtgattcatcttctgagtagaaatctaaagctcaccagccccaacccacccgaaaaacaaaaaggagcaaggatcctgattgtgagctggtagataatttgctacaactatgtttcttaaatagctgggagtagtttgttatctgccttcacctaggacgacgttatgatctgttgtgatgggggtaagggggcatgcaaattttgtctatttttcaaggaatacagaaatggtgaaaatttgatgaagcatacccctcgtttactgacaaaaaaaaaaaaaaaaaa

SEQ ID NO: 96 - DNA, 1539 bp, Zea mays
  source 1..1539: mol_type = other DNA
atggcggactccgaagacgcgccgccagccgtgcaccgccgcccaccgcgccccgctcgcggtgctgctgcagcccagggcttcgcggccgcgttgcgccgccggctgagatccggcgctgcggtggcggcacgcgccagctttgccgcagactccggggacgagtctggccccggcgagccctcttcgtctcgccgccgcgacaacagcgggggcgcctcgtccgccgccggcggccgggccggggcaggggacttctccgcgttcaccttccgcgccgcggcgcctgtccaccggaaagccaaggagagccctctgagctccgacgccatcttcaagcagagtcatgcaggccttttcaacctatgtattgttgttctggttgcggtgaatagcaggctcattattgagaacctgatgaagtatggcttattaataagatctggcttttggtttaatgctacatcattgcgagactggccactgctaatgtgttgccttagtctacccatatttccccttggtgcatttgcagtcgaaaagttggcattcaacaatctcattagtgatcctgctactacctgttttcacatcctttttacaacatttgaaattgtatatccagtgctcgtgattcttaagtgtgattctgcagttttatcaggctttgtgttgatgtttattgcctgcattgtttggctgaagcttgtatcttttgcacatacaaaccatgatataagaaaactgatcacaagcggcaagaaggttgataatgaactgaccgcggctggcatagataatttacaagctccaactcttgggagtctaacatacttcatgatggctccgacactctgttatcagccaagttatcctcgaacaccttatgttagaaaaggttggctggtccgtcaagttattctatacttgatatttactggtctccaaggattcattattgagcaatacataaatcctattgttgtgaactctcaacatccattgatgggaggattactgaatgctgtagagactgttttgaagctctcattaccaaatgtctacctgtggctttgcatgttttattgccttttccatctgtggttaaacatacttgctgagattcttcgatttggtgaccgagaattctacaaagactggtggaatgcaaagacaattgatgagtactggagaaaatggaacatgcctgtgcataaatggattgttcgtcatatatatttcccttgcatgcgaaatggtatatcaaaggaagttgctgtttttatatcgttctttgtttctgctgtacttcatgagttatgtgttgctgttccctgccacatactcaagttctgggctttcttaggaatcatgcttcagattcccctcatcatattgacatcatacctcaaaaataaattcagtgacacaatggttggcaatatgatcttttggttttttttctgcatatacgggcagccaatgtgtgttctattgtattaccatgatgtgatgaaccggactgagaaggcaaaataa

SEQ ID NO: 97 - DNA, 1548 bp, Sorghum bicolor
  source 1..1548: mol_type = other DNA
atggcggacaccgacgacgcgccgccggccccggccgtgcaccgccgcccaccgcgccccgcccgcggtgctgccgctgcccagggcttcgcggccaagttgcgccgacggctcagctccggcgccgcggcggcggcgcgcgccagcttcgcggcagactccggggacgagtccggccccggggagccctcttcgtcccgccgccgcgacaacggcggggacgcctcgtccgccgccgacggcggccggggcggggcaggggacttctccgctttcacgttccgcgccgcggcgcctgtccaccggaaagccaaggagagccccctcagctccgacgctatcttcaagcagagtcatgcaggccttttcaacctatgtattgttgttctggttgcagtgaatagcaggctcattattgagaacctgatgaagtatggcttattaataaggtctggcttttggtttaatgctacatcattgcgagactggccactgctaatgtgttgccttagtctacctgtgtttccccttggtgcatttgcagttgaaaagttggcattcaacaatctcattactgatgctgccgctacctgttttcacatctttcttacaacacttgaaattgtatatccagtgcttgtgattcttaagtgtgattctgcagttttatcaggctttgtgttgatgtttattgcctgcattgtttggctgaagcttgtatcttttgcacatacaaaccatgatataagaaaactgatcacaagcggcaagaaggttgataatgaactgaccgtggctgacatagataatttacaagctccaactcttgggagtctaacatacttcatgatggctccgacactctgttatcagccaagttatcctcgaacaccttatgttagaaaaggttggctggttcgtcaagttattctatacttgatatttactggtctccaaggattcattattgagcaatacataaatcctattgttgtgaactctcaacatccattgaagggaggattactgaatgctgtagagactgttttgaagctctcattaccaaatgtctacctgtggctttgcatgttctattgccttttccatctatggttaaacatacttgctgagattcttcgatttggtgaccgtgaattctacaaagactggtggaatgcaaaaacaattgatgagtactggagaaaatggaacatgcctgtgcataaatggatgcttcgtcatatatattttccttgcatacgaaatggtatatcaaaggaagttgctgcttttatagcgttctttgtttctgctgtatttcatgagttatgtgttgctgttccctgccacatactcaagttctgggctttcttaggaatcatgcttcagattcccctcatcatactgacatcatacctcaaaaataaattcaatgacacaatggttggcaatatgatcttttggttctttttctgcatttacgggcagccaatgtgcgttctattgtattaccatgatgtgatgaaccggactgagaagacaaaataa

SEQ ID NO: 98 - DNA, 2103 bp, Oryza sativa alphaendornavirus
  source 1..2103: mol_type = other DNA
gtcgtcccgcgtctctcctcacctctcgtctccctcgcgacgtctccccttctccctccacccctccaatggtgggctccgatggcgacggcgacggcggcggaggggaagcacacgcgccggcggcgcccgcgcaccaccaccgccggcccccgcgcccgcggggaggcagcggggccatcgtcgagggcttcgcggcggcgctccgtcgcaggatccgctcgggggccgcggcggccgcgcgggccagcttcgggggcgactccggggacgaggccgcctccggggagccctcctcgtcctcgtcctcgtccccgtcccgccgccgtggcggcgactccaacggggcggaggcgtcctccgccgccgggggcggtggtggccgtggcggcggtggggacttctccgcgttcacgttccgcgcggcggcgccggtgcaccgcaaggccaaggagagccccctcagctccgacgccatcttcaagcagagtcatgcaggccttttcaacctatgcattgttgttctagttgcagtgaacagcaggcttattatcgagaacttaatgaagtatggcttattaataagagctgggttttggtttaatgataaatcattgcgggactggccacttctaatgtgttgtcttagtctgcctgctttccccctgggtgcatttgcagttgaaaagttggcatttaacaatgttattactgatgctgttgctacctgcctccatatcttcctttcaacaaccgaaattgtatatccagtgcttgtgattcttaagtgtgattctgcagttttgtctggctttttgttgatatttattgcctgtattgtttggctgaagcttgtatcttttgcacatacaaaccatgatataaggcaactgaccatgggcggcaagaaggttgataatgaactaagcacagttgacatggataatttacaacctccaactttagggaatctaatatacttcatgatggctcctacactctgttatcagccaagctatccccgaacttcatgtgttagaaaaggttggctgattcgtcaaattattctgtacttgatctttactggtcttcaaggcttcattattgagcaatacataaatccaattgttgtgaattctcagcatccattgaaaggaggactcctaaatgctgtagagactgttttgaaactctcattaccaaatgtttacctgtggctttgcatgttctatgcttttttccatctctggttaagtatacttgctgagattcttcgatttggtgaccgtgaattctacaaagattggtggaatgcaaaaacaattgatgagtattggagaaaatggaatatgcctgtacataaatgggttgttcgccatatttactttccttgcatgcgaaatggtatatcaaaggaagttgctgtcttgatatcattccttgtttctgccgtactccatgagatatgtgtcgctgttccctgccgcattctcaagttctgggcattcttaggaataatgctacagatcccccttatcgtattgacagcatacctcaaaagtaaattcagagatacaatggttggcaacatgatattttggttctttttctgcatctatgggcagccaatgtgccttctcctgtactatcatgatgtgatgaacaggattgagaaggcaagataaatgcgtgttgccatctttttcctctgtttcattttgtaccagcagaagcacaagcaataatccacatgctagccataaaacagcatgattcccaacggtgtggtacagccaaccttcctgttattctattttcttggctgtggtgtagatttagtttttaacttgtggctaaccgcaggaatgcctgtagataagcatctgtcattctgtctggcgacgttctccttattaatgtgtagatgtagaactgtttccgaaaactatatatcttgaatctgttatgcctcgacgaacataatccttttgttaagcttagttggtacagtctagaaaggataagagtcgtggatgtacgatttcgtctgccatatatcacgctcatattggcacaggtaactttgtcgctaccttctatctc

SEQ ID NO: 99 - DNA, 1473 bp, Oryza sativa
  source 1..1473: mol_type = other DNA
atggccccgcccccctcgctcgcccccgatcgcggcggcggcgaacccgacgacgccctccgcctgcgggcccgcgccgccgccgccgccggtgacgctcccgctccgcagcagcagcaggagcagaggcatcaggagcagcagcagcagctgctctggtaccgcgcgtcggcgcccgcccaccgccgcgtcagggagagccccctcagctccgacgccatcttccgccagagccatgcaggccttctgaacctatgcattgttgtgctggttgctgtgaacagcagacttattattgagaatttaatgaagtatggcctactaattagagctggattttggtttagtggaacatcgctggcagattggcctcttctcatgtgctgtctcactttaccaactttcccgcttgctgcacttatggttgagaagttggctcaaagaaaacttattagtaaacatgtggttattcttctccatatcgttattacaacatctgtccttgtctatccagttgttgtgattctaaagtgtgattcggcagtattatctggatttgtgttgatgtttcttgcaagcattatttggttgaagcttgtttcttttgctcatacaaattatgatataagaatgctgtccaaaagtattgaaaagggcgtgacacatgacatttctatagatccggagaacattaaatggccaacctttaaaaggctatcctacttcatgttggccccaacactttgttaccagccaagttatccccgaactacatatattagaaaaggttgggtggtccgacaactgataaaatgccttgtttttacaggcttgatgggttttataattgagcaatacataaatccaattgtgaagaattcgaagcatccattgaaagggaatttcttgaatgctatagagagagtattgaaattatcagtgccaacattatatgtctggctttgcatgttctactgttttttccatctctggttgaatattcttgctgagctcctctgttttggtgatcgtgaattctacaaggactggtggaatgccaaaacagttgaagagtattggagaatgtggaatatgcctgttcacaagtgggtcattcgacatatatattttccatgcataaggaatggtttttcaaagggtgttgctatcctaatctcgttcctggtttcagctgcatttcatgagatatgtattatttatttcattgtccttatcgatttgcagctatgtgttgctgttccatgccacatttttaaattctgggcatttattgggatcatgtttcagattcccctggtattcttgacgaaataccttcaagataaattcaataacacaatggtgggcaacatgatattttggttcttcttcagcatcctggggcaaccaatgtgtgttctcttatactaccatgatgtcatgaacaggcaacaagcccaaacaaatagatag

SEQ ID NO: 100 - DNA, 1590 bp, Sorghum bicolor
  source 1..1590: mol_type = other DNA
atggccccgcccccctccatggccgccgcctccgatcgcgccgtccccggcgccgacgcgaccgaggcgtcctccctccgcctccgccgcgccccctcagccgacgccggcgaccttgccgacgattcctcaggagaccggcgggagaacggcgagccgcaaccgccgcaggagcagcagcagcagcacgagatgctgtactaccgcgcgtcggcgcccgcccaccgccgcgtcaaggagagccccctcagctccgacgccatcttccggcagagccatgctggtcttctgaatctatgcatcgttgttctgattgcagtgaacagcagactcattattgagaatttaatgaagtatggcctattgataagagctggattttggtttagtgcaagatcgctgggtgactggccccttctaatgtgctgcctcactttaccagttttcccacttgttgccctcatggctgagaagctgattagaagaaagctcattggtgaacatgtggttattctactccatatcattattacaacatctgtcattgtctatccagttgttgtgactcttaagtgcgactcagcagtgctatctggattcttgctaatgtttcttgcgagcatcatgtggatgaagcttgtctcttatgcacatacaaattatgatataagggcattgtccaaaagtactgaaaagggtgctgcatatggaaattatgtcgatcctgagagtatgaaagatccaacctttaaaagtctagtgtacttcatgttggccccaacactttgttaccagccaacttatccccgaactacatgtattaggaagggttgggtgacccgacaacttataaagtgcctggtttttacaggcttgatgggcttcataattgagcaatatataaacccaattgtgaagaattccaaacatccactgaaagggaatttcttgaatgctatagaaagagtcttaaaactctcagtgccaacattatatgtatggctttgcatgttctattgcttttttcatttatggctgaacattctagctgaactcctctgtttcggtgaccgtgaattctacaaggactggtggaatgccaaaactgttgaagagtactggaggatgtggaacatgcctgttcataaatggatcatcagacacatatattttccatgtataaggaaaggcttttccaggggtgtagctattctagtctcgtttctggtttcagctgtatttcatgagatatgtattgcggtgccgtgccacattttcaaattctgggcattttctgggatcatgtttcagataccgttggtattcttgacaagatatctccaggctacgttcaagaatataatggtgggcaacatgatattttggttcttcttcagtatagtcgggcagccgatgtgtgtccttttatactaccatgatgtcatgaacaggcaggcccagcaagtagataattcggcagaaacatgtactttaagacaagttatcagaagcagactggagcgacgcagcaggaagcagcagcagcagcaggccagcagcccccctttgccattgttaccagctagctag

SEQ ID NO: 101 - DNA, 1485 bp, Zea mays
  source 1..1485: mol_type = other DNA
atggccccgcccccctccatgcctgccgcctccgatcgcgccggccctggccgcgacgcgggcgactcgtcctcccttcgcctccgccgcgccccctcagccgacgccggcgaccttgccggcgattcctcgggaggcttgcgggagaacggcgagccgcaatcgccgacgaatccgccgccgcaggagcagcagcagcacgagatgctatactaccgcgcgtcggcgcccgcccaccgccgcgtcaaggagagccccctcagctctgacgccatcttccggcagagccatgctggtcttctgaatctatgcattgttgttctgatcgcagtgaacagcagactcattattgagaatttaatgaagtatggcctgttgataagagctggattttggtttagtgcaagatcgctgggtgactggccccttctaatgtgctgcctcactctaccagttttcccactagttgcactcatggctgagaagctgatcacaagaaagctcattggtgaacatgtggttattctactccatatcattattacaacatctgccattgtctatccagttgttgtgactcttaagtgtgactcagcagtactatctggatttgtgctaatgtttcttgcgagcatcatgtggatgaagcttgtctcttatgcacatacaaattatgatataagggtattgtccaaaagtactgaaaagggtgctgcatatggaaattatgtcgatcctgagaatatgaaagatccaacctttaaaagtctagtgtactttatgttggccccaacactttgttaccagccaacttatcctcaaactacatgtattagaaagggttgggtgacccagcaactcataaagtgcgtggtttttacaggcttgatgggcttcataattgagcaatatataaacccaattgtgaagaattccaaacatccactgaaagggaattttttgaatgctatagaaagagtcttaaaactctcagtgccaacattatatgtatggctttgcatgttctattgcttttttcatttatggctgaacattgtagctgaactcctctgtttcggtgaccgtgaattctataaggactggtggaatgccaaaactgttgaagagtactggaggatgtggaacatgcctgttcataagtggatcatcagacacatatattttccatgtataaggaaaggcttttccaggggtgtagctattctaatctcgtttctggtttcagctgtatttcatgagatatgtattgcggtgccttgccacattttcaaattctgggcattttctgggatcatgtttcagatacccttggtattcttgacaagatatctccatgctacgttcaagcatgtaatggtgggcaacatgatattttggttcttcttcagtatagtcggacagccgatgtgtgtccttctatactaccatgacgtcatgaacaggcaggcccaggcaagtagatag

SEQ ID NO: 102 - DNA, 1912 bp, Physcomitrella patens
  source 1..1912: mol_type = other DNA
atgcctgtgaagagtagcaatctggcgggggagagggctgccaccagccatattaatgccaacacgaaattcgacctgcgggggtgtacgcctgcgcatcgcgttaggagggaaagcccgcttagttcagacgcaatttttcatcagagtcatgctgggttgttcaatctctgtattgttgttttgatagctgttaacagccggcttattattgagaacttaatgaagtatggactactcattagaactggtttctggtttagctccaagtctgcacgcgattggccactcttgatgtgcggtttgagtttaccgacttttccctttgcagcgttactagtggaaaaactatgctggaaaaatgaaaacgggaaatggttgattttcgtactacacctcataatcagcactgtagggatactgtatcctggatatgttatacacagggtgcaatccgcactgctgcctggtcttgtattgatactcattgcagtgactgggtggatgaagcttatatcttacgctcatgtcaacaaggacatgcgagaacttttgagagccaaagaaaagctacctgaggcaccacagtacgcagataaaatagaggttccggaccaccttacgatccaaaatattgcttatttcatgctcgcgcccacactttgctaccagttgagctaccctcgttcggacacaattcgaaaaagttgggtgttacggcaagccgggaaattggttgtgttcttgggtttagggggattcatcattgagcagtacataaatcccactgtgaagaattcacagcacccgctcaggggcaactatcttcaagcactggagagggttttaaagctttcgttgccagttctttatgtttggctgtgcttgttctactgtttatttcatctttggttgaatattgtggcggagctacttcgctttggggacagggaattttataaggactggtggaatgctcaaacagttgaagagtattggcgaatgtggaacatgcctgtgcacaagtggatggtgcggcatatttattttccctccattcgagctggcttatcaaagaaagcagcagtactactggtgtttgcaatttcagctttgtttcatgaggttatcattggtgttccgtgtcatatgcttcgatgctgggcttttcttggtatcatgatgcaggttccgttggtgtacttgacaaacgtgataaaagagcggtaccatagctctatggttggaaatatggtattttggttcttcttttgcattgtcgggcaacccatgtgcttgcttctctactaccacgacgttttcaacaactttcccagtacctgaactgagatcacccattcgtgcagttgttaatcttgtgaatagcactcctcatctgtaccttgttatggcttccacttctcgagaatcgactaaaccgcaactcatatgtttgtcaataacgattcattcgtaggcgggttggtgtgagattacaagagaggaaactttcgtcgtaagccagcagtgtagatacagtccaggtaggagtgtaacgggcacttgcttcaagggcagatccttgtcaacgcaagcttttgttggagcttgttggagcttgtttcgcacttagtattcttttctagctgtagttttaggtgacgttagtctattcttggtcccatcatccatcctgtaagatgctgggcgtgctcacgtgcagaagctgcctcgaatcctacgttacaatttcatgttggataccgcgtggatgtccgcttcagaatctgcgtcatgatgatgcacaccatttttttctattggaaactgaacagagggtagtgatacgtaagaacattttgggaaccgtgcctgaaaatcgtcggagcaaataatgatgtggttttgcagc

SEQ ID NO: 103 - DNA, 1278 bp, Selaginella sp.
  source 1..1278: mol_type = other DNA
atgcggccctcgctcccggcgcaccggcggagcaaggagagcccgctcagctccgacgccatcttcacgcagagccacgccggcctcttcaacctctgcatcgtcgtgctggtggcggtgaacagccggctcatcatcgagaatctcatgaagtatggcctcctcatccaggccgaggtcctcttcagctccaagtcgctcaaggactggccgctcctcatgtgtggcctctcgctgctgatcttccccctcgccgcctatgtcatcgagaagatcaaggcccgccgccccgccaccgccgtggcgccgctccacttgatcaacctcgccgccgcgctgctctacccgatctacgtgatcgagatgttccagtcggatctcctctcggggctggttctcatgctcatcgccgtcaccggctggctcaagctcgtctcctacgcgcacaccaacgccgacatccgcgcggtgaagaaggacggtggcaagatcgagctccccgccgaggcgccggcgatcgactacccggacaacatctcgctcaagaacatcgcctacttcatggcagcgccgacgctgtgctaccagctgagctacccgcgctcgccgcggattagaaccgggtgggtgctccggcagctgggcaagtggatcgtcttcaacggattcatgggcttcatcatcgggcagtacatgaatccgatcatccggaactcgacgcacccgctcaaggggaactacctctacgcgatcgagcgcgtgctcaagctgtccatccccacgctctacgtctggctcggcttcttctactgcttcttccacctgtggctcaacatcgtggcggagatcctgtgcttcggcgaccgcgagttctacaaggactggtggaacgccaagtcggtggacgagtactggcggctgtggaacatgcccgtccaccgctggctcgtccgccacgtctacttcccgtgcctccggctgggcctccacaagcagttcgccattttggtggtgttcgtcatctccgggatctttcacgagatttgcatcgcggtgccgtgccacatgctgcggggctgggcgtttctggggatcatgttccaggtgccgctggttctggtgaccaacgtcctccagcgcaagttccagagctccatggtcggcaacatgatcttctggttcttcttttgcatcgtcgggcagccgatgtgcgtgctgctctactatcacgacgttgtcaacaggcagcagctccagctagctgggcggtccaaataa

SEQ ID NO: 104 - DNA, 1524 bp, Euonymus alatus
  source 1..1524: mol_type = other DNA
atggctgctaacttgaacgaagcctcggatcttaatttttcgcttcggaggagaactggtggcatctcaagtacgactgtgcctgattctagttccgagacaagttcgtcggaggcggactatttggacggaggcaaaggtgccgcggacgtcaaagatcgtggggatggtgcggtggagtttcagaattcgatgaagaacgtggagaggattgagaaacatgagagccgagtaggattggattcgagattcacgtataggccatcggtcccggctcatcgcacaataaaggagagcccgcttagctcggacgcaatattcaaacagagtcacgcaggtctcttcaatctctgtatagtagttctggttgctgtgaacagcaggctgatcattgaaaatctaatgaagtatggatggttaataaggagtgggttttggttcagctcaagatcattgagagactggcccctttttatgtgttgtctcacactaccagtattccctcttgctgcttttctgtttgagaagttggctcaaaaaaatttaatatctgaacctgttgttgttttgcttcatatagtaaacactacagctgccgttttataccctgttttggtgattctaaggtgtgattctgcctttatgtctggggttacgttgatgctctttgcttgtattgtgtggttaaagttggtatcttatgctcataccaactatgatatgagagccctcaccaagtctgttgaaaagggggacacgccgttgagctctcagaacatggattactcgtttgatgtcaatatcaagagtttggcatattttatggttgctcccacattatgttaccagattagctatcctcgtaccccatatgttcgcaagggttgggtggttcgtcaatttgtcaagttaataatatttactggacttatgggatttataattgaacaatatatcaatcctattgtccagaattcacaacaccctttgaaaggaaactttttgtatgccattgagagagttttgaagctttcagtcccaaacctttatgtttggctctgcatgttctactgcctttttcatctctggttaaacatacttgctgaacttctttgttttggtgatcgtgagttctacaaggattggtggaacgctaaaactgttgaggagtactggagaatgtggaacatgcctgttcataagtggatggttcgtcatatctacttcccatgcttgaggaacgggatacccaagggtgttgcttttgtcatttccttcttagtttctgccgtcttccatgagctatgcattgctgttccctgccacatcttcaagttatgggctttctttggaataatgcttcaggttcccttggtgttgatcacaagttatctgcaaaataagttcagaagctcaatggtgggaaatatgatgttttggttctctttctgcatttttggtcaacctatgtgcttacttctatattaccatgatttgatgaatcgcaatgggaagatggagtag

SEQ ID NO: 105 - DNA, 1557 bp, Vitis vinifera
  source 1..1557: mol_type = other DNA
atggcgatctgcaactcgcctgtcagtgtgaccacgtcgtcatcaagctctcacgccgattcagatctcgacttttccattcggaagaggttcggcgggaaggggaaggccgtggcggattcgtcgctggagacggagacggaggcggcggcggcggcggtgctcgaagcagagaagtcggtgggcgaggtggggagtggcggtgatcgaggggaatcggggagtcaggtggtgaggaatggggagaacggagtggctgaggttgccgcgaaattcgcgtaccggccgtgtgcgccggctcaccggaaagtgaaggaaagtcctctcagttctgacgccattttcagacagagtcatgcgggtctcttcaacctctgtatagtagtgcttgtagctgtaaacagccggcttatcattgagaatcttatgaagtatggttggttaatcagggctggtttttggtttagttcaaaatcattgagagattggccactctttatgtgctgtttaaccctcccaatctttccacttgctgcttttgtggttgaaaagttggctcaacaaaagtatatctctgagcaggttgttgtctctcttcacatcataattactacagctgcagttttgtttccagttttggtgattctaaggtgtgattcagctgttctctctggtgtcacactaatgctctttgcttgcattgtgtggttaaaattggtatcttttgcacatacaaattatgacatgagagcagttgccaagttaattgataagggggatgacttgtccacttcattgaatatggattacccttatgatgtcaacttcaagagtttggcatacttcatggttgcccccacgctatgttaccagccaagctatcctcgcagcacatgcattcggaagggttgggtctttcgccaatttgtcaagttggcaatatttacaggtgttatgggatttataatagaacagtatattaatccaattgttcagaattctcagcaccctttgaaggggaattttttttatgcattggagaggattttgaagctttctgttccaaatttatatgtgtggctctgcatgttctactgctttttccacctctggttaaatatacttgctgagcttcttcgttttggggaccgtgagttctataaagattggtggaatgcaaaaacagttgaggagtattggagaatgtggaatatgcctgttcataaatggatggttcgccatctctattttccatgtctacggaatgggatatctaagggagtttctgtggtgattgcctttgccatatctgccatattccatgagctatgcattgctgtaccttgtcacatgtttaagctttgggctttcattggaattatgttccaggttcccttggttttggtcacaaattacttgcaaaataagttcagaaattctatggtgggaaatatgatcttctggctgtttttcagcattcttggtcagccaatgtgtgtgcttctatattaccatgacttgatgaatcgaaaagagacaactgaatcaagcctctga

SEQ ID NO: 106 - DNA, 1880 bp, Glycine max
  source 1..1880: mol_type = other DNA
gaagagaagactgagttagtaaacacgctcgctcggtcttcttttccaatggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactgaaggtgcacgtggataagcttttctgtttttggagtgtataattgatgtcgatatgttgatcaatattggtttccacgagtactttcatctaccatggcagtggctgctctgaaggatttccacctgatataccaggtcgcgaggctaattcatcttgatctatgtacttaatcaactctcctctggcaattgtatcgatatatgcaattttgagagccatacactggcattgataactgccaaggaacagtgntagctgtttttctgttaaatgttaattagtagagagctagatgtaaataaatttatgctcaaaaaaaaaaaaaaaaaaaaaa

SEQ ID NO: 107 - DNA, 1960 bp, Glycine max
  source 1..1960: mol_type = other DNA
acgcggggggagaagagaagactgagttagaaaacacgctcggtcttcttctccaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacagcgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagctcaaagtcattgagagactggccccttttcatgtgttgtctttctcttgtggtatttcctttcgctgcctttatagtggagaagttggcacaacggaagtgtatacccgaaccagttgttgttgtacttcatataatcattacctcaacttcgcttttctatccagttttagttattctcaggtgtgattctgcttttgtatcaggtgtcacgttaatgctgttttcttgtgttgtatggttaaaattggtgtcttatgcacatacaaactatgatatgagagcacttaccaaattagttgaaaagggagaagcactgctcgatactctgaacatggactatccttacaacgtaagcttcaagagcttggcatatttcctggttgcccctacattatgttaccagccaagctatcctcgcacaccttatattcgaaagggttggttgtttcgccaacttgtcaagctgataatatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcctggcagagcttcttcgatttggtgatcgtgaattctacaaggattggtggaatgccaaaactgtcgaagattattggaggatgtggaatatgcctgttcacaaatggatgatccgccacctatattatccatgtttaaggcacggtctcccaaaggctgctgctcttttaattgccttcctggtttctgctttattccatgagctgtgcattgctgttccttgccacatattcaagttgtgggctttcggtggaattatgtttcaggttcctttggtcttgatcactaattatctgcaaaataaattcagaaactcaatggttggaaatatgattttttggttcatattcagtatccttggtcaacctatgtgtgtactgctatactaccatgacttgatgaataggaaaggcaaacttgactgaagctacggccattacattttaaaggtgcacatggatgagcttttcagttttcagattgtaaaattgatgtggatatgttggtcaatattgttttctacgaatgctttcatctaccatggcattggctgctctgaaggaattccacgggatatgccagttcacgaggctaattcattatcttgatctatgtacttaccaactctcctctggcaattgtatcaaaatatgcaattttgagagccatacactggcattgataactgccaaggaacactctaactgttttctgttagctgttaattagtagagggctagatgtaaatggtttatgctcaatatatttatttcctcctagtcttcaagttccaaaaaaaaaaaaaaaaaaaaaaa

SEQ ID NO: 108 - DNA, 1536 bp, Lotus sp.
  source 1..1536: mol_type = other DNA
atggcgatttccgaagactctgaatctctattcgccgccgccgccgcttcctccgtcatccaaagcggctcttccgtacgccgcaggcccagcgctatctccgccgtcgcgacagtcgaagacgagagttcgagtgaagagccggtgccggtgagggattctggttccgacgtcgacgactccgtaagcagcgagcaacacgtctcccccgccaccgccaatcgagagaagaatcaggtgcatgacatctcagccaccaaattcgcctaccgtccttccgcccccgctcatcgcagagtcaaggagagccccctcagctccgacaacattttccgtcatcatgcgggtctcttcaacctttgtatagttgtgcttgttgcagtgaatagcagacttatcattgagaatttgatgaagtatggttggttgattaggactggcttttggtttagttcaaaatcattgagggattggccactcttcatgtgttgtctcagtcttgcaatatttccttttgccgcctttgtagtcgagaagttggtgcaacagaagtgtatttctgaaccagttgttgttcttcatatattcatttcaacagctgcagttgtctatccagttttagtaatcctcaggactgattctgcttttccatcaggcgtcacattaatgttatttgcttgcattgtatggctaaaactggtgtcttatgcacatacgaactatgatatgagagaacttaccaaatcaattgaaaagggagaagcacttcccaatactctgaatatggactattcttatgatgtgagcttcaagagcttggcatactttatgattgctcctacattatgttaccagccaagatatcctcgcagtccttctatccggaaaggttgggtgcttcgtcaacttgtcaagctgataatatttacaggagtaatgggatttataatagaacaatatattaatcctatagttcaaaattcacagcatcctttgaagggaaacctactatatgccattgaaagagttctgaagctgtctgttccaaacttatatgtgtggctctgcatgttctactgctttttccacctttggttaaatattctcgcagagcttcttagatttggtgatcgcgagttctacaaggattggtggaatgccaaaacttttgaagagtattggaggatgtggaatatgcctgttcacaaatggatgatccgacacctatattttccatgtttaagaaatggtatacccaagggtgttgctattttaattgccttcctggtttctgcattgttccatgagctgtgcattgctgttccttgccacattttcaagttgtgggcttttggtggaattatgtttcaggttcctttgatcttgataacaaattatctgcaaaataagttcagaaactcaatggttggaaacatgattttttggttcatattcagtattcttggtcaaccaatggccgtactgctatactaccacgatttgatgaatcggaaaagcaaacttgaccaaagctag

SEQ ID NO: 109 - DNA, 1620 bp, Lotus sp.
  source 1..1620: mol_type = other DNA
atggcaatttccgatacaccggaaaccactgcaaccgccacagccaccgtaacaaccatcgaaaccgacacagatctcaaacgctcttcactccgacgacgaccgagcgccacatcaaccgccggtggtctcttcgacgcggaaagtgcagctgcagatgccgttcgagattcaggctccgatgattcgttgaacggtaagatcaacaacgaagaagaggttaaagatcgaaaaacggatcatgcagaaggaattgttgacgatgatgatgataatgcggttaagaagaatggtggtaatgacgtcatcaatgatcgtgaaaatgttgctgtcgatttcaaattcacttatcgtccttcagttcccgctcaccggagaagcaaagaaagtcctcttagctccggcaatatttttagacagagtcatgcaggactgttcaatctctgtattgtggtgcttgttgcagtgaatagtaggcttattattgaaaatctgatgaagtatggatggttgattcgatctggcttttggtttagttcaaaatcgcttagagattggcccctcttcatgtgttgtcttagtcttgcaatatttccacttgctgcctttgtagtcgaaaagttggcccaacaaaaacgtatttctgaaccagttattgttctccttcatattgtaattacaactgttatatcttatggatccatggccttgctgtgcaggtgcgattctgcttttttatctggttccacgttaatgctattgacttgcatagtgtggttaaaattggtgtcatatgcacatacaacctatgatatgagagcgcttgctgtttcaaatgaaaagggagaaacaatgcccgatactttcaatatggaggagtacccacacaatgtgagcttccagagtttagcatacttcatggttgctcctacattatgctaccagccaagctatcctcgcacaccttcggttcgaaagggttgggtctgtcgacaacttctcaagctggtcatatttacaggagttatgggatttataatagaacaatatatgaatcctattgtccagaattcacaacatccattgaagggaaaccttctatatgccattgagagagttctgaagctttctgttccaaatgtttatgtgtggctgtgcatgttctattgctttttccatctttggttaaatatacttgcggagcttctccggtttggtgatcgtgagttctacaaagattggtggaatgcccaaacggttgaagagtattggaggatgtggaatatgcctgtgcacaaatggatggttcgtcacgtgtattttccctgcataaggtttggtatacccaagggtgctgctgctttgactgctttcctggtttctgctgtgttccatgagttatgcattgctgttccttgccgcatgttcaagttgtgggcttttattggaattatgttccaggttcctttggtcttgatcaccaattacctgaaaaataaatacagaaactcaatggttggaaatatgattttttggttcatattttgtattcttggtcaacctatgtgtgtactactatactatcatgacttgatgaataggaaaggtgaaattgactga

SEQ ID NO: 110 - DNA, 1566 bp, Jatropha curcas
  source 1..1566: mol_type = other DNA
atgacgattttggagaccactactagcggaggtgatggtgttgctgagtcgtcttccgatcttaacgtatcgcttcgacggagacggaaaggcaccagctcggatggagctttgccggaattgacttcgaatattgttgaattggaatctgaaagcggtggccaggtgatgatggatccaggtatggtgacggaaccggagacagagaaaattaatggaaaagattgcggcggtgacaaggataagattgacaatcgcgagaatcgtgggaggtcggatattaaattcacgtaccggccatcggtgccggctcatcgagcgctcagggagagtccgcttagctctgatgctatatttaaacaaagtcatgcaggtctgttcaacctctgtatagtagtgcttgttgctgttaacagcaggcttatcattgaaaatctaatgaagtatggttggttaattaaaacggggttttggtttagttcaagatcgttgagagattggccccttcttatgtgctgtcttaccctccctatattctctcttgccgcctatctagttgagaagttggcatatcgaaaatatatatctgcacctattgttattttctttcatatgctcattaccacaacagcagttttgtacccagtttctgtgattctcagttgtgggtctgctgttctgtctggtgttgcattgatgctctttgcttgtatcgtgtggttgaaattagtatcttatgcacatacaaactatgacatgagagccattgccaactcagctgacaagggagatgcactatccgatacttcaggtgcagattcttcacgtgatgttagcttcaagagtttggtctacttcatggttgctcctacgctatgttaccagccaagttatcctcgaacagattcagttagaaagggttgggtggttcgtcaatttgtcaagttaataatatttacaggattcatgggatttatcatagaacaatatatcaatcctattgtccagaattcacaacaccccttaaagggggatctattatatgccattgaaagggttttgaagctctcagttccaaacttatatgtgtggctctgcatgttctactgcttttttcatctatggttaaatatacttgctgagctccttcggtttggtgacagagagttctataaagattggtggaatgcaaggacagttgaggagtactggagaatgtggaatatgcctgttcataagtggatggttcgccatatctactttccatgcttgcggcataaaataccaaggggggtagccttgttaattgctttcttcgtttcagctgtatttcatgagttgtgcattgctgttccttgccacatgttcaagctctgggcttttattggaattatgtttcagattccattggtcgggatcactaattacctccagaacaagttcagaagctccatggtgggaaatatgatcttttggttcattttctgcattcttggtcaacccatgtgtgtgctattgtattatcatgacctaatgaatcggaaaggcaatgctgaattaagatga

SEQ ID NO: 111 - DNA, 2084 bp, Vernicia fordii
  source 1..2084: mol_type = other DNA
tctctctctctttgctttacgtgtacatcgaccaccaccacagccatcttgcgactgttcaattatcctataagtaccaccgcattcatcaccgccaatccttaactctaatttgctatactaaacacttgctttatatgcgcttttctatttactcttcactgtaatttcttattggtattcaaagtgttttcaatgacaatccctgaaacgccggataattccacggatgctaccaccagtggcggtgctgagtcctcttccgatcttaacctttctcttcgacggaggaggactgcttcaaactccgatggagctgtcgcggaattggcttccaagattgatgagttggaatctgatgccggaggaggccaggtgattaaggatccgggagcagaaatggattcggggactttgaaaagtaatggaaaagattgcggaaccgttaaggataggattgaaaatcgtgagaatcgtggaggatcggatgttaaattcacgtatcggccgtcggtgccggctcaccgggcgctcaaggagagtccgcttagctctgataatatatttaaacaaagtcatgcaggtctcttcaatctctgtatagtagtgcttgtagcggttaacagtcggcttatcattgaaaacataatgaagtatggttggttaattaagactgggttttggtttagttcaagatcgttgagagactggccacttcttatgtgctgtcttaccctcccaatattttctcttgctgcctatctagttgagaagttggcctgtcggaagtatatatctgcacccactgttgtttttcttcatattcttttctcctcaacagcagttttataccctgtttctgtgattctcagttgtgaatctgctgttttgtccggtgtcgcattgatgctctttgcttgtatcgtgtggttgaaattggtatcttatgcacatacaaactttgatatgagagcaattgctaactcagttgataagggagatgcgctatccaatgcttcgagtgcagagtcctctcatgatgttagcttcaagagtttggtttatttcatggttgctcccacattgtgttaccagccaagttatcctcgaactgcatccattcgaaagggttgggtggttcgtcaatttgttaagttaataatatttacaggattcatgggatttatcatagaacaatatatcaatcctatcgttcagaattcacaacatcctttaaaaggggatctcttatatgccattgagagggttttgaagctctcagttccgaatttatatgtctggctttgcatgttctactgcttttttcacctatggttaaatatacttgctgagctccttcgctttggtgatagagagttctataaagattggtggaatgcaaggacagttgaggagtattggagaatgtggaatatgcctgttcataagtggatggttcgccatatctactttccatgcttgcggcataaaataccaaggggggtggccttattaattactttcttcgtttcagcagtatttcatgagttgtgcattgctgttccttgccacatattcaagctctgggcttttattggaataatgtttcagattcctttggtcgggatcacaaattaccttcaaaacaagttcagaagctcaatggtggggaacatgatcttctggttcattttctgcattcttggtcaaccaatgtgcttgctgttgtattaccatgacctaatgaatcgaaaagggactaccgaatcaagatgacactaactcatcgtgtggtagactctatatatatacatagacttaccagagatgggttgcttccaacatattgtgcacaagaggcaattgttgttctcatcagaagagtgggttaattaattaattaatgtacaagcaattttgaaagtataatcactggcagggactagtgcccgactgtagtactgagattatagaggtattatcaatcgttagtggaaaattgtaaatgtataaagttcaatctttgtattgtttcttttctaatatcatatttttttttattgctcatcaaaaaaaaaaaaaaa

SEQ ID NO: 112 - DNA, 1828 bp, Vernonia galamensis; identical to SEQ ID NO: 93
  source 1..1828: mol_type = other DNA

SEQ ID NO: 113 - DNA, 2020 bp, Ricinus communis
  source 1..2020: mol_type = other DNA
cttcttcttctgctgctgttcctctctcctccaccgccacgctcacctctctttgcatgacataatactattgttcttattatcattttcactctttaaatacaaacatcaattattccttttctatcaaacacatgtattctattctctcgtcgtctagattctcatcttcattgaatcctccttcttagcgtgtctttgtccacttcttttgggcaccgacgtttttaatctccatgacgattctcgaaacgccagaaactcttggcgtcatctcctcctccgccacttccgatctcaacctctctctccgacgtagacggacctcaaatgactccgatggtgcacttgctgatttggcttcgaagtttgatgatgatgacgacgtaagatcggaagattctgctgaaaatattatcgaagatcctgtagcagcggttactgaattggcgacagcaaagagtaacggaaaagactgtgttgccaatagtaataaggataaaattgatagccatggaggatcatcggattttaaacttgcatataggccttcggttccagctcaccggtcacttaaggagagtccgcttagctctgatttaatatttaaacaaagtcatgcaggtctgtttaacctttgtatagtagtgctcgtagctgttaacagcaggctcatcattgagaatttaatgaagtatggctggttaattaagacgggcttttggtttagttcaagatcattgagagattggccgctttttatgtgctgtctttctctcccagtattcccccttgctgcctatctagttgagaaggccgcatatcgaaaatatatatctccgcctattgttattttccttcatgtgatcatcacctcagcagctgttttgtacccagcttctgtaattctcagttgtgaatctgcttttttatctggtgtcacattgatggaacttgcttgtatggtatggttgaaattggtatcctatgcacatacaaactatgatatgagagcgatcgctgacaccattcataaggaagatgcatccaattcttctagtacagagtattgtcatgatgtgagctttaagactttggcgtacttcatggtcgcacccacattatgttaccagccaagttatcctcgcacagcatttattagaaagggctgggtgttccgtcaatttgtcaaactaataatttttacaggattcatgggatttatcatagaacaatacatcaatcctatcgtccagaattctcaacaccctttaaaaggggatctcttatatgccattgagagggttctgaagctctcagttccgaatttatatgtgtggctctgcttgttctactgcttttttcacctgtggttgaatatagttgctgagctccttcgcttcggtgaccgggagttctacaaagattggtggaatgcaaaaactgttgaggagtactggaggatgtggaatatgcctgttcacaagtggatggttcgccatatctacttcccatgcctacgtcgtaaaataccaaggggggtagcaatagttattgctttcttcgtttcagctgtatttcatgagttgtgcattgctgttccttgccacatgttcaaactttgggctttttttggaataatgtttcagattcctttagttgtgatcactaattattttcaaaggaagttcagaagctcaatggtgggaaatatgatcttctggttctttttctgcattctcggccaacctatgtgtgtactgttgtattaccatgacctaatgaatcgcgatgggaactgaaccatgggctcagtccagatatgggtacaccttccaagatgttattttcgtgagtgaagactgcaccacagtgttgttcttgttacacaatccccattgacagagtaggttaatcgtcagtttcaggagataagacacaattttgaaagtacagcagaggctgctattaatgtatcatgttgagtttctgttatgttatgttattcttttttaatctc

SEQ ID NO: 114 - DNA, 1444 bp, Populus trichocarpa
  source 1..1444: mol_type = other DNA
aaatcatggcggcgttagagtctccggagaatcatatacgacgtcggatcttaacttctccgttcgacggaggtctactactgtcacggattcgccttcgacggagatgatggaatcggaggatttgaaaagtaatggtaaagaatgcgataaggttacgaatgagaatcgatcggatattaaattcaattatcggccttcaatgcctgctcatcgtggtgttagagagagtcctcttagttctgatgctatttttaaacaaagtcatgcaggtctcttcaatctctgtatagttgtgcttgtggctataaacagcaggcttatcattgaaaatataatcaagtatggttggttaattaacggtggattttggtttagttcaaaatcattaagagactggcccctgtttatgtgctgtcttagccttccagcattccctttcgcggcctatcttgttgagaagttggcatatcaaaattatttacctcaacttgttgttgttttccttcatacaatcatcaccacaggatcacttttatatccagttttagtaattctcaggtgtgattctgcttttctatctggtgtcacgttgatgctcttttcttgcattgtgtggctaaaattggtatcttatgctcatacaaactctgatttgagagcaattgccaagtcaatagatagggaagatgtcccatccatttctccttatgtgggtaatccttatgatacttactttaagagtttggtctacttcatggtggctcccacattatgttaccagtcaagctatcctcgcactgaatctgttcgaaagggatgggtggttcaacaatttgtcaagttaataatatttactggattcatgggatttatcatagaacaatatatcaatcctattgttaagaattcacagcacccttttaaaggaaatctcttgtatgccattgagagggtcttgaagctctcagttcctaatttatatgtatggctttgcatgttctactgctttttccacctgtggttaaatatacttgccgagctcctttgctttggtgatcgggagttctacaaggattggtggaatgcaagaactgttgaagaatattggagaatgtggaatatgccagttcataagtggatggttcgccatatctattttccatgcctacggaataaaataccgaaggggttagctatacttattgccttcttagtttcagctgtatttcacgagctgtgcattgctgttccctgccacgtgttcaagctctgggcatttattggaattatgttacaggttcccttagtggtgatcacaaaatttctccaaaataagttcagaagctccatggtgggaaacatgatcttctggttgtttttcagcattcttggtcaaccaatgtgtgtgcttctgtattaccatgacttgatgaatcggaag

SEQ ID NO: 115 - DNA, 1816 bp, Populus trichocarpa
  source 1..1816: mol_type = other DNA
aacatatttaaatcatggcggagtcagagtcaccggagaatcgtatagcggcaatggaaagtacatcttcctcgacgtcagatctcaacttctctattcgacggaggtctacagtcatggactcggcttcgacggaaatgatgggatcggagggtttgaaaagtagtggtaaagcatgcgataaggttaagattgagaagcaatcagatatgaaattcaattatcggccatcaatgcccgctcatagtggtgttagagagagtcctcttagttctgatgctatttttaaacaaagtcatgcaggtctcttcaatctctgtatagtagtgcttgtggctgtaaacagcaggcttatcattgaaaatttaatcaagtacggctggttaatcaattcaggattttggtttagttcaaaatcattaagagactggcccctgtttatgtgctgtcttagtcttccagcattccctctcgcggcctatctcgttgagaagttggcatatcgaaattgtatatctgaacttgttgttgttttccttcatataatcatcaccacagcatcacttttgtatccagttttagtaattctcaggtgtgattctgctttactatctggtggcacattgatgctctttgcttgcattgtgtggttgaaattggtatcttttgcacatacaagctctgatatgagagcaattgccaagtcaattgataaggaaaataccccatccatttcttcgaaagcagataattcttatgatgctaactttaagagtttggtctacttcatggtggctcccacattatgttaccagtcaagctatcctcgttctgcatctgttcgaaagggttgggtggttcgacaatttgtcaagttaataatatttactggattcatgggatttatcatagaacaatatatcaatcctattgttcagaactcgcagcaccctttgaaaggaaatctcttgtatgccattgagagggtcttgaagctctcagttcctaatttatatgtttggctctgcatgttctactgctttttccacttgtggttaaatatacttgccgagctccttcgctttggtgatcgggagttctacaaggattggtggaatgcaagaactgttgaagagtactggagaatgtggaatatgccagttcataagtggatggttcgccatatctattttccatgtttacggaataaaataccaaagtgggcagccttacttattgccttctttgtctcagctgtatttcatgagttgtgtattgctgttccttgccacatgttcaagctctgggcatttattggaattatgtttcaggttcccttagtggtgatcacaaaattccttcaaaataagttcaaaagctcaatggtgggcaatatgatcttctggttatttttcagcattcttggtcaacctatgtgtgtgcttctatattaccatgacttgatgaatcggaaagggaaaactgaacgaagatgacaaatgcggtatggtagagatcgtcaagatgaacaaaatgcacggttatgatagtagagcaggcattaggtgtgccttttcttatgtattctgcaggagaaattgactcgattttgttgagtcgagagatggtctcttcaggacttttatttttatgtatctcaattgacgtgcaagcaattttggaagtacaagcactggcaattaaaatgccaatgcaacagtggatctgttgtgttggttaatcatttccagaaatttgtaaatgtttcttgttccgtcttttgcttcaaaggaaataaaaaaagaagaaaatttct

SEQ ID NO: 116 - protein, 520 aa, Arabidopsis thaliana; identical to SEQ ID NO: 58

SEQ ID NO: 117 - protein, 518 aa, Tropaeolum majus; identical to SEQ ID NO: 62

SEQ ID NO: 118 - protein, 494 aa, Zea mays; identical to SEQ ID NO: 72

SEQ ID NO: 119 - protein, 314 aa, Arabidopsis thaliana
MGGSREFRAEEHSNQFHSIIAMAIWLGAIHFNVALVLCSLIFLPPSLSLMVLGLLSLFIFIPIDHRSKYGRKLARYICKHACNYFPVSLYVEDYEAFQPNRAYVFGYEPHSVLPIGVVALCDLTGFMPIPNIKVLASSAIFYTPFLRHIWTWLGLTAASRKNFTSLLDSGYSCVLVPGGVQETFHMQHDAENVFLSRRRGFVRIAMEQGSPLVPVFCFGQARVYKWWKPDCDLYLKLSRAIRFTPICFWGVFGSPLPCRQPMHVVVGKPIEVTKTLKPTDEEIAKFHGQYVEALRDLFERHKSRVGYDLELKIL

SEQ ID NO: 120 - protein, 317 aa, Brassica napus
MGKVRDFGAEDHIPSNIFHAVTAISICLSAIYLNLALVLISLFFLPTSLSLLVLGLLSLFIIIPIDDRSKYGLKLARYICKHAASYFPVTLHVEDYEAFKPDRSYVFGYEPHSVWPIGAVALVDLAGFMPLPNIKLLASNAIFYTPFLRHMWAWLGLASASRKSFSSLLESGYSCILVPGGVQETFHLQHDVENVFLSSRRGFVRIAMEQGAPLVPVFCFGQSRAYKWWKPDCDLYFKLARAIRFTPICFWGVFGSPIPYRHPIHVVVGKPIQVAKSLQPTDEEIDELHGQFVEALKDLFERHKAGAGYSDLQLNIL

SEQ ID NO: 121 - protein, 345 aa, Arachis hypogaea
MEVSGAVLRNVTCPSFSVHVSSRRRGGDSCVTVPVRMRKKAVVRCCCGFSDSGHVQYYGDEKKKENGTAMLSTKKKLKMLKKRVLFDDLQGNLTWDAAMVLMKQLEQVRAEEKELKKKRKQEKKEAKLKASKMNTNPDCESSSSSSSSESESESSESECDNEVVDMKKNIKVGVAVAVADSPRKAETMILYTSLVARDVSANHHHHNAVELFSRNNDISVGSINGGLKNENTAVITTEAIPQKRIEVCMGNKCKKSGSIALLQEFERVVGAEGGAAAAVVGCKCMGKCKSAPNVRIQNSTADKIAEGFNDSVKVPANPLCIGVAWRMLKPLWLRFLGENQESTNE

SEQ ID NO: 122 - protein, 671 aa, Arabidopsis thaliana
MPLIHRKKPTEKPSTPPSEEVVHDEDSQKKPHESSKSHHKKSNGGGKWSCIDSCCWFIGCVCVTWWFLLFLYNAMPASFPQYVTERITGPLPDPPGVKLKKEGLKAKHPVVFIPGIVTGGLELWEGKQCADGLFRKRLWGGTFGEVYKRPLCWVEHMSLDNETGLDPAGIRVRAVSGLVAADYFAPGYFVWAVLIANLAHIGYEEKNMYMAAYDWRLSFQNTEVRDQTLSRMKSNIELMVSTNGGKKAVIVPHSMGVLYFLHFMKWVEAPAPLGGGGGPDWCAKYIKAVMNIGGPFLGVPKAVAGLFSAEAKDVAVARAIAPGFLDTDIFRLQTLQHVMRMTRTWDSTMSMLPKGGDTIWGGLDWSPEKGHTCCGKKQKNNETCGEAGENGVSKKSPVNYGRMISFGKEVAEAAPSEINNIDFRGAVKGQSIPNHTCRDVWTEYHDMGIAGIKAIAEYKVYTAGEAIDLLHYVAPKMMARGAAHFSYGIADDLDDTKYQDPKYWSNPLETKLPNAPEMEIYSLYGVGIPTERAYVYKLNQSPDSCIPFQIFTSAHEEDEDSCLKAGVYNVDGDETVPVLSAGYMCAKAWRGKTRFNPSGIKTYIREYNHSPPANLLEGRGTQSGAHVDIMGNFALIEDIMRVAAGGNGSDIGHDQVHSGIFEWSERIDLKL

SEQ ID NO: 123 - protein, 685 aa, Ricinus communis
MPVIRRKKPTSEPNKNSASDSKTPSEEEEHEQEQEQEEDKNNKKKYPKKKSSEINAKKWSCIDSCCWFVGCICVTWWVLLFLYNAVPASLPQYVTEAITGPLPDPPGVKLKKEGLTAKHPVVFVPGIVTAGLELWEGHQCADGLFRKRLWGGTFGEVYKRPLCWVEHMSLDNETGLDPPGIRVRPVSGLVAADYFAPGYFVWAVLIANLARIGYEEKTMFMASYDWRLSFQNTEVRDQTLSRMKSNIELMVSINGGNKAVIVPHSMGVLYFLHFMKWVEAPAPMGGGGGPDWCAKHIKAVMNIGGPFLGVPKAVAGLFSAEARDIAVARAIAPGFLDNDMFRLQTLQHMMRMSRTWDSTMSMIPRGGDTIWGDLDWSPEEGYIPRKKRQRNNATDNVNEGGAESEISQRKIVRYGRMISFGKNIAEAPSYDIERIDFRDAVKGRSVANNTCLDVWTEYHEMGFGGIKAVAEYKVYTAGSTIELLQFVAPKMMERGSAHFSYGIADNLEDPKYEHYKYWSNPLETKLPNAPEMEIFSMYGVGIPTERAYVYEFSPAAECYIPFQIDTSANDGDEDGCLKDGVYTVDGDETVPVLSAGFMCAKAWRGKTRFNPSGSRTYIREYDHSPPANLLEGRGTQSGAHVDIMGNFALIEDIMRVAAGATGEDLGGDQVYSDIFKWSQKIKLPL

SEQ ID NO: 124 - DNA, 2074 bp, Arabidopsis thaliana; identical to SEQ ID NO: 87
  source 1..2074: mol_type = other DNA

SEQ ID NO: 125 - DNA, 2090 bp, Tropaeolum majus; identical to SEQ ID NO: 91
  source 1..2090: mol_type = other DNA

SEQ ID NO: 126 - DNA, 1485 bp, Zea mays Hopscotch virus; identical to SEQ ID NO: 101
  source 1..1485: mol_type = other DNA

SEQ ID NO: 127 - DNA, 1330 bp, Arabidopsis thaliana
  source 1..1330: mol_type = other DNA
agttaaaagattggttatttgggctctgcactcaagtgagagagaagatagatagatctgagtagaatcttcgattcattattcgttgtcgtcgttcatctgtgagaagcggacaaaccaaagaatccaccggagctagtgatatgggtggttccagagagttccgagctgaggaacattcaaatcaattccactctatcatcgccatggccatctggcttggcgccattcacttcaacgtcgctcttgttctctgttctctcattttccttcctccttctctatctctcatggtcttgggcttgctctctctgtttatctttatcccaatcgatcatcgtagcaaatatggtcgtaagctcgctaggtacatatgcaagcacgcgtgtaattatttccccgtctctctgtacgtcgaggattacgaagctttccagcctaatcgtgcctatgtctttggttatgaaccacattcggtgctaccgattggagttgttgctctttgtgatctcacagggtttatgcctattcctaacattaaagttcttgcaagtagtgctatattctacactccctttctaaggcatatatggacatggttagggctcaccgctgcttctaggaagaatttcacttcccttttggattctggctacagttgtgttcttgtacctggtggtgtgcaggagacttttcatatgcaacatgatgctgagaatgtcttcctttcaaggagaagaggatttgtgcgcatagccatggaacaggggagccctctggttccagtattctgctttggtcaggcacgcgtgtacaaatggtggaagccggattgtgatctctatcttaaactatctagagcaatcagattcaccccgatctgcttctggggagtttttggatcaccattaccgtgtcgacagcctatgcatgtggtcgttggtaaaccaatagaagtcacaaaaactctgaagccaactgacgaagagattgctaagtttcatggccagtatgtggaagcgcttagggatctgtttgagaggcacaagtcccgagtcggctatgatcttgagctgaaaattctttgaacaaaatctccaatggaaataattacttgtgtgtatccttcattaattgttaccttggagctggatttggacttaatataaatgactacatcatgtagtctacatgtattgcatgtctttagcatcgactgttgaagtaatggaatacgtttataaagcctgtaaattacatgtcgtcttgcacaagagtatgtggtaataataacatttgacccaaaaataatactagttaaatttttcct

SEQ ID NO: 128 - DNA, 954 bp, Brassica napus
  source 1..954: mol_type = other DNA
atgggcaaagtcagagactttggagctgaggatcatatcccatcaaacatattccatgcagtgaccgccatatccatctgcctcagcgccatttacttgaacctcgctcttgttctcatctccctcttcttcctcccaacttctctctccctcctggtcttgggcctgctctctctgtttatcatcatccctatagatgatcgtagcaagtacggtcttaagctggctaggtacatatgcaagcacgcggctagttacttccccgttactctgcatgtcgaagactacgaagctttcaagcctgatcgctcctatgtatttggttatgaaccacactcggtgtggcccattggagctgttgcacttgttgatctggcagggtttatgcctcttcctaacatcaaacttcttgcaagcaatgctatattctacacgccgtttctaaggcacatgtgggcatggttagggctcgcctctgcttctaggaagagtttctcttctcttctggagtctggctatagttgtatccttgtacctggtggtgtgcaggaaacatttcacttgcaacatgatgttgagaacgtcttcctttcatcgagaagaggatttgtgcgcatcgccatggaacaaggggcacctcttgttccagttttctgctttggtcagtcccgtgcgtacaagtggtggaagccggattgtgacctttattttaaactagcaagagcgatcaggtttactcctatctgtttctggggagttttcggatccccaataccatatagacaccctattcatgtggtggttggtaaaccaatacaagttgcaaagtctctgcagccaactgatgaagagattgatgagttgcatggccagtttgtggaagcgcttaaggatctgtttgagaggcacaaggccggagcaggctactctgatctgcagttgaacattctttga

SEQ ID NO: 129 - DNA, 1637 bp, Arachis hypogaea
  source 1..1637: mol_type = other DNA
aatgaacttgacataaagtggttgtttgtaacaccccatttagtgttttgcttagatgttgagagttctataaacttttgtactatttggtaccccgtaattaatagaaatagaaatgtgataatggttctatgtttcattccagaaaaaaattgtcattttaaaaagttttcttaaattctgaatgggaatgatgatcattcagatcaataaggttaacacttttttatatgatattttatgtaatctgattaatttttttttggtgacaaaaaactcgtgccgaattcggcacgaggtcaaaacctcagaagagagaaaaggagaatttggttcctaattaattctcaccatcaacgatggaggtttcaggcgccgttctaaggaatgtcacgtgcccttccttttctgtgcacgtgagttcccgtcgtcgtggtggtgatagttgtgttacagtgccggtgaggatgagaaaaaaggcggtggtgcgttgttgctgcgggttcagtgattcggggcatgtgcagtattacggggacgagaagaagaaggagaatggaaccgctatgttgagcaccaagaagaagctcaagatgctgaagaaacgtgtccttttcgatgatcttcaaggaaacctgacttgggatgctgctatggttttgatgaagcagctagagcaagtaagggcagaggagaaggaattgaagaaaaaaaggaagcaagagaagaaggaggcaaaactcaaagcctctaagatgaacaccaatcctgattgcgaatcgtcatcgtcatcgtcatcatctgaatctgaatctgaatcaagtgagagtgaatgtgacaatgaggtggttgacatgaagaagaacattaaggttggtgttgccgttgctgttgccgattccccacgaaaggcggaaaccatgattctatacacctcccttgttgcccgagatgttagtgctaatcatcatcatcataatgccgtggaattattctctagaaacaatgacatatcagttggaagcattaatggtggccttaagaatgagaatactgcggttattaccactgaagctattcctcagaagaggattgaggtatgcatgggaaacaagtgcaagaaatccggatctattgcattgttgcaagaatttgagagagtggttggtgctgaaggaggtgctgctgctgcagttgttggatgcaagtgcatggggaagtgcaagagtgcacctaatgtgaggattcagaactctactgcagataaaatagctgaggggttcaatgattcagttaaggttccagctaaccctctttgcattggggttgcatggaggatgttgaaaccattgtggcttagattcttgggcgagaatcaggaaagtactaatgaataatttgctggtatgctgtttggaaaattgtatatacgtagtgccagaacctatcagattgttgttttattttatataaacatagactgcatattgttgtgagattcgatttcctcatttattggaacttccagagcctgatttgtgtccattcgagctcgactcaaagatttacatggcctgctcaatctatgaattcaaatttgagggccctgtttggcattaatattaatatattaatat

SEQ ID NO: 130 - DNA, 2811 bp, Arabidopsis thaliana Art1 virus
  source 1..2811: mol_type = other DNA
cccaagtgtaatattgttattaatacatgggctatactaaaagccccacgaaaagtttactgaactatttgaggcccaacaagagcctatcggattaacgcctactgcagaagaaaatctgtctgcactccacccaagaaaacgcagactaattaatgaaatcaacgaaacggataggtcgggtctaaggttgaccatgaaccgcaacctgaaccaggagcaaagtggtcaagttttgccatccggtccgagtcccttggaggaataataccagaacagaaaaaaacagaaaagtcgacaataaacaaaagagacaaatttgatttgattggttccagaaattcgcagagaaacagctctttgtctctctcgactgatctaacaatccctaatctgtgttctaaattcctggacgagatttgacaaagtccgtatagcttaacctggtttaatttcaagtgacagatatgccccttattcatcggaaaaagccgacggagaaaccatcgacgccgccatctgaagaggtggtgcacgatgaggattcgcaaaagaaaccacacgaatcttccaaatcccaccataagaaatcgaacggaggagggaagtggtcgtgcatcgattcttgttgttggttcattgggtgtgtgtgtgtaacctggtggtttcttctcttcctttacaacgcaatgcctgcgagcttccctcagtatgtaacggagcgaatcacgggtcctttgcctgacccgcccggtgttaagctcaaaaaagaaggtcttaaggcgaaacatcctgttgtcttcattcctgggattgtcaccggtgggctcgagctttgggaaggcaaacaatgcgctgatggtttatttagaaaacgtttgtggggtggaacttttggtgaagtctacaaaaggcctctatgttgggtggaacacatgtcacttgacaatgaaactgggttggatccagctggtattagagttcgagctgtatcaggactcgtggctgctgactactttgctcctggctactttgtctgggcagtgctgattgctaaccttgcacatattggatatgaagagaaaaatatgtacatggctgcatatgactggcggctttcgtttcagaacacagaggtacgtgatcagactcttagccgtatgaaaagtaatatagagttgatggtttctaccaacggtggaaaaaaagcagttatagttccgcattccatgggggtcttgtattttctacattttatgaagtgggttgaggcaccagctcctctgggtggcgggggtgggccagattggtgtgcaaagtatattaaggcggtgatgaacattggtggaccatttcttggtgttccaaaagctgttgcagggcttttctctgctgaagcaaaggatgttgcagttgccagagcgattgccccaggattcttagacaccgatatatttagacttcagaccttgcagcatgtaatgagaatgacacgcacatgggactcaacaatgtctatgttaccgaagggaggtgacacgatatggggcgggcttgattggtcaccggagaaaggccacacctgttgtgggaaaaagcaaaagaacaacgaaacttgtggtgaagcaggtgaaaacggagtttccaagaaaagtcctgttaactatggaaggatgatatcttttgggaaagaagtagcagaggctgcgccatctgagattaataatattgattttcgaggtgctgtcaaaggtcagagtatcccaaatcacacctgtcgtgacgtgtggacagagtaccatgacatgggaattgctgggatcaaagctatcgctgagtataaggtctacactgctggtgaagctatagatctactacattatgttgctcctaagatgatggcgcgtggtgccgctcatttctcttatggaattgctgatgatttggatgacaccaagtatcaagatcccaaatactggtcaaatccgttagagacaaaattaccgaatgctcctgagatggaaatctactcattatacggagtggggataccaacggaacgagcatacgtatacaagcttaaccagtctcccgacagttgcatcccctttcagatattcacttctgctcacgaggaggacgaagatagctgtctgaaagcaggagtttacaatgtggatggggatgaaacagtacccgtcctaagtgccgggtacatgtgtgcaaaagcgtggcgtggcaagacaagattcaacccttccggaatcaagacttatataagagaatacaatcactctccgccggctaacctgttggaagggcgcgggacgcagagtggtgcccatgttgatatcatgggaaactttgctttgatcgaagatatcatgagggttgccgccggaggtaacgggtctgatataggacatgaccaggtccactctggcatatttgaatggtcggagcgtattgacctgaagctgtgaatatcatgatctctttaagctgtcctgtcagcttatgtgaatccaatactttgaaagagagatcatcatcaattcatcatcatcgtcatcatcatgatgctcaactcacaaagaagcctgagaatgatactttggtgcgaaattctcaatacctctttaatattcttattgaatgtaaattatacaatcctatctaatgtttgaacgataacacaaaacttgctgcgccatgtttgtttgtcttgtcaaaagcatcaatttgtgggttatacgtagtgtagaggatgattcaaatttgtgataaatttggtaatcaaagttaattctg

SEQ ID NO: 131 - DNA, 2473 bp, Ricinus communis
  source 1..2473: mol_type = other DNA
cttgaattttgtttcgattactttaaaaaaaattgcctttttctgaaagcgcttctagtttgatcttaatggattatttacttttcagaaattagtagctcaaacaaaattaagagagaaaaagagatcgtgggttttttcaaagaaaaagtttcaaacagaaagcacaaactttccgatgtggcttgcgaggaagttggggatgacataaattcttctgccagtccgaagttttggatataaagcagtggtttttgtattttcctttttccttttctgtatcgtttagtaaagtcacatttggcttattgggtttgttttatttcctctgtatttgctttctgtacaaagactatcaataattagttaataagcttaacaaatttaaaaatcttatttttctttaaacccagaaatgcctgtaattcggaggaaaaaacccacttctgaacccaacaaaaattcagcatcagactcaaaaacgccaagcgaggaagaggaacatgaacaagaacaggaacaagaagaagataaaaataacaaaaagaaatacccaaagaagaagagcagtgaaatcaatgcaaaaaaatggtcatgcatagacagctgttgttggtttgttggttgcatctgcgtgacgtggtgggttttactatttctttacaatgcagtgcctgcgtctttgcctcaatacgtaactgaggcaatcacgggtcctttacccgatccacctggtgttaagctgaaaaaagagggattaacagcaaagcatccagtggtttttgtacctgggattgttaccgcggggcttgaattgtgggaaggccatcagtgtgctgatgggctgtttaggaaacggctctggggtggaacttttggagaagtttataagaggcctctctgctgggtagagcatatgtctctagacaatgaaactggattggatcctcctggtataagggtcaggccagtctctggacttgtggctgctgattactttgctccaggctattttgtgtgggctgttctgattgctaatttggcacgcattggatatgaggagaaaacaatgttcatggcctcatacgattggagactttcatttcagaacactgaggtccgtgaccaaacattaagccggatgaagagtaatatagaacttatggtttctatcaatggtggaaataaggcagttattgttccacattccatgggtgttttgtactttctgcattttatgaagtgggttgaggcaccagctccaatgggaggaggtggtggaccagattggtgtgctaagcatatcaaggcagtcatgaacattggtggtccatttttaggtgttcccaaagctgttgctgggcttttctcggctgaagcaagagatattgcagttgccagggccatagcaccaggtttcttagataatgatatgttccgcctacaaacattgcaacacatgatgaggatgtctcgcacatgggattcgaccatgtcaatgataccaagaggtggggacactatctggggcgatcttgattggtcacctgaagaaggttacattcctagaaagaaaaggcagagaaataatgcaactgataatgtaaacgaaggtggggccgaaagtgagatttctcaaagaaagattgttagatatggaagaatgatatcatttgggaaaaatatagcagaggcaccttcatatgatattgaaaggattgactttagggatgctgttaaaggtcgtagtgtggcaaataatacctgccttgatgtgtggactgaataccatgaaatgggattcggaggtattaaagccgttgcagagtataaggtctacactgctggatctactatagagctgcttcagtttgtcgccccaaaaatgatggagcgtggtagtgctcatttttcttatggaattgctgacaatttggaggacccaaaatatgagcactacaaatactggtcaaatcccctggagacaaagttacctaatgctccagaaatggaaatattttccatgtatggagttggcataccaacagaaagagcttatgtttatgagttttctcctgctgctgagtgctacattccatttcagattgatacatcagctaatgatggcgatgaagatggctgtctgaaagatggagtctatactgttgatggggatgagactgttcctgttttaagtgcaggcttcatgtgtgctaaagcttggcgtgggaaaaccagatttaatccttcaggaagtcgaacatacattagagagtacgatcattctcctccagctaatttgctagagggccgaggcacccaaagtggtgcccatgttgatataatgggtaattttgctttaatcgaggatattatgagggtggcagccggggctacaggagaagatttgggaggcgatcaagtgtattcagatatctttaagtggtctcagaagatcaaattaccactgtaa

SEQ ID NO: 132 - protein, 166 aa, Sesamum indicum
MADRDRPHPHQIQVHPQHPHRYEGGVKSLLPQKGPSTTQILAIITLLPISGTLLCLAGITLVGTLIGLAVATPVFVIFSPVLVPAAILIAGAVTAFLTSGAFGLTGLSSLSWVLNSFRRATGQGPLEYAKRGVQEGTLYVGEKTKQAGEAIKSTAKEGGREGTART

SEQ ID NO: 133 - protein, 144 aa, Sesamum indicum
MADEPHDQRPTDVIKSYLPEKGPSTSQVLAVVTLFPLGAVLLCLAGLILTGTIIGLAVATPLFVIFSPILVPAALTIALAVTGFLTSGAFGITALSSISWLLNYVRRMRGSLPEQLDHARRRVQETVGQKTREAGQRSQDVIRP

SEQ ID NO: 134 - protein, 173 aa, Arabidopsis thaliana
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTT

SEQ ID NO: 135 - protein, 149 aa, Arabidopsis thaliana
MADHQQHQQQQQPIMRSLHESSPSTRQIVRFVTAATIGLSLLVLSGLTLTGTVIGLIVATPLMVLFSPVLVPAVITIGLLTMGFLFSGGCGVAAATALTWIYKYVTGKHPMGADKVDYARMRIAEKAKELGHYTHSQPQQTHQTTTTTH

SEQ ID NO: 136 - protein, 181 aa, Helianthus annuus
TTTTYDRHFTTTQPHYRQDDRSRYDQQTHSQSTSRTLAIIALLPVGGILLGLAALTFIGTLIGLALATPLFVIFSPIIVPAVLTIGLAVTGFLASGTFGLTGLSSLSYLFNMVRQTAGSVPESLDYVKGTLQDAGEYAGQKTKDFGQKIQSTAHEMGDQGQVGVHAQVGGGKEGRKSGDRT

SEQ ID NO: 137 - protein, 165 aa, Brassica napus
MGILRKKKHERKPSFKSVLTAILATHAATFLLLIAGVSLAGTAAAFIATMPLFVVFSPILVPAGITTGLLTTGLAAAGGAGATAVTIILWLYKRATGKAPPKVLEKVLKKIIPGAAAAPAAAPGAAPAAAPAAAPAVAPAAAPAAAPAPKPAAPPAPKPAAAPSI

SEQ ID NO: 138 - protein, 186 aa, Zea mays
MADRDRSGIYGGAHATYGQQQQQGGGGRPMGEQVKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEAHRRMAEAAAHAGHKTAQAGQAIQGRAQEAGAGGGAGAGAGGGGRASS

SEQ ID NO: 139 - protein, 148 aa, Oryza sativa
MGDQHRGVIGGGGYGDRGGQEQQEKQPFMMTALKTVTAATAGGSILVLSGLILAGTVIALTVATPVLVIFSPVLVPAAIALALMAAGFVTSVGLGVAALSVFSWMYKYLTGKHPPGADHLDHTKARVASKLRDIKEAAHHLIDQAQAS

SEQ ID NO: 140 - protein, 216 aa, Brassica oleracea
RFFRMFSFIFPLLNVIKLIIASVTSLVCLAFSCVTLGGSAVALIVSTPLFIIFSPILVPATIATTLLASGLMAGTTLGLTGIGLITGLVRTAGGVTLAESPIRRIIINRIKARLGGGGGSRLAMLKKILGLIKKLRGMSSGGAAPALKQHQQLRPRMELHPRHLHRPNKERWFMLFQYVAHKNCVIINLRIYDSETKKKIALLLSFIQYSFLCNNV

SEQ ID NO: 141 - protein, 148 aa, Coffea arabica
MAEHYQLQQRPTEAVKSFLPQKGPSTSHVLAVVTLLPVAGVLLGLSGLILVGTVIGLAVTTPLFVIFSPILVPAVFALGLALAGFLTSGAFGITALASLSWMLNYIRLMKASSQEQMDLAKWRVQDTAGQVGQKARDVGQRTQDVARA

SEQ ID NO: 142 - protein, 380 aa, Brassica oleracea
MRNEIQNETAQTDQTQGSMFSFFDLFPFLLPMFEVIKMVVASVASVVYLGFAGVTLSGSAVALAVSTPLFIIFSPILLPAIAATTVLAAGLGSKKVAAAPAASPSLSLLGIPESIKPSNVIPESIKPSNIIPESIKPSNIIPESVKPSNIKDKIKDTIGKVKNKINAKKEEKSKGKSEDSSKGKGKSKGEDTTTDEDKPGSGGKHGKGESKHGKGESTHGKGGKHGSEGSSMDEGKHGGKHGSGGSPMGVGKHGSGGKHESGGSPMGGGKHGSGGKHESGGASMGGGKHGSGGRHEGGGSAMGGGKHGSGGKHGSEGKHGGEGSSMGKNSLSKNKKEFHYRDQAMDASSTSESSDGSSDGSSSDGSSSDGSSHGSGGKHI

SEQ ID NO: 143 - protein, 145 aa, Sesamum indicum
MAEHYGQQQQTRAPHLQLQPRAQRVVKAATAVTAGGSLLVLSGLTLAGTVIALTIATPLLVIFSPVLVPAVITIFLLGAGFLASGGFGVAALSVLSWIYRYLTGKHPPGADQLESAKTKLASKAREMKDRAEQFSQQPVAGSQTS

SEQ ID NO: 144 - DNA, 501 bp, Sesamum indicum
  source 1..501: mol_type = other DNA
atggcggaccgcgaccgtccacacccccaccaaattcaagtccaccctcaacatccgcaccgctatgaaggtggcgtcaagtctctcctccctcaaaagggcccctccaccacccagattctcgccataatcaccctccttcccatcagcggcacgcttctttgcctagctgggatcacgctcgtcgggaccctcatcggacttgcagtcgccaccccagtcttcgtgatcttcagccctgttctggttcccgcagccatactgatagccggcgcggtcacggcgtttttgacgtccggggcttttgggctgacggggctttcgtcgctttcttgggttctgaattcattcagacgggcgacggggcaggggccgttggagtacgcgaagcgaggcgtgcaggaggggactttgtatgtgggagagaagacgaagcaagcgggcgaagcgattaagagcacagccaaggaaggagggcgagaagggactgcacggacttga

SEQ ID NO: 145 - DNA, 559 bp, Sesamum indicum
  source 1..559: mol_type = other DNA
ggcacgagcgccgtccccatggcggacgaaccccacgaccagcgccccaccgacgtcatcaagagctacctccccgaaaagggtccctccacctctcaagtcctcgccgtcgtgaccctcttccccctcggcgccgtcctcctctgcctagccggtctcattcttaccgggaccatcatcggcctcgccgtcgccaccccgctcttcgtcatcttcagccccatcttggtccccgccgccctaaccatcgccctagccgtcaccggtttcttgacctccggagctttcggcatcaccgccctgtcctcgatttcgtggttgctgaactacgttaggcgaatgcgggggagcttgccagagcagctggatcatgcacggcggcgcgtgcaggagacggtgggccagaagacaagggaggcggggcagagaagccaagatgtaataagaccgtgaggtttttggatattagatgttggttaatttgtgtgtttaatgtatatatgaggggttgaataagttaataaaattgcggatttggtacaaaaaaaaaaaaaaaaaa

SEQ ID NO: 146 - DNA, 1800 bp, Arabidopsis thaliana
  source 1..1800: mol_type = other DNA
ccatggctatacccaacctcggtcttggtcacaccaggaactctctggtaagctagctccactccccagaaacaaccggcgccaaattgccggaattgctgacctgaagacggaacatcatcgtcgggtccttgggcgattgcggcggaagatgggtcagcttgggcttgaggacgagacccgaatcgagtctgttgaaaggttgttcattgggatttgtatacggagattggtcgtcgagaggtttgagggaaaggacaaatgggtttggctctggagaaagagagtgcggctttagagagagaattgagaggtttagagagagatgcggcggcgatgacgggaggagagacgacgaggacctgcattatcaaagcagtgacgtggtgaaatttggaacttttaagaggcagatagatttattatttgtatccattttcttcattgttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactacttaagttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtacc

SEQ ID NO: 147 - DNA, 450 bp, Arabidopsis thaliana
  source 1..450: mol_type = other DNA
atggcggatcatcaacaacatcagcaacaacaacaaccaataatgaggagtctccatgaatcatcaccatcgactcggcagatagtgagattcgtaacggcagctacgatcggcctatcactcctcgtgctctcaggactaacactaaccggaacggtgatcggtttgatcgtagcgacgccgttgatggttctgttcagcccggtgttggtaccggcagtgataacgatagggcttctgacgatgggattcctattctccggtggttgtggggtggcagcagctacggcgttaacgtggatttataagtacgttaccggaaaacacccgatgggagcggataaggtggattacgcgaggatgaggatagcggagaaagccaaagagttgggacattatacgcactcgcagccacaacaaacacaccaaaccacaacaactactcattag

SEQ ID NO: 148 - DNA, 625 bp, Helianthus annuus
  source 1..625: mol_type = other DNA
actaccaccacttacgaccgtcatttcaccaccacccaaccccactaccgccaagacgacaggtcccgctacgaccagcaaacccattcccagtccaccagcaggacactcgccatcatcgccctacttcctgtcggcggaatcttactcggcttagccgctctcacattcatcgggacgcttatcgggctcgccctcgccaccccgcttttcgtcatattcagcccgatcatcgtgccggccgttctaacaatcgggcttgctgttacaggctttttggcgtcggggacgttcgggttgacgggtttgagctcattgtcgtatttgttcaatatggttaggcagacggctgggtcggtgcccgagtccttggattatgttaaggggacgttgcaggatgccggtgagtatgccgggcagaagacgaaggatttcgggcagaagattcagagcacggctcatgagatgggtgatcaggggcaggttggtgttcatgctcaagttggtggcgggaaagaagggcgaaaaagcggtgatcggacttgaggattcaaggttgatattgtggaataataatgttgatgtaagtttttagtgttatcaaagctttgtttgtttgtttgta

SEQ ID NO: 149 - DNA, 737 bp, Brassica napus
  source 1..737: mol_type = other DNA
gaattccattcaaactagtaaacaatggggatactcaggaagaaaaaacacgagcgaaagccgtcgtttaagagtgttttaaccgcaatattagctacacatgccgcaacattcctcttgttgatcgccggtgtatccctcgccggcacagccgccgcatttatcgctaccatgccactattcgtagtattcagtccgattctcgtaccagctggtattaccactggtttactgactacgggtttagcagccgccggtggcgccggcgcgactgctgtcaccatcatcctgtggctctacaagcgagcaacgggcaaggcgccgccaaaagtcctagaaaaagtcttgaaaaagataataccaggtgctgcagctgcaccagcagccgctccaggagccgctccagcagcggcgccagcagccgcaccagctgtggcgccagcagccgcaccagctgctgcgccagcacctaagccagcagccccaccagcacctaagccagcagccgcaccgagtatatgaaaagaagtggtgggcatgagtaaaggttgatatggaaaactggatacatagaaaaaagagtaatccaacttttaaaaaataaataacaacttcacgtggggatagaaaaattttcaaatattattttactaatggatgtcgcggtacaaaataataacaaatgtaagcctttttattgtatagtattttaagaacgaagctatgtagcgttgaca

SEQ ID NO: 150 - DNA, 1153 bp, Zea mays
  source 1..1153: mol_type = other DNA
aactactccgtcactttgtttgcaaagctcctcctcgatccatcgatcactgcaccggccggcggcaccgcgctcgcaggggctagccaacgagacggcagcaatggcggaccgtgaccgcagcggcatctacggcggcgcccacgccacctacgggcagcagcagcagcagggaggaggcgggcgcccgatgggtgagcaggtgaagggcatgctccacgacaaggggccgacggcgtcgcaggcgctgacggtggcgacgctgttcccgctgggcgggctgctgctggtgctgtcggggctggcgctgacggcctccgtggtggggctggccgtggccacgccggtgttcctgatcttcagccccgtgctggtccccgccgcgctgctcatcgggacggccgtcatggggttcctcacgtcgggcgcgctggggctcgggggcctgtcctcgctcacgtgcctcgccaacacggcgcggcaggcgttccagcgcaccccggactacgtggaggaggcgcaccgcaggatggcggaggccgcggcgcacgcgggccacaagaccgcgcaggcaggccaggccatccagggcagggcgcaggaggccggcgccgggggaggtgcaggtgccggcgctggcggcggcggcagggcttcctcgtaagcaagtcatccatgcatggattatggatagatgcgcgcgtgcgtgtctatcagtatcagcagccagcagggtcgtcgcggaatgctgtgttcctgtacgtgtgggtgaccgtccttccgtccttcgtctttctccccccgagtgtgtgttacgtatgtcctggtgttcgtcgtgtgtgttcatcgccgctccagttgaattccggtgtctgttcatcgccgctccaggtcgtagatgtgaatatactttgctaggggaataagtgataagtctgtctggaaggtaatgtttgagctttgctagtgtggctgggcactctggtcactggttgtgttgtgcatgcatcagctgtatgatcgtcgtctgttgtggaaaattggtcaatgtattctcttgctgaataatttgtgacatctaattgttatgtatcgtctctttgctgaataatcagtttctgatttatcttgcattaaaaaaaaaaaaaaaaaa

SEQ ID NO: 151 - DNA, 447 bp, Oryza sativa
  source 1..447: mol_type = other DNA
atgggtgatcagcacagaggagtgatcggcggtggcggctacggtgaccgtggtggccaggagcagcaggagaagcagcccttcatgatgacggctctgaagacggtgaccgcggcgacggccgggggctcgattctggtgctgtccgggctgatcctggccgggaccgtcatcgcgctcacggtggccaccccggtgttggtcatcttcagccccgtgctggtaccggcggccatcgcgctggcgctcatggcggccgggttcgtcacctcggttggtctcggtgtagccgcgctctccgttttctcgtggatgtacaagtacctcaccgggaagcacccgccgggcgccgaccatctggaccacaccaaggcgagggtcgcgtccaagctccgcgacatcaaggaggcggcgcatcacctcatcgaccaggcgcaggcgtcttag

SEQ ID NO: 152 - DNA, 709 bp, Brassica oleracea
  source 1..709: mol_type = other DNA
gccgttttttcagaatgttctcttttatcttcccattgctgaacgttataaagcttattatagcttccgtgacctccttagtctgcttagcgttttcttgtgtgacactcggtggttcagccgtggcattaatcgtatccacaccacttttcatcatatttagtccaattctcgtacctgccactattgccactaccctcctagccagtgggctcatggcgggtaccaccctcggactgaccggcataggtctcatcacggggctcgttaggacggcaggaggagttacattggccgaatcaccgataagaagaattataataaatagaattaaagcaagacttgggggtggcggcggttcacgtctggcaatgctcaaaaaaattctgggactcattaaaaagttgcgtggtatgtcttcaggtggagcagcacctgcgctgaagcagcaccagcagctgcgcccgcggatggagctgcacccgcggcacctgcaccgacctaacaaagaacgttggttcatgctgttccaatatgtagcacataaaaattgtgtaataattaacttaagaatttatgattcggaaactaaaaagaaaatagcccttttactatcttttatacaatatagttttctatgtaataatgtttaatttgcttataactataaaagactcatgcatagttgattaggaaaaaaaaaaaaaaa

SEQ ID NO: 153 - DNA, 447 bp, Coffea arabica
  source 1..447: mol_type = other DNA
atggctgagcactaccagctgcagcaacgccccacagaggccgtcaaaagcttccttcctcagaagggtccatcaacttcacatgtgttagcagttgtcacgctcctcccagttgcgggagtcctgctaggcctttccgggctgattctcgtcggaacggtcatcggtctggcggtgacaaccccgcttttcgttatctttagccccattttggtcccagctgtatttgccctagggctggccctggccgggttcttgacctccggtgctttcgggatcactgcacttgcttcattgtcgtggatgctgaactacatccgactcatgaaggcgtcttcccaggagcaaatggacctcgcaaagtggcgcgtgcaggacactgccggccaagttggtcagaaagcgagagacgtgggccagagaactcaagatgtagccagagcatga

SEQ ID NO: 154 - DNA, 1000 bp, Brassica oleracea
  source 1..1000: mol_type = other DNA
atgagaaacgaaattcaaaacgaaacagctcagactgatcagacacagggaagtatgttttcttttttcgatttgttccctttcctcctcccaatgtttgaggttatcaagatggttgttgcttccgttgcgtccgtagtctatttaggcttcgccggtgtaacactcagtggttcagccgtggcattagccgtatctacccctcttttcatcatattcagtccgattctcttacctgctattgcagccactactgtcctagccgcagggctcggcagtaaaaaagttgcggcggctccggcagcttcaccatccctatctctgttgggcataccggagagtattaaaccaagtaatgttataccggagagtattaaaccaagtaatattataccggagagtattaaaccaagtaatattataccggagagtgttaaaccaagtaatattaaggacaaaattaaggatacgataggcaaagttaagaataagatcaatgctaaaaaggaagaaaaatcaaaaggtaaaagtgaagattcttccaagggtaaaggtaaatcaaagggtgaagatacgactacggatgaagataaacccggaagtggaggtaaacacggaaagggtgagagtaaacacggaaagggtgagagtacacacggaaagggaggtaaacatggaagtgaaggttcgtcaatggatgaaggtaaacacggaggtaagcatggaagcggaggttcgcctatgggtgtaggtaaacacggaagcggaggtaaacatgaaagtggaggttcgcctatgggtggaggtaaacacggaagcggaggtaaacatgaaagtggaggtgcgtctatgggtggaggtaaacacggaagcggaggtagacatgaaggtggaggttcggctatgggtggaggtaagcacggaagtggaggcaaacacggaagtgaaggtaaacacgggggtgagggctcttctatgggtaaaaatagtctatccaaga

SEQ ID NO: 155 - DNA, 706 bp, Sesamum indicum
  source 1..706: mol_type = other DNA
tcacctcaaagtttgctccgttcttgacagaaatggctgagcattatggtcaacaacagcagaccagggcgcctcacctgcagctgcagccgcgcgcccagcgggtagtgaaggcggccaccgccgtgacagccggcggctcgcttctcgtcctctctggcctcactttagccggaactgttattgcgctcaccatcgccactccgctgcttgtgatctttagccccgttctggtgccggcggtcataaccattttcttgctgggtgcgggttttctggcatccggaggcttcggcgtggcggcgctgagtgtgctgtcgtggatttacagatatctgacagggaaacacccgccgggggcggatcagctggaatcggcaaagacgaagctggcgagcaaggcgcgagagatgaaggatagggcagagcagttctcgcagcagcctgttgcggggtctcaaacttcttgagctctttttctgttgtatgaaaatcttaaactgtgtggcccttcgctagttggtttgatgtctggttcagagtgctgattatttggcgacattcggagggcgttggagatgataagctttcaatttgtttttcgaagttaatatttgttcttgattttgatgtctttggtatatatggtcttttagtacattatgttatcaataaacaagtcattttcaaaaaaaaaaaaaaaaaa

SEQ ID NO: 156 - protein, 335 aa, Mus musculus
MMVEFAPLNTPLARCLQTAAVLQWVLSFLLLVQVCIGIMVMLVLYNYWFLYIPYLVWFYYDWRTPEQGGRRWNWVQSWPVWKYFKEYFPICLVKTQDLDPGHNYIFGFHPHGIFVPGAFGNFCTKYSDFKKLFPGFTSYLHVAKIWFCFPLFREYLMSNGPVSVSKESLSHVLSKDGGGNVSIIVLGGAKEALEAHPGTFTLCIRQRKGFVKMALTHGASLVPVFSFGENDLYKQINNPKGSWLRTIQDAMYDSMGVALPLIYARGIFQHYFGIMPYRKLIYTVVGRPIPVQQILNPTSEQIEELHQTYLEELKKLFNEHKGKYGIPEHETLVFK

SEQ ID NO: 157 - DNA, 1008 bp, Mus musculus
  source 1..1008: mol_type = other DNA
atgatggtcgagttcgcgccactcaacaccccgctggcacggtgcctacagaccgctgcggtgctgcagtgggtcctgtccttcctcctgctcgtgcaggtgtgcattggaattatggtgatgctggtcctgtacaactattggttcctttacatcccatatctggtctggttttactatgactggagaaccccagagcaaggaggcagaagatggaactgggtccaaagctggcctgtgtggaagtattttaaggagtattttccaatctgtcttgtcaaaacgcaggatttggatccgggtcacaattatatatttgggtttcaccctcatggaatattcgtgcctggagcctttggaaatttttgtacaaaatactcggacttcaagaagctatttcctggctttacatcgtatctccacgtggccaagatctggttctgtttcccgttgttccgagaatatctgatgagtaacgggccggtttcagtgtctaaggagagtttgtctcatgtgctgagcaaggatggaggtggcaatgtctcaatcattgtcctcggaggtgcaaaggaggcgctggaggctcacccaggaacattcaccctgtgcatccgccagcgcaaagggtttgttaagatggccttgacccatggtgccagtttggttccagtattttcttttggtgaaaatgatctatataagcaaattaacaaccccaaaggctcctggctacgaactatacaagacgcaatgtatgattcaatgggagtagccttgccactgatatatgccagaggaattttccagcactactttggcataatgccctatcggaagctgatctacactgttgttggccgccctatccctgttcagcagattctgaacccgacctcagagcagattgaagagctgcatcagacatacctagaggagctaaagaaactattcaatgaacacaaagggaaatatgggattccggagcacgaaactctggtatttaaataa

SEQ ID NO: 158 - DNA, 1050 bp, Vigna angularis
  source 1..1050: mol_type = genomic DNA
cattatttgaatgcatttggtagaccttcacgtcgcaataatatccgttaacagataaaccatattccattatttgaatgcatttctatctcattatatcacttagtttttgcctttttccctcaagagtttatctatctaaagaaatattacaaaaagcctcttccatccaataagaagctgccattttagatttgggataagctgtgaatatcagctaaatcaactgtaaaatggaacatggtaaaactatcaagtattcattgctaacatgcacaactgtgtttatgtgtggagcacaatctttgtgcagttgttccaaagtgcttatggccgcagaataaaactttccaaacaaaccattaacactcatatttcatcaacttattatattttagatttctcttattttctaaagataacttagatcaaacaaactatttattaaaaaaagttattaccgtataatgatctattattatttatattgaagtactattattatttatattaaaagtagtgaatcaaccacgtattaattagtttgaattcatgtgacgtgtaatgtaattgccccttggtgagaatagacgcgcgttaccatcacccttcacttggttcttttcacaatttctctgtctgctattcaaaatttgtggttggcatgcaccttgccacaagcgaaaatggatgtggacccaaaaatatctcacaaactcaaatgatggccacatagttgaagccattttccaggtcagttgcaaaatgaataagaaatgagcaataatatcttgtggaatcctgaatcatacgtggaagtcccacattggtgaaaaaacataagagaatctaaccctctagtggattgaaagagtgccaagtgtacaaacacaatctcaacccccaatgagattgtagctttagatagccatagataagagctcaaccacttcatccaccatataaatcctcacccaccatccccacccattcacaactcccacacctcaaagcacagagcacaaagaaaaaggaaagttctttggaaca

SEQ ID NO: 159 - DNA, 1209 bp, Vigna radiata
  source 1..1209: mol_type = genomic DNA
cagaaaagtaagtgttttattatagaaaggggagaatcaaggtaaatgaaaataaaaatatacgagaaaaagtaagtaaattaaatgtatttaattaaataaaataaaaagcgaatagaatatagaacagtgaaatccccaaattttaaattttaaatttcatttcatttttgaaaattagttataccaagtactctcttctaattctatccttcaaaacataaaaagagacgaattaaaggcatgtttatttataaagagagtatgtttagatgaagtttttgataagaaatttaagagggaaaagaaattatataaaaaagtaggcgttgaaatggtaagtgataagtatatgaaagagatagagaaatcaaatattatttgaatgcatttggtagatcttcacgtcgcaataacatccgttaacagataaaccatttcatttgggcaactgtgactcttagagatttctatctcattatatcacttagtttttgcctttttccctcaagagtttatctatctaaagaaatattacaaaaagcttcttccatccaataagaaggtgccattttagatttgcgataagctgtgaatatgagctaaaaataaactgtaaaatggaacatggtaaaagtaaaaagtattcattgtgcagttgtttccaaagtgcttatggccgcagaataaaactttccaagtgacgtattaattagtttgaattcatgtgacgtgtaatgtagttggacgcgcgtaaccatcatccttcacttggttcttttcacaatttctctgtctggaattcaaaatttgtggttggaatgcagcttgccacaagagaaagtggatgtggacccaaaaatatctcacaaactcaaatcatggccacatacttgaagccattttccaggtcacttgcaaaatggaataagaaatcagcaataatatcttgtggaatcctgagtcatacgtggaagtcccacattggtgaaaaaacatatgaggaatctaacccagtagtggattgaaagagtgacaagtgtacaaacacaatctcaacccccaatcagattgaagctttagatagccatagataagagctcaaccacttcatccaccatataaatcctcattcaccattctcaaccattcacaactcccacacctccaactacaaaccacaaagaaaaaggaaagttctttgcaaca

SEQ ID NO: 160 - DNA, 1579 bp, Vigna angularis
  source 1..1579: mol_type = genomic DNA
tgcattctttttgactgattttagtgattacgcattaaaaccaaatagttattattagaagttttttaaaacagcattagaaggataaataaaaacatatatcatataagaacaaataattattaatgttatatcctcattattttcgtcagttattgcaggtattgtaatatatagagctgtcaaaatggtcacattctatgggttcaccatatattcgagctggattgggtttgaaaaaattgtatttttctatgtgggtcagatttcaacccgactcatttaaacctggcttatgcgggttgaatccgtggtgggtcgggttggcctacctatccaattttattttattaaaatttaattttaattttataaaaaatatttattattttttttcttgaaaaaattatttaaattccttactttcataattaattaaaacacccatgttgttgggagtgaaacttgtttagatttgaattatagaaagtttctgaaaaaaatttatttaaaaaaaaattgtaattaagtgagctagtgagtcaacccatttatctaccaacctgtggtggatcgggacgggttcaaatttttctgactcgttaataaatgagtcgggttagatgggctcactaagtgaccaacctgtgatgagccggataaggtcacaccggatgacccgttttcacagttcgtaatattacatctcgacaagtgagtaacccggaagtcaaactcctaaaaaatagtaaatgttttaagtgatgtttccatggcaattatgttgattttaatttgtctatcttaaactcattctcagtttcttactatattttaaatttactaaattaatagttaaaatagcgaatatcattgaaataattgataaaatatttcatcacaactttattttacgataacttttgaaagggtttttgtattgaaggtttaaacagaaactttgttacagtattggtgatgtttttaaccttatttggtgtgctttaattgttttttctaattctcatctaattattacaacttattttagaatttagttcccacataaaccaaatttgtaatgtagcaaagatgagctgccaccatgagttcttggtagccgagtattctgtgtgtggtttcagattctcaggaacaccaactgtttgtttttttgtttgaaaagaaaagaagaaaaagaatgtgatgggtcacgggtttgcactgtagaatgaacccaacccaaactatctgcaatttcaagttgatatttttaattttgatttcttagactttggtgggccctttttctgtggcttacattagccctctcgaaaacatctatggaagaagatctttgcatccacgtggcagaagcagagacagtttagcagcacaaaatatcagcatgagaatccacatccatatccagaaccaatgaagtgttgctgagttgtgcatatcctcatccacaatatcatgccacccttctataaacatttgtagtacccttcttcattctccacaacacactcacaaaaccaaagaaagaagtgacctttaaccacacaacacagcc

SEQ ID NO: 161 - DNA, 1453 bp, Vigna radiata
  source 1..1453: mol_type = genomic DNA
tttataattataataatagtaatagcaataatttattttttattatcaaaaacacatgtatttttattagttaatatagaggtatttttgttaacattttattttactctttatatatcagttttttaagttaaattaattgtttattattttactttttaaaaatttgacttttttttctgaatggatatttttttttagatttaaccatttttaaattaaaataactatttataataaaaattaaaaataaataaaatccatttgtaataaaaatacaaaatttatttatattaataattttattattaaaaaagttaacaaacatgttgactatgtgttttcactctttattaatgccattattttatatgcatgtgtatcagagaaaatttaattttaatgtatatatacacattcaaagaagtgaaaaagaatttgattaaaacaaaataaagttaagagcaaaaaaataaaaagcaaaacaagatggctgaacctttttttatataaaaagatagactaaattaatgtatagctatgggtaaagtttagagtacaaattcattgaaaaaattattattaactttcaatataataaatttcgtgtccaaatattttattaccaatatcacagtttttcaaatgcgtgacagtgtactttgacaaagaaaataaaagaatgttaataaaaatacaacattattcaatttttaaattatgatataaagtggaatatgcattctttttgactgattttagtgattacacgtcaaaaccaaatagttattattagaatattttataaaacggcattacaaggataaataaaaacatatatcatataagaacaacctttctcaattattgataattattgatgttataccctcattattttcgtcatttgttgttgtaatattacatctcgacaagtgaatctgcaaccagaaattttgttacagtatttgtgatgtttttaaccttatttgatatgctttaattgttttttctgattctcatctaattattacaacttattttagaatttaattcccacataaaccaaatttgtaatgtagcaaagatgagctgccaccaggagttcttggtagccgagtattctgtgtgtggtttcagattcgcaggaacaccaactgtttgtttttttgtttgaaaagaaaagaagaaaaagaatgtgatgggtcacgggtttgcactgtagaatgaacccaacccaacccaaactatctgcaatttcaagttgatatttttaattttgatttcttagactttggtgggccctttttgtgtggcttacattaaccctctcgaaaacatctaaggaagaagatctttgcatccacgtggcagaagcagagacagtttagcagcacaaaatatcagcatgagaatccacatccatatctagaaccaatgaagagttgctgag

SEQ ID NO: 162 - DNA, 1477 bp, Vigna angularis
  source 1..1477: mol_type = genomic DNA
atagaaaagtgctgctgaagatggaggaagtatttggggtacatgatttgaaatgggaaggaatgatcaaaagagatacatattttctgtcaacaatgaagcattcatatagtaaatttgtattctgttattatgcagaaaatatcagaagcttttttcatttgtattgtcaacaatggttccttttatatattccctgattccttactctccttttattcatttttgaaccattaactgagttgaatagcattaaatcacgtagatttttgtcttctgtttttgtggggatatgaatgagaaaaattattacaaccagacaaagatgtagctcagaagaaagagtaacaagtccccctgctagagtataactgagccaaaattctttgagaacaatctggaactaacagtttgtaaacaagctgttatacaaaactgataattttacagttcccagaactgaaattttcaaccattaaccattttcatctatatctatcttctttccagtcatgaacttccataccgaacatttgtcgcctgtttctcttattggcagtggcattttgtcatattttgccatactaaattatgacagtcatactctttgaacttcattaaatacctttgattttgtgttttatagttatctcatgcagtgtattttaagataaggttgttgtgaattgtttggttgagttcatggatttactgatgaccatagctatttgtggatattatgttttcaaaatgtatgcttggtcattaatcaaccaagaatgataagccccatcatctgccaacctttctataccatatacacattatgcttatctacattgctcttaaacaaaactaacaaataacatacaaaccaatgaacatttttagtaatattttatgaattaaccaacctaaggtgcagagagaagcatgtttgcagctatacaacaaaagggataagggtgtcagtttatatggttctattgtttttttccaattcaaaatctcctttaaaatataaaatactactgtaacatgtaacaaagagacaaagggtatttattcacttaacccataaatgagaaatggtggtcagaaaatagttaaggtaaaatggaatgaaggcacattagtacaaaccactcacaaaaaatatcaattccaccagcatcacacatttacgttcttccaaggaagggataagataatggaacctgttccacgtgtcagttccacatggtacctaacgataaggctaccattcaaatattttccttactcgtgtggacaatatgctgtaatgtcatcacttatagaatccaacggttgtaacatgtcaggaacagatcccttccatttgaaaccattggattaatactgaacagatcacactattatatatagcaagaaggagaagaagcttggccatctagcagcagaactgcaagtgattcagaagtaagaaggtgaagga

SEQ ID NO: 163 - DNA, 1490 bp, Vigna radiata
  source 1..1490: mol_type = genomic DNA
agtagatgcaaacttcatcttacaagtcgactttatgagattgagttagacttaaagtctactgttaacatcaacaaaagagatacaaatttgctgtcaacacaatgaagcattcatttagtaaatttgtattctgttattatgcagaaaatatcagaagcttttttcatttgtattgtcatcaatggtttctatttatatattccctgattccttactctcctttttttcatttttgaaccattcactgagttgaatagcagtaaatcatgtagatatttgttttctgtttagtggggatatgaatgagaaaaatcattacaaccagacaaagatgtagctcagaagaaagagtaacaagtcgcctgcaagagtataactgagccaaaattctttgagaacaatctggaactaacactttgtaaacaagctgttatacaaaactgataattttacagttcccagaacagaaattttcaaccattaaccattttcatctatatctatcttctttccagtcatgaacttccataccgaaaatttgtcgcctgtttctcttattggcagtggcattttgtcatattttgccatactaaattatgacagtcatactcttttgaacttcattaaatacctttgattttgtgttttatagttatctcatgcagtgtattttaagataaggttgttgtgaattgtttggttgagttcatggatttactgatgaccatagctatttgtggatattatgttttcaaaatgtatgcttggtcattaatcaaccaagaatgataagccccatcatctaccaacctttctataccatatacacattttgcttatctacattgctattgttgctcttaaacaaaactaacaaataacatgcaaacaaatgaacatttttagtaatattttaagaactaaccaacctaaggtgcagagagaagcatgttttcagctatacaacaaaagggataagagtgtcagtttatatggttctattgttttttttccaattcaaaatctcctttaaaatataaaatattactgtaacatgcaacaaagagacaaagggtattttttcactaaacccacaaatgagaaatggtggtcacaaaatatgtaaggtaaaatggaatgaaggcacattagtacaaaccactaacaaataatatcaattccaccacaatcacaaatttacgttcttccaaggaagagataagataatggagcctcctccacgtgtcacttccacatggtacctaacgataaggttatcattcaaatattttccttactcgtgtggacaacatgatgtaatgtcatcacttatagaatccaacggttgtaacatgtcaggaacggatccctttcatttgaaaccattggattagtagtgaagaaatcacagtattatatatagcaagaaggagaagaagcttggtcatctggcagtagaactgcaagtgattcagaaaggtgaagga

SEQ ID NO: 164 - DNA, 1334 bp, Arabidopsis thaliana
  source 1..1334: mol_type = genomic DNA
aagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaacaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 165 - DNA, 1944 bp, Glycine max
  source 1..1944: mol_type = genomic DNA
gtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggctcgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacag

SEQ ID NO: 166 - DNA, 10936 bp, synthetic construct
  source 1..10936: mol_type = other DNA
tggcaggatatattgtggtgtaaacaaattgacgcttagacaacttaataacacattgcggacgtttttaatgtactggggttgaacactctgtgccgaattcggatccagcgaacaatcgacaatacataatagctgcattgtcacccgatacttaaataattgtctcatacaaataaaagattgaagatattcaaacaatatttatatgtgaagataaaaaaaaaccaaaaaaatgaaccaaggaccaaatataatatgatatttttttgcttagggacaaaaaaaaacagcatcatcacaagcatcctaaagaaaatagaagagataaaacagagatataaaactaaagtacacaagacttcttgacacaaccataaggttctaaagagaaaaaagaaaataaaatccacataacaacagatagttcaattcccatctcaactagggaaaatagatttttttgtaacaaactcaccaccacgaaccagataagacaagacacacaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcattcctggttcggctttctttggcccggatgatgatatggcgggttcggttgcgacggcgaaaacggtactaaagccatgacgttgcctgggccaaaattgtcttgtaggaagatacacaaaacggcgaaggctggaagtgaaggagctaggttttgacaggaaagaagaagaaaaaagcagtgaatgactggaagccatggctgtcgagtcaacaatcacagataaatcagaatcaaaatccattacgtaatcttaagtacggatctgaagcaatctcgtgaaaaaaaaccctaatccacggcaatagaatcaacaaacaaggcagttgctaacagatctgagcaatatctctcgaaaaacgatatagaatcaacgaataaggctttttgtcaacggatctgaacaatttcttgaaaaaccacagaagtataaaaaataaggctgttgtttaacggatctgaagtaattcatgaaaaagccctgatctatggttacgtttaacggatctgaagtaatcaatgaaaaaagccctaatctacgattaatgaatcaacaattaaggcttgttgtaaagggatctgacatgaaaaagctctaattagagcaaatctacgattaaagaatcatcaaattaaggacgttgttgtaaagataataaaagctgaatctgattatgaaaattgaagatcgatgaaaccaaacaaaacacaatcaaatcaacatcgtattgatcaaagaacatacacaattcatggaaagaacgaagaagaaaaacagaagaaatcgactaaccttgaagggagaggtagggaaatacacagagagaatggaatatattggaattgaactgcgaaggaaggaggaggggtgtggggcgttttatagcataaggtgtggaacccttctccagtcatattgtgacgcgtgttcatgtatggtgtcacttggtgcctatgacacgggtaatatttggtattggatgcattttcggagagtccttctaggaaattgagaattgcaatttgcgtggtagttggcggtttcgtaatcttgacggagtcacatgcaccttcaagcttcgctttatctgttacgacgtcgttgttatattgggcccaccccgttgcaagtggagcgtccgaagttgatacgggccccactatccattgcctgaagtcggctgcttttacattattaaaaatctatggaaaagctgtcatgctagattttttttattatggaccagaaaagaataattgaaaagttttcttattatggagcaaaaaaatctttaataattattttttaaataaaatcataattattccactaaattctttcttatttaatttcaccttgtaagatatattttcagtagatttttcaagtatttttttaacatacattacttgtatacatttagttacagtttatataaaattattcatttaaaaaatatttagatattttttaaaaaataattcaaaatattttttttaatgaactttcctttttcctaatttttctttcttatttcttattagatgtttttactaccaatcgtttagggaagaatttttttctctaatgaggagatgtgttaaatgatgcaacttaaggacagattcaccaataacataagactaaaagatagctcattgacaaaaacaataatgatgttaaagaaaacgaatccatttccattcggataagccacaccatttacgaacaagttgcctactaataatatatggtgtcagttttttttttttttgggctgaattggaggggaaattatccccaaagtttgattaaaaatcacgcgacaaagtaacaaaacttttattccgctaaaaaaattagtcaaagtcaattaaatctggtattagaattacttttccgctaatgatatatccacaataacaatgtattaatattgaaaaaaataaagtttatagagtgtgactttttctttcatttttacgtaaattgaaaaaaaaatcaagcctaatatataaactttcattgatcctcacctaaatcctaaagttcttgagcccaattaaaaaacttactggaggataaaggcccaaacttctttttttttttaggaacagagcatgtaatgggtaatgaggcagcctaagcccataaccatgttagcccgtatatatttttgttaattagatttccggtttcttatttttttttagatttcagtttttgtttctcaaaaaaataaaataacttttagtgctgtattaactttctgtaaataattttagtctcaataaagattaattctgaataataaaaataaacgaggacaatttttttcacaacttttaattctttatttattttttacacttaattttgggaaacatggacctagtgtcaaagaactcaaactcaacattacaaaaacttactacatattcaaggatcatggtctgaactaatctccacataacatataatcacatttttcaattaaaaagaagaattaataatgtattaaatgatgatcgacaagatcacatgatttgagatttttttttatgatgggaaaaatatgatttgagattaggtaacaatctttccattttagatagacttatgtctccgcaagctagctatatacctgcaggatatatggatcccaacccaatttgcttcaagacgtgctcaaatcactatttccacacccctatatttctattgcactcccttttaactgttttttattacaaaaatgccctggaaaatgcactccctttttgtgtttgttatttagtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccggtaagagacaacaacgtaggtacgtggagtgtcttaggtgacttacccacataacataaagtgacattaacaaacatagctaatgctcctatttgaatagtgcatatcagcatagcttattacatatagataggagcaaactctagctagattgttgagagaggtgacctaggttcattagctggcctgaacgccagatatgcactgttgtccataatctttgatctctctggcgcagtccatgattttatggggggcagaatcgagacagtcggcaccaggagggtatttgcctgtaacgtacctgtagatccaagcaaggacagtgattgcagccacaccgaatcccccactggcaagaaagcccagtgacaacaaagccacagtgatcactgcgggaacaagtacgggggagaagataacaagtggtggtgtaacaatggtcagtgcgataatagttccagcgaggatcaaggaggcgagaatcaggagagatcctccggcagtcactgcagtggcggccttcaccacttggcagctatagcttgtttgttgttcgtaggggcactgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacatttggataacgatgaggatattggtgctggcattggtagtgcaactctgccatggttttttttaagggagcaagcggccgaagcctgcttttttgtacaaacttgtgatggggtacggcgcgccttccttcttcttagttctcaactacttgtttttctgctgccagatatccaagcttctgctcaaacttgctatatataatagtgtgatttgtgtagtactaatccaatggtgtgaaatggagaggattggttgccgaaaagttacaaccgttggattgaataagtgatgacattacagcatataggccacacgagtgaggaaaattttgaatggtagccttattgttaggtaccatgtggaggtgacacgtggaggctccattatcttatctcttccttggaagaacgtaaatgtgtgatggtggtggaattgatattatttgtgtagtttgcacagtgccctcattccatattaacctaacatattttgtgaccatcctttctcattcatggattaagtgaaaactctttgttcctttgttacagtaaattttatattttaaagttggttttttattggaaaagaaacaagagaaccatatcaactgaaactcttatccattttgttgggtagcttaaagcatgcttctctcttcacaattacattggccaatccttataataatattactaaaaatgttcattaacttaacgtattatctgttagacttgtttgagtgtaacagtagcaatgtagataagcaaaatgcgtatatggtatagaaggggtggtagatgatggggcttatcatgcttggttgattaatgaccaagcatacacttttgaaaatataagatccacaagtagctatggtaatcagtaaatcgttgcactcaaccaaacaatgcacaacaaccttatcttaaaagtgcaatgcatgagataacaattataaagcacaaaatgaaaagtcaagtattacgtaagaagttcagagagtgtgactctcataagaagccactgcctataaaagaaataggcaacaaattttcagcatggaaagttcaagactgaagaactgttagtgtttaagatgaaattggttaatggttgagaattgcatctttggaaattgtaaaattctcagtttggtataacagtttgtatacaagatgtgagttctagtgattctagcagtgaggcatgtttctctttcttctgagccacagctttgtctggttgtaaggatatttcgtattcatatccccacaaaacgaaaagaaaaatctacatgattttaccactattccaactcagttaatggttcaaaattcaaaagccgtgttttgaaggaaaggaaggcaacagagaatatatataaaaaagaacagttgggcttctaagatgaatgatatgctgaaaatatcaagagcaacatttgtccaatttactaacacacaacattcctatgaatccttcaatgttggtctgccaattttctatctcttctaatcattctccccttttttcaaatcatgtaccccaaacattccctcccctcttctgcaccacttccactactagcccgggtctgttaatgaacgtaaggagcttctggatgacggctgggacttctttgacaaagagacagagctctaagtgtcttcttttcagtagattatcggtgctagaataaaagagatatgaagggattctgggttctgctctatgtagagtaaagcttggatctttgatctaatctaccgttagtataaaacatctattgagctctcttttatttttcttctcagtgtatagtttgcttattgtttttttggtgtttttttccacctctttgtggtactgacataaacaaagactatcgttttgtcatagaattttgagattttgaagaaaagttggataatttagtatactttttaactgactttataaaacgaattagcttcaattagagcttcacaattctcggattttactgaatgacaagttaacaatttaagctagaaaaaaatatgctctaagcctggacatgttgttagtcatggccagcgtatgtgtacattaggtaaatgaaaacaaaaacaaaaactttgaaggtttctgttaagaaaggaactaatttagcaataagagacatgatattacaagctcaacactgtcgctatatctaccagaagccaacaaatcttccagtgcgtaccattggagccacttttcaggctcattagacaaaagtagcacatataatgtaaagacgaaacgatgataaagagactcacaaggacataatttcaatacagaagaagtttgatgtcattactcattagaaagaaaggtataacacaggaacggaaacatagttgaacaattattcatcaggggctttacaaggccccaaaacacaaaccaccaaagttttaaatgaaacgagacatgaacttcttctgattcataacagagataagtttactagtccctcattatttctctttgaggttgatgaggtcgtggtagtaaagcaacacgcacattggctggccgaggatgcagaagatgaaccagaaaatcatgttgcccaccatggagttggagaacttctcttggaggtagttggtaatgagcacgagtggcacctggaacatgattccgatgaaagcccagagcttgaacacgtggcatggaacagcaatgcacaattcgtggaaggcaccagacacgaggaatgcgataatgatagcgccctcttttggaatgccgttcctcaggcatgggaaataaaggtgcctaaccatccaacggtgcactggcatgttccacatcttccaatactctgccacggtcttagcgttccaccaatccttgtagaactctctgtcgccgaacctaagaagctcagcgaggatattgagccagaggtggaagaatgagtagaacatgcagagccacacgtaaaggtttggcacagagagcttgagcaccctctcaatagcgtagaggaaatcgcccttaagagggtgcttagagttcctcacgatggggttgatgtactgctcgatgatgaagcccatgagtccgatgaacacaatgagcttcacgaactgcctcacaacccatccctttctaatgcaggaggaccttggataagagggctggtagcaaagggttggagccaccataaagtaagcgagggacttaagggacacgtcgtaagagcaagacacgatggtggagtttgggtgagcatcacccttgtatccggacttagcaagggtcctaatatcagaggaggtgtgggcgtaagaaacgagcttgagccacatgatgcagccgaaaagcataagcacaacgccagacatgtacacggagtcgcaggtaagaatcacgatcactgggtaaagcacagcagcggtagaaacgatcacgtgaagaagcacagccacaagctcagcaatgtggttcctctgaacgagcttctcaacgatgaaagcagcaagtgggaaaattgggagggtgaggcagcacatgaaaatggaccagtctccaagggatcttgaggagaaccagaatccggtatcgatgagccagccgtacttcatgaggttctcgataatgagcctggagttcacggcaatgagcaccacaatgcagaggttgaagagtccagcgtgagactgcttgaagatagcatcggaggagagaggagactctctcacccttctatgagctgggaaggatggtctgtaggtgaacctgatatcagtctcgcccacttgcttctcttcgtttccgatcaccacaccaccaccgtttccaatgaggttcacagatccggtagcgttttccatagctcccaccctatcctgatccctgttctcagcaacatgtccagtagctggaacaccgttggtagaggtgaagccagaagaagaaggctcaatcacagagctggagggcttacgccttctgaagttgttaagatcggaatcgccgtggccggacatagtggtagtgttctacaccaaccaaaaatagatccaattagtatttcaaactcatcacattatttgaaatggttgaattaagtattattgatatcaatgacttgttaaccaacagcacttgttatatataaaagataaataagttatatctatagagagaaaaaaggaaacatacttgagatgactcagccacggccatggttttttttacgcgttccttcaccttcttacttctgaatcacttgcagttctgctgctagatggccaagcttcttctccttcttgctatatataatagtgtgatctgttcagtattaatccaatggtttcaaatggaagggatctgttcctgacatgttacaaccgttggattctataagtgatgacattacagcatattgtccacacgagtaaggaaaatatttgaatggtagccttatcgttaggtaccatgtggaactgacacgtggaacaggttccattatcttatcccttccttggaagaacgtaaatgtgtgatgctggtggaattgatattttttgtgagtggtttgtactaatgtgccttcattccattttaccttaactattttctgaccaccatttctcatttatgggttaagtgaataaataccctttgtctctttgttacatgttacagtagtattttatattttaaaggagattttgaattggaaaaaaacaatagaaccatataaactgacacccttatcccttttgttgtatagctgcaaacatgcttctctctgcaccttaggttggttaattcataaaatattactaaaaatgttcattggtttgtatgttatttgttagttttgtttaagagcaatgtagataagcataatgtgtatatggtatagaaaggttggcagatgatggggcttatcattcttggttgattaatgaccaagcatacattttgaaaacataatatccacaaatagctatggtcatcagtaaatccatgaactcaaccaaacaattcacaacaaccttatcttaaaatacactgcatgagataactataaaacacaaaatcaaaggtatttaatgaagttcaaagagtatgactgtcataatttagtatggcaaaatatgacaaaatgccactgccaataagagaaacaggcgacaaatgttcggtatggaagttcatgactggaaagaagatagatatagatgaaaatggttaatggttgaaaatttcagttctgggaactgtaaaattatcagttttgtataacagcttgtttacaaactgttagttccagattgttctcaaagaattttggctcagttatactctagcagggggacttgttactctttcttctgagctacatctttgtctggttgtaataatttttctcattcatatccccacaaaaacagaagacaaaaatctacgtgatttaatgctattcaactcagttaatggttcaaaaatgaataaaaggagagtaaggaatcagggaatatataaaaggaaccattgttgacaatacaaatgaaaaaagcttctgatattttctgcataataacagaatacaaatttactatatgaatgcttcattgttgacagaaaatatgtatctcttttgatcattccttcccatttcaaatcatgtaccccaaatacttcctccatcttcagcagcacttttctatcccggtatatgaattcatattctagtgggacacgaagtgatccgtttaaactatcagtgtttgacaggatatattggcgggtaaac

SEQ ID NO: 167 - DNA, 10936 bp, synthetic construct
  source 1..10936: mol_type = other DNA
tggcaggatatattgtggtgtaaacaaattgacgcttagacaacttaataacacattgcggacgtttttaatgtactggggttgaacactctgtgccgaattcggatccagcgaacaatcgacaatacataatagctgcattgtcacccgatacttaaataattgtctcatacaaataaaagattgaagatattcaaacaatatttatatgtgaagataaaaaaaaaccaaaaaaatgaaccaaggaccaaatataatatgatatttttttgcttagggacaaaaaaaaacagcatcatcacaagcatcctaaagaaaatagaagagataaaacagagatataaaactaaagtacacaagacttcttgacacaaccataaggttctaaagagaaaaaagaaaataaaatccacataacaacagatagttcaattcccatctcaactagggaaaatagatttttttgtaacaaactcaccaccacgaaccagataagacaagacacacaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcattcctggttcggctttctttggcccggatgatgatatggcgggttcggttgcgacggcgaaaacggtactaaagccatgacgttgcctgggccaaaattgtcttgtaggaagatacacaaaacggcgaaggctggaagtgaaggagctaggttttgacaggaaagaagaagaaaaaagcagtgaatgactggaagccatggctgtcgagtcaacaatcacagataaatcagaatcaaaatccattacgtaatcttaagtacggatctgaagcaatctcgtgaaaaaaaaccctaatccacggcaatagaatcaacaaacaaggcagttgctaacagatctgagcaatatctctcgaaaaacgatatagaatcaacgaataaggctttttgtcaacggatctgaacaatttcttgaaaaaccacagaagtataaaaaataaggctgttgtttaacggatctgaagtaattcatgaaaaagccctgatctatggttacgtttaacggatctgaagtaatcaatgaaaaaagccctaatctacgattaatgaatcaacaattaaggcttgttgtaaagggatctgacatgaaaaagctctaattagagcaaatctacgattaaagaatcatcaaattaaggacgttgttgtaaagataataaaagctgaatctgattatgaaaattgaagatcgatgaaaccaaacaaaacacaatcaaatcaacatcgtattgatcaaagaacatacacaattcatggaaagaacgaagaagaaaaacagaagaaatcgactaaccttgaagggagaggtagggaaatacacagagagaatggaatatattggaattgaactgcgaaggaaggaggaggggtgtggggcgttttatagcataaggtgtggaacccttctccagtcatattgtgacgcgtgttcatgtatggtgtcacttggtgcctatgacacgggtaatatttggtattggatgcattttcggagagtccttctaggaaattgagaattgcaatttgcgtggtagttggcggtttcgtaatcttgacggagtcacatgcaccttcaagcttcgctttatctgttacgacgtcgttgttatattgggcccaccccgttgcaagtggagcgtccgaagttgatacgggccccactatccattgcctgaagtcggctgcttttacattattaaaaatctatggaaaagctgtcatgctagattttttttattatggaccagaaaagaataattgaaaagttttcttattatggagcaaaaaaatctttaataattattttttaaataaaatcataattattccactaaattctttcttatttaatttcaccttgtaagatatattttcagtagatttttcaagtatttttttaacatacattacttgtatacatttagttacagtttatataaaattattcatttaaaaaatatttagatattttttaaaaaataattcaaaatattttttttaatgaactttcctttttcctaatttttctttcttatttcttattagatgtttttactaccaatcgtttagggaagaatttttttctctaatgaggagatgtgttaaatgatgcaacttaaggacagattcaccaataacataagactaaaagatagctcattgacaaaaacaataatgatgttaaagaaaacgaatccatttccattcggataagccacaccatttacgaacaagttgcctactaataatatatggtgtcagttttttttttttttgggctgaattggaggggaaattatccccaaagtttgattaaaaatcacgcgacaaagtaacaaaacttttattccgctaaaaaaattagtcaaagtcaattaaatctggtattagaattacttttccgctaatgatatatccacaataacaatgtattaatattgaaaaaaataaagtttatagagtgtgactttttctttcatttttacgtaaattgaaaaaaaaatcaagcctaatatataaactttcattgatcctcacctaaatcctaaagttcttgagcccaattaaaaaacttactggaggataaaggcccaaacttctttttttttttaggaacagagcatgtaatgggtaatgaggcagcctaagcccataaccatgttagcccgtatatatttttgttaattagatttccggtttcttatttttttttagatttcagtttttgtttctcaaaaaaataaaataacttttagtgctgtattaactttctgtaaataattttagtctcaataaagattaattctgaataataaaaataaacgaggacaatttttttcacaacttttaattctttatttattttttacacttaattttgggaaacatggacctagtgtcaaagaactcaaactcaacattacaaaaacttactacatattcaaggatcatggtctgaactaatctccacataacatataatcacatttttcaattaaaaagaagaattaataatgtattaaatgatgatcgacaagatcacatgatttgagatttttttttatgatgggaaaaatatgatttgagattaggtaacaatctttccattttagatagacttatgtctccgcaagctagctatatacctgcaggatatatggatcccaacccaatttgcttcaagacgtgctcaaatcactatttccacacccctatatttctattgcactcccttttaactgttttttattacaaaaatgccctggaaaatgcactccctttttgtgtttgttatttagtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccggtaagagacaacaacgtaggtacgtggagtgtcttaggtgacttacccacataacataaagtgacattaacaaacatagctaatgctcctatttgaatagtgcatatcagcatagcttattacatatagataggagcaaactctagctagattgttgagagaggtgacctaggttcattagctggcctgaacgccagatatgcactgttgtccataatctttgatctctctggcgcagtccatgattttatggggggcagaatcgagacagtcggcaccaggagggtatttgcctgtaacgtagcagtagatccaagcaaggacagtgattgcagccacaccgaatcccccactggcaagaaagcccagtgacaacaaagccacagtgatcactgcgggaacaagtacgggggagaagataacaagtggtggtgtaacaatggtcagtgcgataatagttccagcgaggatcaaggaggcgagaatcaggagagatcctccggcagtcactgcagtggcggccttcaccacttggcagctatagcttgtttgttgttcgtaggggcactgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacatttggataacgatgaggatagcagtgctgttgttggtagtgcaagcatgccatggttttttttaagggagcaagcggccgaagcctgcttttttgtacaaacttgtgatggggtacggcgcgccttccttcttcttagttctcaactacttgtttttctgctgccagatatccaagcttctgctcaaacttgctatatataatagtgtgatttgtgtagtactaatccaatggtgtgaaatggagaggattggttgccgaaaagttacaaccgttggattgaataagtgatgacattacagcatataggccacacgagtgaggaaaattttgaatggtagccttattgttaggtaccatgtggaggtgacacgtggaggctccattatcttatctcttccttggaagaacgtaaatgtgtgatggtggtggaattgatattatttgtgtagtttgcacagtgccctcattccatattaacctaacatattttgtgaccatcctttctcattcatggattaagtgaaaactctttgttcctttgttacagtaaattttatattttaaagttggttttttattggaaaagaaacaagagaaccatatcaactgaaactcttatccattttgttgggtagcttaaagcatgcttctctcttcacaattacattggccaatccttataataatattactaaaaatgttcattaacttaacgtattatctgttagacttgtttgagtgtaacagtagcaatgtagataagcaaaatgcgtatatggtatagaaggggtggtagatgatggggcttatcatgcttggttgattaatgaccaagcatacacttttgaaaatataagatccacaagtagctatggtaatcagtaaatcgttgcactcaaccaaacaatgcacaacaaccttatcttaaaagtgcaatgcatgagataacaattataaagcacaaaatgaaaagtcaagtattacgtaagaagttcagagagtgtgactctcataagaagccactgcctataaaagaaataggcaacaaattttcagcatggaaagttcaagactgaagaactgttagtgtttaagatgaaattggttaatggttgagaattgcatctttggaaattgtaaaattctcagtttggtataacagtttgtatacaagatgtgagttctagtgattctagcagtgaggcatgtttctctttcttctgagccacagctttgtctggttgtaaggatatttcgtattcatatccccacaaaacgaaaagaaaaatctacatgattttaccactattccaactcagttaatggttcaaaattcaaaagccgtgttttgaaggaaaggaaggcaacagagaatatatataaaaaagaacagttgggcttctaagatgaatgatatgctgaaaatatcaagagcaacatttgtccaatttactaacacacaacattcctatgaatccttcaatgttggtctgccaattttctatctcttctaatcattctccccttttttcaaatcatgtaccccaaacattccctcccctcttctgcaccacttccactactagcccgggtctgttaatgaacgtaaggagcttctggatgacggctgggacttctttgacaaagagacagagctctaagtgtcttcttttcagtagattatcggtgctagaataaaagagatatgaagggattctgggttctgctctatgtagagtaaagcttggatctttgatctaatctaccgttagtataaaacatctattgagctctcttttatttttcttctcagtgtatagtttgcttattgtttttttggtgtttttttccacctctttgtggtactgacataaacaaagactatcgttttgtcatagaattttgagattttgaagaaaagttggataatttagtatactttttaactgactttataaaacgaattagcttcaattagagcttcacaattctcggattttactgaatgacaagttaacaatttaagctagaaaaaaatatgctctaagcctggacatgttgttagtcatggccagcgtatgtgtacattaggtaaatgaaaacaaaaacaaaaactttgaaggtttctgttaagaaaggaactaatttagcaataagagacatgatattacaagctcaacactgtcgctatatctaccagaagccaacaaatcttccagtgcgtaccattggagccacttttcaggctcattagacaaaagtagcacatataatgtaaagacgaaacgatgataaagagactcacaaggacataatttcaatacagaagaagtttgatgtcattactcattagaaagaaaggtataacacaggaacggaaacatagttgaacaattattcatcaggggctttacaaggccccaaaacacaaaccaccaaagttttaaatgaaacgagacatgaacttcttctgattcataacagagataagtttactagtccctcattatttctctttgaggttgatgaggtcgtggtagtaaagcaacacgcacattggctggccgaggatgcagaagatgaaccagaaaatcatgttgcccaccatggagttggagaacttctcttggaggtagttggtaatgagcacgagtggcacctggaacatgattccgatgaaagcccagagcttgaacacgtggcatggaacagcaatgcacaattcgtggaaggcaccagacacgaggaatgcgataatgatagcgccctcttttggaatgccgttcctcaggcatgggaaataaaggtgcctaaccatccaacggtgcactggcatgttccacatcttccaatactctgccacggtcttagcgttccaccaatccttgtagaactctctgtcgccgaacctaagaagctcagcgaggatattgagccagaggtggaagaatgagtagaacatgcagagccacacgtaaaggtttggcacagagagcttgagcaccctctcaatagcgtagaggaaatcgcccttaagagggtgcttagagttcctcacgatggggttgatgtactgctcgatgatgaagcccatgagtccgatgaacacaatgagcttcacgaactgcctcacaacccatccctttctaatgcaggaggaccttggataagagggctggtagcaaagggttggagccaccataaagtaagcgagggacttaagggacacgtcgtaagagcaagacacgatggtggagtttgggtgagcatcacccttgtatccggacttagcaagggtcctaatatcagaggaggtgtgggcgtaagaaacgagcttgagccacatgatgcagccgaaaagcataagcacaacgccagacatgtacacggagtcgcaggtaagaatcacgatcactgggtaaagcacagcagcggtagaaacgatcacgtgaagaagcacagccacaagctcagcaatgtggttcctctgaacgagcttctcaacgatgaaagcagcaagtgggaaaattgggagggtgaggcagcacatgaaaatggaccagtctccaagggatcttgaggagaaccagaatccggtatcgatgagccagccgtacttcatgaggttctcgataatgagcctggagttcacggcaatgagcaccacaatgcagaggttgaagagtccagcgtgagactgcttgaagatagcatcggaggagagaggagactctctcacccttctatgagctgggaaggatggtctgtaggtgaacctgatatcagtctcgcccacttgcttctcttcgtttccgatcaccacaccaccaccgtttccaatgaggttcacagatccggtagcgttttccatagctcccaccctatcctgatccctgttctcagcaacatgtccagtagctggaacaccgttggtagaggtgaagccagaagaagaaggctcaatcacagagctggagggcttacgccttctgaagttgttaagatcggaatcgccgtggccggacatagtggtagtgttctacaccaaccaaaaatagatccaattagtatttcaaactcatcacattatttgaaatggttgaattaagtattattgatatcaatgacttgttaaccaacagcacttgttatatataaaagataaataagttatatctatagagagaaaaaaggaaacatacttgagatgactcagccacggccatggttttttttacgcgttccttcaccttcttacttctgaatcacttgcagttctgctgctagatggccaagcttcttctccttcttgctatatataatagtgtgatctgttcagtattaatccaatggtttcaaatggaagggatctgttcctgacatgttacaaccgttggattctataagtgatgacattacagcatattgtccacacgagtaaggaaaatatttgaatggtagccttatcgttaggtaccatgtggaactgacacgtggaacaggttccattatcttatcccttccttggaagaacgtaaatgtgtgatgctggtggaattgatattttttgtgagtggtttgtactaatgtgccttcattccattttaccttaactattttctgaccaccatttctcatttatgggttaagtgaataaataccctttgtctctttgttacatgttacagtagtattttatattttaaaggagattttgaattggaaaaaaacaatagaaccatataaactgacacccttatcccttttgttgtatagctgcaaacatgcttctctctgcaccttaggttggttaattcataaaatattactaaaaatgttcattggtttgtatgttatttgttagttttgtttaagagcaatgtagataagcataatgtgtatatggtatagaaaggttggcagatgatggggcttatcattcttggttgattaatgaccaagcatacattttgaaaacataatatccacaaatagctatggtcatcagtaaatccatgaactcaaccaaacaattcacaacaaccttatcttaaaatacactgcatgagataactataaaacacaaaatcaaaggtatttaatgaagttcaaagagtatgactgtcataatttagtatggcaaaatatgacaaaatgccactgccaataagagaaacaggcgacaaatgttcggtatggaagttcatgactggaaagaagatagatatagatgaaaatggttaatggttgaaaatttcagttctgggaactgtaaaattatcagttttgtataacagcttgtttacaaactgttagttccagattgttctcaaagaattttggctcagttatactctagcagggggacttgttactctttcttctgagctacatctttgtctggttgtaataatttttctcattcatatccccacaaaaacagaagacaaaaatctacgtgatttaatgctattcaactcagttaatggttcaaaaatgaataaaaggagagtaaggaatcagggaatatataaaaggaaccattgttgacaatacaaatgaaaaaagcttctgatattttctgcataataacagaatacaaatttactatatgaatgcttcattgttgacagaaaatatgtatctcttttgatcattccttcccatttcaaatcatgtaccccaaatacttcctccatcttcagcagcacttttctatcccggtatatgaattcatattctagtgggacacgaagtgatccgtttaaactatcagtgtttgacaggatatattggcgggtaaac

SEQ ID NO: 168 - DNA, 10936 bp, synthetic construct
  source 1..10936: mol_type = other DNA
tggcaggatatattgtggtgtaaacaaattgacgcttagacaacttaataacacattgcggacgtttttaatgtactggggttgaacactctgtgccgaattcggatccagcgaacaatcgacaatacataatagctgcattgtcacccgatacttaaataattgtctcatacaaataaaagattgaagatattcaaacaatatttatatgtgaagataaaaaaaaaccaaaaaaatgaaccaaggaccaaatataatatgatatttttttgcttagggacaaaaaaaaacagcatcatcacaagcatcctaaagaaaatagaagagataaaacagagatataaaactaaagtacacaagacttcttgacacaaccataaggttctaaagagaaaaaagaaaataaaatccacataacaacagatagttcaattcccatctcaactagggaaaatagatttttttgtaacaaactcaccaccacgaaccagataagacaagacacacaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcattcctggttcggctttctttggcccggatgatgatatggcgggttcggttgcgacggcgaaaacggtactaaagccatgacgttgcctgggccaaaattgtcttgtaggaagatacacaaaacggcgaaggctggaagtgaaggagctaggttttgacaggaaagaagaagaaaaaagcagtgaatgactggaagccatggctgtcgagtcaacaatcacagataaatcagaatcaaaatccattacgtaatcttaagtacggatctgaagcaatctcgtgaaaaaaaaccctaatccacggcaatagaatcaacaaacaaggcagttgctaacagatctgagcaatatctctcgaaaaacgatatagaatcaacgaataaggctttttgtcaacggatctgaacaatttcttgaaaaaccacagaagtataaaaaataaggctgttgtttaacggatctgaagtaattcatgaaaaagccctgatctatggttacgtttaacggatctgaagtaatcaatgaaaaaagccctaatctacgattaatgaatcaacaattaaggcttgttgtaaagggatctgacatgaaaaagctctaattagagcaaatctacgattaaagaatcatcaaattaaggacgttgttgtaaagataataaaagctgaatctgattatgaaaattgaagatcgatgaaaccaaacaaaacacaatcaaatcaacatcgtattgatcaaagaacatacacaattcatggaaagaacgaagaagaaaaacagaagaaatcgactaaccttgaagggagaggtagggaaatacacagagagaatggaatatattggaattgaactgcgaaggaaggaggaggggtgtggggcgttttatagcataaggtgtggaacccttctccagtcatattgtgacgcgtgttcatgtatggtgtcacttggtgcctatgacacgggtaatatttggtattggatgcattttcggagagtccttctaggaaattgagaattgcaatttgcgtggtagttggcggtttcgtaatcttgacggagtcacatgcaccttcaagcttcgctttatctgttacgacgtcgttgttatattgggcccaccccgttgcaagtggagcgtccgaagttgatacgggccccactatccattgcctgaagtcggctgcttttacattattaaaaatctatggaaaagctgtcatgctagattttttttattatggaccagaaaagaataattgaaaagttttcttattatggagcaaaaaaatctttaataattattttttaaataaaatcataattattccactaaattctttcttatttaatttcaccttgtaagatatattttcagtagatttttcaagtatttttttaacatacattacttgtatacatttagttacagtttatataaaattattcatttaaaaaatatttagatattttttaaaaaataattcaaaatattttttttaatgaactttcctttttcctaatttttctttcttatttcttattagatgtttttactaccaatcgtttagggaagaatttttttctctaatgaggagatgtgttaaatgatgcaacttaaggacagattcaccaataacataagactaaaagatagctcattgacaaaaacaataatgatgttaaagaaaacgaatccatttccattcggataagccacaccatttacgaacaagttgcctactaataatatatggtgtcagttttttttttttttgggctgaattggaggggaaattatccccaaagtttgattaaaaatcacgcgacaaagtaacaaaacttttattccgctaaaaaaattagtcaaagtcaattaaatctggtattagaattacttttccgctaatgatatatccacaataacaatgtattaatattgaaaaaaataaagtttatagagtgtgactttttctttcatttttacgtaaattgaaaaaaaaatcaagcctaatatataaactttcattgatcctcacctaaatcctaaagttcttgagcccaattaaaaaacttactggaggataaaggcccaaacttctttttttttttaggaacagagcatgtaatgggtaatgaggcagcctaagcccataaccatgttagcccgtatatatttttgttaattagatttccggtttcttatttttttttagatttcagtttttgtttctcaaaaaaataaaataacttttagtgctgtattaactttctgtaaataattttagtctcaataaagattaattctgaataataaaaataaacgaggacaatttttttcacaacttttaattctttatttattttttacacttaattttgggaaacatggacctagtgtcaaagaactcaaactcaacattacaaaaacttactacatattcaaggatcatggtctgaactaatctccacataacatataatcacatttttcaattaaaaagaagaattaataatgtattaaatgatgatcgacaagatcacatgatttgagatttttttttatgatgggaaaaatatgatttgagattaggtaacaatctttccattttagatagacttatgtctccgcaagctagctatatacctgcaggatatatggatcccaacccaatttgcttcaagacgtgctcaaatcactatttccacacccctatatttctattgcactcccttttaactgttttttattacaaaaatgccctggaaaatgcactccctttttgtgtttgttatttagtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccggtaagagacaacaacgtaggtacgtggagtgtcttaggtgacttacccacataacataaagtgacattaacaaacatagctaatgctcctatttgaatagtgcatatcagcatagcttattacatatagataggagcaaactctagctagattgttgagagaggtgacctaggttcattagctggcctgaacgccagatatgcactgttgtccataatctttgatctctctggcgcagtccatgattttatggggggcagaatcgagacagtcggcaccaggagggtatttgcctgtaacgtacctgtagatccaagcaaggacagtgattgcagccacaccgaatcccccactggcaagaaagcccagtgacaacaaagccacagtgatcactgcgggaacaagtacgggggagaagataacaagtggtggtgtaacaatggtcagtgcgataatagttccagcgaggatcaaggaggcgagaatcaggagagatcctccggcagtcactgcagtggcggccttcaccacgcaagtgctatagcttgtttgttgttcgtaggggcactgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacatttggataacgatgaggatagcagtgctgttgttggtagtgcaagcatgccatggttttttttaagggagcaagcggccgaagcctgcttttttgtacaaacttgtgatggggtacggcgcgccttccttcttcttagttctcaactacttgtttttctgctgccagatatccaagcttctgctcaaacttgctatatataatagtgtgatttgtgtagtactaatccaatggtgtgaaatggagaggattggttgccgaaaagttacaaccgttggattgaataagtgatgacattacagcatataggccacacgagtgaggaaaattttgaatggtagccttattgttaggtaccatgtggaggtgacacgtggaggctccattatcttatctcttccttggaagaacgtaaatgtgtgatggtggtggaattgatattatttgtgtagtttgcacagtgccctcattccatattaacctaacatattttgtgaccatcctttctcattcatggattaagtgaaaactctttgttcctttgttacagtaaattttatattttaaagttggttttttattggaaaagaaacaagagaaccatatcaactgaaactcttatccattttgttgggtagcttaaagcatgcttctctcttcacaattacattggccaatccttataataatattactaaaaatgttcattaacttaacgtattatctgttagacttgtttgagtgtaacagtagcaatgtagataagcaaaatgcgtatatggtatagaaggggtggtagatgatggggcttatcatgcttggttgattaatgaccaagcatacacttttgaaaatataagatccacaagtagctatggtaatcagtaaatcgttgcactcaaccaaacaatgcacaacaaccttatcttaaaagtgcaatgcatgagataacaattataaagcacaaaatgaaaagtcaagtattacgtaagaagttcagagagtgtgactctcataagaagccactgcctataaaagaaataggcaacaaattttcagcatggaaagttcaagactgaagaactgttagtgtttaagatgaaattggttaatggttgagaattgcatctttggaaattgtaaaattctcagtttggtataacagtttgtatacaagatgtgagttctagtgattctagcagtgaggcatgtttctctttcttctgagccacagctttgtctggttgtaaggatatttcgtattcatatccccacaaaacgaaaagaaaaatctacatgattttaccactattccaactcagttaatggttcaaaattcaaaagccgtgttttgaaggaaaggaaggcaacagagaatatatataaaaaagaacagttgggcttctaagatgaatgatatgctgaaaatatcaagagcaacatttgtccaatttactaacacacaacattcctatgaatccttcaatgttggtctgccaattttctatctcttctaatcattctccccttttttcaaatcatgtaccccaaacattccctcccctcttctgcaccacttccactactagcccgggtctgttaatgaacgtaaggagcttctggatgacggctgggacttctttgacaaagagacagagctctaagtgtcttcttttcagtagattatcggtgctagaataaaagagatatgaagggattctgggttctgctctatgtagagtaaagcttggatctttgatctaatctaccgttagtataaaacatctattgagctctcttttatttttcttctcagtgtatagtttgcttattgtttttttggtgtttttttccacctctttgtggtactgacataaacaaagactatcgttttgtcatagaattttgagattttgaagaaaagttggataatttagtatactttttaactgactttataaaacgaattagcttcaattagagcttcacaattctcggattttactgaatgacaagttaacaatttaagctagaaaaaaatatgctctaagcctggacatgttgttagtcatggccagcgtatgtgtacattaggtaaatgaaaacaaaaacaaaaactttgaaggtttctgttaagaaaggaactaatttagcaataagagacatgatattacaagctcaacactgtcgctatatctaccagaagccaacaaatcttccagtgcgtaccattggagccacttttcaggctcattagacaaaagtagcacatataatgtaaagacgaaacgatgataaagagactcacaaggacataatttcaatacagaagaagtttgatgtcattactcattagaaagaaaggtataacacaggaacggaaacatagttgaacaattattcatcaggggctttacaaggccccaaaacacaaaccaccaaagttttaaatgaaacgagacatgaacttcttctgattcataacagagataagtttactagtccctcattatttctctttgaggttgatgaggtcgtggtagtaaagcaacacgcacattggctggccgaggatgcagaagatgaaccagaaaatcatgttgcccaccatggagttggagaacttctcttggaggtagttggtaatgagcacgagtggcacctggaacatgattccgatgaaagcccagagcttgaacacgtggcatggaacagcaatgcacaattcgtggaaggcaccagacacgaggaatgcgataatgatagcgccctcttttggaatgccgttcctcaggcatgggaaataaaggtgcctaaccatccaacggtgcactggcatgttccacatcttccaatactctgccacggtcttagcgttccaccaatccttgtagaactctctgtcgccgaacctaagaagctcagcgaggatattgagccagaggtggaagaatgagtagaacatgcagagccacacgtaaaggtttggcacagagagcttgagcaccctctcaatagcgtagaggaaatcgcccttaagagggtgcttagagttcctcacgatggggttgatgtactgctcgatgatgaagcccatgagtccgatgaacacaatgagcttcacgaactgcctcacaacccatccctttctaatgcaggaggaccttggataagagggctggtagcaaagggttggagccaccataaagtaagcgagggacttaagggacacgtcgtaagagcaagacacgatggtggagtttgggtgagcatcacccttgtatccggacttagcaagggtcctaatatcagaggaggtgtgggcgtaagaaacgagcttgagccacatgatgcagccgaaaagcataagcacaacgccagacatgtacacggagtcgcaggtaagaatcacgatcactgggtaaagcacagcagcggtagaaacgatcacgtgaagaagcacagccacaagctcagcaatgtggttcctctgaacgagcttctcaacgatgaaagcagcaagtgggaaaattgggagggtgaggcagcacatgaaaatggaccagtctccaagggatcttgaggagaaccagaatccggtatcgatgagccagccgtacttcatgaggttctcgataatgagcctggagttcacggcaatgagcaccacaatgcagaggttgaagagtccagcgtgagactgcttgaagatagcatcggaggagagaggagactctctcacccttctatgagctgggaaggatggtctgtaggtgaacctgatatcagtctcgcccacttgcttctcttcgtttccgatcaccacaccaccaccgtttccaatgaggttcacagatccggtagcgttttccatagctcccaccctatcctgatccctgttctcagcaacatgtccagtagctggaacaccgttggtagaggtgaagccagaagaagaaggctcaatcacagagctggagggcttacgccttctgaagttgttaagatcggaatcgccgtggccggacatagtggtagtgttctacaccaaccaaaaatagatccaattagtatttcaaactcatcacattatttgaaatggttgaattaagtattattgatatcaatgacttgttaaccaacagcacttgttatatataaaagataaataagttatatctatagagagaaaaaaggaaacatacttgagatgactcagccacggccatggttttttttacgcgttccttcaccttcttacttctgaatcacttgcagttctgctgctagatggccaagcttcttctccttcttgctatatataatagtgtgatctgttcagtattaatccaatggtttcaaatggaagggatctgttcctgacatgttacaaccgttggattctataagtgatgacattacagcatattgtccacacgagtaaggaaaatatttgaatggtagccttatcgttaggtaccatgtggaactgacacgtggaacaggttccattatcttatcccttccttggaagaacgtaaatgtgtgatgctggtggaattgatattttttgtgagtggtttgtactaatgtgccttcattccattttaccttaactattttctgaccaccatttctcatttatgggttaagtgaataaataccctttgtctctttgttacatgttacagtagtattttatattttaaaggagattttgaattggaaaaaaacaatagaaccatataaactgacacccttatcccttttgttgtatagctgcaaacatgcttctctctgcaccttaggttggttaattcataaaatattactaaaaatgttcattggtttgtatgttatttgttagttttgtttaagagcaatgtagataagcataatgtgtatatggtatagaaaggttggcagatgatggggcttatcattcttggttgattaatgaccaagcatacattttgaaaacataatatccacaaatagctatggtcatcagtaaatccatgaactcaaccaaacaattcacaacaaccttatcttaaaatacactgcatgagataactataaaacacaaaatcaaaggtatttaatgaagttcaaagagtatgactgtcataatttagtatggcaaaatatgacaaaatgccactgccaataagagaaacaggcgacaaatgttcggtatggaagttcatgactggaaagaagatagatatagatgaaaatggttaatggttgaaaatttcagttctgggaactgtaaaattatcagttttgtataacagcttgtttacaaactgttagttccagattgttctcaaagaattttggctcagttatactctagcagggggacttgttactctttcttctgagctacatctttgtctggttgtaataatttttctcattcatatccccacaaaaacagaagacaaaaatctacgtgatttaatgctattcaactcagttaatggttcaaaaatgaataaaaggagagtaaggaatcagggaatatataaaaggaaccattgttgacaatacaaatgaaaaaagcttctgatattttctgcataataacagaatacaaatttactatatgaatgcttcattgttgacagaaaatatgtatctcttttgatcattccttcccatttcaaatcatgtaccccaaatacttcctccatcttcagcagcacttttctatcccggtatatgaattcatattctagtgggacacgaagtgatccgtttaaactatcagtgtttgacaggatatattggcgggtaaac

SEQ ID NO: 169 - DNA, 9891 bp, synthetic construct
  source 1..9891: mol_type = other DNA
tggcaggatatattgtggtgtaaacaaattgacgcttagacaacttaataacacattgcggacgtttttaatgtactggggttgaacactctgtgccgaattcggatccagcgaacaatcgacaatacataatagctgcattgtcacccgatacttaaataattgtctcatacaaataaaagattgaagatattcaaacaatatttatatgtgaagataaaaaaaaaccaaaaaaatgaaccaaggaccaaatataatatgatatttttttgcttagggacaaaaaaaaacagcatcatcacaagcatcctaaagaaaatagaagagataaaacagagatataaaactaaagtacacaagacttcttgacacaaccataaggttctaaagagaaaaaagaaaataaaatccacataacaacagatagttcaattcccatctcaactagggaaaatagatttttttgtaacaaactcaccaccacgaaccagataagacaagacacacaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcattcctggttcggctttctttggcccggatgatgatatggcgggttcggttgcgacggcgaaaacggtactaaagccatgacgttgcctgggccaaaattgtcttgtaggaagatacacaaaacggcgaaggctggaagtgaaggagctaggttttgacaggaaagaagaagaaaaaagcagtgaatgactggaagccatggctgtcgagtcaacaatcacagataaatcagaatcaaaatccattacgtaatcttaagtacggatctgaagcaatctcgtgaaaaaaaaccctaatccacggcaatagaatcaacaaacaaggcagttgctaacagatctgagcaatatctctcgaaaaacgatatagaatcaacgaataaggctttttgtcaacggatctgaacaatttcttgaaaaaccacagaagtataaaaaataaggctgttgtttaacggatctgaagtaattcatgaaaaagccctgatctatggttacgtttaacggatctgaagtaatcaatgaaaaaagccctaatctacgattaatgaatcaacaattaaggcttgttgtaaagggatctgacatgaaaaagctctaattagagcaaatctacgattaaagaatcatcaaattaaggacgttgttgtaaagataataaaagctgaatctgattatgaaaattgaagatcgatgaaaccaaacaaaacacaatcaaatcaacatcgtattgatcaaagaacatacacaattcatggaaagaacgaagaagaaaaacagaagaaatcgactaaccttgaagggagaggtagggaaatacacagagagaatggaatatattggaattgaactgcgaaggaaggaggaggggtgtggggcgttttatagcataaggtgtggaacccttctccagtcatattgtgacgcgtgttcatgtatggtgtcacttggtgcctatgacacgggtaatatttggtattggatgcattttcggagagtccttctaggaaattgagaattgcaatttgcgtggtagttggcggtttcgtaatcttgacggagtcacatgcaccttcaagcttcgctttatctgttacgacgtcgttgttatattgggcccaccccgttgcaagtggagcgtccgaagttgatacgggccccactatccattgcctgaagtcggctgcttttacattattaaaaatctatggaaaagctgtcatgctagattttttttattatggaccagaaaagaataattgaaaagttttcttattatggagcaaaaaaatctttaataattattttttaaataaaatcataattattccactaaattctttcttatttaatttcaccttgtaagatatattttcagtagatttttcaagtatttttttaacatacattacttgtatacatttagttacagtttatataaaattattcatttaaaaaatatttagatattttttaaaaaataattcaaaatattttttttaatgaactttcctttttcctaatttttctttcttatttcttattagatgtttttactaccaatcgtttagggaagaatttttttctctaatgaggagatgtgttaaatgatgcaacttaaggacagattcaccaataacataagactaaaagatagctcattgacaaaaacaataatgatgttaaagaaaacgaatccatttccattcggataagccacaccatttacgaacaagttgcctactaataatatatggtgtcagttttttttttttttgggctgaattggaggggaaattatccccaaagtttgattaaaaatcacgcgacaaagtaacaaaacttttattccgctaaaaaaattagtcaaagtcaattaaatctggtattagaattacttttccgctaatgatatatccacaataacaatgtattaatattgaaaaaaataaagtttatagagtgtgactttttctttcatttttacgtaaattgaaaaaaaaatcaagcctaatatataaactttcattgatcctcacctaaatcctaaagttcttgagcccaattaaaaaacttactggaggataaaggcccaaacttctttttttttttaggaacagagcatgtaatgggtaatgaggcagcctaagcccataaccatgttagcccgtatatatttttgttaattagatttccggtttcttatttttttttagatttcagtttttgtttctcaaaaaaataaaataacttttagtgctgtattaactttctgtaaataattttagtctcaataaagattaattctgaataataaaaataaacgaggacaatttttttcacaacttttaattctttatttattttttacacttaattttgggaaacatggacctagtgtcaaagaactcaaactcaacattacaaaaacttactacatattcaaggatcatggtctgaactaatctccacataacatataatcacatttttcaattaaaaagaagaattaataatgtattaaatgatgatcgacaagatcacatgatttgagatttttttttatgatgggaaaaatatgatttgagattaggtaacaatctttccattttagatagacttatgtctccgcaagctagctatatacctgcaggatatatggatcccaacccaatttgcttcaagacgtgctcaaatcactatttccacacccctatatttctattgcactcccttttaactgttttttattacaaaaatgccctggaaaatgcactccctttttgtgtttgttatttagtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccggtaagagacaacaacgtaggtacgtggagtgtcttaggtgacttacccacataacataaagtgacattaacaaacatagctaatgctcctatttgaatagtgcatatcagcatagcttattacatatagataggagcaaactctagctagattgttgagagaggtgacctaggttcattagctggcctgaacgccagatatgcactgttgtccataatctttgatctctctggcgcagtccatgattttatggggggcagaatcgagacagtcggcaccaggagggtatttgcctgtaacgtacctgtagatccaagcaaggacagtgattgcagccacaccgaatcccccactggcaagaaagcccagtgacaacaaagccacagtgatcactgcgggaacaagtacgggggagaagataacaagtggtggtgtaacaatggtcagtgcgataatagttccagcgaggatcaaggaggcgagaatcaggagagatcctccggcagtcactgcagtggcggccttcaccacgcaagtgctatagcttgtttgttgttcgtaggggcactgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacatttggataacgatgaggatagcagtgctgttgttggtagtgcaagcatgccatggttttttttaagggagcaagcggccgaagcctgcttttttgtacaaacttgtgatggggtacggcgcgccttccttcttcttagttctcaactacttgtttttctgctgccagatatccaagcttctgctcaaacttgctatatataatagtgtgatttgtgtagtactaatccaatggtgtgaaatggagaggattggttgccgaaaagttacaaccgttggattgaataagtgatgacattacagcatataggccacacgagtgaggaaaattttgaatggtagccttattgttaggtaccatgtggaggtgacacgtggaggctccattatcttatctcttccttggaagaacgtaaatgtgtgatggtggtggaattgatattatttgtgtagtttgcacagtgccctcattccatattaacctaacatattttgtgaccatcctttctcattcatggattaagtgaaaactctttgttcctttgttacagtaaattttatattttaaagttggttttttattggaaaagaaacaagagaaccatatcaactgaaactcttatccattttgttgggtagcttaaagcatgcttctctcttcacaattacattggccaatccttataataatattactaaaaatgttcattaacttaacgtattatctgttagacttgtttgagtgtaacagtagcaatgtagataagcaaaatgcgtatatggtatagaaggggtggtagatgatggggcttatcatgcttggttgattaatgaccaagcatacacttttgaaaatataagatccacaagtagctatggtaatcagtaaatcgttgcactcaaccaaacaatgcacaacaaccttatcttaaaagtgcaatgcatgagataacaattataaagcacaaaatgaaaagtcaagtattacgtaagaagttcagagagtgtgactctcataagaagccactgcctataaaagaaataggcaacaaattttcagcatggaaagttcaagactgaagaactgttagtgtttaagatgaaattggttaatggttgagaattgcatctttggaaattgtaaaattctcagtttggtataacagtttgtatacaagatgtgagttctagtgattctagcagtgaggcatgtttctctttcttctgagccacagctttgtctggttgtaaggatatttcgtattcatatccccacaaaacgaaaagaaaaatctacatgattttaccactattccaactcagttaatggttcaaaattcaaaagccgtgttttgaaggaaaggaaggcaacagagaatatatataaaaaagaacagttgggcttctaagatgaatgatatgctgaaaatatcaagagcaacatttgtccaatttactaacacacaacattcctatgaatccttcaatgttggtctgccaattttctatctcttctaatcattctccccttttttcaaatcatgtaccccaaacattccctcccctcttctgcaccacttccactactagcccgggtctgttaatgaacgtaaggagcttctggatgacggctgggacttctttgacaaagagacagagctctaagtgtcttcttttcagtagattatcggtgctagaataaaagagatatgaagggattctgggttctgctctatgtagagtaaagcttggatctttgatctaatctaccgttagtataaaacatctattgagctctcttttatttttcttctcagtgtatagtttgcttattgtttttttggtgtttttttccacctctttgtggtactgacataaacaaagactatcgttttgtcatagaattttgagattttgaagaaaagttggataatttagtatactttttaactgactttataaaacgaattagcttcaattagagcttcacaattctcggattttactgaatgacaagttaacaatttaagctagaaaaaaatatgctctaagcctggacatgttgttagtcatggccagcgtatgtgtacattaggtaaatgaaaacaaaaacaaaaactttgaaggtttctgttaagaaaggaactaatttagcaataagagacatgatattacaagctcaacactgtcgctatatctaccagaagccaacaaatcttccagtgcgtaccattggagccacttttcaggctcattagacaaaagtagcacatataatgtaaagacgaaacgatgataaagagactcacaaggacataatttcaatacagaagaagtttgatgtcattactcattagaaagaaaggtataacacaggaacggaaacatagttgaacaattattcatcaggggctttacaaggccccaaaacacaaaccaccaaagttttaaatgaaacgagacatgaacttcttctgattcataacagagataagtttactagtccctcattatttctctttgaggttgatgaggtcgtggtagtaaagcaacacgcacattggctggccgaggatgcagaagatgaaccagaaaatcatgttgcccaccatggagttggagaacttctcttggaggtagttggtaatgagcacgagtggcacctggaacatgattccgatgaaagcccagagcttgaacacgtggcatggaacagcaatgcacaattcgtggaaggcaccagacacgaggaatgcgataatgatagcgccctcttttggaatgccgttcctcaggcatgggaaataaaggtgcctaaccatccaacggtgcactggcatgttccacatcttccaatactctgccacggtcttagcgttccaccaatccttgtagaactctctgtcgccgaacctaagaagctcagcgaggatattgagccagaggtggaagaatgagtagaacatgcagagccacacgtaaaggtttggcacagagagcttgagcaccctctcaatagcgtagaggaaatcgcccttaagagggtgcttagagttcctcacgatggggttgatgtactgctcgatgatgaagcccatgagtccgatgaacacaatgagcttcacgaactgcctcacaacccatccctttctaatgcaggaggaccttggataagagggctggtagcaaagggttggagccaccataaagtaagcgagggacttaagggacacgtcgtaagagcaagacacgatggtggagtttgggtgagcatcacccttgtatccggacttagcaagggtcctaatatcagaggaggtgtgggcgtaagaaacgagcttgagccacatgatgcagccgaaaagcataagcacaacgccagacatgtacacggagtcgcaggtaagaatcacgatcactgggtaaagcacagcagcggtagaaacgatcacgtgaagaagcacagccacaagctcagcaatgtggttcctctgaacgagcttctcaacgatgaaagcagcaagtgggaaaattgggagggtgaggcagcacatgaaaatggaccagtctccaagggatcttgaggagaaccagaatccggtatcgatgagccagccgtacttcatgaggttctcgataatgagcctggagttcacggcaatgagcaccacaatgcagaggttgaagagtccagcgtgagactgcttgaagatagcatcggaggagagaggagactctctcacccttctatgagctgggaaggatggtctgtaggtgaacctgatatcagtctcgcccacttgcttctcttcgtttccgatcaccacaccaccaccgtttccaatgaggttcacagatccggtagcgttttccatagctcccaccctatcctgatccctgttctcagcaacatgtccagtagctggaacaccgttggtagaggtgaagccagaagaagaaggctcaatcacagagctggagggcttacgccttctgaagttgttaagatcggaatcgccgtggccggacatagtggtagtgttctacaccaaccaaaaatagatccaattagtatttcaaactcatcacattatttgaaatggttgaattaagtattattgatatcaatgacttgttaaccaacagcacttgttatatataaaagataaataagttatatctatagagagaaaaaaggaaacatacttgagatgactcagccacggccatggttttttttacgcgtttttctcacttctgtatgaattgcaaagcttctgctaaaacttgctatatataatgtttttatttatctatttccaatattatggtcttaatattgtggtacttagctactgcctagtaccaatcttgttagatagtgaaaatgataaagtttgcagccatattaaccacacaagatttgaaattttgtgtgctagccttatcataagccaccaataggacagtgccatgtggatattacctcactgatcttatcttttcatgtgaagagtgtaaatgtgtgatggtggttatgtcgatatttttattttgtaagaattgtatccatgaaaattcaattgtagtatgttggcctcatggaattttgtgactgttacttcacaaatatcttttatgtatgggtaacatttgttcctgtccaagcttttggatcaccggtatatgaattcatattctagtgggacacgaagtgatccgtttaaactatcagtgtttgacaggatatattggcgggtaaac

SEQ ID NO: 170 - DNA, 10936 bp, synthetic construct
  source 1..10936: mol_type = other DNA
tggcaggatatattgtggtgtaaacaaattgacgcttagacaacttaataacacattgcggacgtttttaatgtactggggttgaacactctgtgccgaattcggatccagcgaacaatcgacaatacataatagctgcattgtcacccgatacttaaataattgtctcatacaaataaaagattgaagatattcaaacaatatttatatgtgaagataaaaaaaaaccaaaaaaatgaaccaaggaccaaatataatatgatatttttttgcttagggacaaaaaaaaacagcatcatcacaagcatcctaaagaaaatagaagagataaaacagagatataaaactaaagtacacaagacttcttgacacaaccataaggttctaaagagaaaaaagaaaataaaatccacataacaacagatagttcaattcccatctcaactagggaaaatagatttttttgtaacaaactcaccaccacgaaccagataagacaagacacacaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcattcctggttcggctttctttggcccggatgatgatatggcgggttcggttgcgacggcgaaaacggtactaaagccatgacgttgcctgggccaaaattgtcttgtaggaagatacacaaaacggcgaaggctggaagtgaaggagctaggttttgacaggaaagaagaagaaaaaagcagtgaatgactggaagccatggctgtcgagtcaacaatcacagataaatcagaatcaaaatccattacgtaatcttaagtacggatctgaagcaatctcgtgaaaaaaaaccctaatccacggcaatagaatcaacaaacaaggcagttgctaacagatctgagcaatatctctcgaaaaacgatatagaatcaacgaataaggctttttgtcaacggatctgaacaatttcttgaaaaaccacagaagtataaaaaataaggctgttgtttaacggatctgaagtaattcatgaaaaagccctgatctatggttacgtttaacggatctgaagtaatcaatgaaaaaagccctaatctacgattaatgaatcaacaattaaggcttgttgtaaagggatctgacatgaaaaagctctaattagagcaaatctacgattaaagaatcatcaaattaaggacgttgttgtaaagataataaaagctgaatctgattatgaaaattgaagatcgatgaaaccaaacaaaacacaatcaaatcaacatcgtattgatcaaagaacatacacaattcatggaaagaacgaagaagaaaaacagaagaaatcgactaaccttgaagggagaggtagggaaatacacagagagaatggaatatattggaattgaactgcgaaggaaggaggaggggtgtggggcgttttatagcataaggtgtggaacccttctccagtcatattgtgacgcgtgttcatgtatggtgtcacttggtgcctatgacacgggtaatatttggtattggatgcattttcggagagtccttctaggaaattgagaattgcaatttgcgtggtagttggcggtttcgtaatcttgacggagtcacatgcaccttcaagcttcgctttatctgttacgacgtcgttgttatattgggcccaccccgttgcaagtggagcgtccgaagttgatacgggccccactatccattgcctgaagtcggctgcttttacattattaaaaatctatggaaaagctgtcatgctagattttttttattatggaccagaaaagaataattgaaaagttttcttattatggagcaaaaaaatctttaataattattttttaaataaaatcataattattccactaaattctttcttatttaatttcaccttgtaagatatattttcagtagatttttcaagtatttttttaacatacattacttgtatacatttagttacagtttatataaaattattcatttaaaaaatatttagatattttttaaaaaataattcaaaatattttttttaatgaactttcctttttcctaatttttctttcttatttcttattagatgtttttactaccaatcgtttagggaagaatttttttctctaatgaggagatgtgttaaatgatgcaacttaaggacagattcaccaataacataagactaaaagatagctcattgacaaaaacaataatgatgttaaagaaaacgaatccatttccattcggataagccacaccatttacgaacaagttgcctactaataatatatggtgtcagttttttttttttttgggctgaattggaggggaaattatccccaaagtttgattaaaaatcacgcgacaaagtaacaaaacttttattccgctaaaaaaattagtcaaagtcaattaaatctggtattagaattacttttccgctaatgatatatccacaataacaatgtattaatattgaaaaaaataaagtttatagagtgtgactttttctttcatttttacgtaaattgaaaaaaaaatcaagcctaatatataaactttcattgatcctcacctaaatcctaaagttcttgagcccaattaaaaaacttactggaggataaaggcccaaacttctttttttttttaggaacagagcatgtaatgggtaatgaggcagcctaagcccataaccatgttagcccgtatatatttttgttaattagatttccggtttcttatttttttttagatttcagtttttgtttctcaaaaaaataaaataacttttagtgctgtattaactttctgtaaataattttagtctcaataaagattaattctgaataataaaaataaacgaggacaatttttttcacaacttttaattctttatttattttttacacttaattttgggaaacatggacctagtgtcaaagaactcaaactcaacattacaaaaacttactacatattcaaggatcatggtctgaactaatctccacataacatataatcacatttttcaattaaaaagaagaattaataatgtattaaatgatgatcgacaagatcacatgatttgagatttttttttatgatgggaaaaatatgatttgagattaggtaacaatctttccattttagatagacttatgtctccgcaagctagctatatacctgcaggatatatggatcccaacccaatttgcttcaagacgtgctcaaatcactatttccacacccctatatttctattgcactcccttttaactgttttttattacaaaaatgccctggaaaatgcactccctttttgtgtttgttatttagtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccggtaagagacaacaacgtaggtacgtggagtgtcttaggtgacttacccacataacataaagtgacattaacaaacatagctaatgctcctatttgaatagtgcatatcagcatagcttattacatatagataggagcaaactctagctagattgttgagagaggtgacctaggttcattagctggcctgaacgccagatatgcactgttgtccataatctttgatctctctggcgcagtccatgattttatggggggcagaatcgagacagtcggcaccaggagggtagcagcctgtaacgtacctgtagatccaagcaaggacagtgattgcagccacaccgaatcccccactggcaagaaagcccagtgacaacaaagccacagtgatcactgcgggaacaagtacgggggagaagataacaagtggtggtgtaacaatggtcagtgcgataatagttccagcgaggatcaaggaggcgagaatcaggagagatcctccggcagtcactgcagtggcggccttcaccacgcaagtgctatagcttgtttgttgttcgtaggggcactgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacatttggataacgatgaggatagcagtgctgttgttggtagtgcaagcatgccatggttttttttaagggagcaagcggccgaagcctgcttttttgtacaaacttgtgatggggtacggcgcgccttccttcttcttagttctcaactacttgtttttctgctgccagatatccaagcttctgctcaaacttgctatatataatagtgtgatttgtgtagtactaatccaatggtgtgaaatggagaggattggttgccgaaaagttacaaccgttggattgaataagtgatgacattacagcatataggccacacgagtgaggaaaattttgaatggtagccttattgttaggtaccatgtggaggtgacacgtggaggctccattatcttatctcttccttggaagaacgtaaatgtgtgatggtggtggaattgatattatttgtgtagtttgcacagtgccctcattccatattaacctaacatattttgtgaccatcctttctcattcatggattaagtgaaaactctttgttcctttgttacagtaaattttatattttaaagttggttttttattggaaaagaaacaagagaaccatatcaactgaaactcttatccattttgttgggtagcttaaagcatgcttctctcttcacaattacattggccaatccttataataatattactaaaaatgttcattaacttaacgtattatctgttagacttgtttgagtgtaacagtagcaatgtagataagcaaaatgcgtatatggtatagaaggggtggtagatgatggggcttatcatgcttggttgattaatgaccaagcatacacttttgaaaatataagatccacaagtagctatggtaatcagtaaatcgttgcactcaaccaaacaatgcacaacaaccttatcttaaaagtgcaatgcatgagataacaattataaagcacaaaatgaaaagtcaagtattacgtaagaagttcagagagtgtgactctcataagaagccactgcctataaaagaaataggcaacaaattttcagcatggaaagttcaagactgaagaactgttagtgtttaagatgaaattggttaatggttgagaattgcatctttggaaattgtaaaattctcagtttggtataacagtttgtatacaagatgtgagttctagtgattctagcagtgaggcatgtttctctttcttctgagccacagctttgtctggttgtaaggatatttcgtattcatatccccacaaaacgaaaagaaaaatctacatgattttaccactattccaactcagttaatggttcaaaattcaaaagccgtgttttgaaggaaaggaaggcaacagagaatatatataaaaaagaacagttgggcttctaagatgaatgatatgctgaaaatatcaagagcaacatttgtccaatttactaacacacaacattcctatgaatccttcaatgttggtctgccaattttctatctcttctaatcattctccccttttttcaaatcatgtaccccaaacattccctcccctcttctgcaccacttccactactagcccgggtctgttaatgaacgtaaggagcttctggatgacggctgggacttctttgacaaagagacagagctctaagtgtcttcttttcagtagattatcggtgctagaataaaagagatatgaagggattctgggttctgctctatgtagagtaaagcttggatctttgatctaatctaccgttagtataaaacatctattgagctctcttttatttttcttctcagtgtatagtttgcttattgtttttttggtgtttttttccacctctttgtggtactgacataaacaaagactatcgttttgtcatagaattttgagattttgaagaaaagttggataatttagtatactttttaactgactttataaaacgaattagcttcaattagagcttcacaattctcggattttactgaatgacaagttaacaatttaagctagaaaaaaatatgctctaagcctggacatgttgttagtcatggccagcgtatgtgtacattaggtaaatgaaaacaaaaacaaaaactttgaaggtttctgttaagaaaggaactaatttagcaataagagacatgatattacaagctcaacactgtcgctatatctaccagaagccaacaaatcttccagtgcgtaccattggagccacttttcaggctcattagacaaaagtagcacatataatgtaaagacgaaacgatgataaagagactcacaaggacataatttcaatacagaagaagtttgatgtcattactcattagaaagaaaggtataacacaggaacggaaacatagttgaacaattattcatcaggggctttacaaggccccaaaacacaaaccaccaaagttttaaatgaaacgagacatgaacttcttctgattcataacagagataagtttactagtccctcattatttctctttgaggttgatgaggtcgtggtagtaaagcaacacgcacattggctggccgaggatgcagaagatgaaccagaaaatcatgttgcccaccatggagttggagaacttctcttggaggtagttggtaatgagcacgagtggcacctggaacatgattccgatgaaagcccagagcttgaacacgtggcatggaacagcaatgcacaattcgtggaaggcaccagacacgaggaatgcgataatgatagcgccctcttttggaatgccgttcctcaggcatgggaaataaaggtgcctaaccatccaacggtgcactggcatgttccacatcttccaatactctgccacggtcttagcgttccaccaatccttgtagaactctctgtcgccgaacctaagaagctcagcgaggatattgagccagaggtggaagaatgagtagaacatgcagagccacacgtaaaggtttggcacagagagcttgagcaccctctcaatagcgtagaggaaatcgcccttaagagggtgcttagagttcctcacgatggggttgatgtactgctcgatgatgaagcccatgagtccgatgaacacaatgagcttcacgaactgcctcacaacccatccctttctaatgcaggaggaccttggataagagggctggtagcaaagggttggagccaccataaagtaagcgagggacttaagggacacgtcgtaagagcaagacacgatggtggagtttgggtgagcatcacccttgtatccggacttagcaagggtcctaatatcagaggaggtgtgggcgtaagaaacgagcttgagccacatgatgcagccgaaaagcataagcacaacgccagacatgtacacggagtcgcaggtaagaatcacgatcactgggtaaagcacagcagcggtagaaacgatcacgtgaagaagcacagccacaagctcagcaatgtggttcctctgaacgagcttctcaacgatgaaagcagcaagtgggaaaattgggagggtgaggcagcacatgaaaatggaccagtctccaagggatcttgaggagaaccagaatccggtatcgatgagccagccgtacttcatgaggttctcgataatgagcctggagttcacggcaatgagcaccacaatgcagaggttgaagagtccagcgtgagactgcttgaagatagcatcggaggagagaggagactctctcacccttctatgagctgggaaggatggtctgtaggtgaacctgatatcagtctcgcccacttgcttctcttcgtttccgatcaccacaccaccaccgtttccaatgaggttcacagatccggtagcgttttccatagctcccaccctatcctgatccctgttctcagcaacatgtccagtagctggaacaccgttggtagaggtgaagccagaagaagaaggctcaatcacagagctggagggcttacgccttctgaagttgttaagatcggaatcgccgtggccggacatagtggtagtgttctacaccaaccaaaaatagatccaattagtatttcaaactcatcacattatttgaaatggttgaattaagtattattgatatcaatgacttgttaaccaacagcacttgttatatataaaagataaataagttatatctatagagagaaaaaaggaaacatacttgagatgactcagccacggccatggttttttttacgcgttccttcaccttcttacttctgaatcacttgcagttctgctgctagatggccaagcttcttctccttcttgctatatataatagtgtgatctgttcagtattaatccaatggtttcaaatggaagggatctgttcctgacatgttacaaccgttggattctataagtgatgacattacagcatattgtccacacgagtaaggaaaatatttgaatggtagccttatcgttaggtaccatgtggaactgacacgtggaacaggttccattatcttatcccttccttggaagaacgtaaatgtgtgatgctggtggaattgatattttttgtgagtggtttgtactaatgtgccttcattccattttaccttaactattttctgaccaccatttctcatttatgggttaagtgaataaataccctttgtctctttgttacatgttacagtagtattttatattttaaaggagattttgaattggaaaaaaacaatagaaccatataaactgacacccttatcccttttgttgtatagctgcaaacatgcttctctctgcaccttaggttggttaattcataaaatattactaaaaatgttcattggtttgtatgttatttgttagttttgtttaagagcaatgtagataagcataatgtgtatatggtatagaaaggttggcagatgatggggcttatcattcttggttgattaatgaccaagcatacattttgaaaacataatatccacaaatagctatggtcatcagtaaatccatgaactcaaccaaacaattcacaacaaccttatcttaaaatacactgcatgagataactataaaacacaaaatcaaaggtatttaatgaagttcaaagagtatgactgtcataatttagtatggcaaaatatgacaaaatgccactgccaataagagaaacaggcgacaaatgttcggtatggaagttcatgactggaaagaagatagatatagatgaaaatggttaatggttgaaaatttcagttctgggaactgtaaaattatcagttttgtataacagcttgtttacaaactgttagttccagattgttctcaaagaattttggctcagttatactctagcagggggacttgttactctttcttctgagctacatctttgtctggttgtaataatttttctcattcatatccccacaaaaacagaagacaaaaatctacgtgatttaatgctattcaactcagttaatggttcaaaaatgaataaaaggagagtaaggaatcagggaatatataaaaggaaccattgttgacaatacaaatgaaaaaagcttctgatattttctgcataataacagaatacaaatttactatatgaatgcttcattgttgacagaaaatatgtatctcttttgatcattccttcccatttcaaatcatgtaccccaaatacttcctccatcttcagcagcacttttctatcccggtatatgaattcatattctagtgggacacgaagtgatccgtttaaactatcagtgtttgacaggatatattggcgggtaaac

SEQ ID NO: 171 - DNA, 10874 bp, synthetic construct
  source 1..10874: mol_type = other DNA
tggcaggatatattgtggtgtaaacaaattgacgcttagacaacttaataacacattgcggacgtttttaatgtactggggttgaacactctgtgccgaattcggatccagcgaacaatcgacaatacataatagctgcattgtcacccgatacttaaataattgtctcatacaaataaaagattgaagatattcaaacaatatttatatgtgaagataaaaaaaaaccaaaaaaatgaaccaaggaccaaatataatatgatatttttttgcttagggacaaaaaaaaacagcatcatcacaagcatcctaaagaaaatagaagagataaaacagagatataaaactaaagtacacaagacttcttgacacaaccataaggttctaaagagaaaaaagaaaataaaatccacataacaacagatagttcaattcccatctcaactagggaaaatagatttttttgtaacaaactcaccaccacgaaccagataagacaagacacacaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcattcctggttcggctttctttggcccggatgatgatatggcgggttcggttgcgacggcgaaaacggtactaaagccatgacgttgcctgggccaaaattgtcttgtaggaagatacacaaaacggcgaaggctggaagtgaaggagctaggttttgacaggaaagaagaagaaaaaagcagtgaatgactggaagccatggctgtcgagtcaacaatcacagataaatcagaatcaaaatccattacgtaatcttaagtacggatctgaagcaatctcgtgaaaaaaaaccctaatccacggcaatagaatcaacaaacaaggcagttgctaacagatctgagcaatatctctcgaaaaacgatatagaatcaacgaataaggctttttgtcaacggatctgaacaatttcttgaaaaaccacagaagtataaaaaataaggctgttgtttaacggatctgaagtaattcatgaaaaagccctgatctatggttacgtttaacggatctgaagtaatcaatgaaaaaagccctaatctacgattaatgaatcaacaattaaggcttgttgtaaagggatctgacatgaaaaagctctaattagagcaaatctacgattaaagaatcatcaaattaaggacgttgttgtaaagataataaaagctgaatctgattatgaaaattgaagatcgatgaaaccaaacaaaacacaatcaaatcaacatcgtattgatcaaagaacatacacaattcatggaaagaacgaagaagaaaaacagaagaaatcgactaaccttgaagggagaggtagggaaatacacagagagaatggaatatattggaattgaactgcgaaggaaggaggaggggtgtggggcgttttatagcataaggtgtggaacccttctccagtcatattgtgacgcgtgttcatgtatggtgtcacttggtgcctatgacacgggtaatatttggtattggatgcattttcggagagtccttctaggaaattgagaattgcaatttgcgtggtagttggcggtttcgtaatcttgacggagtcacatgcaccttcaagcttcgctttatctgttacgacgtcgttgttatattgggcccaccccgttgcaagtggagcgtccgaagttgatacgggccccactatccattgcctgaagtcggctgcttttacattattaaaaatctatggaaaagctgtcatgctagattttttttattatggaccagaaaagaataattgaaaagttttcttattatggagcaaaaaaatctttaataattattttttaaataaaatcataattattccactaaattctttcttatttaatttcaccttgtaagatatattttcagtagatttttcaagtatttttttaacatacattacttgtatacatttagttacagtttatataaaattattcatttaaaaaatatttagatattttttaaaaaataattcaaaatattttttttaatgaactttcctttttcctaatttttctttcttatttcttattagatgtttttactaccaatcgtttagggaagaatttttttctctaatgaggagatgtgttaaatgatgcaacttaaggacagattcaccaataacataagactaaaagatagctcattgacaaaaacaataatgatgttaaagaaaacgaatccatttccattcggataagccacaccatttacgaacaagttgcctactaataatatatggtgtcagttttttttttttttgggctgaattggaggggaaattatccccaaagtttgattaaaaatcacgcgacaaagtaacaaaacttttattccgctaaaaaaattagtcaaagtcaattaaatctggtattagaattacttttccgctaatgatatatccacaataacaatgtattaatattgaaaaaaataaagtttatagagtgtgactttttctttcatttttacgtaaattgaaaaaaaaatcaagcctaatatataaactttcattgatcctcacctaaatcctaaagttcttgagcccaattaaaaaacttactggaggataaaggcccaaacttctttttttttttaggaacagagcatgtaatgggtaatgaggcagcctaagcccataaccatgttagcccgtatatatttttgttaattagatttccggtttcttatttttttttagatttcagtttttgtttctcaaaaaaataaaataacttttagtgctgtattaactttctgtaaataattttagtctcaataaagattaattctgaataataaaaataaacgaggacaatttttttcacaacttttaattctttatttattttttacacttaattttgggaaacatggacctagtgtcaaagaactcaaactcaacattacaaaaacttactacatattcaaggatcatggtctgaactaatctccacataacatataatcacatttttcaattaaaaagaagaattaataatgtattaaatgatgatcgacaagatcacatgatttgagatttttttttatgatgggaaaaatatgatttgagattaggtaacaatctttccattttagatagacttatgtctccgcaatatatagaagacttcatgttgcatagcgatggtactgcttaaggtcctgcaggtatataggatcctataatgctagcagaattagagctagaaatggaatggctgccacttgaccaataagtgtcatataggactcataacctttataaatgtactgattgatttaattctttgttttttttttttctctccatttgttggaaattttcaaaaatctctttgcatttttttcctcatttcagaggtaaatgtattttgatgtttgtataaaccaatggattactatatgatgttttgattgatggaaaaagggtgaaatatccacttatgtaacatacactttcacttgtgatcatcaatttacttgagagtcgtttcttcaaagtttcaaactttccacaacagaaactctacttaagatgatcaaggcattgtcaaatgaaatgctttggaaggaagaaactgcctctccttcgttaaaccaagctcagtatcttgcagtttcctctcatcagacatatcaatgcttggacaaaggtacgaaccattttctacatttgtgggacattacaggcatttcttagcaacaacgaatgttcatcatactcagaacctaaaagattaacatgttcaagccatccttagaacccaacagctcaacactttcgctacatctattactagaggccaacaattggactatttacaagtttcgcagaactgcactaaacagagtcgagtcactttgcaggcgtattaaacaaaagaagcacagataataaaaacgaagcgttgataaagaagaagttcgacttgtcattagaaagaaagagataacaggaacggaaacatagtagaacacttattcatcagggattatacaaggccccaaaacacaaaccaccaaagttttacatgaaacgaaacattgaacttcttaagcataacagtgacgagttcattagctggcctgaacgccagatatgcactgttgtccataatctttgatctctctggcgcagtccatgattttatggggggcagaatcgagacagtcggcaccaggagggtagcagcctgtaacgtacctgtagatccaagcaaggacagtgattgcagccacaccgaatcccccactggcaagaaagcccagtgacaacaaagccacagtgatcactgcgggaacaagtacgggggagaagataacaagtggtggtgtaacaatggtcagtgcgataatagttccagcgaggatcaaggaggcgagaatcaggagagatcctccggcagtcactgcagtggcggccttcaccacgcaagtgctatagcttgtttgttgttcgtaggggcactgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacatttggataacgatgaggatagcagtgctgttgttggtagtgcaagcatgccatggctgtgttgtgtggttaaaggtcacttctttctttggttttgtgagtgtgttgtggagaatgaagaagggtactacaaatgtttatagaagggtggcatgatattgtggatgaggatatgcacaactcagcaacacttcattggttctggatatggatgtggattctcatgctgatattttgtgctgctaaactgtctctgcttctgccacgtggatgcaaagatcttcttccatagatgttttcgagagggctaatgtaagccacagaaaaagggcccaccaaagtctaagaaatcaaaattaaaaatatcaacttgaaattgcagatagtttgggttgggttcattctacagtgcaaacccgtgacccatcacattctttttcttcttttcttttcaaacaaaaaaacaaacagttggtgttcctgagaatctgaaaccacacacagaatactcggctaccaagaactcatggtggcagctcatctttgctacattacaaatttggtttatgtgggaactaaattctaaaataagttgtaataattagatgagaattagaaaaaacaattaaagcacaccaaataaggttaaaaacatcaccaatactgtaacaaagtttctgtttaaaccttcaatacaaaaaccctttcaaaagttatcgtaaaataaagttgtgatgaaatattttatcaattatttcaatgatattcgctattttaactattaatttagtaaatttaaaatatagtaagaaactgagaatgagtttaagatagacaaattaaaatcaacataattgccatggaaacatcacttaaaacatttactattttttaggagtttgacttccgggttactcacttgtcgagatgtaatattacgaactgtgaaaacgggtcatccggtgtgaccttatccggctcatcacaggttggtcacttagtgagcccatctaacccgactcatttattaacgagtcagaaaaatttgaacccgtcccgatccaccacaggttggtagataaatgggttgactcactagctcacttaattacaatttttttttaaataaatttttttcagaaactttctataattcaaatctaaacaagtttcactcccaacaacatgggtgttttaattaattatgaaagtaaggaatttaaataattttttcaagaaaaaaaataataaatattttttataaaattaaaattaaattttaataaaataaaattggataggtaggccaacccgacccaccacggattcaacccgcataagccaggtttaaatgagtcgggttgaaatctgacccacatagaaaaatacaattttttcaaacccaatccagctcgaatatatggtgaacccatagaatgtgaccattttgacagctctatatattacaatacctgcaataactgacgaaaataatgaggatataacattaataattatttgttcttatatgatatatgtttttatttatccttctaatgctgttttaaaaaacttctaataataactatttggttttaatgcgtaatcactaaaatcagtcaaaaagaatgcagtcgtctgttaatgaacgtaaggagcttctggatgacggctgggacttctttgacaaagagacagagctctaagtgacttcttttcagtagattatcggtgctagaataaaagagatatgaagggattctgggttctgctctatgtagagtaaagcttggatctttgatctaatctaccgttagtataaaacatctattgagctctcttttatttttcttctcagtgtatagtttgcttattgtttttttggtgtttttttccacctctttgtggtactgacataaacaaagactatcgttttgtcatagaattttgagattttgaagaaaagttggataatttagtatactttttaactgactttataaaacgaattagcttcaattagagcttcacaattctcggattttactgaatgacaagttaacaatttaagctagaaaaaaatatgctctaagcctggacatgttgttagtcatggccagcgtatgtgtacattaggtaaatgaaaacaaaaacaaaaactttgaaggtttctgttaagaaaggaactaatttagcaataagagacatgatattacaagctcaacactgtcgctatatctaccagaagccaacaaatcttccagtgcgtaccattggagccacttttcaggctcattagacaaaagtagcacatataatgtaaagacgaaacgatgataaagagactcacaaggacataatttcaatacagaagaagtttgatgtcattactcattagaaagaaaggtataacacaggaacggaaacatagttgaacaattattcatcaggggctttacaaggccccaaaacacaaaccaccaaagttttaaatgaaacgagacatgaacttcttctgattcataacagagataagttttcattatttctctttgaggttgatgaggtcgtggtagtaaagcaacacgcacattggctggccgaggatgcagaagatgaaccagaaaatcatgttgcccaccatggagttggagaacttctcttggaggtagttggtaatgagcacgagtggcacctggaacatgattccgatgaaagcccagagcttgaacacgtggcatggaacagcaatgcacaattcgtggaaggcaccagacacgaggaatgcgataatgatagcgccctcttttggaatgccgttcctcaggcatgggaaataaaggtgcctaaccatccaacggtgcactggcatgttccacatcttccaatactctgccacggtcttagcgttccaccaatccttgtagaactctctgtcgccgaacctaagaagctcagcgaggatattgagccagaggtggaagaatgagtagaacatgcagagccacacgtaaaggtttggcacagagagcttgagcaccctctcaatagcgtagaggaaatcgcccttaagagggtgcttagagttcctcacgatggggttgatgtactgctcgatgatgaagcccatgagtccgatgaacacaatgagcttcacgaactgcctcacaacccatccctttctaatgcaggaggaccttggataagagggctggtagcaaagggttggagccaccataaagtaagcgagggacttaagggacacgtcgtaagagcaagacacgatggtggagtttgggtgagcatcacccttgtatccggacttagcaagggtcctaatatcagaggaggtgtgggcgtaagaaacgagcttgagccacatgatgcagccgaaaagcataagcacaacgccagacatgtacacggagtcgcaggtaagaatcacgatcactgggtaaagcacagcagcggtagaaacgatcacgtgaagaagcacagccacaagctcagcaatgtggttcctctgaacgagcttctcaacgatgaaagcagcaagtgggaaaattgggagggtgaggcagcacatgaaaatggaccagtctccaagggatcttgaggagaaccagaatccggtatcgatgagccagccgtacttcatgaggttctcgataatgagcctggagttcacggcaatgagcaccacaatgcagaggttgaagagtccagcgtgagactgcttgaagatagcatcggaggagagaggagactctctcacccttctatgagctgggaaggatggtctgtaggtgaacctgatatcagtctcgcccacttgcttctcttcgtttccgatcaccacaccaccaccgtttccaatgaggttcacagatccggtagcgttttccatagctcccaccctatcctgatccctgttctcagcaacatgtccagtagctggaacaccgttggtagaggtgaagccagaagaagaaggctcaatcacagagctggagggcttacgccttctgaagttgttaagatcggaatcgccgtggccggacatagtggtagtgttctacaccaaccaaaaatagatccaattagtatttcaaactcatcacattatttgaaatggttgaattaagtattattgatatcaatgacttgttaaccaacagcacttgttatatataaaagataaataagttatatctatagagagaaaaaaggaaacatacttgagatgactcagccacggccattgttccaaagaactttcctttttctttgtgctctgtgctttgaggtgtgggagttgtgaatgggtggggatggtgggtgaggatttatatggtggatgaagtggttgagctcttatctatggctatctaaagctacaatctcattgggggttgagattgtgtttgtacacttggcactctttcaatccactagagggttagattctcttatgttttttcaccaatgtgggacttccacgtatgattcaggattccacaagatattattgctcatttcttattcattttgcaactgacctggaaaatggcttcaactatgtggccatcatttgagtttgtgagatatttttgggtccacatccattttcgcttgtggcaaggtgcatgccaaccacaaattttgaatagcagacagagaaattgtgaaaagaaccaagtgaagggtgatggtaacgcgcgtctattctcaccaaggggcaattacattacacgtcacatgaattcaaactaattaatacgtggttgattcactacttttaatataaataataatagtacttcaatataaataataatagatcattatacggtaataactttttttaataaatagtttgtttgatctaagttatctttagaaaataagagaaatctaaaatataataagttgatgaaatatgagtgttaatggtttgtttggaaagttttattctgcggccataagcactttggaacaactgcacaaagattgtgctccacacataaacacagttgtgcatgttagcaatgaatacttgatagttttaccatgttccattttacagttgatttagctgatattcacagcttatcccaaatctaaaatggcagcttcttattggatggaagaggctttttgtaatatttctttagatagataaactcttgagggaaaaaggcaaaaactaagtgatataatgagatagaaatgcattcaaataatggaatatggtttatctgttaacggatattattgcgacgtgaaggtctaccaaatgcattcaaataatggatctgagacgtatcgacgtctcttgcagggacacgaagtgatccgtttaaactatcagtgtttgacaggatatattggcgggtaaac

SEQ ID NO: 172 - DNA, 10874 bp, synthetic construct
  source 1..10874: mol_type = other DNA
tggcaggatatattgtggtgtaaacaaattgacgcttagacaacttaataacacattgcggacgtttttaatgtactggggttgaacactctgtgccgaattcggatccagcgaacaatcgacaatacataatagctgcattgtcacccgatacttaaataattgtctcatacaaataaaagattgaagatattcaaacaatatttatatgtgaagataaaaaaaaaccaaaaaaatgaaccaaggaccaaatataatatgatatttttttgcttagggacaaaaaaaaacagcatcatcacaagcatcctaaagaaaatagaagagataaaacagagatataaaactaaagtacacaagacttcttgacacaaccataaggttctaaagagaaaaaagaaaataaaatccacataacaacagatagttcaattcccatctcaactagggaaaatagatttttttgtaacaaactcaccaccacgaaccagataagacaagacacacaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcattcctggttcggctttctttggcccggatgatgatatggcgggttcggttgcgacggcgaaaacggtactaaagccatgacgttgcctgggccaaaattgtcttgtaggaagatacacaaaacggcgaaggctggaagtgaaggagctaggttttgacaggaaagaagaagaaaaaagcagtgaatgactggaagccatggctgtcgagtcaacaatcacagataaatcagaatcaaaatccattacgtaatcttaagtacggatctgaagcaatctcgtgaaaaaaaaccctaatccacggcaatagaatcaacaaacaaggcagttgctaacagatctgagcaatatctctcgaaaaacgatatagaatcaacgaataaggctttttgtcaacggatctgaacaatttcttgaaaaaccacagaagtataaaaaataaggctgttgtttaacggatctgaagtaattcatgaaaaagccctgatctatggttacgtttaacggatctgaagtaatcaatgaaaaaagccctaatctacgattaatgaatcaacaattaaggcttgttgtaaagggatctgacatgaaaaagctctaattagagcaaatctacgattaaagaatcatcaaattaaggacgttgttgtaaagataataaaagctgaatctgattatgaaaattgaagatcgatgaaaccaaacaaaacacaatcaaatcaacatcgtattgatcaaagaacatacacaattcatggaaagaacgaagaagaaaaacagaagaaatcgactaaccttgaagggagaggtagggaaatacacagagagaatggaatatattggaattgaactgcgaaggaaggaggaggggtgtggggcgttttatagcataaggtgtggaacccttctccagtcatattgtgacgcgtgttcatgtatggtgtcacttggtgcctatgacacgggtaatatttggtattggatgcattttcggagagtccttctaggaaattgagaattgcaatttgcgtggtagttggcggtttcgtaatcttgacggagtcacatgcaccttcaagcttcgctttatctgttacgacgtcgttgttatattgggcccaccccgttgcaagtggagcgtccgaagttgatacgggccccactatccattgcctgaagtcggctgcttttacattattaaaaatctatggaaaagctgtcatgctagattttttttattatggaccagaaaagaataattgaaaagttttcttattatggagcaaaaaaatctttaataattattttttaaataaaatcataattattccactaaattctttcttatttaatttcaccttgtaagatatattttcagtagatttttcaagtatttttttaacatacattacttgtatacatttagttacagtttatataaaattattcatttaaaaaatatttagatattttttaaaaaataattcaaaatattttttttaatgaactttcctttttcctaatttttctttcttatttcttattagatgtttttactaccaatcgtttagggaagaatttttttctctaatgaggagatgtgttaaatgatgcaacttaaggacagattcaccaataacataagactaaaagatagctcattgacaaaaacaataatgatgttaaagaaaacgaatccatttccattcggataagccacaccatttacgaacaagttgcctactaataatatatggtgtcagttttttttttttttgggctgaattggaggggaaattatccccaaagtttgattaaaaatcacgcgacaaagtaacaaaacttttattccgctaaaaaaattagtcaaagtcaattaaatctggtattagaattacttttccgctaatgatatatccacaataacaatgtattaatattgaaaaaaataaagtttatagagtgtgactttttctttcatttttacgtaaattgaaaaaaaaatcaagcctaatatataaactttcattgatcctcacctaaatcctaaagttcttgagcccaattaaaaaacttactggaggataaaggcccaaacttctttttttttttaggaacagagcatgtaatgggtaatgaggcagcctaagcccataaccatgttagcccgtatatatttttgttaattagatttccggtttcttatttttttttagatttcagtttttgtttctcaaaaaaataaaataacttttagtgctgtattaactttctgtaaataattttagtctcaataaagattaattctgaataataaaaataaacgaggacaatttttttcacaacttttaattctttatttattttttacacttaattttgggaaacatggacctagtgtcaaagaactcaaactcaacattacaaaaacttactacatattcaaggatcatggtctgaactaatctccacataacatataatcacatttttcaattaaaaagaagaattaataatgtattaaatgatgatcgacaagatcacatgatttgagatttttttttatgatgggaaaaatatgatttgagattaggtaacaatctttccattttagatagacttatgtctccgcaatatatagaagacttcatgttgcatagcgatggtactgcttaaggtcctgcaggtatataggatcctataatgctagcagaattagagctagaaatggaatggctgccacttgaccaataagtgtcatataggactcataacctttataaatgtactgattgatttaattctttgttttttttttttctctccatttgttggaaattttcaaaaatctctttgcatttttttcctcatttcagaggtaaatgtattttgatgtttgtataaaccaatggattactatatgatgttttgattgatggaaaaagggtgaaatatccacttatgtaacatacactttcacttgtgatcatcaatttacttgagagtcgtttcttcaaagtttcaaactttccacaacagaaactctacttaagatgatcaaggcattgtcaaatgaaatgctttggaaggaagaaactgcctctccttcgttaaaccaagctcagtatcttgcagtttcctctcatcagacatatcaatgcttggacaaaggtacgaaccattttctacatttgtgggacattacaggcatttcttagcaacaacgaatgttcatcatactcagaacctaaaagattaacatgttcaagccatccttagaacccaacagctcaacactttcgctacatctattactagaggccaacaattggactatttacaagtttcgcagaactgcactaaacagagtcgagtcactttgcaggcgtattaaacaaaagaagcacagataataaaaacgaagcgttgataaagaagaagttcgacttgtcattagaaagaaagagataacaggaacggaaacatagtagaacacttattcatcagggattatacaaggccccaaaacacaaaccaccaaagttttacatgaaacgaaacattgaacttcttaagcataacagtgacgagttcattagctggcctgaacgccagatatgcactgttgtccataatctttgatctctctggcgcagtccatgattttatggggggcagaatcgagacagtcggcaccaggagggtagcagcctgtaacgtacctgtagatccaagcaaggacagtgattgcagccacaccgaatcccccactggcaagaaagcccagtgacaacaaagccacagtgatcactgcgggaacaagtacgggggagaagataacaagtggtggtgtaacaatggtcagtgcgataatagttccagcgaggatcaaggaggcgagaatcaggagagatcctccggcagtcactgcagtggcggccttcaccacgcaagtgctatagcttgtttgttgttcgtaggggcactgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacatttggataacgatgaggatagcagtgctgttgttggtagtgcaagcatgccattgttccaaagaactttcctttttctttgtgctctgtgctttgaggtgtgggagttgtgaatgggtggggatggtgggtgaggatttatatggtggatgaagtggttgagctcttatctatggctatctaaagctacaatctcattgggggttgagattgtgtttgtacacttggcactctttcaatccactagagggttagattctcttatgttttttcaccaatgtgggacttccacgtatgattcaggattccacaagatattattgctcatttcttattcattttgcaactgacctggaaaatggcttcaactatgtggccatcatttgagtttgtgagatatttttgggtccacatccattttcgcttgtggcaaggtgcatgccaaccacaaattttgaatagcagacagagaaattgtgaaaagaaccaagtgaagggtgatggtaacgcgcgtctattctcaccaaggggcaattacattacacgtcacatgaattcaaactaattaatacgtggttgattcactacttttaatataaataataatagtacttcaatataaataataatagatcattatacggtaataactttttttaataaatagtttgtttgatctaagttatctttagaaaataagagaaatctaaaatataataagttgatgaaatatgagtgttaatggtttgtttggaaagttttattctgcggccataagcactttggaacaactgcacaaagattgtgctccacacataaacacagttgtgcatgttagcaatgaatacttgatagttttaccatgttccattttacagttgatttagctgatattcacagcttatcccaaatctaaaatggcagcttcttattggatggaagaggctttttgtaatatttctttagatagataaactcttgagggaaaaaggcaaaaactaagtgatataatgagatagaaatgcattcaaataatggaatatggtttatctgttaacggatattattgcgacgtgaaggtctaccaaatgcattcaaataatggtcgtctgttaatgaacgtaaggagcttctggatgacggctgggacttctttgacaaagagacagagctctaagtgacttcttttcagtagattatcggtgctagaataaaagagatatgaagggattctgggttctgctctatgtagagtaaagcttggatctttgatctaatctaccgttagtataaaacatctattgagctctcttttatttttcttctcagtgtatagtttgcttattgtttttttggtgtttttttccacctctttgtggtactgacataaacaaagactatcgttttgtcatagaattttgagattttgaagaaaagttggataatttagtatactttttaactgactttataaaacgaattagcttcaattagagcttcacaattctcggattttactgaatgacaagttaacaatttaagctagaaaaaaatatgctctaagcctggacatgttgttagtcatggccagcgtatgtgtacattaggtaaatgaaaacaaaaacaaaaactttgaaggtttctgttaagaaaggaactaatttagcaataagagacatgatattacaagctcaacactgtcgctatatctaccagaagccaacaaatcttccagtgcgtaccattggagccacttttcaggctcattagacaaaagtagcacatataatgtaaagacgaaacgatgataaagagactcacaaggacataatttcaatacagaagaagtttgatgtcattactcattagaaagaaaggtataacacaggaacggaaacatagttgaacaattattcatcaggggctttacaaggccccaaaacacaaaccaccaaagttttaaatgaaacgagacatgaacttcttctgattcataacagagataagttttcattatttctctttgaggttgatgaggtcgtggtagtaaagcaacacgcacattggctggccgaggatgcagaagatgaaccagaaaatcatgttgcccaccatggagttggagaacttctcttggaggtagttggtaatgagcacgagtggcacctggaacatgattccgatgaaagcccagagcttgaacacgtggcatggaacagcaatgcacaattcgtggaaggcaccagacacgaggaatgcgataatgatagcgccctcttttggaatgccgttcctcaggcatgggaaataaaggtgcctaaccatccaacggtgcactggcatgttccacatcttccaatactctgccacggtcttagcgttccaccaatccttgtagaactctctgtcgccgaacctaagaagctcagcgaggatattgagccagaggtggaagaatgagtagaacatgcagagccacacgtaaaggtttggcacagagagcttgagcaccctctcaatagcgtagaggaaatcgcccttaagagggtgcttagagttcctcacgatggggttgatgtactgctcgatgatgaagcccatgagtccgatgaacacaatgagcttcacgaactgcctcacaacccatccctttctaatgcaggaggaccttggataagagggctggtagcaaagggttggagccaccataaagtaagcgagggacttaagggacacgtcgtaagagcaagacacgatggtggagtttgggtgagcatcacccttgtatccggacttagcaagggtcctaatatcagaggaggtgtgggcgtaagaaacgagcttgagccacatgatgcagccgaaaagcataagcacaacgccagacatgtacacggagtcgcaggtaagaatcacgatcactgggtaaagcacagcagcggtagaaacgatcacgtgaagaagcacagccacaagctcagcaatgtggttcctctgaacgagcttctcaacgatgaaagcagcaagtgggaaaattgggagggtgaggcagcacatgaaaatggaccagtctccaagggatcttgaggagaaccagaatccggtatcgatgagccagccgtacttcatgaggttctcgataatgagcctggagttcacggcaatgagcaccacaatgcagaggttgaagagtccagcgtgagactgcttgaagatagcatcggaggagagaggagactctctcacccttctatgagctgggaaggatggtctgtaggtgaacctgatatcagtctcgcccacttgcttctcttcgtttccgatcaccacaccaccaccgtttccaatgaggttcacagatccggtagcgttttccatagctcccaccctatcctgatccctgttctcagcaacatgtccagtagctggaacaccgttggtagaggtgaagccagaagaagaaggctcaatcacagagctggagggcttacgccttctgaagttgttaagatcggaatcgccgtggccggacatagtggtagtgttctacaccaaccaaaaatagatccaattagtatttcaaactcatcacattatttgaaatggttgaattaagtattattgatatcaatgacttgttaaccaacagcacttgttatatataaaagataaataagttatatctatagagagaaaaaaggaaacatacttgagatgactcagccacggccatggctgtgttgtgtggttaaaggtcacttctttctttggttttgtgagtgtgttgtggagaatgaagaagggtactacaaatgtttatagaagggtggcatgatattgtggatgaggatatgcacaactcagcaacacttcattggttctggatatggatgtggattctcatgctgatattttgtgctgctaaactgtctctgcttctgccacgtggatgcaaagatcttcttccatagatgttttcgagagggctaatgtaagccacagaaaaagggcccaccaaagtctaagaaatcaaaattaaaaatatcaacttgaaattgcagatagtttgggttgggttcattctacagtgcaaacccgtgacccatcacattctttttcttcttttcttttcaaacaaaaaaacaaacagttggtgttcctgagaatctgaaaccacacacagaatactcggctaccaagaactcatggtggcagctcatctttgctacattacaaatttggtttatgtgggaactaaattctaaaataagttgtaataattagatgagaattagaaaaaacaattaaagcacaccaaataaggttaaaaacatcaccaatactgtaacaaagtttctgtttaaaccttcaatacaaaaaccctttcaaaagttatcgtaaaataaagttgtgatgaaatattttatcaattatttcaatgatattcgctattttaactattaatttagtaaatttaaaatatagtaagaaactgagaatgagtttaagatagacaaattaaaatcaacataattgccatggaaacatcacttaaaacatttactattttttaggagtttgacttccgggttactcacttgtcgagatgtaatattacgaactgtgaaaacgggtcatccggtgtgaccttatccggctcatcacaggttggtcacttagtgagcccatctaacccgactcatttattaacgagtcagaaaaatttgaacccgtcccgatccaccacaggttggtagataaatgggttgactcactagctcacttaattacaatttttttttaaataaatttttttcagaaactttctataattcaaatctaaacaagtttcactcccaacaacatgggtgttttaattaattatgaaagtaaggaatttaaataattttttcaagaaaaaaaataataaatattttttataaaattaaaattaaattttaataaaataaaattggataggtaggccaacccgacccaccacggattcaacccgcataagccaggtttaaatgagtcgggttgaaatctgacccacatagaaaaatacaattttttcaaacccaatccagctcgaatatatggtgaacccatagaatgtgaccattttgacagctctatatattacaatacctgcaataactgacgaaaataatgaggatataacattaataattatttgttcttatatgatatatgtttttatttatccttctaatgctgttttaaaaaacttctaataataactatttggttttaatgcgtaatcactaaaatcagtcaaaaagaatgcagatctgagacgtatcgacgtctcttgcagggacacgaagtgatccgtttaaactatcagtgtttgacaggatatattggcgggtaaac

SEQ ID NO: 173 - protein, 226 aa, Glycine max
MTTQVPPHSVQVHTTTTHRYEAGVVPPGARFETSYEAGVKAASIYHSERGPTTSQVLAVLAGLPVGGILLLLAGLTLAGTLTGLAVATPLFVLFSPVLVPATVAIGLAVAGFLTSGAFGLTALSSFSWILNYIRETQPASENLAAAAKHHLAEAAEYVGQKTKEVGQKTKEVGQDIQSKAQDTREAAARDAREAAARDAREAAARDAKVEARDVKRTTVTATTATA

SEQ ID NO: 174 - protein, 223 aa, Glycine max
MTTVPPHSVQVHTTTHRYEAGVVPPARFEAPRYEAGIKAPSSIYHSERGPTTSQVLAVVAGLPVGGILLLLAGLTLAGTLTGLVVATPLFIIFSPVLIPATVAIGLAVAGFLTSGVFGLTALSSFSWILNYIRETQPASENLAAAAKHHLAEAAEYVGQKTKEVGQKTKEVGQDIQSKAQDTREAAARDARDAREAAARDARDAKVEARDVKRTTVTATTATA

SEQ ID NO: 175 - protein, 165 aa, Glycine sp.
MATISDQPRGSYSYGTSYGAPYGTTYETNTSINNPPSRQTVKFITAATIGITLLLLSGLTLTGTVIGLIIATPLLVIFSPILVPAAFVLFLVASGFLFSGGCGVAAIAALSWIYNYVSGNQPAGYDTLDYAKGYLADKARDVKERAKDYGSYAQGRINEATQGAY

SEQ ID NO: 176 - protein, 166 aa, Glycine sp.
MATISTDQPRGSYSYGTSYGAPYGTTYETNSSINNPPSRQTVKFITAATIGITLLLLSGLTLTGTVIGLIIATPLLVIFSPILVPAAFVLFLVASGFLFSGGCGVAAIAALSWIYNYVSGNQPAGSDTLDYAKGYLTDKARDVKERAKDYGSYAQGRINEATQGTY

SEQ ID NO: 177 - DNA, 1058 bp, Glycine max
  source 1..1058: mol_type = other DNA
aacacacaccccactaacaattccttcacttgcagcactgttgcatcatcatcttcattgcaaaaccctaaacttcaccttcaaccatgaccacacaagtaccaccacacagtgtccaagtccacacaacaacacaccgctacgaagctggcgtcgtcccccccggagcccgtttcgaaccgccgcgttatgaagccggcgtcaaagcgccctccatttaccattccgagagaggtccaacgacttcccaggtcctcgccgtcctcgccggcctcccggtcggtggcatcctgctcctcctggcgggattgaccctggcaggaaccctcaccggactggcagttgccacgccgctcttcgtcctcttcagcccggtgctggttccagccacggtcgccatcggcctggccgtggccgggttcctgacgtcgggggccttcgggctcacggcgctgtcttccttctcctggatcctgaactacatccgggagacccagccagcctcggagaacctcgccgccgccgcgaagcatcacctcgccgaggcggcggagtacgtggggcagaagacgaaggaagtggggcagaagaccaaggaggttgggcaggatattcagagcaaggcacaggatacaagggaggcagctgcaagggatgcaagggaggcagctgcaagggatgcaagggaagctgctgcaagggatgcaaaggtggaggcaagggatgtaaagagaacaacagtgacagcaacaaccgcaaccgcatgatgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacttccattgttgaattcaatgtcaaatgtgttttgcgttaaaaaaaaaaaaaaaaaa

SEQ ID NO: 178 - DNA, 1041 bp, Glycine max
  source 1..1041: mol_type = other DNA
aacacacacatacccccaactaacaataattccttcacttgcagaacttagttctctgttgcatcatcatcatcttcattagtgttagccctaaacttcaccttcaaccatgaccacagtgccaccacacagtgtccaagtgcacacaacaacacaccgctacgaagccggcgtcgtcccgccggctcgttttgaggcgccgcgttacgaagccggcatcaaggcgccctcttccatttaccattccgagagaggtccgacgacctctcaggttctcgcagttgtcgccggccttccggtcggcggcatcctcctgctcctggcaggactgaccctggctggaactctcaccgggctggtggtggcaacaccgctcttcatcatcttcagcccggtgctgattccggccacggtcgcaattggcctggccgtggccgggttcctgacgtcgggagtctttgggctcacggcgctgtcgtccttctcctggatcctgaactacatccgggagacccagccggcgtcggagaatctggccgctgcggcgaagcaccatctggcggaggcggcggagtacgtggggcagaagaccaaagaagtagggcagaaaaccaaggaagttgggcaggatattcagagcaaggcacaagatacaagggaagcagctgcaagagatgcaagagatgcaagggaagcagctgcaagagatgcaagggatgcaaaggtggaggcgagagatgtaaagagaacaacagtgacagcaacaaccgcaaccgcatgaacgtgatgagtattaatgtgttgttatgaacttatgatgttggtttatgtggggaaataaatgatgtatgtacctcttcttgcctatgtagtaggtttgggtgttttgttgtctagctttgcttatttagtaattagtagaagggacgttcgttcgtgtctcataaaaaggggtactaccactctggcaatgtgatttgtatttgatgaattcaatgtgaaatgtgttttgcgttggtgtatgtaaaaaaaaaaaaaaaa

SEQ ID NO: 179 - DNA, 837 bp, Glycine sp.
  source 1..837: mol_type = other DNA
ctctcacactcgttactctcatcacattattctatatcaattttggttaagagtcttgaaaaagcaagagaaaaaaaaataccaaccatggcaaccatttctgatcaacctagagggtcctactcctatggaacctcatatggagcaccctatggaaccacgtatgagaccaacaccagcattaacaacccaccttcacgccaaacagtgaagttcataactgctgccactattggcatcacactcttgctcctgtctgggttgaccctcacaggcactgtcataggtttgatcattgcaacccctcttcttgttatcttcagccccatccttgtccctgctgcgtttgtgctgttcctagttgcttctggctttttgttctctgggggctgtggtgtggctgccattgctgctttgtcttggatttacaactatgtgtctgggaaccagcctgcgggttatgacacccttgactatgcaaaagggtaccttgccgataaggcaagggatgtaaaggagagggcaaaggattatggaagttatgctcaaggtagaattaatgaggccacacaaggagcttattagtcatgcctgcttatatatgcatgagtgtatgaggttgaattgaaggtggtgatggtggtggtacccttgtggatttgcatgttcaagttctgcagtttattttgaatcttgtttgcattgttctatgtagttctggtttttgtaatctcgtttgtttttgaagctaaatcgttcgccttatttgtgtaaccatcatgttaataaatctcttttgtagctagatcaatgattccttccctcttgttgttaaaa

SEQ ID NO: 180 - DNA, 501 bp, Glycine sp.
  source 1..501: mol_type = other DNA
atggcaaccatttctactgatcaaccaagaggatcctactcctatggaacctcctatggagcaccctatggaaccacctatgagaccaacagcagcattaacaaccctccttcacgccaaaccgtgaagttcataactgctgcaactattggcatcacactcttactcctgtctgggttgaccctcacaggcactgtcataggtttgatcattgcaacccctcttcttgttatcttcagccccatccttgtccctgctgcgtttgtgctgttcctagttgcttctggctttttgttctctgggggctgtggtgtggctgccattgctgctttatcttggatttacaactacgtttctgggaaccagcctgcgggttctgacacccttgactatgcaaaagggtaccttactgataaggcaagggatgtgaaggagagggcaaaggattatggaagttatgctcagggtagaattaatgaggccacacaaggaacttattag